(19) United States
(12) Patent Application Publication
(10) Pub. No.: US 2023/0149487 A1
(43) Pub. Date: May 18, 2023

(54) DELIVERY OF SIALIDASE TO CANCER CELLS, IMMUNE CELLS AND THE TUMOR MICROENVIRONMENT

(71) Applicant: Ansun Biopharma, Inc., San Diego, CA (US)

(72) Inventor: Nancy CHANG, Houston, TX (US)

(21) Appl. No.: 17/794,197

(22) PCT Filed: Jan. 20, 2021
(86) PCT No.: PCT/US2021/014225

§ 371 Date: Jul. 20, 2022

Related U.S. Application Data
(60) Provisional application No. 63/132,420, filed on Dec. 30, 2020.
(60) Provisional application No. 62/964,082, filed on Jan. 21, 2020.

(51) Int. Cl. A61K 35/768; A61K 35/761; C12N 15/86; C12N 9/24; A61P 35/00

(52) U.S. Cl. CPC A61K 35/768; A61K 35/761; C12N 15/86; C12N 9/2402; A61P 35/00; C12Y 302/01018; C12N 2710/10332; C12N 2710/10343; C12N 2710/24171

(57) ABSTRACT

The present application provides methods and compositions for treating cancers (such as solid tumors) using a recombinant oncolytic virus encoding a sialidase. In some embodiments, the oncolytic virus further encodes one or more other heterologous proteins. In some embodiments, the recombinant oncolytic virus is delivered via an engineered immune cell. In some embodiments, the present application provides methods and compositions for treating cancers using a recombinant oncolytic vims encoding a sialidase or another heterologous protein and an engineered immune cell (e.g., a CAR-T, CAR-NK, or CAR-NKT cell) expressing a chimeric receptor capable of binding to the sialidase or other heterologous protein.

### CROSS REFERENCE TO RELATED APPLICATIONS

This application claims priority benefit of U.S. Provisional Application 62/964,082 filed Jan. 21, 2020 and U.S. Provisional Application 63/132,420 filed Dec. 30, 2020, the contents of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 208712000640SEQLIST.TXT, date recorded: Jan. 19, 2021, size: 253 KB).

### FIELD

The present application relates to methods and compositions for treating cancer with an oncolytic virus (e.g., vaccinia virus) encoding a sialidase.

### BACKGROUND

Cancer is the second leading cause of death in the United States. In recent years, great progress has been made in cancer immunotherapy, including immune checkpoint inhibitors, T cells with chimeric antigen receptors, and oncolytic viruses.

Oncolytic viruses are naturally occurring or genetically modified viruses that infect, replicate in, and eventually kill cancer cells while leaving healthy cells unharmed. A recently completed Phase III clinical trial of the oncolytic herpes simplex virus T-VEC in 436 patients with unresectable stage IIIB, IIIC or IV melanoma was reported to meet its primary end point, with a durable response rate of 16.3% in patients receiving T-VEC compared to 2.1% in patients receiving GM-CSF. Based on the results from this trial, FDA approved T-VEC in 2015.

Oncolytic virus constructs from at least eight different species have been tested in various phases of clinical trials, including adenovirus, herpes simplex virus-1, Newcastle disease virus, reovirus, measles virus, coxsackievirus, Seneca Valley virus, and vaccinia virus. It has become clear that oncolytic viruses are well tolerated in patients with cancer. The clinical benefits of oncolytic viruses as stand-alone treatments, however, remain limited. Due to concerns on the safety of oncolytic viruses, only highly attenuated oncolytic viruses (either naturally avirulent or attenuated through genetic engineering) have been used in both preclinical and clinical studies. Since the safety of oncolytic viruses has now been well established it is time to design and test oncolytic viruses with maximal anti-tumor potency. Oncolytic viruses with a robust oncolytic effect will release abundant tumor antigens to prime or activate immune cells including T and NK cells, resulting in a strong immunotherapeutic effect.

### BRIEF SUMMARY

The present application provides methods and compositions for delivery of an oncolytic virus expressing a heterologous protein or nucleic acid to cancer cells.

One aspect of the present application provides a recombinant oncolytic virus comprising a nucleotide sequence encoding one or more human or bacterial sialidases or a protein containing a sialidase catalytic domain thereof. The oncolytic viruses can be derived from a poxvirus, an adenovirus, a herpes virus or any other suitable oncolytic virus. Suitable recombinant oncolytic viruses can be created by inserting an expression cassette that includes a sequence encoding a sialidase or a portion thereof with sialidase activity into an oncolytic virus. In some embodiments, the nucleotide sequence encoding the sialidase is operably linked to a promoter.

Many cancer cells are hypersialylated. The recombinant oncolytic viruses described herein are capable of delivering sialidase to tumor cells and the tumor microenvironment. The delivered sialidase can reduce sialic acid present on tumor cells and render the tumor cells more vulnerable to killing by immune cells, immune cell-based therapies and other therapeutic agents whose effectiveness is diminished by hypersialylation of cancer cells. For instance, a group of receptors called Siglect (Sialic acid-binding immunoglobulin like lectins) on immune cells will bind its inhibitory receptor ligands, which are sialylated glycoconjugates on tumor cells. In some embodiments, the removal of sialic acid prevents binding of such ligands to Siglect on immune cells and thus abolishes the suppression of immunity against tumor cells.

Also provided are methods for delivering a sialidase to the tumor microenvironment. Within the tumor microenvironment the sialidase can remove terminal sialic acid residues on cancer cells, thereby reducing the barrier for entry of immune cells or immunotherapy reagents and promote cellular immunity against cancer cells.

In some embodiments, the oncolytic virus is a virus selected from the group consisting of: vaccinia virus, reovirus, Seneca Valley virus (SVV), vesicular stomatitis virus (VSV), Newcastle disease virus (NDV), herpes simplex virus (HSV), morbillivirus virus, retrovirus, influenza virus, Sinbis virus, poxvirus, measles virus, cytomegalovirus (CMV), lentivirus, adenovirus, and derivatives thereof. In some embodiments, the virus is Talimogene Laherparepvec. In some embodiments, the virus is a reovirus. In some embodiments, the virus is an adenovirus having an E1ACR2 deletion.

In some embodiments according to any one of the recombinant oncolytic viruses described above, the oncolytic virus is a poxvirus. In some embodiments, the poxvirus is a vaccinia virus. In some embodiments, the vaccinia virus is of a strain selected from the group consisting of Dryvax, Lister, M63, LIVP, Tian Tan, Modified Vaccinia Ankara, New York City Board of Health (NYCBOH), Dairen, Ikeda, LC16M8, Tashkent, IHD-J, Brighton, Dairen I, Connaught, Elstree, Wyeth, Copenhagen, Western Reserve, Elstree, CL, Lederle-Chorioallantoic, AS, and derivatives thereof. In some embodiments, the virus is vaccinia virus Western Reserve.

In some embodiments according to any one of the recombinant oncolytic viruses described above, the recombinant oncolytic virus comprises one or more mutations that reduce immunogenicity of the virus compared to a corresponding wild-type strain. In some embodiments, the virus is a vaccinia virus, and the one or more mutations are in one or more proteins selected from the group consisting of A14, A17, A13, L1, H3, D8, A33, B5, A56, F13, A28, and A27. In some embodiments, the one or more mutations are in one or more proteins selected from the group consisting of A27L, H3L, D8L and L1R.

In some embodiments, the virus is a vaccinia virus, and the virus comprises one or more proteins selected from the group consisting of: (a) a variant vaccinia virus (VV) H3L protein that comprises an amino acid sequence having at least 90% amino acid sequence identity to any one of SEQ ID NOS: 06-69; (b) a variant vaccinia virus (VV) D8L protein that comprises an amino acid sequence having at least 90% amino acid sequence identity to any one of SEQ ID NOS: 70-72 or 85; (c) a variant vaccinia virus (VV) A27L protein that comprises an amino acid sequence having at least 90% amino acid sequence identity to SEQ ID NO: 73; and (d) a variant vaccinia virus (VV) L1R protein that comprises an amino acid sequence having at least 90% amino acid sequence identity to SEQ ID NO: 74.

In some embodiments according to any one of the recombinant oncolytic viruses described above, the sialidase is a Neu5Ac alpha(2,6)-Gal sialidase, a Neu5Ac alpha(2,3)-Gal sialidase, or a Neu5Ac alpha(2,8)-Gal sialidase.

In some embodiments according to any one of the recombinant oncolytic viruses described above, the sialidase is any protein having exo-sialidase activity (Enzyme Commission EC 3.2.1.18) including bacterial, human, fungal, viral sialidase and derivatives thereof. In some embodiments, the bacterial sialidase is selected from the group consisting of: Clostridium perfringens sialidase, Actinomyces viscosus sialidase, and Arthrobacter ureafaciens sialidase, Salmonella typhimurium sialidase and Vibrio cholera sialidase.

In some embodiments according to any one of the recombinant oncolytic viruses described above, the sialidase is a human sialidase or a derivative thereof. In some embodiments, the sialidase is NEU1, NEU2, NEU3, or NEU4.

In some embodiments according to any one of the recombinant oncolytic viruses described above, the sialidase is a naturally occurring sialidase.

In some embodiments according to any one of the recombinant oncolytic viruses described above, the sialidase comprises an anchoring domain. In some embodiments, the sialidase is a fusion protein comprising a sialidase catalytic domain fused to an anchoring domain. In some embodiments, the anchoring domain is positively charged at physiologic pH. In some embodiments, the anchoring domain is a glycosaminoglycan (GAG)-binding domain.

In some embodiments according to any one of the recombinant oncolytic viruses described above, the sialidase is a protein having exo-sialidase activity as defined by Enzyme Commission EC 3.2.1.18.

In some embodiments according to any one of the recombinant oncolytic viruses described above, the sialidase is an anhydrosialidase as defined by Enzyme Commission EC 4.2.2.15.

In some embodiments according to any one of the recombinant oncolytic viruses described above, the sialidase comprises an amino acid sequence having at least about 80% sequence identity to an amino acid sequence selected from the group consisting of SEQ ID NOs: 1-33 or 53-54. In some embodiments, the sialidase comprises an amino acid sequence having at least about 80% sequence identity to the amino acid sequence of SEQ ID NO: 2. In some embodiments, the sialidase is DAS181.

In some embodiments according to any one of the recombinant oncolytic viruses described above, the nucleotide sequence encoding the sialidase further encodes a secretion sequence operably linked to the sialidase. In some embodiments, the secretion sequence comprises the amino acid sequence of SEQ ID NO: 40.

In some embodiments according to any one of the recombinant oncolytic viruses described above, the sialidase comprises a transmembrane domain. In some embodiments, the sialidase comprises from the N-terminus to the C-terminus: a sialidase catalytic domain, a hinge region, and a transmembrane domain.

In some embodiments according to any one of the recombinant oncolytic viruses described above, the sialidase comprises an anchoring domain or a transmembrane domain located at the carboxy terminus of the sialidase.

In some embodiments according to any one of the recombinant oncolytic viruses described above, the promotor is a viral promoter that can be an early promoter, an intermediate promoter, or a late promoter

or an early/late hybrid promoter. In some embodiments, the oncolytic virus is a poxvirus and the promoter is a poxvirus early promoter, a late promoter or a hybrid early/late promoter.

In some embodiments according to any one of the recombinant oncolytic viruses described above, the promotor is a viral late promoter. In some embodiments, the promoter is an F17R late promoter (SEQ ID NO: 61).

In some embodiments according to any one of the recombinant oncolytic viruses described above, the promoter is a hybrid early-late promoter.

In some embodiments according to any one of the recombinant oncolytic viruses described above, the promoter comprises a partial or complete nucleotide sequence of a human promoter. In some embodiments, the human promoter is a tissue or tumor-specific promoter.

In some embodiments according to any one of the recombinant oncolytic viruses described above, the oncolytic virus further comprises a second nucleotide sequence encoding a heterologous protein or nucleic acid. In some embodiments, the second nucleotide sequence encodes a heterologous protein.

In some embodiments according to any one of the recombinant oncolytic viruses described above, the heterologous protein is an immune checkpoint inhibitor. In some embodiments, the immune checkpoint inhibitor is an inhibitor of CTLA-4, PD-1, PD-L1, TIGIT, LAG3, TIM-3, VISTA, B7-H4, or HLA-G. In some embodiments, the immune checkpoint inhibitor is an antibody.

In some embodiments according to any one of the recombinant oncolytic viruses described above, the heterologous protein is an inhibitor of an immune suppressive receptor. In some embodiments, the immune suppressive receptor is LILRB, TYRO3, AXL, or MERTK. In some embodiments, the inhibitor of an immune suppressive receptor is an anti-LILRB antibody.

In some embodiments according to any one of the recombinant oncolytic viruses described above, the heterologous protein is a multi-specific immune cell engager. In some embodiments, the heterologous protein is a bispecific T cell engager (BiTE).

In some embodiments according to any one of the recombinant oncolytic viruses described above, the heterologous protein is selected from the group consisting of cytokines, costimulatory molecules, tumor antigen presenting proteins, anti-angiogenic factors, tumor-associated antigens, foreign antigens, and matrix metalloproteases (MMP).

In some embodiments according to any one of the recombinant oncolytic viruses described above, the heterologous protein is an inhibitor of CD55 or CD59.

In some embodiments according to any one of the recombinant oncolytic viruses described above, the heterologous protein is IL-15, IL-12, IL2, modified IL-2 with reduced toxicity or better function, IL18, modified IL-18 with less or no binding to the IL-18 binding protein, Flt3L, CCL5, CXCL10, or CCL4 and any modified formed of such cytokines that still have the anti-tumor immunity, or an inhibitor of any binding proteins that can block and neutralize these cytokine function and activities.

In some embodiments according to any one of the recombinant oncolytic viruses described above, the heterologous protein is a bacterial polypeptide.

In some embodiments according to any one of the recombinant oncolytic viruses described above, the heterologous protein is a tumor-associated antigen selected from the group consisting of carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1, WT1, NY-ESO-1, Fibulin-3, CDH17, and other tumor antigens with clinical significance

In some embodiments according to any one of the recombinant oncolytic viruses described above, the virus comprises two or more additional nucleotide sequences, wherein each nucleotide sequence encodes a heterologous protein.

One aspect of the present application provides a pharmaceutical composition comprising the recombinant oncolytic virus of any one of the preceding claims and a pharmaceutically acceptable carrier.

One aspect of the present application provides a carrier cell comprising any one of the recombinant oncolytic viruses described above. In some embodiments, the carrier cell is an engineered immune cell or a stem cell (e.g., a mesenchymal stem cell). In some embodiments, the engineered immune cell is a Chimeric Antigen Receptor (CAR)-T, CAR-NK, or CAR-NKT cell.

One aspect of the present application provides a method of treating a cancer in an individual in need thereof, comprising administering to the individual an effective amount of any one of the recombinant oncolytic viruses, pharmaceutical compositions, or carrier cells described above.

In some embodiments, the method comprises administering to the individual an effective amount of any one of the recombinant oncolytic viruses described above. In some embodiments, the recombinant oncolytic virus is administered via a carrier cell (e.g., an immune cell or a stem cell, such as a mesenchymal stem cell).

In some embodiments, the recombinant oncolytic virus is administered as a naked virus. In some embodiments, the recombinant oncolytic virus is administered via direct intratumoral injection. In some embodiments, the method further comprises administering to the individual an effective amount of an immunotherapeutic agent. In some embodiments, the immunotherapeutic agent is selected from the group consisting of a mono or multi-specific antibody, a cell therapy, a cancer vaccine (e.g., a dendritic cell-based cancer vaccine), a cytokine, PI3Kgamma inhibitor, a TLR9 ligand, an HDAC inhibitor, a LILRB2 inhibitor, a MARCO inhibitor, and an immune checkpoint inhibitor.

In some embodiments according to any one of the methods described above, the immunotherapeutic agent is a cell therapy. In some embodiments, the cell therapy comprises administering to the individual an effective amount of engineered immune cells expressing a chimeric receptor.

One aspect of the present application provides a method of treating a cancer in an individual in need thereof, comprising administering to the individual an effective amount of engineered immune cells comprising any one of the recombinant oncolytic viruses described above and expressing a chimeric receptor.

One aspect of the present application provides a method of treating a tumor in an individual in need thereof comprising administering to the individual: (a) an effective amount of a recombinant oncolytic virus comprising a nucleotide sequence encoding a foreign antigen, and (b) an effective amount of an engineered immune cell expressing a chimeric receptor specifically recognizing said foreign antigen.

One aspect of the present application provides a method of sensitizing a tumor to an immunotherapy, comprising administering to the individual an effective amount of any one of the recombinant oncolytic viruses, pharmaceutical compositions, or engineered immune cells described above.

One aspect of the present application provides a method of reducing sialylation of cancer cells in an individual, comprising administering to the individual an effective amount of any one of the recombinant oncolytic viruses, pharmaceutical compositions, or engineered immune cells described above.

In some embodiments according to any one of the methods described above, the chimeric receptor is a Chimeric Antigen Receptor (CAR). In some embodiments, the engineered immune cells expressing the CAR are T cells, Natural Killer (NK) cells, or NKT cells.

In some embodiments according to any one of the methods described above, the engineered immune cells express a chimeric receptor, wherein the chimeric receptor specifically recognizes one or more tumor antigens selected from the group consisting of carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1, WT1, NY-ESO-1, Fibulin-3, CDH17, and other tumor antigens with clinical significance

In some embodiments according to any one of the methods described above, the engineered immune cells express a chimeric receptor, wherein the chimeric receptor specifically recognizes the sialidase. In some embodiments, the sialidase is DAS181 or a derivative thereof, and the chimeric receptor comprises an anti-DAS181 antibody that is not cross-reactive with human native neuraminidase.

In some embodiments according to any one of the methods described above, the engineered immune cells and the recombinant oncolytic virus are administered simultaneously.

In some embodiments according to any one of the methods described above, the recombinant oncolytic virus is administered prior to administration of the engineered immune cells.

Also provided are compositions, kits and articles of manufacture for use in any one the methods described above.

### BRIEF DESCRIPTION OF THE DRAWINGS

FIG. 1: Detection of 2,6 sialic acid (by FITC-SNA) on A549 and MCF cells by fluorescence microscopy. A549 and MCF cells were fixed and incubated with FITC-SNA for one hour at 37° C. before imaged under fluorescence microscope to show the FITC-SNA labeled cells (left) and overlay with brightfield cells (right)

FIG. 2: Effective removal of 2,6 sialic acid, 2,3 sialic acid, and exposure of galactose on A549 cells by DAS181 treatment. A549 were treated with DAS181 for two hours at 37° C. and incubated with staining reagents one hour before imaged under fluorescence microscope to show effective removal of sialic acids on tumor cells.

FIG. 3: Effective removal of 2,6 sialic acid on A549 cells by DAS181 but not DAS185 treatment. A549 were treated with DAS181 for 30 minutes or two hours at 37° C. and incubated with FITC-SNA for one hour before examined using flow cytometry to show effective removal of 2,6 sialic acids on tumor cells.

FIG. 4: Effective removal of 2,3 sialic acid on A549 cells by DAS181 but not DAS185 treatment. A549 were treated with DAS181 for 30 minutes or two hours at 37° C. and incubated with FITC-MALII for one hour before examined using flow cytometry to show effective removal of 2,3 sialic acids on tumor cells

FIG. 5: Effective exposure of galactose on A549 cells by DAS181 but not DAS185 treatment. A549 were treated with DAS181 for 30 minutes or two hours at 37° C. and incubated with FITC-PNA for one hour before examined using flow cytometry to show effective exposure of galactose on tumor cells

FIG. 6: DAS181 treatment and PBMC stimulation regimen do not affect A549-red cell proliferation. A549-Red cells were seeded at 2k/well overnight, followed by replacement of medium containing reagents listed on the left. Scan by IncuCyte was initiated immediately after the reagents were added (0 hr) and scheduled for every 3 hr. A549-red cell proliferation is monitored by analyzing the nuclear (red) counts. Kinetic readouts reveal no effect on A549 cell proliferation by vehicle. DAS181, or various stimulation reagents, without the presence of PBMCs.

FIG. 7: Detection of cytotoxicity in A549-red cells following co-culturing with PBMCs from Donor 1 with or without DAS181 treatment. These results showed that DAS181 treatment significantly boost anti-tumor cytotoxicity by PBMCs from Donor 1. A549-Red cells were seeded at 2k/well overnight, followed by co-culturing with 100K/well Donor-1 PBMCs (E:T=50:1) in the presence of medium (no activation), CD3+CD28+IL-2 (T cell activation), or CD3+CD29+IL-2+IL-15+IL-21 (T and NK cell activation). Representative images were taken by IncuCyte at 0 hr and 72 hrs post adding PBMCs.

FIG. 8: Detection of cytotoxicity in A549-red cells following co-culturing with PBMCs from Donor 2 with or without DAS181 treatment. These results showed that DAS181 treatment significantly boost anti-tumor cytotoxicity by PBMCs from Donor 2. A549-Red cells were seeded at 2k/well overnight, followed by co-culturing with 100k/well Donor-1 PBMCs (E:T=50:1) in the presence of medium (no activation), CD3+CD28+IL-2 (T cell activation), or CD3+CD29+IL-2+IL-15+IL-21 (T and NK cell activation). Representative images were taken by IncuCyte at 0 hr and 72 hrs post adding PBMCs.

FIGS. 9A-9C: Detection of cytotoxicity in A549-red cells following co-culturing with PBMCs from Donor 1 with or without DAS181 treatment. These results showed that DAS181 treatment significantly boost anti-tumor cytotoxicity by PBMCs from Donor 1. A549-red tumor cells were seeded at 2k cells/well in 96-well plate. After overnight incubation, PBMCs from Donor 1 mixed with (A) medium (B) CD3/CD28/IL-2, or (C) CD3/CD28/IL-2/IL-15/IL-21 were added into each well as indicated E:T ratio. At mean time, DAS 181 (100 nM) was added. Plates were scanned by IncuCyte every 3 hr for total 72 hrs. Proliferation is monitored by analyzing RFP cell counts.

FIGS. 10A-10C: Detection of cytotoxicity in A549-red cells following co-culturing with PBMCs from Donor 2 with or without DAS181 treatment. These results showed that DAS181 treatment significantly boost anti-tumor cytotoxicity by PBMCs from Donor 2. A549-red tumor cells were seeded at 2k cells/well in 96-well plate. After overnight incubation, PBMCs from Donor 2 mixed with (A) medium, (B) CD3/CD28/IL-2, or (C) CD3/CD28/IL-2/IL-15/IL-21 were added into each well as indicated E:T ratio. At mean time, DAS 181 (100 nM) was added. Plates were scanned by IncuCyte every 3 hr for total 72 hrs. Proliferation is monitored by analyzing RFP cell counts.

FIG. 11: DAS181 enhances NK-mediated tumor lysis by vaccinia virus, measured by MTS assay. [symbol] =T-test P value<0.05, suggesting that DAS181 alone boosts NK cell-mediated U87 tumor killing in vitro, compared to enzyme-dead DAS185. *=T-Test P value<0.05.

FIG. 12: DAS181 increases NK-mediated tumor killing by vaccinia virus as measured by MTS assay. *=T-test P value<0.05, suggesting that DAS181 increases NK cell-mediated killing of U87 cells by VV in vitro.

FIG. 13: DAS181 significantly enhanced expression of maturation markers (CD80, CD86, HLA-Dr, HLA-ABC) in human DC cells that were cultured alone or exposed to VV-infected tumor cells. *=T-test P value<0.05.

FIG. 14: DAS181 significantly enhanced TNF-alpha production by THP-1 derived macrophages. *=T-test P value<0.05

FIG. 15: DAS181 treatment promotes oncolytic adenovirus-mediated tumor cell killing and growth prohibition. A549-red tumor cells were seeded at 2K cells/well in 96-well plates. After overnight incubation, DAS181 vehicle, oncolytic adenovirus, and DAS181 were added as indicated. CD3/CD28/IL-2 were also added into each well with the amount described previously. Graph showed that DAS181 plus oncolytic adenovirus effectively reduced tumor cell proliferation.

FIGS. 16A-16B: DAS181 treatment enhances PBMC-mediated tumor cell killing by oncolytic virus. A549-red tumor cells were seeded at 2K cells/well in 96-well plate. After overnight incubation, fresh PBMCs were added at densities of 10K/well (A) or 40K/well (B). CD3, CD28, IL-2, DAS181, and oncolytic adenovirus were added as indicated in the graph following with the timed scans by IncuCyte. Graph showed that DAS181 plus oncolytic adenovirus dramatically enhanced human PBMC-mediated tumor cell eradication.

FIG. 17: Schematic of a portion of a vaccinia virus construct encoding a sialidase.

FIGS. 18A-18B: DAS181 expressed by Sialidase-VV has in vitro activity towards sialic acid-containing substrates. (A) Standard curve of DAS181 activity at 0.5 nM, 1 nM and 2 nM. (B) 1×10⁶ cells infected with Sialidase-VV express DAS181 equivalent to 0.78 nM-1.21 nM DAS181 in 1 ml medium in vitro.

FIG. 19: Sialidase-VV enhances Dendritic cell maturation. GM-CSF/IL4 derived human DC were cultured with Sial-VV or VV infected U87 tumor cell lysate for 24 hours. LPS was used as control. DC were collected and stained with antibodies against CD80, CD86, HLA-DR, and HLA-ABC. The expression of DC maturation markers was determined by flow analysis. The results suggested that Sial-VV enhanced DC maturation. *=T-test P value<0.05

FIG. 20: Sialidase-VV induced IFN-gamma and IL2 expression by T cells. CD3 antibody-activated human T cells were co-cultured with A594 tumor cells in the presence of Sial-VV- or VV-infected tumor cells lysate for 24 hours, and cytokine IFNr or IL-2 expression was measured by ELISA. The results suggested that Sial-VV-infected tumor cell lysate induced IFNr and IL2 expression by human T cells. *=T-test P value<0.05

FIG. 21: Sialidase-VV enhances T cell-mediated tumor cell lytic activity. CD3 Ab activated human T cells were co-cultured with Sial-VV- or VV-infected A594 tumor cells for 24 hours, and tumor cell viability was determined by MTS assay. The results suggested that Sial-VV infection of tumor cells resulted in enhanced tumor killing. *=T-test P value<0.05.

FIGS. 22A-22C: Impact of DAS181 and secreted sialidase Constructs 1, 2, and 3 on cell surface α2,3 sialic acid (FIG. 22A); α2,6 sialic acid (FIG. 22B) and galactose (FIG. 22C). FIG. 22A: A549-red cells were transfected by Construct-1, 2 or 3. After overnight incubation, transfected cells were lifted and re-seeded in 24-well plate. After additional 24 hrs, 48 hrs and 72 hrs, cells were fixed and stained with MALII-FITC for 1 hr before performing flow. Treat non-transfected cells with 100 nM DAS181 for 2 hrs before fixed. Vehicle prepared for DAS181 was used to treat another set of non-transfected cells as control. FIG. 22B: A549-red cells were transfected by Construct-1, 2 and 3. After overnight incubation, transfected cells were lifted and re-seeded in 24-well plate. After additional 24 hrs, 48 hrs and 72 hrs, cells were fixed and stained with SNA-FITC for 1 hr before performing flow. Treat non-transfected cells with 100 nM DAS181 for 2 hrs before fixed. Vehicle prepared for DAS181 was used to treat another set of non-transfected cells as control. FIG. 22C: A549-red cells were transfected by Construct-1, 2 and 3. After overnight incubation, transfected cells were lifted and re-seeded in 24-well plate. After additional 24 hrs, 48 hrs and 72 hrs, cells were fixed and stained with PNA-FITC for 1 hr before performing flow. Treat non-transfected cells with 100 nM DAS181 for 2 hrs before fixed. Vehicle prepared for DAS181 was used to treat another set of non-transfected cells as control.

FIGS. 23A-23C: Impact of DAS181 and transmembrane sialidase Constructs 1, 4, 5 and 6 on cell surface α2,3 sialic acid (FIG. 23A); α2,6 sialic acid (FIG. 23B); and galactose (FIG. 23C). FIG. 23A: A549-red cells were transfected by Construct-1, 4, 5, and 6. After overnight incubation, transfected cells were lifted and re-seeded in 24-well plate. After additional 24 hrs, 48 hrs and 72 hrs. cells were fixed and stained with MALII-Biotinylated for 1 hr followed by FITC-streptavidin for an additional 1 hr. The 2, 3-sialic acid level was detected by flow cytometry. FIG. 23B: A549-red cells were transfected by Construct-1, 4, 5, and 6. After overnight incubation, transfected cells were lifted and re-seeded in 24-well plate. In additional 24 hrs, 48 hrs and 72 hrs, cells were fixed and stained with SNA-FITC for 1 hr. The 2, 6-sialic acid level was detected by flow cytometry. FIG. 23C: A549-red cells were transfected by Construct-1, 4, 5, and 6. After overnight incubation, transfected cells were lifted and re-seeded in 24-well plate. After additional 24 hrs, 48 hrs and 72 hrs, cells were fixed and stained with PNA-FITC for 1 hr. The galactose level was detected by flow cytometry.

FIG. 24: Stable expression of Construct 1 increases oncolytic virus and PBMC-mediated A549 cell killing. Freshly isolated PBMCs were incubated with A549-red parental cells only or with cells stable expressing Construct-1 or cells stable expressing Construct-1 with 1 MOI or 5 MOI on two separated plates (Plate 2 and 4).

FIG. 25: Stable expression of Construct 4 increases oncolytic virus and PBMC-mediated A549 cell killing. Fresh isolated PBMCs were activated and incubated with A549-red cells only or with cells stable expressing Construct-4 or cells stable expressing Construct-4 with 1 MOI or 5 MOI OL in two separated plates (Plate 2 and 4).

FIG. 26: Design of exemplary sialidase expression constructs for recombination into the TK gene of Western Reserve VV to generate oncolytic virus encoding a sialidase. Exemplary constructs are shown for endocellular sialidase, secreted sialidase with an anchoring domain, and cell surface expressed sialidase with a transmembrane domain.

FIG. 27: PCR detection of Sialidase expression: CV-1 cells were infected with Sialidase-VV at an MOI of 0.2. After 48 hours, CV-1 cells were collected, and DNA were extracted using Wizard® SV Genomic DNA Purification System and used as template for Sialidase PCR amplification. PCR was conducted using standard PCR protocol. Expected PCR product size is 1251 bp.

FIG. 28: U87 or CV-1 cells were infected with control VV. SP-, Endo- or TM-Sial-VVs at MOI 1. The cells were collected at 24, 48, 72, or 96 hours. Virus titers were determined by plaque assay.

FIG. 29: U87 tumor cells were infected with control VV, SP-, Endo- or TM-Sial-VVs at MOI 0.1, 1, or 5. Tumor killing was measured by MTS assay.

FIG. 30: The expression of DC maturation marker HLA-ABC is enhanced by culture with oncolytic virus encoding secreted or transmembrane sialidase.

FIG. 31: The expression of DC maturation marker HLA-DR is enhanced by culture with oncolytic virus encoding secreted or transmembrane sialidase.

FIG. 32: The expression of DC maturation marker CD80 is enhanced by culture with oncolytic virus encoding secreted or transmembrane sialidase.

FIG. 33: The expression of DC maturation marker CD86 is enhanced by culture with oncolytic virus encoding secreted or transmembrane sialidase.

FIG. 34: Sial-VV enhances NK-mediated tumor lysis in vitro. Negative selected human NK cells (Astarte, WA) and VV-U87 cells (ATCC, VA) were co-cultured, and tumor killing efficacy was measured by LDH assay (Abcam, MA). The results suggested that Sial-VVs enhanced NK cell-mediated U87 tumor killing in vitro. (* P value, the Sial-VV vs Mock VV in U87 and NK culture).

FIG. 35: Results indicate that TM-sial-VV significantly inhibited tumor growth compared to control VV in vivo (tumor cells inoculated in right flank of mouse).

FIG. 36 Results indicate that TM-sial-VV significantly inhibited tumor growth compared to control VV in vivo (tumor cells inoculated in left flank of mouse).

FIG. 37: Mouse body weight was unaffected by treatment with Sial-VV or VV The results didn't show the difference on the mouse body weight.

FIGS. 38A-38B: Sialidase armed oncolytic vaccinia virus significantly enhanced CD8+ and CD4+ T cell infiltration within tumor. * p value: treatment group vs control VV group. FIG. 38A shows quantification of the results. FIG. 38B shows the FACS plots.

FIG. 39: TM-Sial-VV decreased the ratio of Treg/CD4+ T cells within the tumor, compared to control VV. * p value: treatment group vs control VV group.

FIG. 40: Sialidase armed oncolytic vaccinia virus significantly enhanced NK and NKT cell infiltration within tumor. * p value: treatment group vs control VV group.

FIG. 41: TM-Sial-VV significantly increased PD-L1 expression within tumor cells (p<0.05).

### DETAILED DESCRIPTION

The present application provides compositions and methods for treating cancers with an oncolytic virus (e.g., vaccinia virus) encoding a sialidase. The recombinant oncolytic viruses described herein are capable of delivering sialidase to tumor cells and/or the tumor cell environment. In some embodiments, the delivered sialidase can reduce sialic acid present on tumor cells or immune cells and render the tumor cells more vulnerable to killing by immune cells, immune cell-based therapies and/or other therapeutic agents whose effectiveness is diminished by hypersialylation of cancer cells. In some embodiments, the delivered sialidase reduces or prevents binding of Siglects on immune cells with their inhibitory receptor ligands (sialylated glycoconjugates). Thus, in some embodiments the delivered sialidase reduces or abolishes suppression of immunity against tumor cells. In some embodiments, the delivered sialidase (e.g., a bacterial sialidase) serves as a foreign antigen, and its expression on tumor cells enhances an immune response against the tumor cells. In some embodiments, the recombinant oncolytic virus is delivered via carrier cells (e.g., engineered immune cells or stem cells) expressing the virus. In some embodiments, the method further comprises administering engineered immune cells that enhance the anti-tumor effect of the recombinant oncolytic virus (e.g., by expressing a chimeric receptor targeting a foreign antigen, such as a sialidase, delivered by the oncolytic virus).

### I. Definitions

Terms are used herein as generally used in the art, unless otherwise defined as follows.

As used herein, “treatment” or “treating” is an approach for obtaining beneficial or desired results including clinical results. For purposes of this application, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, preventing or delaying the occurrence or recurrence of the disease, delay or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the present application contemplate any one or more of these aspects of treatment.

The terms “individual,” “subject” and “patient” are used interchangeably herein to describe a mammal, including humans. In some embodiments, the individual is human. In some embodiments, an individual suffers from a cancer. In some embodiments, the individual is in need of treatment.

As is understood in the art, an “effective amount” refers to an amount of a composition sufficient to produce a desired therapeutic outcome (e.g., reducing the severity or duration of, stabilizing the severity of, or eliminating one or more symptoms of cancer). For therapeutic use, beneficial or desired results include, e.g., decreasing one or more symptoms resulting from the disease (biochemical, histologic and/or behavioral), including its complications and intermediate pathological phenotypes presented during development of the disease, increasing the quality of life of those suffering from the disease, decreasing the dose of other medications required to treat the disease, enhancing effect of another medication, delaying the progression of the disease, and/or prolonging survival of patients. In some embodiments, an effective amount of the therapeutic agent may extend survival (including overall survival and progression free survival), result in an objective response (including a complete response or a partial response); relieve to some extent one or more signs or symptoms of the disease or condition; and/or improve the quality of life of the subject.

As used herein the term “wild type” is a term of the art understood by skilled persons and means the typical form of an organism, strain, gene or characteristic as it occurs in nature as distinguished from mutant or variant forms.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, “sialidase” refers to a naturally occurring or engineered sialidase that is capable of catalyzing the cleavage of terminal sialic acids from carbohydrates on glycoproteins or glycolipids. As used herein, “sialidase” can refer to a domain of a naturally occurring or non-naturally occurring sialidase that is capable of catalyzing cleavage of terminal sialic acids from carbohydrates on glycoproteins or glycolipids. The term “sialidase” also encompasses fusion proteins comprising a naturally occurring or non-naturally occurring sialidase protein or an enzymatically active fragment or domain thereof and another polypeptide, fragment or domain thereof, e.g., an anchoring domain or a transmembrane domain.

The term “sialidase” as used herein encompasses sialidase catalytic domain proteins. A “sialidase catalytic domain protein” is a protein that comprises the catalytic domain of a sialidase, or an amino acid sequence that is substantially homologous to the catalytic domain of a sialidase, but does not comprise the entire amino acid sequence of the sialidase. The catalytic domain is derived from, wherein the sialidase catalytic domain protein retains substantially the functional activity as the intact sialidase the catalytic domain is derived from. A sialidase catalytic domain protein can comprise amino acid sequences that are not derived from a sialidase. A sialidase catalytic domain protein can comprise amino acid sequences that are derived from or substantially homologous to amino acid sequences of one or more other known proteins, or can comprise one or more amino acids that are not derived from or substantially homologous to amino acid sequences of other known proteins.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “antibody” is used in its broadest sense and encompasses various antibody structures, including but not limited to monoclonal antibodies, polyclonal antibodies, multi-specific antibodies (e.g., bispecific antibodies, trispecific antibodies, etc), humanized antibodies, chimeric antibodies, full-length antibodies and antigen-binding fragments, single chain Fv, nanobodies, Fc fusion proteins, thereof, so long as they exhibit the desired antigen-binding activity. Antibodies and/or antibody fragments may be derived from murine antibodies, rabbit antibodies, chicken antibodies, human antibodies, fully humanized antibodies, camelid antibody variable domains and humanized versions, shark antibody variable domains and humanized versions, and camelized antibody variable domains.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified.

The terms “virus” or “virus particle” are used according to its plain ordinary meaning within Virology and refers to a virion including the viral genome (e.g. DNA, RNA, single strand, double strand), viral capsid and associated proteins, and in the case of enveloped viruses (e.g. herpesvirus, poxvirus), an envelope including lipids and optionally components of host cell membranes, and/or viral proteins.

As used herein, “oncolytic viruses” refer to viruses that selectively replicate in and selectively kill tumor cells in subjects having a tumor. These include viruses that naturally preferentially replicate and accumulate in tumor cells, such as poxviruses, and viruses that have been engineered to do so. Some oncolytic viruses can kill a tumor cell following infection of the tumor cell. For example, an oncolytic virus can cause death of the tumor cell by lysing the tumor cell or inducing cell death of the tumor cell. Exemplary oncolytic viruses include, but are not limited to, poxviruses, herpesviruses, adenoviruses, adeno-associated viruses, lentiviruses, retroviruses, rhabdoviruses, papillomaviruses, vesicular stomatitis virus, measles virus, Newcastle disease virus, picomavirus, Sindbis virus, papillomavirus, parvovirus, reovirus, and coxsackievirus.

The term “poxvirus” is used according to its plain ordinary meaning within Virology and refers to a member of Poxviridae family capable of infecting vertebrates and invertebrates which replicate in the cytoplasm of their host. In embodiments, poxvirus virions have a size of about 200 nm in diameter and about 300 nm in length and possess a genome in a single, linear, double-stranded segment of DNA, typically 130-375 kilobase. The term poxvirus includes, without limitation, all genera of poxviridae (e.g., betaentomopoxvirus, yatapoxvirus, cervidpoxvirus, gammaentomopoxvirus, leporipoxvirus, suipoxvirus, molluscipoxvirus, crocodylidpoxvirus, alphaentomopoxvirus, capripoxvirus, orthopoxvirus, avipoxvirus, and parapoxvirus). In embodiments, the poxvirus is an orthopoxvirus (e.g., smallpox virus, vaccinia virus, cowpox virus, monkeypox virus), parapoxvirus (e.g., orf virus, pseudocowpox virus, bovine popular stomatitis virus), yatapoxvirus (e.g., tanapox virus, yaba monkey tumor virus) or molluscipoxvirus (e.g., molluscum contagiosum virus). In embodiments, the poxvirus is an orthopoxvirus (e.g., cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, or vaccinia virus strain AS). In embodiments, the poxvirus is a parapoxvirus (e.g., orf virus strain NZ2 or pseudocowpox virus strain TJS).

As used herein, a “modified virus” or a “recombinant virus” refers to a virus that is altered in its genome compared to a parental strain of the virus. Typically modified viruses have one or more truncations, substitutions (replacement), mutations, insertions (addition) or deletions (truncation) of nucleotides in the genome of a parental strain of virus. A modified virus can have one or more endogenous viral genes modified and/or one or more intergenic regions modified. Exemplary modified viruses can have one or more heterologous nucleotide sequences inserted into the genome of the virus. Modified viruses can contain one or more heterologous nucleotide sequences in the form of a gene expression cassette for the expression of a heterologous gene. Modifications can be made using any method known to one of skill in the art, including as provided herein, such as genetic engineering and recombinant DNA methods.

“Percent (%) amino acid sequence identity” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004, Edgar, R. C., BMC Bioinformatics 5(1):113, 2004, each of which are incorporated herein by reference in their entirety for all purposes).

The term “epitope” as used herein refers to the specific group of atoms or amino acids on an antigen to which an antibody or diabody binds. Two antibodies or antibody moieties may bind the same epitope within an antigen if they exhibit competitive binding for the antigen.

The terms “polypeptide” or “peptide” are used herein to encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation. ADP-ribosylation, pegylation, biotinylation, etc.).

As use herein, the terms “specifically binds,” “specifically recognizing,” and “is specific for” refer to measurable and reproducible interactions, such as binding between a target and an antibody (such as a diabody). In certain embodiments, specific binding is determinative of the presence of the target in the presence of a heterogeneous population of molecules, including biological molecules (e.g., cell surface receptors). For example, an antibody that specifically recognizes a target (which can be an epitope) is an antibody (such as a diabody) that binds this target with greater affinity, avidity, more readily, and/or with greater duration than its bindings to other molecules. In some embodiments, the extent of binding of an antibody to an unrelated molecule is less than about 10% of the binding of the antibody to the target as measured. e.g., by a radioimmunoassay (RIA). In some embodiments, an antibody that specifically binds a target has a dissociation constant (KD) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, an antibody specifically binds an epitope on a protein that is conserved among the protein from different species. In some embodiments, specific binding can include, but does not require exclusive binding. Binding specificity of the antibody or antigen-binding domain can be determined experimentally by methods known in the art. Such methods comprise, but are not limited to Western blots, ELISA, RIA, ECL, IRMA, EIA, BIACORE™ and peptide scans.

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to one or more ingredients in a pharmaceutical formulation, other than an active ingredient, which is nontoxic to a subject. A pharmaceutically acceptable carrier includes, but is not limited to, a buffer, excipient, stabilizer, cryoprotectant, tonicity agent, preservative, and combinations thereof. Pharmaceutically acceptable carriers or excipients have preferably met the required standards of toxicological and manufacturing testing and/or are included on the Inactive Ingredient Guide prepared by the U.S. Food and Drug administration or other state/federal government or listed in the U.S. Pharmacopeia or other generally recognized pharmacopeia for use in mammals, and more particularly in humans.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or condition (e.g., cancer), or a probe for specifically detecting a biomarker described herein. In certain embodiments, the manufacture or kit is promoted, distributed, or sold as a unit for performing the methods described herein.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat disease of type X means the method is used to treat disease of types other than X.

The term “about X-Y” used herein has the same meaning as “about X to about Y.”

As used herein and in the appended claims, the singular forms “a,” “an,” or “the” include plural referents unless the context clearly dictates otherwise.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Compositions

The present application provides recombinant oncolytic viruses for treating a cancer in an individual in need thereof. In some embodiments, the present application provides a recombinant oncolytic virus comprising a nucleotide sequence encoding a sialidase. In some embodiments, the nucleotide sequence encoding the sialidase is operably linked to a promoter. In some embodiments, the recombinant oncolytic virus further comprises a second nucleotide sequence encoding a heterologous protein or nucleic acid.

In some embodiments, the present application provides a recombinant oncolytic virus comprising a first nucleotide sequence encoding a sialidase and a second nucleotide sequence encoding a heterologous protein or nucleic acid, wherein the first nucleotide sequence is operably linked to a promoter and the second nucleotide sequence is operably linked to a promoter. In some embodiments, the first nucleotide sequence and the second nucleotide sequence are operably linked to the same promoter. In some embodiments, the first nucleotide sequence and the second nucleotide sequence are operably linked to different promoters. In some embodiments, the recombinant oncolytic virus comprises two or more nucleotide sequences, wherein each nucleotide sequence encodes a heterologous protein or nucleic acid. In some embodiments, the second nucleotide sequence encodes a heterologous protein selected from the group consisting of immune checkpoint inhibitors, inhibitors of immune suppressive receptors, multi-specific immune cell engager (e.g., a BiTE), cytokines, costimulatory molecules, tumor antigen presenting proteins, anti-angiogenic factors, tumor-associated antigens, foreign antigens, and matrix metalloproteases (MMP), Regulatory molecules of Macrophage or monocyte functions (antibodies to LILRBs), antibodies to folate receptor beta, tumor cell specific antigens (CD19. CDH17, etc) or antibodies to tumor scaffold (FAP, fibulin-3, etc).

In some embodiments, the oncolytic virus is a virus selected from the group consisting of: vaccinia virus, reovirus, Seneca Valley virus (SVV), vesicular stomatitis virus (VSV), Newcastle disease virus (NDV), herpes simplex virus (HSV), morbillivirus virus, retrovirus, influenza virus, Sinbis virus, poxvirus, measles virus, cytomegalovirus (CMV), lentivirus, adenovirus (Ad), and derivatives thereof. In some embodiments, the oncolytic virus is modified to reduce immunogenicity of the virus. Suitable oncolytic viruses and derivatives thereof are described in the “Oncolytic Viruses” subsection below.

In some embodiments, there is provided a recombinant vaccinia virus comprising a first nucleotide sequence encoding a sialidase, wherein the first nucleotide sequence is operably linked to a promoter. In some embodiments, the vaccinia virus further comprises a second nucleotide encoding a heterologous protein, e.g., an immune checkpoint inhibitor, an inhibitor of an immune suppressive receptor, a cytokine, a costimulatory molecule, a tumor antigen presenting protein, an anti-angiogenic factor, a tumor-associated antigen, a foreign antigen, or a matrix metalloprotease (MMP), Regulatory molecules of Macrophage or monocyte functions (antibodies to LILRBs), antibodies to folate receptor beta, tumor cell specific antigens (CD19, CDH17, etc) or antibodies to tumor scaffold (FAP, fibulin-3, etc) wherein the second nucleotide sequence is operably linked to the same or a different promoter. In some embodiments, the virus is vaccinia virus Western Reserve. In some embodiments, the virus is a vaccinia virus, and the one or more mutations are in one or more proteins selected from the group consisting of A14, A17, A13, L1, H3, D8, A33, B5, A56, F13, A28, and A27. In some embodiments, the one or more mutations are in one or more proteins selected from the group consisting of A27L, H3L, D8L and L1R.

In some embodiments, there is provided a recombinant vaccinia virus comprising a first nucleotide sequence encoding a sialidase, wherein the first nucleotide sequence is operably linked to a promoter. In some embodiments, the vaccinia virus further comprises a second nucleotide encoding a heterologous protein, wherein the heterologous protein is a membrane-bound complement activation modulator such as CD55, CD59, CD46, CD35, factor H, C4-binding protein, or other identified complement activation modulators, and wherein the second nucleotide sequence is operably linked to the same or a different promoter. In some embodiments, the virus is vaccinia virus Western Reserve. In some embodiments, the virus is a vaccinia virus, and the one or more mutations are in one or more proteins selected from the group consisting of A14, A17, A13, L1, H3, D8, A33, B5, A56, F13, A28, and A27. In some embodiments, the one or more mutations are in one or more proteins selected from the group consisting of A27L, H3L, D8L and L1R.

The present application provides recombinant oncolytic viruses (e.g., vaccinia virus) encoding heterologous proteins or nucleic acids as described below. In some embodiments, the recombinant oncolytic virus encodes a sialidase. In some embodiments, the sialidase is human or bacterial sialidase. In some embodiments, the sialidase is a secreted sialidase. In some embodiments, the sialidase comprises a membrane anchoring moiety or a transmembrane domain. Suitable sialidases and derivatives or variants thereof are described in the “Sialidase” subsection below. In some embodiments, the recombinant oncolytic virus encodes one or more heterologous proteins or nucleic acids that promote an immune response or inhibit an immune suppressive protein, as described in the “Other heterologous proteins or nucleic acids” subsection below.

In some embodiments, there is provided a recombinant oncolytic viruses (e.g., vaccinia virus) comprising a first nucleotide sequence encoding a Actinomyces viscosus sialidase or a derivative thereof, wherein the first nucleotide sequence is operably linked to a promoter. In some embodiments, the oncolytic virus further comprises a second nucleotide sequence encoding a heterologous protein (e.g., an immune checkpoint inhibitor, an inhibitor of an immune suppressive receptor, a cytokine, a costimulatory molecule, a tumor antigen presenting protein, an anti-angiogenic factor, a tumor-associated antigen, a foreign antigen, or a matrix metalloprotease (MMP)), wherein the second nucleotide sequence is operably linked to the same or a different promoter. In some embodiments, the recombinant oncolytic virus is an enveloped virus (e.g., vaccinia virus) and the heterologous protein is a membrane-bound complement activation modulator such as CD55. CD59, CD46, CD35, factor H, C4-binding protein, or other identified complement activation modulators. In some embodiments, the sialidase comprises an amino acid sequence having at least about 80% sequence identity to the amino acid sequence of SEQ ID NO: 1 or 26.

In some embodiments, there is provided a recombinant oncolytic viruses (e.g., vaccinia virus) encoding a sialidase comprising an anchoring domain (e.g., DAS181). In some embodiments, the oncolytic virus further comprises a second nucleotide sequence encoding a heterologous protein or nucleic acid. In some embodiments, the anchoring domain is a glycosaminoglycan (GAG)-binding domain. In some embodiments, the anchoring domain is positively charged at physiologic pH. In some embodiments, the anchoring domain is located at the carboxy terminus of the sialidase. In some embodiments, the sialidase is derived from a Actinomyces viscosus sialidase. In some embodiments, the sialidase is DAS181. In some embodiments, the nucleotide sequence encoding the sialidase further encodes a secretion sequence operably linked to the sialidase. In some embodiments, the secretion sequence is operably linked to the amino terminus of the sialidase.

In some embodiments, there is provided a recombinant oncolytic viruses (e.g., vaccinia virus) encoding a sialidase comprising a transmembrane domain. In some embodiments, the transmembrane domain comprises an amino acid sequence selected from SEQ ID NOs: 45-52. In some embodiments, the oncolytic virus further comprises a second nucleotide sequence encoding a heterologous protein or nucleic acid. In some embodiments, the sialidase is derived from a Actinomyces viscosus sialidase. In some embodiments, the nucleotide sequence encoding the sialidase further encodes a secretion sequence operably linked to the sialidase.

The nucleotide sequence encoding the heterologous protein or nucleic acid (e.g., sialidase protein) is operably linked to a promoter. In some embodiments, the promoter is a viral promoter, such as an early, late, or early/late viral promoter. In some embodiments, the promoter is a hybrid promoter. In some embodiments, the promoter is comprises a promoter sequence of a human promoter (e.g., a tissue- or tumor-specific promoter). Suitable promoters are described in the “Promoters for expression of heterologous proteins or nucleic acids” subsection below.

The present application further provides engineered immune cells for treatment of a cancer in an individual in need thereof. In some embodiments, the engineered immune cells comprise chimeric receptors that specifically recognize a tumor antigen. In some embodiments, the engineered immune cells comprise chimeric receptors that specifically recognize a foreign antigen (e.g., a bacterial sialidase) encoded by any one of the recombinant oncolytic viruses described herein. Suitable engineered immune cells are described in the “Engineered immune cells” subsection below.

In some embodiments, there is provided a composition comprising an engineered immune cell comprising a recombinant oncolytic virus encoding a sialidase. In some embodiments, the recombinant oncolytic virus is a vaccinia virus. In some embodiments, the vaccinia virus is a Western Reserve strain. In some embodiments, the vaccinia virus is a modified vaccinia virus (e.g., a vaccinia virus comprising one or more mutations, wherein the mutations are in one or more proteins such as A14, A17, A13, L1, H3, D8, A33, B5, A56, F13, or A28). In some embodiments, the sialidase is derived from a Actinomyces viscosus sialidase. In some embodiments, the sialidase is DAS181. In some embodiments, the nucleotide sequence encoding the sialidase further encodes a secretion sequence operably linked to the sialidase. In some embodiments, the sialidase further comprises a transmembrane domain. In some embodiments, the engineered immune cell encodes a chimeric receptor. In some embodiments, the chimeric receptor is a chimeric antigen receptor. In some embodiments, the engineered immune cell is a cytotoxic T cell, a helper T cell, a suppressor T cell, an NK cell, and an NK-T cell. In some embodiments, the engineered immune cell is an autologous cell of a patient or an allogeneic cell.

In some embodiments, there is provided a composition comprising (a) a recombinant oncolytic virus comprising a nucleotide sequence encoding a foreign antigen; and (b) an engineered immune cell expressing a chimeric receptor specifically recognizing said foreign antigen. In some embodiments, the foreign antigen is a bacterial antigen. In some embodiments, the foreign antigen is a sialidase.

The present application further provides immune cells comprising any one of the recombinant oncolytic viruses provided herein. In some embodiments, the immune cells comprising a recombinant oncolytic virus are prepared by incubating the immune cells with the recombinant oncolytic virus. In some embodiments, the immune cells comprising a recombinant oncolytic virus are prepared by engineering a nucleotide sequence encoding the recombinant oncolytic virus into the cells (e.g., by transducing or transfecting the cells with the construct). Suitable immune cells expressing recombinant oncolytic virus and methods of preparation thereof are described in the “Oncolytic virus and engineered immune cells” subsection below.

### A. Oncolytic Viruses

The present application provides recombinant oncolytic viruses for use in treating a cancer, comprising at least one nucleotide sequence encoding a heterologous protein. In some embodiments, the heterologous protein is operably linked to a promoter. In some embodiments, the heterologous protein is a sialidase.

Numerous oncolytic viruses, including Vaccinia virus, Coxsackie virus, Adenovirus, Measles, Newcastle disease virus, Seneca Valley virus, Coxsackie A21, Vesicular stomatitis virus, Parvovirus H1. Reovirus, Herpes virus, Lentivirus, and Poliovirus, and Parvovirus. Vaccinia Virus Western Reserve, GLV-1h68, ACAM2000, and OncoVEX GFP, are available. The genomes of these oncolytic virus can be genetically modified to insert a nucleotide sequence encoding a protein that includes all or a catalytic portion of a sialidase. The nucleotide sequence encoding a protein that includes all or a catalytically active portion of a sialidase is placed under the control of a viral expression cassette so that the sialidase is expressed by infected cells.

Oncolytic viruses (OVs) have the ability to preferentially accumulate in and replicate in and kill tumor cells, relative to normal cells. This ability can be a native feature of the virus (e.g., pox virus, reovirus, Newcastle disease virus and mumps virus), or the viruses can be modified or selected for this property. Viruses can be genetically attenuated or modified so that they can circumvent antiviral immune and other defenses in the subject (e.g., vesicular stomatitis virus, herpes simplex virus, adenovirus) so that they preferentially accumulate in tumor cells or the tumor microenvironment, and/or the preference for tumor cells can be selected for or engineered into the virus using, for example, tumor-specific cell surface molecules, transcription factors and tissue-specific microRNAs (see, e.g., Cattaneo el al, Nat. Rev. Microbiol., 6(7):529-540 (2008); Dorer et al, Adv. Drug Deliv. Rev., 61(7-8):554-571 (2009); Kelly et al., Mol. Ther., 17(3):409-416 (2009); and Naik et al., Expert Opin. Biol. Ther., 9(9): 1163-1176 (2009)).

Delivery of oncolytic viruses can be achieved via direct intratumoral injection. While direct intratumoral delivery can minimize the exposure of normal cells to the virus, there often are limitations due to, e.g., inaccessibility of the tumor site (e.g., brain tumors) or for tumors that are in the form of several small nodules spread out over a large area or for metastatic disease. Viruses can be delivered via systemic or local delivery, such as by intravenous administration, or intraperitoneal administration, and other such routes. Systemic delivery can deliver virus not only to the primary tumor site, but also to disseminated metastases.

Numerous oncolytic viruses, including Vaccinia virus, Coxsackie virus, Adenovirus, Measles, Newcastle disease virus, Seneca Valley virus, Coxsackie A21, Vesicular stomatitis virus, Parvovirus H1. Reovirus, Herpes virus, Lentivirus, and Poliovirus, and Parvovirus. Vaccinia Virus Western Reserve, GLV-1h68, ACAM2000, and OncoVEX GFP, are available. The genomes of these oncolytic virus can be genetically modified to insert a nucleotide sequence encoding a protein that includes all or a catalytic portion of a sialidase. The nucleotide sequence encoding a protein that includes all or a catalytically active portion of a sialidase is placed under the control of a viral expression cassette so that the sialidase is expressed by infected cells.

Other unmodified oncolytic viruses include any known to those of skill in the art, including those selected from among viruses designated GLV-1h68, JX-594, JX-954, ColoAd1, MV-CEA, MV-NIS, ONYX-015, B18R, H101, OncoVEX GM-CSF, Reolysin, NTX-010, CCTG-102, Cavatak, Oncorine, and TNFerade.

Suitable oncolytic viruses have been described, for example, in WO2020097269, which is incorporated herein by reference in its entirety. Oncolytic viruses described herein include for example, vesicular stomatitis virus, see, e.g., U.S. Pat. Nos. 7,731,974, 7,153,510, 6,653,103 and U.S. Pat. Pub. Nos. 2010/0178684, 2010/0172877, 2010/0113567, 2007/0098743, 20050260601, 20050220818 and EP Pat. Nos. 1385466, 1606411 and 1520175: herpes simplex virus, see, e.g., U.S. Pat. Nos. 7,897,146, 7,731,952, 7,550,296, 7,537,924, 6,723,316, 6,428,968 and U.S. Pat. Pub. Nos. 2011/0177032, 2011/0158948, 2010/0092515, 2009/0274728, 2009/0285860, 2009/0215147, 2009/0010889, 2007/0110720, 2006/0039894 and 20040009604; retroviruses, see, e.g., U.S. Pat. Nos. 6,689,871, 6,635,472, 6,639,139, 5,851,529, 5,716,826, 5,716,613 and U.S. Pat. Pub. No. 20110212530; and adeno-associated viruses, see, e.g., U.S. Pat. Nos. 8,007,780, 7,968,340, 7,943,374, 7,906,111, 7,927,585, 7,811,814, 7,662,627, 7,241,447, 7,238,526, 7,172,893, 7,033,826, 7,001,765, 6,897,045, and 6,632,670.

In some embodiments, the oncolytic virus is a vesicular stomatitis virus (VSV). VSV has been used in multiple oncolytic virus applications. In addition, VSV has been engineered to express an antigenic protein of human papilloma virus (HPV) as a method to treat HPV positive cervical cancers via vaccination (REF 18337377, 29998190) and to express pro-inflammatory factors to increase the immune reaction to tumors (REF 12885903). Various methods for engineering VSV to encode an additional gene have been described (REF 7753828). Briefly, the VSV RNA genome is reverse transcribed to a complementary, doubled stranded-DNA with an upstream T7 RNA polymerase promoter and an appropriate location within the VSV genome for gene insertion is identified (e.g., within the noncoding 5′ or 3′ regions flanking VSV glycoprotein (G) (REF 12885903). Restriction enzyme digestion can be accomplished, e.g., with Mlu I and Nhe I, yielding a linearized DNA molecule. An insert consisting of a DNA molecule encoding the gene of interest flanked by appropriate restriction sites can be ligated into the linearized VSV genomic DNA. The resulting DNA can be transcribed with T7 polymerase, yielding a complete VSV genomic RNA containing the inserted gene of interest. Introduction of this RNA molecule to a mammalian cell, e.g., via transfection and incubation results in viral progeny expressing the protein encoded by the gene of interest.

In some embodiments, the recombinant oncolytic virus is an adenovirus. In some embodiments, the adenovirus is an adenovirus serotype 5 virus (Ad5). Ad5 contains a human E2F-1 promoter, which is a retinoblastoma (Rb) pathway-defective tumor specific transcription regulatory element that drives expression of the essential Ela viral genes, restricting viral replication and cytotoxicity to Rb pathway-defective tumor cells (REF 16397056). A hallmark of tumor cells is Rb pathway defects. Engineering a gene of interest into Ad5 is accomplished through ligation into Ad5 genome. A plasmid containing the gene of interest is generated via and digested, e.g., with AsiSI and PacI. An Ad5 DNA plasmid, e.g., PSF-AD5 (REF Sigma OGS268) is digested with AsiSI and PacI and ligated with recombinant bacterial ligase or co-transformed with RE digested gene of interest into permissive E. coli as has been reported for the generation of human granulocyte macrophage colony stimulating factor (GM-CSF) expressing Ad5 (REF 16397056). Recovery of the DNA and transfection into a permissive host, e.g., human embryonic kidney cells (HEK293) or HeLa yields virus encoding the gene of interest.

In some embodiments, the recombinant oncolytic virus is a modified oncolytic virus (e.g., a derivative of any one of the viruses described herein). In some embodiments, the recombinant oncolytic virus comprises one or more mutations that reduce immunogenicity of the virus compared to a corresponding wild-type strain.

### Vaccinia Virus (V7)

In some embodiments, the recombinant oncolytic virus is a vaccinia virus (VV). Various strains of VV have been used as templates for OV therapeutics; the unifying feature is deletion of the viral thymidine kinase (TK) gene, rendering a virus dependent upon actively replicating cells, i.e. neoplastic cells, for productive replication and thus these VVs have preferential infectivity of cancer cells exemplified by the Western Reserve (WR) strain of VV (REF 25876464). Production of VV's with a gene of interest inserted in the genome may be accomplished with homologous recombination utilizing lox sites.

In some embodiments, the virus is a modified vaccinia virus. In some embodiments, the virus is a modified vaccinia virus comprising one or more mutations. In some embodiments, the one or more mutations are in one or more proteins such as A14, A17, A13, L1, H3, D8, A33, B5, A56, F13, A28, and A27. In some embodiments, the one or more mutations are in one or more proteins selected from the group consisting of A27L, H3L, D8L and L1R. Exemplary mutations have been described, for example, in international patent publication WO2020086423, which is incorporated herein by reference in its entirety.

A limiting factor in the use of VVs as cancer treatment delivery vectors is the strong neutralizing antibody (Nab) response induced by the injection of VV into the bloodstream that limits the ability of the virus to persist and spread and prevents vector re-dosing. The NAbs recognize and bind viral glycoproteins embedded in the VV envelope, thus preventing virus interaction with host cell receptors. A number of VV glycoproteins involved in host cell receptor recognition have been identified. Among them, proteins H3L, L1R, A27L, D8L, A33R, and B5R have been shown to be targeted by NAbs, with A27L, H3L, D8L and L1R being the main NAb antigens presented on the surface of mature viral particles. A27L, H3L, and D8L are the adhesion molecules that bind to host glycosaminoglycans (GAGs) heparan sulfate (HS) (A27L and H3L) and chondroitin sulfate (CS) (D8L) and mediate endocytosis of the virus into the host cell. L1R protein is involved in virus maturation. Modified vaccinia viruses comprising mutations in one or more of these proteins have been described in international patent publication WO2020086423, which is herein incorporated by reference in its entirety.

In some embodiments, the modified vaccinia virus comprises one or more proteins selected from the group consisting of: (a) a variant vaccinia virus (VV) H3L protein that comprises an amino acid sequence having at least 90% (e.g., at least 91%, 92%, 93%, 94%, 95%, 96%, 97%, 98%, 99%, or 100%) amino acid sequence identity to any one of SEQ ID NOS: 66-69; (b) a variant vaccinia virus (VV) D8L protein that comprises an amino acid sequence having at least 90% (e.g., at least 91%, 92%, 93%, 94%, 95%, 96%, 97%, 98%, 99%, or 100%) amino acid sequence identity to any one of SEQ ID NOS: 70-72 or 85; (c) a variant vaccinia virus (VV) A27L protein that comprises an amino acid sequence having at least 90% (e.g., at least 91%, 92%, 93%, 94%, 95%, 96%, 97%, 98%, 99%, or 100%) amino acid sequence identity to SEQ ID NO: 73; and (d) a variant vaccinia virus (VV) L1R protein that comprises an amino acid sequence having at least 90% (e.g., at least 91%, 92%, 93%, 94%, 95%, 96%, 97%, 98%, 99%, or 100%) amino acid sequence identity to SEQ ID NO: 74.

In some embodiments, the variant VV H3L protein comprises amino acid substitution or deletion at one or more of the following amino acid residues: 14, 15, 16, 33, 34, 35, 38, 40, 44, 45, 52, 131, 134, 135, 136, 137, 154, 155, 156, 161, 166, 167, 168, 198, 227, 250, 253, 254, 255, and 256, wherein the amino acid numbering is based on SEQ ID NO: 66. In some embodiments, the variant VV H3L comprises one or more amino acid mutations selected from the group consisting of I14A, D15A, R16A, K38A, P44A, E45A, V52A, E131A, T134A, L136A, R137A, R154A, E155A, I156A, M168A, I198A, E250A, K253A, P254A, N255A, and F256A, wherein the amino acid numbering is based on SEQ ID NO: 66.

In some embodiments, the variant VV D8L protein comprises amino acid substitution or deletion at one or more of the following amino acid residues: 44, 48, 98, 108, 117, and 220, wherein the amino acid numbering is based on SEQ ID NO: 70. In some embodiments, the variant VV D8L construct comprises one or more amino acid mutations selected from the group consisting of R44A, K48A, K98A, K108A, K117A, and R220A, wherein the amino acid numbering is based on SEQ ID NO: 70.

In some embodiments, the variant VV A27L protein comprises amino acid substitution or deletion at one or more of the following amino acid residues: 27, 30, 32, 33, 34, 35, 36, 37, 39, 40, 107, 108, and 109, wherein the amino acid numbering is based on SEQ ID NO: 73. In some embodiments, the variant A27L construct comprises one or more amino acid mutations selected from the group consisting of K27A, A30D, R32A, E33A, A34D, I35A, V36A, K37A, D39A, E40A, R107A, P108A, and Y109A, wherein the amino acid numbering is based on SEQ ID NO: 73.

In some embodiments, the variant VV L1R protein comprises amino acid substitution or deletion at one or more of the following amino acid residues: 25, 27, 31, 32, 33, 35, 58, 60, 62, 125, and 127, wherein the amino acid numbering is based on SEQ ID NO: 74. In some embodiments, the variant L1R construct comprises one or more amino acid mutations selected from the group consisting of E25A, N27A, Q31A, T32A, K33A, D35A, S58A, D60A, D62A, K125A, and K127A, wherein the amino acid numbering is based on SEQ ID NO: 74.

In some embodiments, the variant VV H3L protein comprises amino acid substitution or deletion at one or more of the following amino acid residues: 14, 15, 16, 33, 34, 35, 38, 40, 44, 45, 52, 131, 132, 134, 135, 136, 137, 154, 155, 156, 161, 166, 167, 168, 195, 198, 199, 227, 250, 251, 252, 253, 254, 255, 256, 258, 262, 264, 266, 268, 272, 273, 275, and 277, wherein the amino acid numbering is based on SEQ ID NO: 68. In some embodiments, the variant H3L construct comprises one or more amino acid mutations selected from the group consisting of 114A, D15A, R16A, K33A, F34A, D35A, K38A, N40A, P44A, E45A, V52A, E131A, D132A, T134A, F135A, L136A, R137A, R154A, E155A, 1156A, K161A, L166A, VI 67 A, M168A, E195A, I198A, V199A, R227A, E250A, N251A, M252A, K253A, P254A, N255A, F256A, S258A, T262P, A264T, K2661, Y268C, M272K, Y273N, F275N, and T277A, wherein the amino acid numbering is based on SEQ ID NO: 68.

In some embodiments, the variant VV D8L protein comprises amino acid substitution or deletion at one or more of the following amino acid residues: 43, 44, 48, 53, 54, 55, 98, 108, 109, 144, 168, 177, 196, 199, 203, 207, 212, 218, 220, 222, and 227, wherein the amino acid numbering is based on SEQ ID NO: 72. In some embodiments, the variant VV D8L construct comprises one or more amino acid mutations selected from the group consisting of V43A, R44A. K48A, S53A, G54A, G55A, K98A, K108A, K109A, A144G. T168A, S177A, L196A, F199A, L203A, N207A, P212A, N218A, R220A, P222A, and D227A, wherein the amino acid numbering is based on SEQ ID NO: 72.

### B. Heterologous Proteins or Nucleic Acids

### 1. Sialidase

In some embodiments, the recombinant oncolytic virus encodes a heterologous protein that includes all or a catalytic portion of a sialidase that is capable of removing sialic acid (N-acetyneuraminic acid (Neu5Ac)), e.g., from a glycan on a human cell. In general, Neu5Ac is linked via an alpha 2,3, an alpha 2,6 or alpha 2,8 linkage to the penultimate sugar in glycan on a protein by any of a variety of sialyl transferases. The common human sialyltransferases are summarized in Table 1.

TABLE 1
Nomenclature of Neu5Ac sialyltransferases

| | | | EC | |
|---|---|---|---|---|
| Abbreviation | Resulting Group | Substrate | Number | HGNC |
| ST3Gal I | Neu5Ac-α-(2,3) Gal | Gal-β-1,3-GalNAc | 2.4.99.4 | 10862 |
| ST3Gal II | Neu5Ac-α-(2,3) Gal | Gal-β-1,3-GalNAc | 2.4.99.4 | 10863 |
| ST3Gal III | Neu5Ac-α-(2,3) Gal | Gal-β-1,3(4)-GlcNAc | 2.4.99.6 | 10866 |
| ST3Gal IV | Neu5Ac-α-(2,3) Gal | Gal-β-1,4-GlcNAc | 2.4.99.9 | 10864 |
| ST3Gal V | Neu5Ac-α-(2,3) Gal | Gal-β-1,4-Glc | 2.4.99.9 | 10872 |
| ST3Gal VI | Neu5Ac-α-(2,3) Gal | Gal-β-1,4-GlcNAc | 2.4.99.9 | 18080 |
| ST6Gal I | Neu5Ac-α-(2,6) Gal | Gal-β-1,4-GlcNAc | 2.4.99.1 | 10860 |
| ST6Gal II | Neu5Ac-α-(2,6) Gal | Gal-β-1,4-GlcNAc | 2.4.99.2 | 10861 |
| ST6GalNAc I | Neu5Ac-α-(2,6) | GalNAc-α-1,O-Ser/Thr | 2.4.99.7 | 23614 |
| | GalNAc | | | |
| ST6GalNAc | Neu5Ac-α-(2,6) | Gal-β-1,3-GalNAc-α-1,O-Ser/Thr | 2.4.99.7 | 10867 |
| II | GalNAc | | | |
| ST6GalNAc | Neu5Ac-α-(2,6) | Neu5Ac-α-2,3-Gal-β-1,3-GalNAc | 2.4.99.7 | 19343 |
| III | GalNAc | | | |
| ST6GalNAc | Neu5Ac-α-(2,6) | Neu5Ac-α-2,3Gal-β-1,3-GalNAc | 2.4.99.7 | 17846 |
| IV | GalNAc | | | |
| ST6GalNAc | Neu5Ac-α-(2,6) | Neu5Ac-α-2,6-GalNAc-β-1,3-GalNAc | 2.4.99.7 | 19342 |
| V | GalNAc | | | |
| ST6GalNAc | Neu5Ac-α-(2,6) | All α-series gangliosides | 2.4.99.7 | 23364 |
| VI | GalNAc | | | |
| ST8Sia I | Neu5Ac-α-(2,8)- | Neu5Ac-α-2,3-Gal-β-1,4-Glc-β-1,1Cer | 2.4.99.8 | 10869 |
| | Neu5Ac | (GM5) | | |
| ST8Sia II | Neu5Ac-α-(2,8)- | Neu5Ac-α-2,3-Gal-β-1,4-GlcNAc | 2.4.99.8 | 10870 |
| | Neu5Ac | | | |
| ST8Sia III | Neu5Ac-α-(2,8)- | Neu5Ac-α-2,3-Gal-β-1,4-GlcNAc | 2.4.99.8 | 14269 |
| | Neu5Ac | | | |
| ST8Sia IV | Neu5Ac-α-(2,8)- | (Neu5Ac-α-2,8)nNeu5Ac-α-2,3-Gal- | 2.4.99.8 | 10871 |
| | Neu5Ac | β-1-R | | |
| ST8Sia V | Neu5Ac-α-(2,8)- | GM1b, GT1b, GD1a, GD3 | 2.4.99.8 | 17827 |
| | Neu5Ac | | | |
| ST8Sia VI | Neu5Ac-α-(2,8)- | Neu5Ac-α-2,3(6)-Gal | 2.4.99.8 | 23317 |
| | Neu5Ac | | | |

HGNC: Hugo Gene Community Nomenclature (world wide web.genenames.org)

The heterologous protein, in addition to a naturally occurring sialidase or catalytic portion thereof can, optionally, include peptide or protein sequences that contribute to the therapeutic activity of the protein. For example, the protein can include an anchoring domain that promotes interaction between the protein and a cell surface. The anchoring domain and sialidase domain can be arranged in any appropriate way that allows the protein to bind at or near a target cell membrane such that the therapeutic sialidase can exhibit an extracellular activity that removes sialic acid residues. The protein can have more than one anchoring domains. In cases in which the polypeptide has more than one anchoring domain, the anchoring domains can be the same or different. The protein can comprise one or more transmembrane domains (e.g., one or more transmembrane alpha helices). The protein can have more than one sialidase domain. In cases in which a compound has more than one sialidase domain, the sialidase domains can be the same or different. Where the protein comprises multiple anchoring domains, the anchoring domains can be arranged in tandem (with or without linkers) or on alternate sides of other domains, such as sialidase domains. Where a compound comprises multiple sialidase domains, the sialidase domains can be arranged in tandem (with or without linkers) or on alternate sides of other domains.

### Sialidase Catalytic Activity

In some embodiments, the sialidase has exo-sialidase activity as defined by Enzyme Commission EC 3.2.1.18. In some embodiments, the sialidase is an anhydrosialidase as defined by Enzyme Commission EC 4.2.2.15.

In some embodiments, the sialidase expressed by the oncolytic virus can be specific for Neu5Ac linked via alpha 2,3 linkage, specific for Neu5Ac linked via an alpha 2,6 specific for Neu5Ac linked via alpha 2,8 linkage, or can cleave Neu5Ac linked via an alpha 2,3 linkage or an alpha 2.6 linkage. In some embodiments, the sialidase can cleave Neu5Ac linked via an alpha 2,3 linkage, an alpha 2,6 linkage, or an alpha 2,8 linkage. A variety of sialidases are described in Tables 2-5.

A sialidase that can cleave more than one type of linkage between a sialic acid residue and the remainder of a substrate molecule, in particular, a sialidase that can cleave both alpha(2,6)-Gal and alpha(2,3)-Gal linkages or both alpha(2,6)-Gal and alpha(2,3)-Gal linkages and alpha(2,8)-Gal linkages can be used in the compounds of the disclosure. Sialidases included are the large bacterial sialidases that can degrade the receptor sialic acids Neu5Ac alpha(2,6)-Gal and Neu5Ac alpha(2,3)-Gal. For example, the bacterial sialidase enzymes from Clostridium perfringens (Genbank Accession Number X87369), Actinomyces viscosus (GenBankX62276), Arthrobacter ureafaciens GenBank (AY934539), or Micromonospora viridifaciens (Genbank Accession Number D01045) can be used.

In some embodiments, the sialidase comprises all or a portion of the amino acid sequence of a large bacterial sialidase or can comprise amino acid sequences having at least 80%, at least 85%, at least 90%, at least 95%, at least 98%, at least 99%, or 100% sequence identity to all or a portion of the amino acid sequence of a large bacterial sialidase. In some embodiments, the sialidase domain comprises SEQ ID NO: 2 or 27, or a sialidase sequence having at least 80%, at least 85%, at least 90%, at least 95%, at least 98%, at least 99%, or 100% sequence identity to SEQ ID NO: 12. In some embodiments, a sialidase domain comprises the catalytic domain of the Actinomyces viscosus sialidase extending from amino acids 274-666 of SEQ ID NO: 26, having at least 80%, at least 85%, at least 90%, at least 95%, at least 98%, at least 99%, or 100% sequence identity to amino acids 274-666 of SEQ ID NO: 26.

Additional sialidases include the human sialidases such as those encoded by the genes NEU2 (SEQ ID NO: 4: Genbank Accession Number Y16535; Monti, E, Preti, Rossi, E., Ballabio, A and Borsani G. (1999) Genomics 57:137-143) and NEU4 (SEQ ID NO: 6; Genbank Accession Number NM080741; Monti et al. (2002) Neurochem Res 27:646-663). Sialidase domains of compounds of the present disclosure can comprise all or a portion of the amino acid sequences of a sialidase or can comprise amino acid sequences having at least 75%, at least 80%, at least 85%, at least 90%, at least 95%, at least 98%, or at least 99% sequence identity to all or a portion of the amino acid sequences of a sialidase. In some embodiments, where a sialidase domain comprises a portion of the amino acid sequences of a naturally occurring sialidase, or sequences having at least 75%, at least 80%, at least 85%, at least 90%, at least 95%, at least 98%, or at least 99% sequence identity to a portion of the amino acid sequences of a naturally occurring sialidase, the portion comprises essentially the same activity as the intact sialidase. In some embodiments, the sialidase expressed by the recombinant oncolytic virus is a sialidase catalytic domain protein. As used herein a “sialidase catalytic domain protein” comprises a catalytic domain of a sialidase but does not comprise the entire amino acid sequence of the sialidase from which the catalytic domain is derived. A “sialidase catalytic domain protein” has sialidase activity, and the term as used herein is interchangeable with a “sialidase”. In some embodiments, a sialidase catalytic domain protein comprises at least 10%, at least 20%, at least 50%, at least 70% of the activity of the sialidase from which the catalytic domain sequence is derived. In some embodiments, a sialidase catalytic domain protein comprises at least 90% of the activity of the sialidase from which the catalytic domain sequence is derived.

A sialidase catalytic domain protein can include other amino acid sequences, such as but not limited to additional sialidase sequences, sequences derived from other proteins, or sequences that are not derived from sequences of naturally occurring proteins. Additional amino acid sequences can perform any of a number of functions, including contributing other activities to the catalytic domain protein, enhancing the expression, processing, folding, or stability of the sialidase catalytic domain protein, or even providing a desirable size or spacing of the protein.

In some embodiments, the sialidase catalytic domain protein is a protein that comprises the catalytic domain of the A. viscosus sialidase. In some embodiments, an A. viscosus sialidase catalytic domain protein comprises amino acids 270-666 of the A. viscosus sialidase sequence (SEQ ID NO: 26; GenBank WP_003789074). In some embodiments, an A. viscosus sialidase catalytic domain protein comprises an amino acid sequence that begins at any of the amino acids from amino acid 270 to amino acid 290 of the A. viscosus sialidase sequence (SEQ ID NO: 26) and ends at any of the amino acids from amino acid 665 to amino acid 901 of said A. viscosus sialidase sequence (SEQ ID NO: 26), and lacks any A. viscosus sialidase protein sequence extending from amino acid I to amino acid 269.

In some embodiments, an A. viscosus sialidase catalytic domain protein comprises amino acids 274-681 of the A. viscosus sialidase sequence (SEQ ID NO: 26) and lacks other A. viscosus sialidase sequence. In some embodiments, an A. viscosus sialidase catalytic domain protein comprises amino acids 274-666 of the A. viscosus sialidase sequence (SEQ ID NO: 26) and lacks any other A. viscosus sialidase sequence. In some embodiments, an A. viscosus sialidase catalytic domain protein comprises amino acids 290-666 of the A. viscosus sialidase sequence (SEQ ID NO: 26) and lacks any other A. viscosus sialidase sequence. In yet other embodiments, an A. viscosus sialidase catalytic domain protein comprises amino acids 290-681 of the A. viscosus sialidase sequence (SEQ ID NO: 26) and lacks any other A. viscosus sialidase sequence.

In some embodiments, useful sialidase polypeptides for expression by an oncolytic virus include polypeptides comprising a sequence that is 90%, 95%, 96%, 97%, 98%, 99% or 100% identical to SEQ ID NO: 27 or comprises 375, 376, 377, 380, 381, 382, 383, 384, 385, 386, 387, 388, 389, 390, 391, or 392 contiguous amino acids of SEQ ID NO: 27.

In some embodiments, the sialidase is DAS181, a functional derivative thereof (e.g., a fragment thereof), or a biosimilar thereof. In some embodiments, the sialidase comprises an amino acid sequence that is at least about 80% (e.g., at least about any one of 85%, 86%, 87%, 88%, 89%, 90%, 91%, 92%, 93%, 94%, 95%, 96%, 97%, 98%, or 99%) or 100% identical to SEQ ID NO: 2. In some embodiments, the sialidase comprises 414, 413, 412, 411, or 410 contiguous amino acids of SEQ ID NO: 2. In some embodiments, the sialidase comprises a fragment of DAS181 without the anchoring domain (AR domain). In some embodiments, the sialidase comprises an amino acid sequence that is at least about 80% (e.g., at least about any one of 85%, 86%, 87%, 88%, 89%, 90%, 91%, 92%, 93%, 94%, 95%, 96%, 97%, 98%, or 99%) or 100% identical to SEQ ID NO: 27.

DAS181 is a recombinant sialidase fusion protein with a heparin-binding anchoring domain. DAS181 and methods for preparing and formulating DAS181 are described in U.S. Pat. Nos. 7,645,448, 9,700,602 and 10,351,828, each of which is herein incorporated by reference in their entirety for any and all purposes.

In some embodiments, the sialidase is a secreted form of DAS181, a functional derivative thereof, or a biosimilar thereof. In some embodiments, the nucleotide sequence encoding a secreted form of DAS181 encodes a secretion sequence operably linked to DAS181, wherein the secretion sequence is enables secretion of the protein from eukaryotic cells. In some embodiments, the sialidase comprises an amino acid sequence that is at least about 80% (e.g., at least about any one of 85%, 86%, 87%, 88%, 89%, 90%, 91%, 92%, 93%, 94%, 95%, 96%, 97%, 98%, or 99%) or 100% identical to SEQ ID NO: 28. In some embodiments, the sialidase comprises an amino acid sequence that is at least about 80% (e.g., at least about any one of 85%, 86%, 87%, 88%, 89%, 90%, 91%, 92%, 93%, 94%, 95%, 96%, 97%, 98%, or 99%) or 100% identical to SEQ ID NO: 28. In some embodiments, the sialidase comprises 414, 413, 412, 411, or 410 contiguous amino acids of SEQ ID NO: 28. An exemplary secreted form of DAS181 is described in Example 11.

In some embodiments, the sialidase is a transmembrane form of DAS181, a functional derivative thereof, or a biosimilar thereof. In some embodiments, the sialidase comprises an amino acid sequence that is at least about 80% (e.g., at least about any one of 85%, 86%, 87%, 88%, 89%, 90%, 91%, 92%, 93%, 94%, 95%, 96%, 97%, 98%, or 99%) or 100% identical to SEQ ID NO: 31. In some embodiments, the sialidase comprises an amino acid sequence that is at least about 80% (e.g., at least about any one of 85%, 86%, 87%, 88%, 89%, 90%, 91%, 92%, 93%, 94%, 95%, 96%, 97%, 98%, or 99%) or 100% identical to SEQ ID NO: 31. In some embodiments, the sialidase comprises 414, 413, 412, 411, or 410 contiguous amino acids of SEQ ID NO: 31. An exemplary transmembrane form of DAS181 is described in Example 11.

TABLE 2
Engineered Sialidases

| Name | Sequence |
|---|---|
| A. viscosus | mtshspfsrr hlpallgslp laatgliaaa ppahavptsd gladvtitqv |
| sialidase | napadglysv gdvmtfnitl tntsgeahsy apastnlsgn vskcrwrnvp |
| | agttktdctg lathtvtaed lkaggftpqi ayevkaveya gkalstpeti |
| | kgatspvkan slrvesitps sskeyyklgd tvtytvrvrs vsdktinvaa |
| | tessfddlgr qchwgglkpg kgavynckpl thtitqadvd agrwtpsitl |
| | tatgtdgtal qtltatgnpi nvvgdhpqat papapdaste lpasmsqaqh |
| | vapntatdny ripaittapn gdllisyder pkdngnggsd apnpnhivqr |
| | rstdggktws aptyihqgte tgkkvgysdp syvvdhqtgt ifnfhvksyd |
| | hgwgnsqagt dpenrgiiqa evststdngw twthrtitad itkdnpwtar |
| | faasgqgiqi qhgphagrlv qqytirtagg avqavsvysd dhgktwqagt |
| | pvgtgmdenk vvelsdgslm lnsrasdssg frkvahstdg gqtwsepvsd |
| | knlpdsvdna qiirafpnaa pddprakvll lshspnpkpw srdrgtisms |
| | cddgaswtts kvfhepfvgy ttiavqsdgs igllsedahd ganyggiwyr |
| | nftmnwlgeq cgqkpaepsp apsptaapsa apseqpapsa apsteptqap |
| | apssapepsa vpepssapap epttapstep tptpapssap epsagptaap |
| | apetssapaa eptqaptvap saeptqvpga qpsaapsekp gaqpssapkp |
| | datgrapsvv npkataapsg kasssaspap srsatatskp gmepdeidrp |
| | sdgamaqptg gasapsaapt qaakagsrls rtgtnallvl glaqvavvgg |
| | tkkkrarrsk n (SEQ ID NO: 26) |
| AvCD | MGDHPQATPA PAPDASTELP ASMSQAQHLA ANTATDNYRT PAITTAPNGD |
| | LLISYDERPK DNGNGGSDAP NPNHIVQRRS TDGGKTWSAP TYIHQGTETG |
| | KKVGYSDPSY VVDHQTGTIF NFHVKSYDQG WGGSRGGTDP ENRGIIQAEV |
| | STSTDNGWTW THRTITADIT KDKPWTARFA ASGQGIQIQH GPHAGRLVQQ |
| | YTIRTAGGAV QAVSVYSDDH GKTWQAGTPT GTGMDENKVV ELSDGSLMLN |
| | SRASDGSGFR KVAHSTDGGQ TWSEPVSDKN LPDSVDNAQTIRAFPNAAPD |
| | DPRAKVLLLS HSPNPRPWSR DRGTISMSCD DGASWTTSKV FHEPFVGYTT |
| | IAVQSDGSIG LLSEDAHNGA DYGGIWYRNF TMNWLGEQCG QKPAE (SEQ ID NO: 1) |
| DAS181 | MGDHPQATPA PAPDASTELP ASMSQAQHLA ANTATDNYRT PAITTAPNGD |
| | LLISYDERPK DNGNGGSDAP NPNHIVQRRS TDGGKTWSAP TYIHQGTETG |
| | KKVGYSDPSY VVDHQTGTIF NFHVKSYDQG WGGSRGGTDP ENRGIIQAEV |
| | STSTDNGWTW THRTITADIT KDKPWTAREA ASGQGIQIQH GPHAGRLVQQ |
| | YTIRTAGGAV QAVSVYSDDH GKTWQAGTPT GTGMDENKVV ELSDGSLMLN |
| | SRASDGSGFR KVAHSTDGGQ TWSEPVSDKN LPDSVDNAQT IRAFPNAAPD |
| | DPRAKVLLLS HSPNPRPWSR DRGTISMSCD DGASWTTSKV FHEPFVGYTT |
| | IAVQSDGSIG LLSEDAHNGA DYGGIWYRNF TMNWLGEQCG QKPAKRKKKG |
| | GKNGKNRRNR KKKNP (SEQ ID NO: 2) |
| DAS181 | GDHPQATPAP APDASTELPA SMSQAQHLAA NTATDNYRIP AITTAPNGDL |
| without | LISYDERPKD NGNGGSDAPN PNHIVQRRST DGGKTWSAPT YIHQGTETGK |
| initial Met | KVGYSDPSYV VDHQTGTIFN FHVKSYDQGW GGSRGGTDPE NRGIIQAEVS |
| and without | TSTDNGWTWT HRTITADITK DKPWTARFAA SGQGIQIQHG PHAGRLVQQY |
| anchoring | TIRTAGGAVQ AVSVYSDDHG KTWQAGTPIG TGMDENKVVE LSDGSLMLNS |
| domain | RASDGSGFRK VAHSTDGGQT WSEPVSDKNL PDSVDNAQIT RAFPNAAPDD |
| | PRAKVLLLSH SPNPRPWSRD RGTISMSCDD GASWTTSKVF HEPFVGYTTI |
| | AVQSDGSIGL LSEDAHNGAD YGGIWYRNFT MNWLGEQCGQ KPA (SEQ ID NO: 27) |
| Membrane | METDTLLLWVLLLWVPGSTGDMGDHPQATPAPAPDASTELPASMSQAQHLAANTATD |
| Bound | NYRIPAITTAPNGDLLISYDERPKDNGNGGSDAPNPNHIVQRRSTDGGKTWSAPTYI |
| sialidase | HQGTETGKKVGYSDPSYVVDHQTGTIFNFHVKSYDQGWGGSRGGTDPENRGIIQAEV |
| (secretion | STSTDNGWTWTHRTITADITKDKPWTARFAASGQGIQIQHGPHAGRLVQQYTIRTAG |
| sequence | GAVQAVSVYSDDHGKTWQAGTPIGTGMDENKVVELSDGSLMLNSRASDGSGFRKVAH |
| and TM | STDGGQTWSEPVSDKNLPDSVDNAQIIRAFPNAAPDDPRAKVLLLSHSPNPRPWSRD |
| underlined) | RGTISMSCDDGASWTTSKVFHEPFVGFTTIAVQSDGSIGLLSEDAHNGADYGGIWYR |
| | NFTMNWLGEQCGQKPANAVGQDTQEVIVVPHSLPFKVVVISAILALVVLTIISLIIL |
| | IMLWQKKPR (SEQ ID NO: 31) |
| Secreted | METDTLLLWVLLLWVPGSTGDGDHPQATPAPAPDASTELPASMSQAQHLAANTATDNYRIPAI |
| sialidase | TTAPNGDLLISYDERPKDNGNGGSDAPNPNHIVQRRSTDGGKTWSAPTYIHQGTETGKKVGYS |
| (secretion | DPSYVVDHQTGTIFNFHVKSYDQGWGGSRGGTDPENRGIIQAEVSTSTDNGWTWTHRTITADI |
| sequence | TKDKPWTARFAASGQGIQIQHGPHAGRLVQQYTIRTAGGAVQAVSVYSDDHGKTWQAGTPIGT |
| underlined) | GMDENKVVELSDGSLMLNSRASDGSGFRKVAHSTDGGQTWSEPVSDKNLPDSVDNAQIIRAFP |
| | NAAPDDPRAKVLLLSHSPNPRPWSRDRGTISMSCDDGASWTTSKVFHEPFVGYTTIAVQSDGS |
| | IGLLSEDAHNGADYGGIWYRNFTMNWLGEQCGQKPAKRKKKGGKNGKNRRNRKKKNP (SEQ |
| | ID NO: 28) |

TABLE 3
Human Sialidases

| Name | Uniprot identifier | SEQ ID NO |
|---|---|---|
| Human Neu 1 | Q99519 | 3 |
| Human Neu 2 | Q9Y3R4 | 4 |
| Human Neu 3 | Q9UQ49 | 5 |
| Human Neu 4 | Q8WWR8 | 6 |
| Human Neu 4 Isoform 2 | Q8WWR8 | 7 |
| Human Neu 4 Isoform 3 | Q8WWR8 | 8 |

TABLE 4
Sialidases in organisms that are largely commensal with humans

| | Uniprot/ | | |
|---|---|---|---|
| Organism | Genbank ID | Gene name | SEQ ID NO |
| Actinomyces viscosus | Q59164 | nanH | 9 |
| Actinoinyces viscosus | A0A448PLN7 | nanA | 10 |
| Streptococcus oralis | A0A081R4G6 | nanA | 11 |
| Streptococcus oralis | D4FUA3 | nanH | 12 |
| Streptococcus mitis | A0A081Q016 | nanA | 13 |
| Streptococcus mitis | A0A3R9LET9 | nanA_1 | 14 |
| Streptococcus mitis | A0A3R9J1C3 | nanA_2 | 15 |
| Streptococcus mitis | A0A3R9IIK2 | nanA_3 | 16 |
| Streptococcus mitis | A0A3R9IXG7 | nanA_4 | 17 |
| Streptococcus mitis | A0A3R9K5C5 | nanA_5 | 18 |
| Streptococcus mitis | J1H2U0 | nanH | 19 |
| Porphyroinonas gingivalis | B2RL82 | | 20 |
| Tannerella forsythia | Q84BM9 | siaHI | 21 |
| Tannerella forsythia | A0A1D3USB1 | nanH | 22 |
| Akkermansia Muciniphila | B2UPI5 | | 23 |
| Akkermansia Muciniphila | B2UN42 | | 24 |
| Bacteroides | Q8AAK9 | | 25 |
| thetaiotaomicron | | | |

TABLE 5
Additional sialidases

| | Uniprot/ |
|---|---|
| Organism | Genbank ID |
| Actinotignum schaalii | S2VK03 |
| Anaerotruncus colihominis | B0PE27 |
| Ruminococcus gnavus | A0A2N5NZH2 |
| Clostridium difficile | Q185B3 |
| Clostridium septicum | P29767 |
| Clostridium perfringens | P10481 |
| Clostridium perfringens | Q8XMY5 |
| Clostridium perfringens | A0A2Z3TZA2 |
| Vibrio cholerae | P0C6E9 |
| Salmonella typhimurium | P29768 |
| Paeniclostridium sordellii | A0A446I8A2 |
| Streptococcus pneumoniae (NanA) | P62576 |
| Streptococcus pneumoniae (NanB) | Q54727 |
| Pseudomonas aeruginosa | A0A2X4HZU8 |
| Aspergillus fumigatus | Q4WQS0 |
| Arthrobacter ureafaciens | Q5W7Q2 |
| Micromonospora viridifaciens | Q02834 |

### Anchoring Domain

In some embodiments, the sialidase comprises an anchoring domain. As used herein, an “extracellular anchoring domain” or “anchoring domain” is any moiety that interacts with an entity that is at or on the exterior surface of a target cell or is in close proximity to the exterior surface of a target cell. An anchoring domain can serve to retain a sialidase of the present disclosure at or near the external surface of a target cell. An extracellular anchoring domain may bind 1) a molecule expressed on the surface of a cancer cell, or a moiety, domain, or epitope of a molecule expressed on the surface of a cancer cell, 2) a chemical entity attached to a molecule expressed on the surface of a cancer cell, or 3) a molecule of the extracellular matrix surrounding a cancer cell.

An exemplary anchoring domain binds to heparin/sulfate, a type of GAG that is ubiquitously present on cell membranes. Many proteins specifically bind to heparin/heparan sulfate, and the GAG-binding sequences in these proteins have been identified (Meyer. F A, King, M and Gelman, R A. (1975) Biochimica et Biophysica Acta 392: 223-232; Schauer, S. ed., pp 233. Sialic Acids Chemistry. Metabolism and Function. Springer-Verlag, 1982). For example, the GAG-binding sequences of human platelet factor 4 (PF4) (SEQ ID NO:77), human interleukin 8 (IL8)(SEQ ID NO:78), human antithrombin III (AT III) (SEQ ID NO:80), human apoprotein E (ApoE) (SEQ ID NO:80), human angio-associated migratory cell protein (AAMP) (SEQ ID NO:81), or human amphiregulin (SEQ ID NO:82) have been shown to have very high affinity to heparin.

In some embodiments, the anchoring domain is anon-protein anchoring moiety, such as a phosphatidylinositol (GPI) linker.

### Linkers

A protein that includes a sialidase or a catalytic domain thereof can optionally include one or more polypeptide linkers that can join various domains of the sialidase. Linkers can be used to provide optimal spacing or folding of the domains of a protein. The domains of a protein joined by linkers can be sialidase domains, anchoring domains, transmembrane domains, or any other domains or moieties of the compound that provide additional functions such as enhancing protein stability, facilitating purification, etc. Some preferred linkers include the amino acid glycine. For example, linkers having the sequence: (GGGGS (SEQ ID NO: 55))n, where n is 1-20. In some embodiments, the linker is a hinge region of an immunoglobulin. Any hinge or linker sequence capable of keeping the catalytic domain free of steric hindrance can be used to link a domain of a sialidase to another domain (e.g., a transmembrane domain or an anchoring domain). In some embodiments, the linker is a hinge domain comprising the sequence of SEQ ID NO: 62.

### Secretion Sequence

In some embodiments, the nucleotide sequence encoding the sialidase further encodes a secretion sequence (e.g., a signal sequence or signal peptide) operably linked to the sialidase. The terms “secretion sequence,” “signal sequence,” and “signal peptide” are used interchangeably. In some embodiments, the secretion sequence is a signal peptide operably linked to the N-terminus of the protein. In some embodiments, the length of the secretion sequence ranges between 10 and 30 amino acids (e.g., between 15 and 25 amino acids, between 15 and 22 amino acids, or between 20 and 25 amino acids). In some embodiments, the secretion sequence enables secretion of the protein from eukaryotic cells. During translocation across the endoplasmic reticulum membrane, the secretion sequence is usually cleaved off and the protein enters the secretory pathway. In some embodiments, the nucleotide sequence encodes, from N-terminus to C-terminus, a secretion sequence, a sialidase, and a transmembrane domain, wherein the sialidase is operably linked to the secretion sequence and the transmembrane domain. In some embodiments, the N-terminal secretion sequence is cleaved resulting in a protein with an N-terminal extracellular domain. An exemplary secretion sequence is provided in SEQ ID NO: 40.

### Transmembrane Domain

In some embodiments, the sialidase comprises a transmembrane domain. In some embodiments, the sialidase domain can be joined to a mammalian (preferably human) transmembrane (TM) domain. This arrangement permits the sialidase to be expressed on the cell surface. Suitable transmembrane domain include, but are not limited to a sequence comprising human CD28 TM domain (NM_006139; FWVLVVVGGVLACYSLLVTVAFIIFWV (SEQ ID NO: 46), human CD4 TM domain (M35160; MALIVLGGVAGLLLFIGLGIFF (SEQ ID NO: 47); human CD8 TM1 domain (NM_001768; IYIWAPLAGTCGVLLLSLVIT (SEQ ID NO: 48); human CD8 TM2 domain (NM_001768; IYIWAPLAGTCGVLLLSLVITLY (SEQ ID NO: 49); human CD8 TM3 domain (NM_001768; IYIWAPLAGTCGVLLLSLVITLYC (SEQ ID NO: 50); human 41BB TM domain (NM_001561; IISFFLALTSTALLFLLFF LTLRFSVV (SEQ ID NO: 51); human PDGFR TM1 domain (VVISAILA LVVLTIISLIILI; SEQ ID NO:52); and human PDGFR TM2 domain NAVGQDTQEVIVVPHSLPFKVVVISAILALVVLTIISLIILIMLWQKKPR; SEQ ID NO; 45)

In some embodiments, the nucleotide sequence encoding a sialidase encodes a protein comprising, from amino terminus to carboxy terminus, a secretion sequence (e.g., SEQ ID NO: 40), a sialidase (e.g., a sialidase comprising an amino acid sequence selected from SEQ ID NOs: 1-27, and a transmembrane domain (e.g., a transmembrane domain selected from SEQ ID NOs: 45-52). However, any suitable secretion sequence, sialidase domain sequence, or transmembrane domain may be used. In some embodiments, the nucleotide sequence encoding a sialidase encodes a protein comprising, from amino terminus to carboxy terminus, a secretion sequence (e.g., SEQ ID NO: 40), the sialidase of SEQ ID NO: 27, and a transmembrane domain (e.g., a transmembrane domain selected from SEQ ID NOs: 45-52).

In some embodiments, the sialidase has at least 50%, at least 60%, at least 65%, 80% (e.g., at least about any one of 85%, 86%, 87%, 88%, 89%) or at least 90% (e.g., at least about any one of 91%, 92%, 94%, 96%, 98%, or 99%) sequence identity to a sequence selected from SEQ ID NOs: 31. In some embodiments, the sialidase comprises a sequence selected from SEQ ID NOs: 31. In some embodiments, the sialidase comprises the amino acid sequence of SEQ ID NO. 31.

### 2. Other Heterologous Proteins or Nucleotide Sequences

In some embodiments according to any one of the recombinant oncolytic viruses described above, the oncolytic virus further comprises a second nucleotide sequence encoding a heterologous protein or nucleic acid. In some embodiments, the second nucleotide sequence encodes a heterologous protein.

In some embodiments according to any one of the recombinant oncolytic viruses described above, the heterologous protein is an immune checkpoint inhibitor. In some embodiments, the immune checkpoint inhibitor is an inhibitor of CTLA-4, PD-1, PD-L1, TIGIT, LAG3, TIM-3, VISTA, B7-H4, or HLA-G. In some embodiments, the immune checkpoint inhibitor is an antibody. In some embodiments, the immune checkpoint modulator is an immune checkpoint inhibitor, such as an inhibitor or an antagonist antibody or a decoy ligand of PD-1, PD-L1, PD-L2, CD47, CXCR4, CSF1R, LAG-3, TIM-3, HHLA2, BTLA, CD160, CD73, CTLA-4, B7-H4, TIGIT, VISTA, or 2B4 In some embodiments, the immune checkpoint modulator is an inhibitor of PD-1. In some embodiments, the immune checkpoint inhibitor is an antibody against an immune checkpoint molecule, such as an anti-PD-1 antibody. In some embodiments, the immune checkpoint inhibitor is a ligand that binds to the immune checkpoint molecule, such as soluble or free PD-L1/PD-L2. In some embodiments, the immune checkpoint inhibitor is an extracellular domain of PD-1 fused to an Fc fragment of an immunoglobulin (such as IgG4 Fc)) that can block PDL-1 on tumor cell surface binding to the immune check point PD-1 on immune cells. In some embodiments, the immune checkpoint inhibitor is a ligand that binds to HHLA2. In some embodiments, the immune checkpoint inhibitor is an extracellular domain of TMIGD2 fused to an Fc fragment of an immunoglobulin, such as IgG4 Fc. In some embodiments, the immune checkpoint inhibitor is a ligand that binds to at least two different inhibitory immune checkpoint molecules (e.g. bispecific), such as a ligand that binds to both CD47 and CXCR4. In some embodiments, the immune checkpoint inhibitor comprises an extracellular domain of SIRPα and a CXCL12 fragment fused to an Fc fragment of an immunoglobulin, such as IgG4 Fc. These molecules can bind to CD47 on cancer cell, thus stopping its interaction with SIRPalpha to block the “don't eat me” signal to macrophages and dendritic cells.

In some embodiments, the heterologous protein is an inhibitor of an immune suppressive receptor. The immune suppressive receptor can be any receptor expressed by an immune effector cell that inhibits or reduces an immune response to tumor cells. Exemplary effector cell includes without limitation a T lymphocyte, a B lymphocyte, a natural killer (NK) cell, a dendritic cell (DC), a macrophage, a monocyte, a neutrophil, an NKT-cell, or the like. In some embodiments, the immune suppressive receptor is LILRB, TYRO3, AXL, Folate receptor beta or MERTK. In some embodiments, the inhibitor of an immune suppressive receptor is an anti-LILRB antibody.

In some embodiments, the heterologous protein is a multi-specific immune cell engager. In some embodiments, the multi-specific immune cell engager is a bispecific immune cell engager. In some embodiments, the heterologous protein is a bispecific T cell engager (BiTE). Exemplary bispecific immune cell engagers have been described, for example, in international patent publication WO2018049261, herein incorporated by reference in its entirety. In some embodiments, the bispecific immune cell engager comprises a first antigen-binding domain (such as scFv) specifically recognizing a tumor antigen (such as EpCAM, FAP, or EGFR, etc) and a second antigen-binding domain (such as scFv) specifically recognizing a cell surface molecule on an effector cell (such as CD3 or 4-1BB on T lymphocytes). Tumor antigens can be a tumor-associated antigen (TAA) or a tumor-specific antigen (TSA). In some embodiments, TAA or TSA is expressed on a cell of a solid tumor. Tumor antigens include, but are not limited to, EpCAM, FAP, EphA2, HER2, GD2, EGFR, VEGFR2, and Glypican-3 (GPC3), CDH17, Fibulin-3, HHLA2. Folate receptors, etc. In some embodiments, the tumor antigen is EpCAM. In some embodiments, the tumor antigen is FAP. In some embodiments, the tumor antigen is EGFR.

As described above, effector cells include, but are not limited to T lymphocyte, B lymphocyte, natural killer (NK) cell, dendritic cell (DC), macrophage, monocyte, neutrophil, NKT-cell, or the like. In some embodiments, the effector cell is a T lymphocyte. In some embodiments, the effector cell is a cytotoxic T lymphocyte. Cell surface molecules on an effector cell include, but are not limited to CD3, CD4, CD5, CD8, CD16, CD28, CD40, CD64, CD89, CD134, CD137, NKp46, NKG2D, or the like. In some embodiments, the cell surface molecule is CD3.

A cell surface molecule on an effector cell of the present application is a molecule found on the external cell wall or plasma membrane of a specific cell type or a limited number of cell types. Examples of cell surface molecules include, but are not limited to, membrane proteins such as receptors, transporters, ion channels, proton pumps, and G protein-coupled receptors: extracellular matrix molecules such as adhesion molecules (e.g., integrins, cadherins, selectins, or NCAMS); see, e.g., U.S. Pat. No. 7,556,928, which is incorporated herein by reference in its entirety. Cell surface molecules on an effector cell include but not limited to CD3, CD4, CD5, CD8, CD16, CD27, CD28, CD38, CD64, CD89, CD134, CD137, CD154, CD226, CD278, NKp46, NKp44, NKp30, NKG2D, and an invariant TCR.

The cell surface molecule-binding domain of an engager molecule can provide activation to immune effector cells. The skilled artisan recognizes that immune cells have different cell surface molecules. For example CD3 is a cell surface molecule on T-cells, whereas CD16, NKG2D, or NKp30 are cell surface molecules on NK cells, and CD3 or an invariant TCR are the cell surface molecules on NKT-cells. Engager molecules that activate T-cells may therefore have a different cell surface molecule-binding domain than engager molecules that activate NK cells. In some embodiments, e.g., wherein the immune cell is a T-cell, the activation molecule is one or more of CD3. e.g., CD3γ, CD3δ or CD3ε; or CD27, CD28, CD40, CD134, CD137, and CD278. In other some embodiments, e.g., wherein the immune cell is a NK cell, the cell surface molecule is CD16, NKG2D, or NKp30, or wherein the immune cell is a NKT-cell, the cell surface molecule is CD3 or an invariant TCR

CD3 comprises three different polypeptide chains (ε, δ and γ chains), is an antigen expressed by T cells. The three CD3 polypeptide chains associate with the T-cell receptor (TCR) and the ζ-chain to form the TCR complex, which has the function of activating signaling cascades in T cells. Currently, many therapeutic strategies target the TCR signal transduction to treat diseases using anti-human CD3 monoclonal antibodies. The CD3 specific antibody OKT3 is the first monoclonal antibody approved for human therapeutic use, and is clinically used as an immunomodulator for the treatment of allogenic transplant rejections.

In some embodiments, the heterologous protein reduces neutralization of the recombinant oncolytic virus by the immune system of the individual. In some embodiments, the recombinant oncolytic virus is an enveloped virus (e.g., vaccinia virus), and the heterologous protein is a complement activation modulator (e.g., CD55 or CD59). Complement is a key component of the innate immune system, targeting the virus for neutralization and clearance from the circulatory system. Complement activation results in cleavage and activation of C3 and deposition of opsonic C3 fragments on surfaces. Subsequent cleavage of C5 leads to assembly of the membrane attack complex (C5b, 6, 7, 8, 9), which disrupts lipid bilayers.

In some embodiments, recombinant oncolytic virus is an enveloped virus (e.g., vaccinia virus), and the heterologous protein is a complement activation modulator such as CD55, CD59, CD46, CD35, factor H. C4-binding protein, or other identified complement activation modulators. Without wishing to be bound by theory, expression of the complement activation modulators on the virus envelope surface (e.g., the vaccinia virus envelope) results in a virus having the ability to modulate complement activation and reduce complement-mediated virus neutralization as compared to the wild-type virus. In some embodiments, the heterologous nucleotide sequence encodes a domain of human CD55, CD59, CD46. CD35, factor H, C4-binding protein, or other identified complement activation modulators. In another embodiment, the heterologous nucleic acid encodes a CD55 protein that comprises an amino acid sequence having the sequence of SEQ ID NO: 58. In view of the disclosure presented herein, one of ordinary skill in the art would readily employ other complement activation modulators (e.g. CD59, CD46, CD35, factor H, C4-binding protein etc) in any one of the enveloped recombinant oncolytic viruses (e.g., vaccinia virus) presented herein.

In some embodiments, the heterologous protein is a cytokine. In some embodiments, the heterologous protein is IL-15, IL-12, IL-2, IL-18, CXCL10, or CCL4, or a modified protein (e.g., a fusion protein) derived from of any of the aforementioned proteins. In some embodiments, the heterologous protein is a derivative of IL-2 that is modified to have reduced side effects. In some embodiments, the heterologous protein is modified IL-18 that lacks binding to IL18-BP. In some embodiments, the heterologous protein is a fusion protein comprising an inflammatory cytokine and a stabilizing domain. The stabilizing domain can be any suitable domain that stabilizes the inhibitory polypeptide. In some embodiments, the stabilizing domain extends the half-life of the inhibitory polypeptide in vivo. In some embodiments, the stabilizing domain is an Fc domain. In some embodiments, the stabilizing domain is an albumin domain.

In some embodiments, the Fc domain is selected from the group consisting of Fc fragments of IgG, IgA, IgD, IgE, IgM, and combinations and hybrids thereof. In some embodiments, the Fc domain is derived from a human IgG. In some embodiments, the Fc domain comprises the Fc domain of human IgG1, IgG2, IgG3, IgG4, or a combination or hybrid IgG. In some embodiments (e.g., a fusion protein derived from IL-12 or IL-2), the Fc domain has a reduced effector function as compared to corresponding wildtype Fc domain (such as at least about 30%, 40%, 50%, 60%, 70%, 80%, 85%, 90%, or 95% reduced effector function as measured by the level of antibody-dependent cellular cytotoxicity (ADCC)).

In some embodiments, the inflammatory cytokine and the stabilization domain are fused to each other via a linker, such as a peptide linker. A peptide linker may have a naturally occurring sequence, or a non-naturally occurring sequence. For example, a sequence derived from the hinge region of heavy chain only antibodies may be used as the linker. The peptide linker can be of any suitable length. In some embodiments, the peptide linker tends not to adopt a rigid three-dimensional structure, but rather provide flexibility to a polypeptide. In some embodiments, the peptide linker is a flexible linker. Exemplary flexible linkers include glycine polymers, glycine-serine polymers, glycine-alanine polymers, alanine-serine polymers, and other flexible linkers known in the art.

In some embodiments, the heterologous protein is a bacterial or a viral polypeptide. In some embodiments the heterologous protein is a tumor-associated antigen selected from carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1, WT1, NY-ESO-1, CDH17, and other tumor antigens with clinical significance.

In some embodiments, the recombinant oncolytic virus comprises two or more additional nucleotide sequences, wherein each nucleotide sequence encodes any one of the heterologous proteins or nucleic acids described herein.

### Antagonists or Inhibitors

Antagonist, as used herein, is interchangeable with inhibitor. In some embodiments, the heterologous protein is an inhibitor (i.e., an antagonist) of a target protein, wherein the target protein is an immune suppressive protein (e.g., a checkpoint inhibitor or other inhibitor of immune cell activation). In some embodiments, the target protein is an immune checkpoint protein. In some embodiments, the target protein is PD-1, PD-L1, PD-L2, CD47, CXCR4, CSF1R, LAG-3, TIM-3, HHLA2, BTLA, CD160, CD73, CTLA-4, B7-H4, TIGIT, VISTA, or 2B4. In some embodiments, the target protein is CTLA-4, PD-1, PD-L1, B7-H4, or HLA-G. In some embodiments, the target protein is an immune suppressive receptor selected from LILRB, TYRO3, AXL, or MERTK.

The antagonist inhibits the expression and/or activity of the target protein (e.g., an immune suppressive receptor or an immune checkpoint protein). In some embodiments, the antagonist inhibits expression of the target protein (e.g., mRNA or protein level) by at least about any one of 5%, 10%, 15%, 20%, 25%, 30%, 35%, 40%, 55%, 60%, 65%, 70%, 75%, 80%, 85%, 90%, 95% or more. Expression levels of a target protein can be determined using known methods in the art, including, for example, quantitative Polymerase Chain Reaction (qPCR), microarray, and RNA sequencing for determining RNA levels; and Western blots and enzyme-linked immunosorbent assays (ELISA) for determining protein levels.

In some embodiments, the antagonist inhibits activity (e.g., binding to a ligand or receptor of the target protein, or enzymatic activity) of the target protein by at least about any one of 5%, 10%, 15%, 20%, 25%, 30%, 35%, 40%, 55%, 60%, 65%, 70%, 75%, 80%, 85%, 90%, 95% or more. Binding can be assessed using known methods in the art, including, for example. Surface Plasmon Resonance (SPR) assays, and gel shift assays.

The antagonist may be of any suitable molecular modalities, including, but are not limited to, small molecule inhibitors, oligopeptides, peptidomimetics, RNAi molecules (e.g., small interfering RNAs (siRNA), short hairpin RNAs (shRNA), microRNAs (miRNA)), antisense oligonucleotides, ribozymes, proteins (e.g., antibodies, inhibitory polypeptides, fusion proteins, etc.), and gene editing systems.

i. Antibodies

In some embodiments, the antagonist inhibits binding of the target protein (e.g., an immune checkpoint protein or immune suppressive protein) to a ligand or a receptor. In some embodiments, the antagonist is an antibody that specifically binds to the target protein (e.g., CTLA-4, PD-1, PD-L1, B7-H4, HLA-G, LILRB, TYRO3, AXL, or MERTK, Folate receptor beta, etc), or an antigen-binding fragment thereof. In some embodiments, the antagonist is a polyclonal antibody. In some embodiments, the antagonist is a monoclonal antibody. In some embodiments, the antagonist is a full-length antibody, or an immunoglobulin derivative. In some embodiments, the antagonist is an antigen-binding fragment. Exemplary antigen-binding fragments include, but are not limited to, a single-chain Fv (scFv), a Fab, a Fab′, a F(ab′)₂, a Fv, a disulfide stabilized Fv fragment (dsFv), a (dsFv)₂, a single-domain antibody (e.g., VHH), a Fv-Fc fusion, a scFv-Fc fusion, a scFv-Fv fusion, a diabody, a tribody, and a tetrabody. In some embodiments, the antagonist is a scFv. In some embodiments, the antagonist is a Fab or Fab′. In some embodiments, the antagonist is a chimeric, human, partially humanized, fully humanized, or semi-synthetic antibody. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains. In some embodiments, the antagonist is a bi-specific molecule (e.g., a bi-specific antibody or bi-specific Fab, bi-specific scFv, antibody-Fc fusion protein Fv, etc) or a tri-specific molecule (e.g., a tri-specific antibody comprised of Fab, scFv, VH or Fc fusion proteins etc.).

In some embodiments, the antibody comprises one or more antibody constant regions, such as human antibody constant regions. In some embodiments, the heavy chain constant region is of an isotype selected from IgA, IgG, IgD, IgE, and IgM. In some embodiments, the human light chain constant region is of an isotype selected from κ and λ. In some embodiments, the antibody comprises an IgG constant region, such as a human IgG1, IgG2, IgG3, or IgG4 constant region. In some embodiments, when effector function is desirable, an antibody comprising a human IgG1 heavy chain constant region or a human IgG3 heavy chain constant region may be selected. In some embodiments, when effector function is not desirable, an antibody comprising a human IgG4 or IgG2 heavy chain constant region, or IgG1 heavy chain with mutations, such as N297A/Q, negatively impacting FcγR bindings may be selected. In some embodiments, the antibody comprises a human IgG4 heavy chain constant region. In some embodiments, the antibody comprises an S241P mutation in the human IgG4 constant region.

In some embodiments, the antibody comprises an Fc domain. The term “Fc region,” “Fc domain” or “Fc” refers to a C-terminal non-antigen binding region of an immunoglobulin heavy chain that contains at least a portion of the constant region. The term includes native Fc regions and variant Fc regions. In some embodiments, a human IgG heavy chain Fc region extends from Cys226 to the carboxyl-terminus of the heavy chain. However, the C-terminal lysine (Lys447) of the Fc region may or may not be present, without affecting the structure or stability of the Fc region. Unless otherwise specified herein, numbering of amino acid residues in the IgG or Fc region is according to the EU numbering system for antibodies, also called the EU index, as described in Kabat et al., Sequences of Proteins of Immunological Interest, 5th Ed. Public Health Service, National Institutes of Health, Bethesda, Md., 1991. In some embodiments, the antibody comprises a variant Fc region has at least one amino acid substitution compared to the Fc region of a wild type IgG or a wild-type antibody.

In some embodiments, the antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Antibodies that specifically bind to a target protein can be obtained using methods known in the art, such as by immunizing a non-human mammal and obtaining hybridomas therefrom, or by cloning a library of antibodies using molecular biology techniques known in the art and subsequence selection or by using phage display.

ii. Nucleic Acid Agents

In some embodiments, the heterologous nucleic acid is a nucleic acid agent that downregulates the target protein. In some embodiments, the antagonist inhibits expression (e.g., mRNA or protein expression) of the target protein. In some embodiments, the antagonist is a siRNA, a shRNA, a miRNA, an antisense oligonucleotide, or a gene editing system.

In some embodiments, the antagonist is an RNAi molecule. In some embodiments, the antagonist is a siRNA. In some embodiments, the antagonist is a shRNA. In some embodiments, the antagonist is a miRNA.

A skilled in the art may could readily design an RNAi molecule or a nucleic acid encoding an RNAi molecule to downregulate the target protein. The term “RNAi” or “RNA interference” as used herein refers to biological process in which RNA molecules inhibit gene expression or translation by specific binding to a target mRNA molecule. See for example Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zemicka-Goetz et al., International PCT Publication No. WO 01/36646; Fire, International PCT Publication No. WO 99/32619; Plaetinck et al., International PCT Publication No. WO00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). Exemplary RNAi molecules include siRNA, miRNA and shRNA.

A siRNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleotide sequence or a portion thereof. In some embodiments, the siRNA comprises one or more hairpin or asymmetric hairpin secondary structures. In some embodiments, the siRNA may be constructed in a scaffold of a naturally occurring miRNA. The siRNA molecules need not be limited to those molecules containing only RNA, but further encompasses chemically modified nucleotides and non-nucleotides.

RNAi may be designed using known methods in the art. For example, siRNA may be designed by classifying RNAi sequences, for example 1000 sequences, based on functionality, with a functional group being classified as having greater than 85% knockdown activity and a non-functional group with less than 85% knockdown activity. The distribution of base composition was calculated for entire the entire RNAi target sequence for both the functional group and the non-functional group. The ratio of base distribution of functional and non-functional group may then be used to build a score matrix for each position of RNAi sequence. For a given target sequence, the base for each position is scored, and then the log ratio of the multiplication of all the positions is taken as a final score. Using this score system, a very strong correlation may be found of the functional knockdown activity and the log ratio score. Once the target sequence is selected, it may be filtered through both fast NCBI blast and slow Smith Waterman algorithm search against the Unigene database to identify the gene-specific RNAi or siRNA. Sequences with at least one mismatch in the last 12 bases may be selected.

In some embodiments, the antagonist is an antisense oligonucleotide, e.g., antisense RNA, DNA or PNA. In some embodiments, the antagonist is a ribozyme. An “antisense” nucleic acid refers to a nucleotide sequence complementary to a “sense” nucleic acid encoding a target protein or fragment (e.g., complementary to the coding strand of a double-stranded cDNA molecule or complementary to an mRNA sequence). The antisense nucleic acid can be complementary to an entire coding strand, or to a portion thereof or a substantially identical sequence thereof. For example, the antisense oligonucleotide can be complementary to the region surrounding the translation start site of the mRNA, e.g., between the −10 and +10 regions of the target gene nucleotide sequence of interest. In some embodiments, the antisense nucleic acid molecule is antisense to a “noncoding region” of the coding strand of a nucleotide sequence. An antisense oligonucleotide can be, for example, about 7, 10, 15, 20, 25, 30, 35, 40, 45, 50, 55, 60, 65, 70, 75, 80, or more nucleotides in length. An antisense nucleic acid can be constructed using chemical synthesis or enzyme ligation reactions using standard procedures. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used). Antisense nucleic acid also can be produced biologically using an expression vector into which a nucleic acid has been subcloned in an antisense orientation.

An antisense nucleic acid is a ribozyme in some embodiments. A ribozyme having specificity for a target nucleotide sequence can include one or more sequences complementary to such a nucleotide sequence, and a sequence having a known catalytic region responsible for mRNA cleavage (e.g., U.S. Pat. No. 5,093,246 or Haselhoff and Gerlach, Nature 334: 585-591 (1988)). For example, a derivative of a Tetrahymena L-19 IVS RNA is sometimes utilized in which the nucleotide sequence of the active site is complementary to the nucleotide sequence to be cleaved in an mRNA (e.g., Cech et al. U.S. Pat. No. 4,987,071; and Cech et al. U.S. Pat. No. 5,116,742). Target mRNA sequences may be used to select a catalytic RNA having a specific ribonuclease activity from a pool of RNA molecules (e.g., Bartel & Szostak, Science 261: 1411-1418 (1993)).

In some embodiments, the antagonist is a gene-editing system, such as a CRISPR/Cas gene editing system, Transcription activator-like effector nuclease or TALEN gene editing system, Zinc-finger gene editing system, etc. In some embodiments, the antagonist is a gene-editing system that knocks-down a target protein, e.g., in a tissue-specific manner. In some embodiments, the antagonist is a gene-editing system that silences expression of the target protein.

In some embodiments, the gene-editing system comprises a guided nuclease such as an engineered (e.g., programmable or targetable) nuclease to induce gene editing of a target sequence (e.g., DNA sequence or RNA sequence) encoding the target protein. Any suitable guided nucleases can be used including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof. In some embodiments, the gene-editing system comprises a guided nuclease fused to a transcription suppressor. In some embodiments, the gene-editing system further comprises an engineered nucleic acid that hybridizes to a target sequence encoding the target protein. In some embodiments, the gene-editing system is a CRISPR-Cas system comprising a Cas nuclease (e.g., Cas9) and a guide RNA (i.e., gRNA).

### 3. Promoters for Expression of Heterologous Proteins or Nucleic Acids

The nucleotide sequences encoding heterologous proteins (e.g., sialidase) or nucleic acids described herein can be operably linked to a promoter. In some embodiments, at least a first nucleotide sequence encoding the sialidase and a second nucleotide sequence encoding an additional heterologous protein or nucleic acid are operably linked to the same promoter. In some embodiments, all of the nucleic acids encoding the heterologous proteins or nucleic acids are operably linked to the same promoter. In some embodiments, all of the nucleic acids encoding the heterologous proteins or nucleic acids are operably linked to different promoters.

In some embodiments, the promoter is a viral promoter. Viral promoters can include, but are not limited to, VV promoter, poxvirus promoter, adenovirus late promoter, Cowpox ATI promoter, or T7 promoter. The promoter may be a vaccinia virus promoter, a synthetic promoter, a promoter that directs transcription during at least the early phase of infection, a promoter that directs transcription during at least the intermediate phase of infection, a promoter that directs transcription during early/late phase of infection, or a promoter that directs transcription during at least the late phase of infection.

In some embodiments, the promoter described herein is a constitutive promoter. In some embodiments, the promoter described herein is an inducible promoter.

Promoters suitable for constitutive expression in mammalian cells include but are not limited to the cytomegalovirus (CMV) immediate early promoter (U.S. Pat. No. 5,168,062), the RSV promoter, the adenovirus major late promoter, the phosphoglycerate kinase (PGK) promoter (Adra et al., 1987, Gene 60: 65-74), the thymidine kinase (TK) promoter of herpes simplex virus (HSV)-1 and the T7 polymerase promoter (WO98/10088). Vaccinia virus promoters are particularly adapted for expression in oncolytic poxviruses. Representative examples include without limitation the vaccinia 7.5K, H5R, 11K7.5 (Erbs et al., 2008, Cancer Gene Ther. 15(1): 18-28), TK, p28, p11, pB2R, pA35R and K1L promoters, as well as synthetic promoters such as those described in Chakrabarti et al. (1997, Biotechniques 23: 1094-7; Hammond et al, 1997, J. Virol Methods 66: 135-8; and Kumar and Boyle, 1990, Virology 179: 151-8) as well as early/late chimeric promoters. Promoters suitable for oncolytic measles viruses include without limitation any promoter directing expression of measles transcription units (Brandler and Tangy, 2008, CIMID 31: 271).

Inducible promoters belong to the category of regulated promoters. The inducible promoter can be induced by one or more conditions, such as a physical condition, microenvironment of the host cell, or the physiological state of the host cell, an inducer (i.e., an inducing agent), or a combination thereof.

Appropriate promoters for expression can be tested in vitro (e.g. in a suitable cultured cell line) or in vivo (e.g. in a suitable animal model or in the subject). When the encoded immune checkpoint modulator(s) comprise(s) an antibody and especially a mAb, examples of suitable promoters for expressing the heavy component of said immune checkpoint modulator comprise CMV, SV and vaccinia virus pH5R, F17R and p11K7.5 promoters; examples of suitable promoters for expressing the light component of said immune checkpoint modulator comprise PGK, beta-actin and vaccinia virus p7.5K, F17R and pA35R promoters.

Promoters can be replaced by stronger or weaker promoters, where replacement results in a change in the attenuation of the virus. As used herein, replacement of a promoter with a stronger promoter refers to removing a promoter from a genome and replacing it with a promoter that effects an increased the level of transcription initiation relative to the promoter that is replaced. Typically, a stronger promoter has an improved ability to bind polymerase complexes relative to the promoter that is replaced. As a result, an open reading frame that is operably linked to the stronger promoter has a higher level of gene expression. Similarly, replacement of a promoter with a weaker promoter refers to removing a promoter from a genome and replacing it with a promoter that decreases the level of transcription initiation relative to the promoter that is replaced. Typically, a weaker promoter has a lessened ability to bind polymerase complexes relative to the promoter that is replaced. As a result, an open reading frame that is operably linked to the weaker promoter has a lower level of gene expression. The viruses can exhibit differences in characteristics, such as attenuation, as a result of using a stronger promoter versus a weaker promoter. For example, in vaccinia virus, synthetic early/late and late promoters are relatively strong promoters, whereas vaccinia synthetic early. P7.5k early/late, P7.5k early, and P28 late promoters are relatively weaker promoters (see e.g., Chakrabarti et al. (1997) BioTechniques 23 (6) 1094-1097). In some embodiments, the promoter described herein is a weak promoter. In some embodiments, the promoter described herein is a strong promoter.

In some embodiments, the promoter is a viral promoter of the oncolytic virus. In some embodiments, the promoter is an early viral promoter, a late viral promoter, an intermediate viral promoter, or an early/late viral promoter. In some embodiments, the promoter is a synthetic viral promoter, such as a synthetic early, early/late, or late viral promoter.

In some embodiments, the promoter is a vaccinia virus promoter. Exemplary vaccinia viral promoters for use in the present invention can include, but are not limited to, P_{7.5k}, P₁₁ₖ, P_{SE}, P_{SEL}, P_{SL}, H5R, TK, P28, C11R G8R, F17R, 13L, 18R, A1L, A2L, A3L, H1L, H3L, H5L, H6R, H8R, DI R, D4R, D5R, D9R, D11L, D12L, D13L, M1L, N2L, P4b or K1 promoters.

Exemplary vaccinia early, intermediate and late stage promoters include, for example, vaccinia P_{7.5k} early/late promoter, vaccinia P_{EL} early/late promoter, vaccinia P₁₃ early promoter, vaccinia P₁₁ₖ late promoter and vaccinia promoters listed elsewhere herein. Exemplary synthetic promoters include, for example, P_{SE} synthetic early promoter, P_{SEL} synthetic early/late promoter. P_{SL} synthetic late promoter, vaccinia synthetic promoters listed elsewhere herein (Patel et al., Proc. Natl. Acad. Sci. USA 85: 9431-9435 (1988); Davison and Moss, J Mol Biol 210: 749-769 (1989): Davison et al., Nucleic Acids Res. 18: 4285-4286 (1990); Chakrabarti et al., BioTechniques 23: 1094-1097 (1997)). Combinations of different promoters can be used to express different gene products in the same virus or two different viruses.

In some embodiments, the promoter directs transcription during at least the late phase of infection (such as F17R promoter, shown in SEQ ID NO: 61) is employed. In some embodiments, the late promoter is selected from the group consisting of F17R, I2L late promoter, L4R late promoter, P_{7.5k} early/late promoter, P_{EL} early/late promoter, P₁₁ₖ late promoter, P_{SEL} synthetic early/late promoter, and P_{SL} synthetic late promoter. The late vaccinia viral promoter F17R is only activated after VV infection in tumor cells, thus tumor selective expression of the heterologous protein or nucleic acid from VV will be further enhanced by the use of F17R promoter. In some embodiments, the late expression of a heterologous protein or nucleic acid of the present invention allows for sufficient viral replication before T-cell activation and mediated tumor lysis.

In some embodiments, the promoter is a hybrid promoter. In some embodiments, the hybrid promoter is a synthetic early/late viral promoter. In some embodiments, the promoter comprises a partial or complete nucleotide sequence of a human promoter. In some embodiments, the human promoter is a tissue or tumor-specific promoter. In some embodiments, the tumor-specific promoter can be a promoter that drives enhanced expression in tumor cells, or that drives expression specifically in tumor cells (e.g., a promoter that drives expression of a tumor tumor-associated antigen (TAA) or a tumor-specific antigen (TSA)). In some embodiments, the hybrid promoter comprises a partial or complete nucleotide sequence of a tissue or tumor-specific promoter and a nucleotide sequence (e.g., a CMV promoter sequence) that increase the strength of the hybrid promoter relative to the tissue- or tumor-specific promoter. Non-limiting examples of hybrid promoters comprising tissue- or tumor-specific promoters include hTERT and CMV hybrid promoters or AFP and CMV hybrid promoters.

### C. Engineered Immune Cells

In some aspects of the present application, provided are engineered immune cells expressing a chimeric receptor. In some embodiments, the immune cell is selected from the group consisting of a cytotoxic T cell, a helper T cell, a suppressor T cell, an NK cell, and an NK-T cell. In some embodiments, the engineered immune cell is an NK cell. In some embodiments, the engineered immune cell is a T cell. In some embodiments, the engineered immune cell is an NKT cell.

Some embodiments of the engineered immune cells described herein comprise one or more engineered chimeric receptors, which are capable of activating an immune cell (e.g., T cell or NK cell) directly or indirectly against a tumor cell expressing a target antigen. Exemplary engineered receptors include, but are not limited to, chimeric antigen receptor (CAR), engineered T cell receptor, and TCR fusion protein.

In some embodiments, the engineered immune cells are autologous cells (cells obtained from the subject to be treated). In some embodiments, the engineered immune cells are allogeneic cells, which can include a variety of readily isolable and/or commercially available cells/cell lines.

### Chimeric Antigen Receptor (CAR)

“Chimeric antigen receptor” or “CAR” as used herein refers to an engineered receptor that can be used to graft one or more target-binding specificities onto an immune cell, such as T cells or NK cells. In some embodiments, the chimeric antigen receptor comprises an extracellular target binding domain, a transmembrane domain, and an intracellular signaling domain of a T cell receptor and/or other receptors.

Some embodiments of the engineered immune cells described herein comprise a chimeric antigen receptor (CAR). In some embodiments, the CAR comprises an antigen-binding moiety and an effector protein or fragment thereof comprising a primary immune cell signaling molecule or a primary immune cell signaling domain that activates the immune cell expressing the CAR directly or indirectly. In some embodiments, the CAR comprises an antigen-binding domain, a transmembrane domain, and an intracellular signaling domain. Also provided an engineered immune cells (e.g., T cell or NK cell) comprising the CAR. The antigen-binding moiety and the effector protein or fragment thereof may be present in one or more polypeptide chains. Exemplary CAR constructs have been described, for example, in U.S. Pat. No. 9,765,342B2, WO2002/077029, and WO2015/142675, which are hereby incorporated by reference. Any one of the known CAR constructs may be used in the present application.

In some embodiments, the primary immune cell signaling molecule or primary immune cell signaling domain comprises an intracellular domain of a molecule selected from the group consisting of CD3ζ, FcRγ, FcRβ, CD3γ, CD3δ, CD3ε, CD5, CD22, CD79a, CD79b, and CD66d. In some embodiments, the intracellular signaling domain consists of or consists essentially of a primary immune cell signaling domain. In some embodiments, the intracellular signaling domain comprises an intracellular signaling domain of CD3ζ. In some embodiments, the CAR further comprises a costimulatory molecule or fragment thereof. In some embodiments, the costimulatory molecule or fragment thereof is derived from a molecule selected from the group consisting of CD27, CD28, 4-1BB, OX40. CD30, CD40, PD-1, ICOS, LFA-1, CD2, CD7, LIGHT, NKG2C, B7-H3, and a ligand that specifically binds CD83. In some embodiments, the intracellular signaling domain further comprises a co-stimulatory domain comprising a CD28 intracellular signaling sequence. In some embodiments, the intracellular signaling domain comprises a CD28 intracellular signaling sequence and an intracellular signaling sequence of CD3ζ.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the CD28, CD3ε, CD3ζ, CD45. CD4, CD5, CD8, CD9, CD16, CD22, CD33, CD37, CD64. CD80, CD86, CD134, CD137, or CD154. In some embodiments, the CAR is a CD-19 CAR comprising including CD19 scFv from clone FMC63 (Nicholson I C, et al. Mol Immunol. 1997), a CH2-CH3 spacer, a CD28-TM, 41BB, and CD3ζ. In some embodiments, the transmembrane domain may be synthetic, in which case it may comprise predominantly hydrophobic residues such as leucine and valine. In some embodiments, a triplet of phenylalanine, tryptophan and valine may be found at each end of a synthetic transmembrane domain. In some embodiments, a short oligo- or polypeptide linker, having a length of, for example, between about 2 and about 10 (such as about any of 2, 3, 4, 5, 6, 7, 8, 9, or 10) amino acids in length may form the linkage between the transmembrane domain and the intracellular signaling domain. In some embodiments, the linker is a glycine-serine doublet.

In some embodiments, the transmembrane domain that is naturally associated with one of the sequences in the intracellular domain is used (e.g., if an intracellular domain comprises a CD28 co-stimulatory sequence, the transmembrane domain is derived from the CD28 transmembrane domain). In some embodiments, the transmembrane domain can be selected or modified by amino acid substitution to avoid binding of such domains to the transmembrane domains of the same or different surface membrane proteins to minimize interactions with other members of the receptor complex.

The intracellular signaling domain of the CAR is responsible for activation of at least one of the normal effector functions of the immune cell in which the CAR has been placed in. Effector function of a T cell, for example, may be cytolytic activity or helper activity including the secretion of cytokines. Thus, the term “intracellular signaling domain” refers to the portion of a protein, which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term “intracellular signaling sequence” is thus meant to include any truncated portion of the intracellular signaling domain sufficient to transduce the effector function signal.

Examples of intracellular signaling domains for use in the CAR of the present application include the cytoplasmic sequences of the TCR and co-receptors that act in concert to initiate signal transduction following antigen receptor engagement, as well as any derivative or variant of these sequences and any synthetic sequence that has the same functional capability.

It is known that signals generated through the TCR alone may be insufficient for full activation of the T cell and that a secondary or co-stimulatory signal may also be required. Thus, T cell activation can be said to be mediated by two distinct classes of intracellular signaling sequence: those that initiate antigen-dependent primary activation through the TCR (primary signaling sequences) and those that act in an antigen-independent manner to provide a secondary or co-stimulatory signal (co-stimulatory signaling sequences).

Primary signaling sequences regulate primary activation of the TCR complex either in a stimulatory way, or in an inhibitory way. Primary signaling sequences that act in a stimulatory manner may contain signaling motifs, which are known as immunoreceptor tyrosine-based activation motifs or ITAMs. The CAR constructs in some embodiments comprise one or more ITAMs. Examples of ITAM containing primary signaling sequences that are of particular use in the invention include those derived from CD3ζ, FcRγ, FcRβ, CD3γ, CD3δ, CD3ε, CD5, CD22, CD79a, CD79b, and CD66d.

In some embodiments, the CAR comprises a primary signaling sequence derived from CD3ζ. For example, the intracellular signaling domain of the CAR can comprise the CD3ζ intracellular signaling sequence by itself or combined with any other desired intracellular signaling sequence(s) useful in the context of the CAR described herein. For example, the intracellular domain of the CAR can comprise a CD3ζ intracellular signaling sequence and a costimulatory signaling sequence. The costimulatory signaling sequence can be a portion of the intracellular domain of a costimulatory molecule including, for example, CD27, CD28, 4-1BB (CD137), OX40. CD30, CD40, ICOS, lymphocyte function-associated antigen-1 (LFA-1), CD2, CD7, LIGHT, NKG2C, B7-H3, a ligand that specifically binds with CD83, and the like.

In some embodiments, the intracellular signaling domain of the CAR comprises the intracellular signaling sequence of CD3ζ and the intracellular signaling sequence of CD28. In some embodiments, the intracellular signaling domain of the CAR comprises the intracellular signaling sequence of CD3ζ and the intracellular signaling sequence of 4-1BB. In some embodiments, the intracellular signaling domain of the CAR comprises the intracellular signaling sequence of CD3ζ and the intracellular signaling sequences of CD28 and 4-1BB.

In some embodiments, the antigen binding moiety comprises an scFv or a Fab. In some embodiments, the antigen binding moiety is targeted to a tumor-associated or tumor-specific antigen, such as, without limitation: carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1. WT1, NY-ESO-1. CDH17, and other tumor antigens with clinical significance. In some embodiments, the antigen binding moiety is directed to a foreign antigen that is delivered to tumor cells (e.g., by a recombinant oncolytic virus). In some embodiments, the foreign antigen is DAS181 or its derivatives (e.g. a transmembrane form of the sialidase domain of DAS181 without anchoring domain, as described in Examples 11 and 15).

In some embodiments, the sialidase domain (e.g., a non-human sialidase or a derivative thereof, such as a sialidase domain of DAS181) delivered to tumor cells using an oncolytic virus functions both to remove sialic acid from the surface of tumor cells and as a foreign antigen that enhances immune cell-mediated killing of tumor cells. In some embodiments, the sialidase-armed oncolytic virus is combined with an engineered immune cell that specifically targets the sialidase domain (e.g., DAS181), thereby enhancing killing of tumor cells infected by the oncolytic virus.

Also provided herein are engineered immune cells (such as lymphocytes, e.g., T cells, NK cells) expressing any one of the CARs described herein. Also provided is a method of producing an engineered immune cell expressing any one of the CARs described herein, the method comprising introducing a vector comprising a nucleic acid encoding the CAR into the immune cell. In some embodiments, introducing the vector into the immune cell comprises transducing the immune cell with the vector. In some embodiments, introducing the vector into the immune cell comprises transfecting the immune cell with the vector. Transduction or transfection of the vector into the immune cell can be carried about using any method known in the art.

### Engineered T Cell Receptor

In some embodiments, the chimeric receptor is a T cell receptor. In some embodiments, wherein the engineered immune cell is a T cell, the T cell receptor is an endogenous T cell receptor. In some embodiments, the engineered immune cell with the TCR is pre-selected. In some embodiments, the T cell receptor is a recombinant TCR. In some embodiments, the TCR is specific for a tumor antigen. In some embodiments, the tumor antigen is selected from the group consisting of carcinoembryonic antigen, alphafetoprotein, MUC16, survivin, glypican-3, B7 family members, LILRB, CD19, BCMA. NY-ESO-1, CD20, CD22, CD33, CD38, CEA, EGFR (such as EGFRvIII), GD2, HER2, IGF1R, mesothelin, PSMA, ROR1, WT1, NY-ESO-1, Fibulin-3, CDH17, and other tumor antigens with clinical significance. Many TCRs specific for tumor antigens (including tumor-associated antigens) have been described, including, for example, NY-ESO-1 cancer-testis antigen, the p53 tumor suppressor antigens, TCRs for tumor antigens in melanoma (e.g., MARTI, gp 100), leukemia (e.g., WT1, minor histocompatibility antigens), and breast cancer (HER2, NY-BR1, for example). Any of the TCRs known in the art may be used in the present application. In some embodiments, the TCR has an enhanced affinity to the tumor antigen. Exemplary TCRs and methods for introducing the TCRs to immune cells have been described, for example, in U.S. Pat. No. 5,830,755, and Kessels et al. Immunotherapy through TCR gene transfer. Nat. Immunol. 2, 957-961 (2001). In some embodiments, the engineered immune cell is a TCR-T cell.

### TCR Fusion Protein (TFP)

In some embodiments, the engineered immune cell comprises a TCR fusion protein (TFP). “TCR fusion protein” or “TFP” as used herein refers to an engineered receptor comprising an extracellular target-binding domain fused to a subunit of the TCR-CD3 complex or a portion thereof, including TCRα chain, TCRβ chain, TCRγ chain, TCRδ chain, CD3ε, CD3δ, or CD3γ. The subunit of the TCR-CD3 complex or portion thereof comprise a transmembrane domain and at least a portion of the intracellular domain of the naturally occurring TCR-CD3 subunit. The TFP comprises the extracellular domain of the TCR-CD3 subunit or a portion thereof.

Exemplary TFP constructs comprising an antibody fragment as the target-binding moiety have been described, for example, in WO2016187349 and WO2018098365, which are hereby incorporated by reference.

### Targeting Engineered Immune Cells to Tumor-Associated Antigens.

Engineered immune cells can be targeted to any of a variety of tumor-associated antigens (TAAs) or immune cell receptors, which may include without limitation. EGFRvIII, PD-L1, EpCAM, carcinoembryonic antigen, alphafetoprotein, MUC16, survivin, glypican-3, B7 family members, LILRB, CD-19, etc. In some embodiments, engineered immune cells can be used to deliver recombinant oncolytic viruses provided herein to cancer cells expressing these or any number of known cancer antigens. In some embodiments, engineered immune cells can be targeted to a foreign antigen (e.g., a bacterial peptide or a bacterial sialidase) that is delivered to tumor cells using a recombinant oncolytic virus. Engineered immune cells can also be targeted to a variety of immune cells expressing various immune cell antigens, such as, without limitation: carcinoembryonic antigen, alphafetoprotein, MUC16, survivin, glypican-3, B7 family members, LILRB, CD19, BCMA, NY-ESO-1, CD20, CD22, CD33, CD38, CEA, EGFR (such as EGFRvIII), GD2, HER2, IGF1R, mesothelin, PSMA, ROR1, WT1, NY-ESO-1, Fibulin-3, CDH17, and other tumor antigens with clinical significance

Engineered immune cells can be delivered to the patient in any way known in the art for delivering engineered immune cells (e.g., CART-T, CAR-NK, or CAR-NKT cells). In some embodiments, sialidase expressed on the surface of or secreted by sialidase expressing engineered immune cells may remove sialic acids from sialoglycans expressed on immune cells and/or tumor cells. The removal of the sialic acid on tumor cell can further activate the Dendritic cells, macrophages, T and NK cell that are no longer engaged with the inhibitory signals of the tumor cells via Siglecs/sialic acid axis and other Selectins interactions. These interactions can further enhance immune activation against cancer and change the tumor microenvironment (TME). With respect to tumor cells, as they are desialylated, they become exposed to attack by activated NK cells and T cells and other immune cells, resulting in reduction in tumor size.

In some embodiments, the engineered immune cells set forth herein can be engineered to express sialidase, such as, without limitation, sialidase domain of DAS181 fused to a transmembrane domain, on the immune cell surface membrane, such that the sialidase is membrane bound. In some embodiments, the sialidase can be fused to a e.g. transmembrane domain.

Without being bound by any theory or hypothesis, membrane bound sialidases will not be freely circulating and will only come into contact with the target cells of the CAR-T, namely tumor cells expressing the antigens that the CAR-T receptor targets. For example, if the CAR-T is a CD-19 receptor or mAb to CD19 expressing CAR-T, then the membrane bound sialidases will primarily only come into contact with tumor cells that express CD-19. In this way, the sialidases will not desialylate non-targeted cells, such as erythrocytes, but will instead eliminate sialic acid primarily only from tumor cells. The CAR-Ts set forth herein can also be engineered so that they express secreted sialidase, such as, without limitation, secreted form of DAS181.

### D. Oncolytic Virus and Carrier Cell

In some embodiments, the present application provides a carrier cell comprising any one of the recombinant oncolytic viruses described herein. In some embodiments, the carrier cell is an immune cell or a stem cell (e.g., a mesenchymal stem cell). In some embodiments, the immune cell is an engineered immune cell, such as any of the engineered immune cells described in subsection C above.

The population of carrier cells (e.g., immune cells or stem cells) can be infected with the oncolytic virus. The sialidase containing virus may be administered in any appropriate physiologically acceptable cell carrier. The multiplicity of infection will generally be in the range of about 0.001 to 1000, e.g., in the range of 0.001 to 100. The virus-containing cells may be administered one or more times. Alternatively, viral DNA may be used to transfect the effector cells, employing liposomes, general transfection methods that are well known in the art (such as calcium phosphate precipitation and electroporation), etc. Due to the high efficiency of transfection of viruses, one can achieve a high level of modified cells. In some embodiments, the engineered immune cell comprising the recombinant oncolytic virus can be prepared by incubating the immune cell with the virus for a period of time. In some embodiments, the immune cell can be incubated with the virus for a time sufficient for infection of the cell with virus, and expression of the one or more virally encoded heterologous protein(s) (e.g., sialidase and/or any of the immunomodulatory proteins described herein).

The population of carrier cells (e.g., immune cells or stem cells) comprising the recombinant oncolytic virus may be injected into the recipient. Determination of suitability of administering cells of the invention will depend, inter alia, on assessable clinical parameters such as serological indications and histological examination of tissue biopsies. Generally, a pharmaceutical composition is administered. Routes of administration include systemic injection, e.g. intravascular, subcutaneous, or intraperitoneal injection, intratumor injection, etc.

### III. Methods of Treatment

The present application provides methods of treating a cancer (e.g., solid tumor) in an individual in need thereof, comprising administering to the individual an effective amount of any one of the recombinant oncolytic viruses, pharmaceutical compositions, or engineered immune cells described herein.

In some embodiments, there is provided a method of treating a cancer in an individual in need thereof, comprising administering to the individual an effective amount of a recombinant oncolytic virus comprising a nucleotide sequence encoding a sialidase, wherein the nucleotide sequence encoding the heterologous protein is operably linked to a promoter. In some embodiments, the oncolytic virus is a vaccinia virus, reovirus, Seneca Valley virus (SVV), vesicular stomatitis virus (VSV), Newcastle disease virus (NDV), herpes simplex virus (HSV), morbillivirus virus, retrovirus, influenza virus, Sinbis virus, poxvirus, measles virus, cytomegalovirus (CMV), lentivirus, adenovirus, or coxsackievirus, or a derivative thereof. In some embodiments, the oncolytic virus is Talimogene Laherparepvec. In some embodiments, the oncolytic virus is a reovirus. In some embodiments, the oncolytic virus is an adenovirus (e.g., an adenovirus having an E1ACR2 deletion).

In some embodiments, the oncolytic virus is a poxvirus. In some embodiments, the poxvirus is a vaccinia virus. In some embodiments, the vaccinia virus is of a strain such as Dryvax, Lister, M63, LIVP, Tian Tan, Modified Vaccinia Ankara, New York City Board of Health (NYCBOH), Dairen, Ikeda, LC16M8, Tashkent, IHD-J, Brighton, Dairen I, Connaught, Elstree, Wyeth, Copenhagen. Western Reserve, Elstree, CL, Lederle-Chorioallantoic, or AS, or a derivative thereof. In some embodiments, the virus is vaccinia virus Western Reserve.

In some embodiments, the recombinant oncolytic virus is administered via a carrier cell (e.g., an immune cell or stem cell, such as a mesenchymal stem cell). In some embodiments, the recombinant oncolytic virus is administered as a naked virus. In some embodiments, the recombinant oncolytic virus is administered via intratumoral injection.

In some embodiments, the method comprises administering a recombinant oncolytic virus comprising a nucleotide sequence encoding a sialidase, wherein the nucleotide sequence encoding the heterologous protein is operably linked to a promoter, and wherein the recombinant oncolytic virus comprises one or more mutations that reduce immunogenicity of the virus compared to a corresponding wild-type strain. In some embodiments, the virus is a vaccinia virus (e.g., a vaccinia virus Western Reserve), and the one or more mutations are in one or more proteins selected from the group consisting of A27L, H3L, D8L and L1R or other immunogenic proteins (e.g., A14, A17, A13, L1, H3, D8, A33, B5, A56, F13, A28, and A27). In some embodiments, the one or more mutations are in one or more proteins selected from the group consisting of A27L, H3L, D8L and L1R In some embodiments, the virus comprises one or more proteins selected from the group consisting of: (a) a variant vaccinia virus (VV) H3L protein that comprises an amino acid sequence having at least 90% amino acid sequence identity to any one of SEQ ID NOS: 66-69; (b) a variant vaccinia virus (VV) D8L protein that comprises an amino acid sequence having at least 90% amino acid sequence identity to any one of SEQ ID NOS: 70-72 or 85; (c) a variant vaccinia virus (VV) A27L protein that comprises an amino acid sequence having at least 90% amino acid sequence identity to SEQ ID NO: 73; and (d) a variant vaccinia virus (VV) L1R protein that comprises an amino acid sequence having at least 90% amino acid sequence identity to SEQ ID NO: 74.

In some embodiments, the method comprises administering a recombinant oncolytic virus comprising a nucleotide sequence encoding a sialidase, wherein the sialidase is operably linked to a promoter. In some embodiments, the sialidase is a Neu5Ac alpha(2,6)-Gal sialidase or a Neu5Ac alpha(2,3)-Gal sialidase. In some embodiments, the sialidase is a bacterial sialidase (e.g., a Clostridium perfringens sialidase, Actinomyces viscosus sialidase, and Arthrobacter ureafaciens sialidase, Salmonella typhimurium sialidase or Vibrio cholera sialidase) or a derivative thereof.

In some embodiments, the sialidase comprises all or a portion of the amino acid sequence of a large bacterial sialidase or can comprise amino acid sequences having at least 80%, at least 85%, at least 90%, at least 95%, at least 98%, at least 99%, or 100% sequence identity to all or a portion of the amino acid sequence of a large bacterial sialidase. In some embodiments, the sialidase domain comprises SEQ ID NO: 2 or 27, or a sialidase sequence having at least 80%, at least 85%, at least 90%, at least 95%, at least 98%, at least 99%, or 100% sequence identity to SEQ ID NO: 12. In some embodiments, a sialidase domain comprises the catalytic domain of the Actinomyces viscosus sialidase extending from amino acids 274-666 of SEQ ID NO: 26, having at least 80%, at least 85%, at least 90%, at least 95%, at least 98%, at least 99%, or 100% sequence identity to amino acids 274-666 of SEQ ID NO: 26.

In some embodiments, the sialidase is a human sialidase (e.g., NEU1, NEU2, NEU3, or NEU4), or a derivative thereof.

In some embodiments, the sialidase is a naturally occurring sialidase. In some embodiments, the sialidase is a fusion protein comprising a sialidase catalytic domain.

In some embodiments, the sialidase comprises an anchoring moiety. In some embodiments, the sialidase is a fusion protein comprising a sialidase catalytic domain fused to an anchoring domain. In some embodiments, the anchoring domain is positively charged at physiologic pH. In some embodiments, the anchoring domain is a glycosaminoglycan (GAG)-binding domain.

In some embodiments, the sialidase comprises an amino acid sequence having at least about 80% (e.g., at least about 85%, 90%, or 95%) sequence identity to an amino acid sequence selected from the group consisting of SEQ ID NOs: 1-33 or 53-54. In some embodiments, the sialidase comprises an amino acid sequence having at least about 80% (e.g., at least about 85%, 90%, or 95%) sequence identity to the amino acid sequence of SEQ ID NO: 2. In some embodiments, the sialidase is DAS181.

In some embodiments, the nucleotide sequence encoding the sialidase including a secretory peptide (e.g., a signal sequence or signal peptide operably linked to the sialidase). In some embodiments, the secretion sequence comprises the amino acid sequence of SEQ ID NO: 40. In some embodiments, the sialidase comprises a transmembrane domain. In some embodiments, the anchoring domain or the transmembrane domain is located at the carboxy terminus of the sialidase.

In some embodiments, there is provided a method of treating a cancer in an individual in need thereof, comprising administering to the individual an effective amount of a carrier cell (e.g., an immune cell or a stem cell such as a mesenchymal stem cell) comprising a recombinant oncolytic virus, wherein the recombinant oncolytic virus comprises a nucleotide sequence encoding a sialidase. In some embodiments, the sialidase is a bacterial sialidase (e.g., a Clostridium perfringens sialidase, Actinomyces viscosus sialidase, and Arthrobacter ureafaciens sialidase, Salmonella typhimurium sialidase or Vibrio cholera sialidase) or a derivative thereof. In some embodiments, the sialidase is derived from a Actinomyces viscosus sialidase. In some embodiments, the sialidase is DAS181 or a derivative thereof. In some embodiments, the nucleotide sequence encoding the sialidase further encodes a secretion sequence (e.g., a secretory sequence or secretory peptide) operably linked to the sialidase. In some embodiments, the molecule comprises a sialidase linked to a transmembrane domain. In some embodiments, the carrier cell is an engineered immune cell. In some embodiments, the engineered immune cell expresses a chimeric receptor, such as a CAR. In some embodiments, the chimeric receptor specifically recognizes a tumor associated antigen and encoded other molecule that can stimulate antitumor immune response and tumor killing functions.

In some embodiments, there is provided a method of treating a cancer in an individual in need thereof, comprising administering to the individual: (a) an effective amount of a recombinant oncolytic virus comprising a nucleotide sequence encoding a sialidase, or an effective amount of carrier cells comprising the recombinant oncolytic virus: and (b) an effective amount of engineered immune cells expressing a chimeric receptor. In some embodiments, the sialidase is a bacterial sialidase (e.g., Clostridium perfringens sialidase, Actinomyces viscosus sialidase, and Arthrobacter ureafaciens sialidase. Salmonella typhimurium sialidase or Vibrio cholera sialidase). In some embodiments, the sialidase comprises an anchoring domain. In some embodiments, the anchoring domain is a GAG-binding protein domain, e.g., the epithelial anchoring domain of human amphiregulin. In some embodiments, the anchoring domain is positively charged at physiologic pH. In some embodiments, the anchoring domain is a GPI linker. In some embodiments, the sialidase is DAS181. In some embodiments, the sialidase comprises a transmembrane domain. In some embodiments, the chimeric receptor recognizes a tumor-associated antigen or tumor-specific antigen. In some embodiments, the engineered immune cells are T cells or NK cells. In some embodiments, the chimeric receptor is a CAR.

In some embodiments, there is provided a method of treating a cancer in an individual in need thereof, comprising administering to the individual: (a) an effective amount of a recombinant oncolytic virus comprising a nucleotide sequence encoding a sialidase, or an effective amount of carrier cells comprising the recombinant oncolytic virus, and (b) an effective amount of engineered immune cells expressing a chimeric receptor specifically recognizing the sialidase. In some embodiments, the sialidase is a bacterial sialidase (e.g., Clostridium perfringens sialidase, Actinomyces viscosus sialidase, and Arthrobacter ureafaciens sialidase, Salmonella typhimurium sialidase or Vibrio cholera sialidase). In some embodiments, the sialidase comprises an anchoring domain. In some embodiments, the anchoring domain is a GAG-binding protein domain, e.g., the epithelial anchoring domain of human amphiregulin. In some embodiments, the anchoring domain is positively charged at physiologic pH. In some embodiments, the anchoring domain is a GPI linker. In some embodiments, the sialidase is DAS181. In some embodiments, the sialidase comprises a transmembrane domain. In some embodiments, the chimeric receptor specifically recognizes the sialidase (e.g., DAS181) and is not cross-reactive with human native amphiregulin or any other human antigen. In some embodiments, the engineered immune cells are T cells or NK cells. In some embodiments, the chimeric receptor is a CAR.

In some embodiments, there is provided a method of delivering a foreign antigen to cancer cells in an individual, comprising administering to the individual an effective amount of a recombinant oncolytic virus comprising a nucleotide sequence encoding a foreign antigen. In some embodiments, the foreign antigen is a bacterial protein. In some embodiments, the foreign antigen is a sialidase. In some embodiments, the foreign antigen is a bacterial sialidase (e.g., Clostridium perfringens sialidase, Actinomyces viscosus sialidase, and Arthrobacter ureafaciens sialidase. Salmonella typhimurium sialidase or Vibrio cholera sialidase). In some embodiments, the sialidase is a sialidase catalytic domain of DAS181. In some embodiments, the method further comprises administering engineered immune cells. In some embodiments, the engineered immune cells express a chimeric receptor specifically recognizing the foreign antigen or any relevant tumor associated antigen or tumor specific antigen of the tumor being treated.

In some embodiments, there is provided a method of treating a cancer in an individual in need thereof comprising administering to the individual: (a) an effective amount of a recombinant oncolytic virus comprising a nucleotide sequence encoding a foreign antigen; and (b) an effective amount of engineered immune cells expressing a chimeric receptor specifically recognizing said foreign antigen.

In some embodiments, there is provided a method of treating cancer, comprising administering to the individual: (a) an effective amount of a recombinant oncolytic viruses comprising a nucleotide sequence encoding a sialidase, and (b) an effective amount of an immunotherapy.

In some embodiments, there is provided a method of sensitizing a tumor in an individual to an immunotherapy, comprising administering to the individual an effective amount of any one of the recombinant oncolytic viruses comprising a nucleotide sequence encoding a sialidase described above. In some embodiments, the sialidase is a bacterial sialidase (e.g., a Clostridium perfringens sialidase, Actinomyces viscosus sialidase, and Arthrobacter ureafaciens sialidase, Salmonella typhimurium sialidase or Vibrio cholera sialidase) or a derivative thereof. In some embodiments, the sialidase is derived from a Actinomyces viscosus sialidase. In some embodiments, the sialidase is DAS181. In some embodiments, the nucleotide sequence encoding the sialidase further encodes a secretion sequence (e.g., a secretory signal peptide) operably linked to the sialidase. In some embodiments, the sialidase further comprises a transmembrane domain. In some embodiments, the method further comprises administering an effective amount of the immunotherapy to the individual. In some embodiments, the immunotherapy is a multi-specific immune cell engager (e.g., a bispecific molecule), a cell therapy, a cancer vaccine (e.g., a dendritic cell (DC) cancer vaccine), a cytokine (e.g., IL-15, IL-12, modified IL-2 having no or reduced binding to the alpha receptor, modified IL-18 with no or reduced binding to IL-18 BP, CXCL10, or CCL4), an immune checkpoint inhibitor (e.g., an inhibitor of CTLA-4, PD-1, PD-L1, B7-H4, or HLA), a master switch anti-LILRB, and bispecific anti-LILRB-4-1BB, Anti-FAP-CD3, a PI3Kgamma inhibitor, a TLR9 ligand, an HDAC inhibitor, a LILRB2 inhibitor, a MARCO inhibitor, etc.

In some embodiments, the immunotherapy is a cell therapy. A cell therapy comprises administering an effective amount of live cells (e.g., immune cells) to the individual. In non-limiting examples, the immune cells can be T-cells, natural killer (NK) cells, natural killer T (NKT) cells, dendritic cells (DC), cytokine-induced killer (CIK) cells, cytokine-induced natural killer (CINK) cells, lymphokine-activated killer (LAK) cells, tumor-infiltrating lymphocytes (TILs), macrophages, or combinations thereof. In some embodiments, the cell therapy can comprise administering a developmental intermediate (e.g., a progenitor) of any one of the immune cell types described herein. In some embodiments, the cell therapy agents can comprise indiscrete heterogeneous cell populations, such as expanded PBMCs that have proliferated and acquired killing activity on ex vivo culture. Suitable cell therapies have been described, for example, in Hayes, C. “Cellular immunotherapies for cancer.” Ir J Med Sci (2020). In some embodiments, the cell therapy comprises PBMC cells that have been stimulated with various cytokine and antibody combinations to activate effector T cells (CD3, CD38 and IL-2) or, in some cases, T cells and NK cells (CD3, CD28, IL-15 and IL-21). Examples 3, 5, and 6 provide results demonstrating enhanced tumor cell killing using a combination of a recombinant oncolytic virus encoding a sialidase and a cell therapy.

In some embodiments, the cell therapy comprises administering to the individual an effective amount of immune cells, wherein the immune cells have been primed to respond to a tumor antigen, e.g, by exposure to the antigen either in vivo or ex vivo.

In some embodiments, the cell therapy comprises administering to the individual an effective amount of engineered immune cells expressing a chimeric receptor, such as any one of the chimeric receptors described in the “Engineered immune cells” section above. In some embodiments, the cell therapy comprises administering an effective amount of CAR-T, CAR-NK, or CAR-NKT cells. In some embodiments, the chimeric receptor recognizes an antigen expressed by tumor cells, such as an endogenous tumor-associated or tumor-specific antigen. In non-limiting examples, the chimeric receptor can recognize tumor antigens such as carcinoembryonic antigen, alphafetoprotein, MUC16, survivin, glypican-3, B7 family members, LILRB, CD19. BCMA, NY-ESO-1, CD20, CD22. CD33, CD38, CEA, EGFR (such as EGFRvIII), GD2, HER2, IGF1R, mesothelin, PSMA, ROR1, WT1, NY-ESO-1, Fibulin-3, CDH17, and other tumor antigens with clinical significance. In some embodiments, the chimeric receptor recognizes a foreign antigen expressed by tumor cells, such as a heterologous protein delivered to the tumor cells via any one of the recombinant oncolytic viruses provided herein. In some embodiments, the foreign antigen delivered by the recombinant oncolytic virus is a bacterial peptide or a bacterial sialidase, e.g., DAS181 (SEQ ID NO: 2). In some embodiments, the foreign antigen is a sialidase comprising a transmembrane domain. In some embodiments, the foreign antigen is DAS181 without an AR tag and fused to a C-terminal transmembrane domain (e.g., SEQ ID NO: 31).

In some embodiments, there is provided a method of increasing efficacy of an immunotherapy in an individual in need of the immunotherapy, comprising administering an effective amount of a recombinant oncolytic virus encoding a sialidase and an effective amount of an immunotherapy. In some embodiments, the immunotherapy is a multi-specific immune cell engager (e.g., a BiTE), a cell therapy, a cancer vaccine (e.g., a dendritic cell (DC) cancer vaccine), a cytokine (e.g., IL-15, IL-12, modified IL-2, modified IL-18, CXCL10, or CCL4), and an immune checkpoint inhibitor (e.g., an inhibitor of CTLA-4, PD-1, PD-L1, B7-H4, TIGIT, LAG3, TIM3 or HLA-G). In some embodiments, the immunotherapy is cell therapy, e.g., a cell therapy comprising T-cells, natural killer (NK) cells, natural killer T (NKT) cells, dendritic cells (DC), cytokine-induced killer (CIK) cells, cytokine-induced natural killer (CINK) cells, lymphokine-activated killer (LAK) cells, tumor-infiltrating lymphocytes (TILs), macrophages, or combinations thereof. In some embodiments, the recombinant oncolytic virus is administered before, after, or simultaneously with the immunotherapy. In some embodiments, administering the recombinant oncolytic virus increases tumor cell killing by at least 10%, 15%, 20%, 25%, 30%, 35%, 40%, 50%, 60%, 70%, 80%, 85%, 90%, 95%, or 100% compared to the immunotherapy alone.

In some embodiments, there is provided a method of treating a cancer in an individual in need thereof, comprising administering to the individual an effective amount of engineered immune cells, wherein the immune cells express a recombinant oncolytic virus encoding a heterologous protein. In some embodiments, the immune cells express a chimeric receptor that specifically recognizes a target molecule associated with the cancer. In some embodiments, the immune cells express a chimeric receptor that specifically recognizes the sialidase encoded by the virus.

In some embodiments, there is provided a method of treating a cancer in an individual in need thereof, comprising administering to the individual an effective amount of engineered immune cells, wherein the immune cells express a recombinant oncolytic virus encoding a heterologous protein, wherein the heterologous protein is a sialidase. In some embodiments, the immune cells express a chimeric receptor that specifically recognizes a target molecule associated with the cancer. In some embodiments, the immune cells express a chimeric receptor that specifically recognizes the sialidase encoded by the virus.

One aspect of the present application provides methods of reducing sialylation of cancer cells in an individual, comprising administering to the individual an effective amount of any one of the recombinant oncolytic viruses, pharmaceutical compositions, or engineered immune cells described above. In some embodiments, the sialidase reduces surface sialic acid on tumor cells. In some embodiments the sialidase reduces surface sialic acid on tumor cells by at least 10%, 15%, 20%, 30%, 40%, 50%, 60%, 70%, 75%, 80%, 85%, or 90%. In some embodiments, the sialidase cleaves both α2,3 and α2,6 sialic acids from the cell surface of tumor cells. In some embodiments, the sialidase increases cleavage of both α2,3 and α2,6 sialic acids by at least 10%, 15%, 20%, 30%, 40%, 50%, 60%, 70%, 75%, 80%, 85%, or 90%.

In some embodiments, there is provided a method of promoting an immune response in an individual, comprising administering an effective amount of a recombinant oncolytic virus encoding a sialidase. In some embodiments, the method promotes a local immune response in a tumor microenvironment of the individual. In some embodiments, there is provided a method of promoting dendritic cell (DC) maturation in an individual, comprising administering an effective amount of a recombinant oncolytic virus encoding a sialidase (e.g., DAS181). DC maturation can be determined based on the expression of dendritic cell markers, such as CD80 and DC MHC I and MHC-II proteins. In some embodiments, the recombinant oncolytic virus increases DC maturation by at least at least 5%, 10%, 15%, 20%, 30%, 40%, or 50%. Example 9 provides results demonstrating increased DC maturation following administration of a recombinant oncolytic virus encoding a sialidase.

In some embodiments, there is provided a method of increasing immune cell killing of tumor cells in an individual, comprising administering an effective amount of a recombinant oncolytic virus encoding a sialidase. In some embodiments, the method increases killing by NK cells. In some embodiments, the recombinant oncolytic virus encoding a sialidase increases killing by NK cells by at least at least 5%, 10%, 15%, 20%, 30%, 40%, or 50%. In some embodiments, the recombinant oncolytic virus encoding a sialidase increases killing by NK cells by at least at least 5%, 10%, 15%, 20%, 30%, 40%, or 50% compared to recombinant oncolytic virus lacking sialidase. Example 3 demonstrates enhanced NK cell-mediated killing of tumor cells with administration of a recombinant oncolytic virus encoding sialidase. In some embodiments, the method increases killing by T cells. In some embodiments, the recombinant oncolytic virus encoding a sialidase increases killing by T cells by at least at least 5%, 10%, 15%, 20%, 30%, 40%, or 50%. In some embodiments, the recombinant oncolytic virus encoding a sialidase increases killing by T cells by at least at least 5%, 10%, 15%, 20%, 30%, 40%, or 50% compared to recombinant oncolytic virus lacking sialidase. Example 10 demonstrates enhanced NK cell-mediated killing of tumor cells with administration of a recombinant oncolytic virus encoding sialidase. In some embodiments, the method increases killing by PBMCs. In some embodiments, the recombinant oncolytic virus encoding a sialidase increases killing by PBMCs by at least at least 5%, 10%, 15%, 20%, 30%, 40%, or 50%. In some embodiments, the recombinant oncolytic virus encoding a sialidase increases killing by PBMCs by at least at least 5%, 10%, 15%, 20%, 30%, 40%, or 50% compared to recombinant oncolytic virus lacking sialidase. Example 6 demonstrates enhanced PBMC-mediated killing of tumor cells with administration of a recombinant oncolytic virus encoding sialidase.

In some embodiments, there is provided a method of increasing oncolytic killing of an oncolytic virus in an individual, comprising administering an effective amount of a sialidase. In some embodiments, the sialidase is encoded by the oncolytic virus. In some embodiments, oncolytic killing by a recombinant oncolytic virus encoding a sialidase is increased by at least at least 5%, 10%, 20%, 30%, 40%, or 50% compared to recombinant oncolytic virus lacking sialidase. Example 5 provides results demonstrating enhanced oncolytic killing by a recombinant oncolytic virus encoding a sialidase.

In some embodiments, there is provided a method of enhancing cytokine production and oncolytic activity in an individual, comprising administering an effective amount of a recombinant oncolytic virus encoding a sialidase. In some embodiments, the method enhances cytokine production by T-lymphocytes. In some embodiments, method enhances T-lymphocyte mediated cytokine production locally in a tumor microenvironment of the individual. In some embodiments, the cytokines include IL2 and IFN-gamma. In some embodiments, administering recombinant oncolytic virus encoding a sialidase increases cytokine production by at least at least 5%, 10%, 20%, 30%, 40%, or 50% compared to administering an oncolytic virus lacking sialidase. In some embodiments, administering recombinant oncolytic virus encoding a sialidase increases IL2 production by at least 2.5-fold, at least 3-fold, or at least 4-fold compared to administering an oncolytic virus lacking sialidase. In some embodiments, administering recombinant oncolytic virus encoding a sialidase increases IFN-gamma production by at least 5%, 10%, 20%, 30%, 40%, or 50% compared to administering an oncolytic virus lacking sialidase. Example 10 demonstrates enhanced cytokine production and killing by T-lymphocytes following administration of a recombinant oncolytic virus encoding a sialidase.

As used herein, cancer is a term for diseases caused by or characterized by any type of malignant tumor or hematological malignancy, including metastatic cancers, solid tumors, lymphatic tumors, and blood cancers.

Cancers include leukemias, lymphomas (Hodgkins and non-Hodgkins), sarcomas, melanomas, adenomas, carcinomas of solid tissue including breast cancer and pancreatic cancer, hypoxic tumors, squamous cell carcinomas of the mouth, throat, larynx, and lung, genitourinary cancers such as cervical and bladder cancer, hematopoietic cancers, head and neck cancers, and nervous system cancers, such as gliomas, astrocytomas, meningiomas, etc., benign lesions such as papillomas, and the like.

In some embodiments, delivery of the sialidase can reduce sialic acid present on tumor cells and render the tumor cells more vulnerable to killing by immune cells, immune cell-based therapies and other therapeutic agents whose effectiveness is diminished by hyper sialylation of cancer cells.

In some embodiments, the method further comprises administering to the individual an effective amount of an immunotherapeutic agent. In non-limiting examples, the immunotherapeutic agent can be a multi-specific immune cell engager, a cell therapy, a cancer vaccine, a cytokine, a PI3Kgamma inhibitor, a TLR9 ligand, an HDAC inhibitor, a LILRB2 inhibitor, a MARCO inhibitor, or an immune checkpoint inhibitor. Suitable immune cell engagers and immune checkpoint inhibitors are described in the “Other heterologous proteins or nucleic acids” subsection above.

In some embodiments, the cancer comprises a solid tumor. In some embodiments of any of the methods provided herein, the cancer is an adenocarcinoma, a metastatic cancer and/or is a refractory cancer. In certain embodiments of any of the foregoing methods, the cancer is a breast, colon or colorectal, lung, ovarian, pancreatic, prostate, cervical, endometrial, head and neck, liver, renal, skin, stomach, testicular, thyroid or urothelial cancer. In certain embodiments of any of the foregoing methods, the cancer is an epithelial cancer, e.g., an endometrial cancer, ovarian cancer, cervical cancer, vulvar cancer, uterine cancer, fallopian tube cancer, breast cancer, prostate cancer, lung cancer, pancreatic cancer, urinary cancer, bladder cancer, head and neck cancer, oral cancer or liver cancer. In some embodiments, the cancer is selected from human alveolar basal epithelial adenocarcinoma, human mammillary epithelial adenocarcinoma, and glioblastoma.

In some embodiments, the method comprises administering to the individual an effective amount of any one of the recombinant oncolytic viruses, pharmaceutical compositions, or engineered immune cells described above and an effective amount of engineered immune cells expressing a chimeric receptor. In some embodiments, the chimeric receptor targets a heterologous protein expressed by the recombinant oncolytic virus. In some embodiments, the heterologous protein is a sialidase (e.g., DAS181 or a derivative thereof, such as a membrane-bound form of DAS181), and the chimeric receptor specifically recognizes the sialidase. In some embodiments, the sialidase is DAS181 or a derivative thereof, and wherein the chimeric receptor comprises an anti-DAS181 antibody that is not cross-reactive with human native amphiregulin or any other human antigen.

In one aspect, the present application provides a method of treating a tumor in an individual in need thereof comprising administering to the individual: (a) an effective amount of a recombinant oncolytic virus comprising a nucleotide sequence encoding a foreign antigen; and (b) an effective amount of an engineered immune cell expressing a chimeric receptor specifically recognizing said foreign antigen. In some embodiments, the foreign antigen is a non-human protein (e.g., a bacterial protein).

In some embodiments, the engineered immune cells and the recombinant oncolytic virus are administered separately (e.g., as monotherapy) or together simultaneously (e.g., in the same or separate formulations) as combination therapy. In some embodiments, the recombinant oncolytic virus is administered prior to administration of the engineered immune cells. In non-limiting examples, the recombinant oncolytic virus can be administered 1 or more, 2 or more, 4 or more, 6 or more, 8 or more, 10 or more, 12 or more, 24 or more, or 48 or more hours prior to the engineered immune cells comprising the chimeric receptor. In some embodiments, a population of engineered immune cells expressing the recombinant oncolytic virus is administered prior to a population of engineered immune cells expressing a chimeric antigen receptor targeting a heterologous protein expressed by the recombinant oncolytic virus. In non-limiting examples, the engineered immune cells comprising the recombinant oncolytic virus can be administered 1 or more, 2 or more, 4 or more, 6 or more, 8 or more, 10 or more, 12 or more, 24 or more, or 48 or more hours prior to the engineered immune cells comprising the chimeric receptor targeting a heterologous protein expressed by the recombinant oncolytic virus. In some embodiments, the time period between administration of the recombinant oncolytic virus (e.g., in a pharmaceutical composition or a carrier cell comprising the recombinant oncolytic virus) and administration of the engineered immune cells expressing the chimeric receptor is sufficient to allow the virus to express the heterologous protein or nucleic acid in the tumor cells.

The recombinant oncolytic virus, and in some embodiments, the engineered immune cells and/or additional immunotherapeutic agent(s) may be administered using any suitable routes of administration and suitable dosages. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 4246.

In some embodiments, the recombinant oncolytic virus, the engineered immune cells and/or additional immunotherapeutic agent(s) are administered sequentially (e.g., the recombinant oncolytic virus can be administered prior to the engineered immune cells, and/or prior to other therapeutic agents such as bi-specific antibody of FAP/CD3, bi-specific or trispecific antibody of LILRB-4-1BB, PD-1 antibody, etc as described above). In some embodiments, the recombinant oncolytic virus, the engineered immune cells and/or additional immunotherapeutic agent(s) are administered simultaneously or concurrently. In some embodiments, the recombinant oncolytic virus, the engineered immune cells and/or additional immunotherapeutic agent(s) are administered in a single formulation. In some embodiments, the recombinant oncolytic virus, the engineered immune cells and/or additional immunotherapeutic agent(s) are administered as separate formulations.

The methods of the present invention may be combined with conventional chemotherapeutic, radiologic and/or surgical methods of cancer treatment.

### IV. Pharmaceutical Compositions, Kits and Articles of Manufacture

Further provided by the present application are pharmaceutical compositions comprising any one of the recombinant oncolytic viruses, carrier cells comprising a recombinant oncolytic virus, and/or engineered immune cells (s) described herein, and a pharmaceutically acceptable carrier.

In some embodiments, the present application provides a pharmaceutical composition comprising an oncolytic virus (such as VV) comprising a first nucleotide sequence encoding a sialidase and/or any one of the other heterologous proteins or nucleic acids described herein, and an engineered immune cell expressing a chimeric receptor (e.g., a CAR-T. CAR-NK, or CAR-NKT cell) or any of the heterologous proteins or nucleic acids described herein that can modulate and enhance immune cell function such as anti LILRB, Anti-folate receptor beta, bi-specific antibody such as anti-LILRB/4-1BB, etc.

In some embodiments, the present application provides a first pharmaceutical composition comprising a recombinant oncolytic virus (such as VV) comprising a first nucleotide sequence encoding a sialidase and/or any one of the other heterologous proteins or nucleic acids described herein, and optionally a pharmaceutically acceptable carrier; and a second pharmaceutical composition comprising an engineered immune cell expressing a chimeric receptor (e.g., a CAR-T, CAR-NK, or CAR-NKT cell), and optionally a pharmaceutically acceptable carrier.

Pharmaceutical compositions can be prepared by mixing the recombinant oncolytic viruses and/or engineered immune cells described herein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers (e.g. sodium chloride), stabilizers, metal complexes (e.g. Zn-protein complexes), chelating agents such as EDTA and/or non-ionic surfactants.

The formulation can include a carrier. The carrier is a macromolecule which is soluble in the circulatory system and which is physiologically acceptable where physiological acceptance means that those of skill in the art would accept injection of said carrier into a patient as part of a therapeutic regime. The carrier preferably is relatively stable in the circulatory system with an acceptable plasma half-life for clearance. Such macromolecules include but are not limited to soy lecithin, oleic acid and sorbitan trioleate.

The formulations can also include other agents useful for pH maintenance, solution stabilization, or for the regulation of osmotic pressure. Examples of the agents include but are not limited to salts, such as sodium chloride, or potassium chloride, and carbohydrates, such as glucose, galactose or mannose, and the like.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

In some embodiments, the systems provided herein can be stably and indefinitely stored under cryopreservation conditions, such as, for example, at −80° C., and can be thawed as needed or desired prior to administration. For example, the systems provided herein can be stored at a preserving temperature, such as −20° C. or −80° C. for at least or between about a few hours, 1, 2, 3, 4 or 5 hours, or days, including at least or between about a few years, such as, but not limited to, 1, 2, 3 or more years, for example for at least or about 1, 2, 3, 4 or 5 hours to at least or about 6, 7, 8, 9, 10, 1 1, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71 or 72 hours or 4, 5, 6, 7, 8, 9, 10, 15, 20, 25 or 30 days or 1.5, 2, 2.5, 3, 3.5, 4, 4.5, 5, 5.5, 6, 6.5, 7, 7.5, 8, 8.5, 9, 9.5, 10, 10.5, 11, 11.5 or 12 months or 1, 2, 3, 4 or 5 or more years prior to thawing for administration. The systems provided herein also stably can be stored under refrigeration conditions such as, at 4° C. and/or transported on ice to the site of administration for treatment. For example, the systems provided herein can be stored at 4° C. or on ice for at least or between about a few hours, such as, but not limited to, 1, 2, 3, 4 or 5 hours, to at least or about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 or more hours prior to administration for treatment.

The present application further provides kits and articles of manufacture for use in any embodiment of the treatment methods described herein. The kits and articles of manufacture may comprise any one of the formulations and pharmaceutical compositions described herein.

In some embodiments, there is provided a kit comprising one or more nucleic acid constructs for expression any one of the recombinant oncolytic viruses described herein, and instructions for producing the recombinant oncolytic virus. In some embodiments, the kit further comprises instructions for treating a cancer.

In some embodiments, there is provided a kit comprising any one of the recombinant oncolytic viruses described herein, and instructions for treating a cancer. In some embodiments, the kit further comprises an immunotherapeutic agent (e.g., a cell therapy or any one of the immunotherapies described herein). In some embodiments, the kit further comprises one or more additional therapeutic agents for treating the cancer. In some embodiments, the antagonist, the recombinant oncolytic virus and/or the one or more immunotherapeutic agents are in a single composition (e.g., a composition comprising a cell therapy and a recombinant oncolytic virus). In some embodiments, the recombinant oncolytic virus and optionally the one or more additional immunotherapeutic agents and/or additional therapeutic agents for treating the cancer are in separate compositions.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

### EXAMPLES

The examples below are intended to be purely exemplary of the invention and should therefore not be considered to limit the invention in any way. The following examples and detailed description are offered by way of illustration and not by way of limitation.

### Example 1: DAS181 Treatment Reduces Surface Sialic Acid on Tumor Cells

In this study the impact of DAS181 on the sialic acid burden of certain tumor cells was examined. Briefly, FACs and image-based quantitation of α-2,3 and α-2,6 sialic acid modifications on A549 (human alveolar basal epithelial adenocarcinoma) and MCF (human mammillary epithelial adenocarcinoma) tumor cells were conducted. Galactose exposure after sialic acid removal in A549 and MCF7 cells was detected by PNA-FITC using flow cytometry analysis and imaging approaches. As discussed above, there are two sialic acid is most often attached to the penultimate sugar by an α-2,3 linkage or an α-2,6 linkage, which can that can be detected by Maackia amurensis Lectin II (MAL II) and Sambucus nigra Lectin (SNA), respectively. In addition, surface galactose (e.g., galactose exposed after sialic acid removal) can be detected using Peanut Agglutinin (PNA).

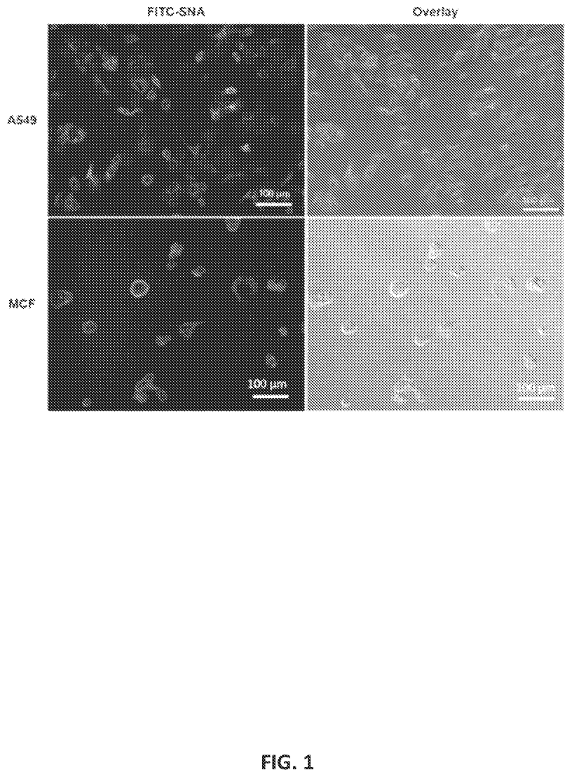

FIG. 1 depicts the detection of α-2,6 sialic acid by FITC-SNA on A549 and MCF cells by fluorescence imaging.

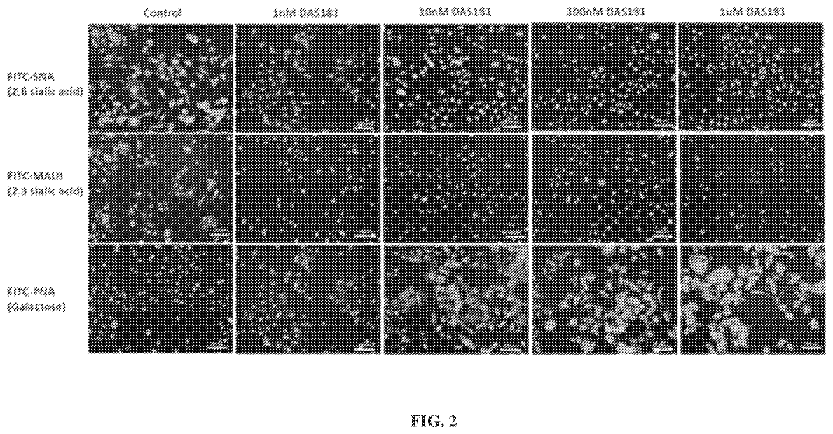

A549 cells were treated with various concentrations of DAS181 and them stained to image 2,6 linked sialic acid (FITC-SNA), α-2,3 linked sialic acid (FITC-MALII) or galactose (FITC-PNA). As can be seen in FIG. 2, DAS181 effectively removed both 2,3 and 2,6 linked sialic acid and exposed galactose.

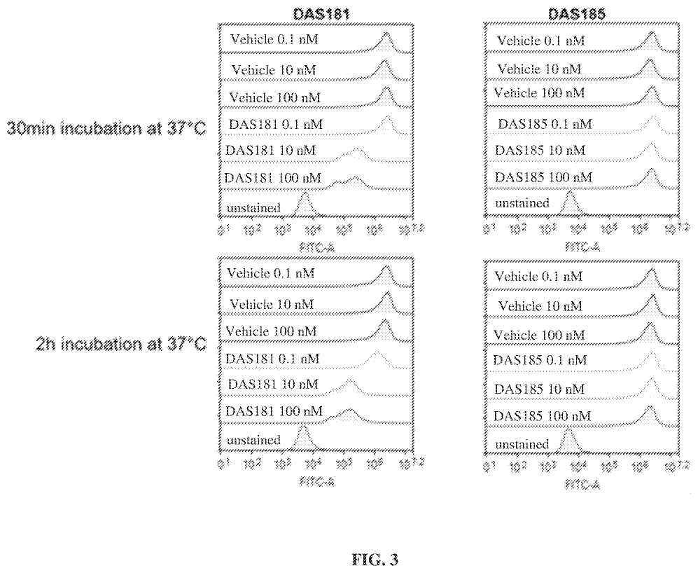

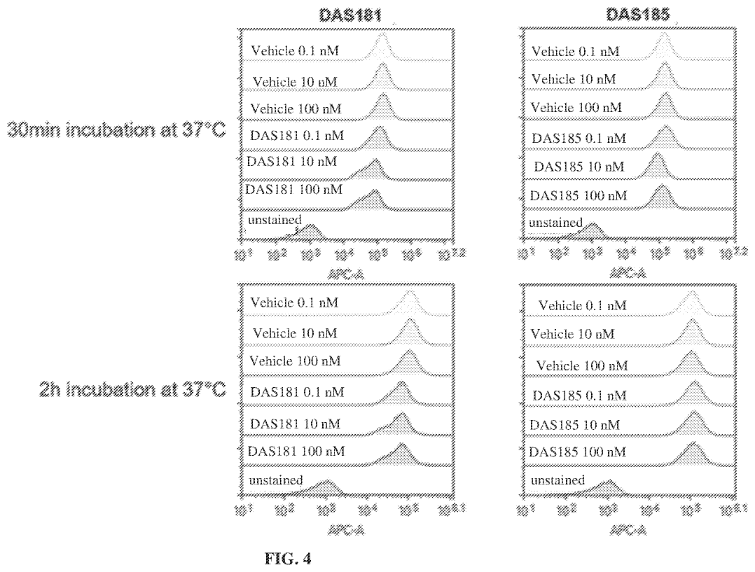

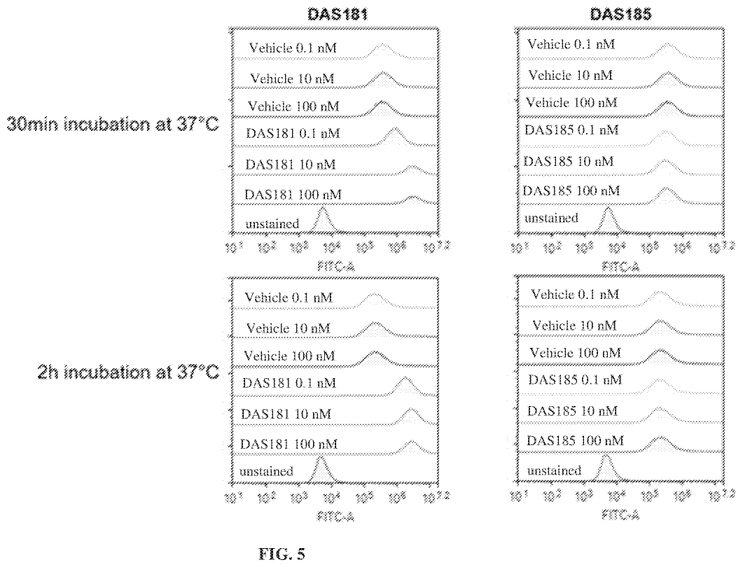

In contrast, DAS185, a variant of DAS181 lacking sialidase activity due to Y348F mutation, was not able to remove α-2,6 linked sialic acid or α-2,3 linked sialic acid. As shown in FIG. 3, incubation of A549 cells with DAS185 had essentially no impact on surface α-2,3 linked sialic acid, while DAS181 reduced surface α-2,3 linked sialic acid in a concentration dependent manner (cells stained with FITC-MALII; results shown in FIG. 3). Similarly, incubation of A549 cells with DAS185 had essentially no impact on surface α2,6 linked sialic acid, while DAS181 reduced surface α-2,6 linked sialic acid in a concentration dependent manner (cells stained with FITC-SNA: results shown in FIG. 4). Consistent with these results, incubation of A549 cells with DAS185 had essentially no impact on surface galactose, while DAS181 increased surface galactose in a concentration dependent manner (cells stained with FITC-PNA; results shown in FIG. 5).

### Example 2: DAS181 Treatment Increases PBMC-Mediated Tumor Cell Killing

Example 1 demonstrated that DAS181 effectively reduces the sialic acid burden of tumor cells with broad specificity (e.g., cleaving both α-2,3 vs. α-2,6 linkages). Example 2 demonstrates that treatment of tumor cells with DAS181 significantly enhances PBMC-mediated killing of the treated tumor cells compared to untreated tumor cells.

Briefly, FACs and image-based quantitation of α-2,3 and α-2,6 sialic acid

A549 cells were genetically labelled with a red fluorescent protein (A549-red). Fresh human PBMCs were harvested and stimulated with various cytokine and antibody combinations to activate effector T cells (CD3, CD38 and IL-2) or, in some cases, T cells and NK cells (CD3, CD28, IL-15 and IL-21). Activated PBMCs were then co-cultured with A549-red cells that had been exposed to DAS181 (100 nM). Tumor cell killing by PBMCs was monitored by live cell imaging and quantification with IncuCyte. The cell culture medium was collected and analyzed by ELISA to assess cytokine production by PBMCs.

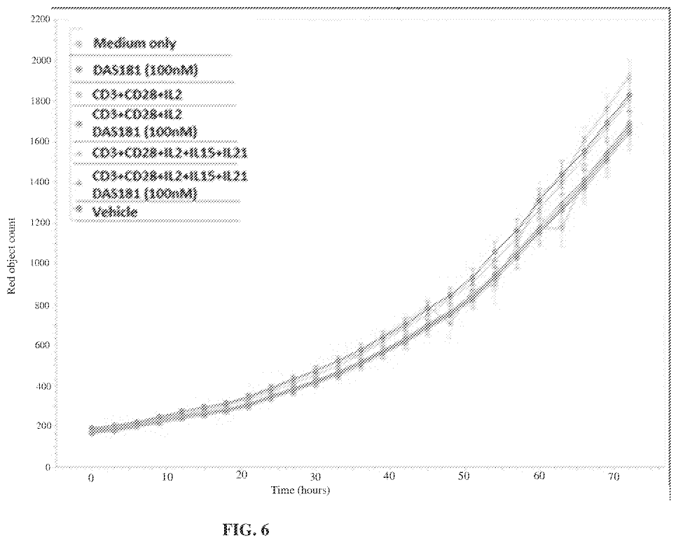

FIG. 6 shows that neither the treatments used to stimulate PBMC nor DAS181 in combination with treatment used to stimulate PBMC impact A549-red cell proliferation.

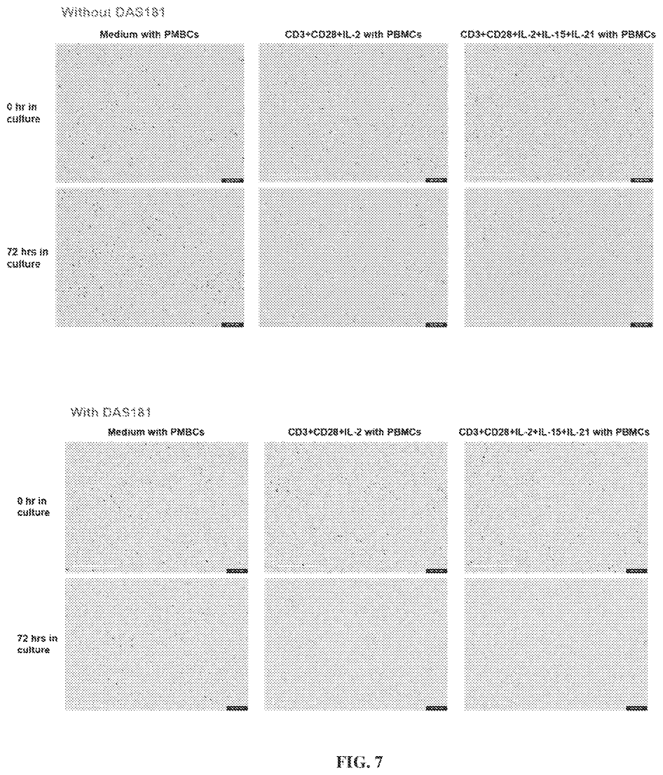

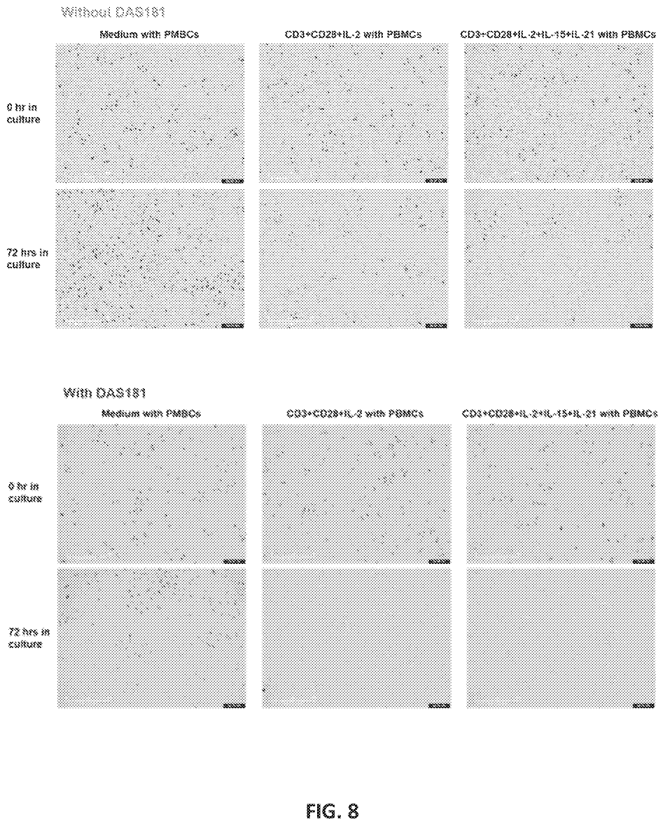

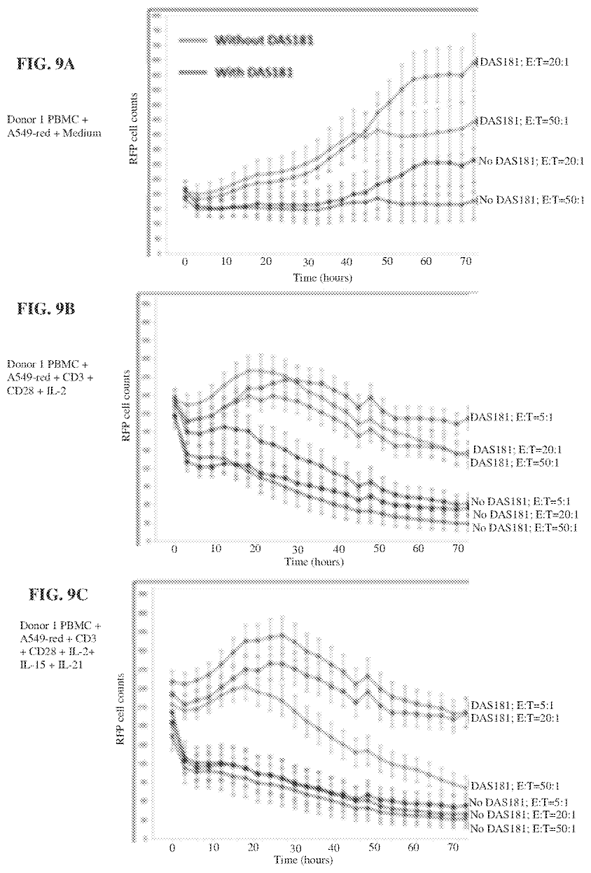

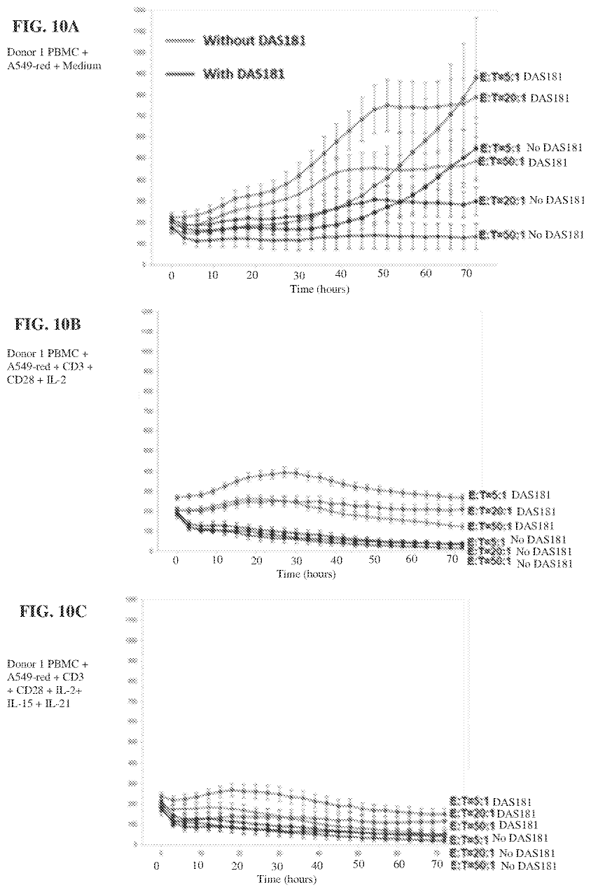

FIG. 7 shows that DAS181 significantly increases tumor cell toxicity mediated by PBMC (Donor 1), both T cell mediated and NK cell mediated, compared to a vehicle only control. Similar results were observed using PBMC from a different donor (Donor 2; FIG. 8). FIGS. 9A-C presents a quantification of the data presented in FIG. 7. FIG. 9A shows quantification of A549-red cells following treatment with PBMCs with or without DAS181 at the indicated effector cell:tumor cell ratios. FIG. 9B shows quantification of A549-red cells following treatment with PBMCs stimulated with CD3, CD38 and IL-2 to activate effector T cells with or without DAS181 at the indicated effector cell:tumor cell ratios. FIG. 9C shows quantification of A549-red cells following treatment with PBMCs stimulated with CD3, CD28, IL-15 and IL-21 to activate effector T and NK cells with or without DAS181 at the indicated effector cell:tumor cell ratios. FIGS. 10A-10C show the same quantifications, respectively, using PBMCs from a different donor (Donor 2).

### Example 3: NK Cell Mediated Killing of Tumor Cells by Oncolytic Vaccinia Virus and DAS181

In this study the impact of an oncolytic vaccinia virus (Western Reserve, VV) and DAS181 on NK cell-mediated killing was examined. DAS185, a variant protein lacking sialidase activity was used as a control. This Example demonstrates that exposure to DAS181 increases tumor cell killing by an oncolytic virus.

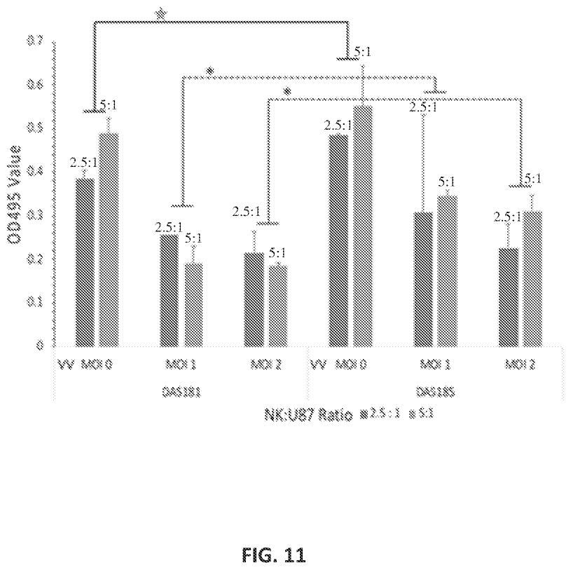

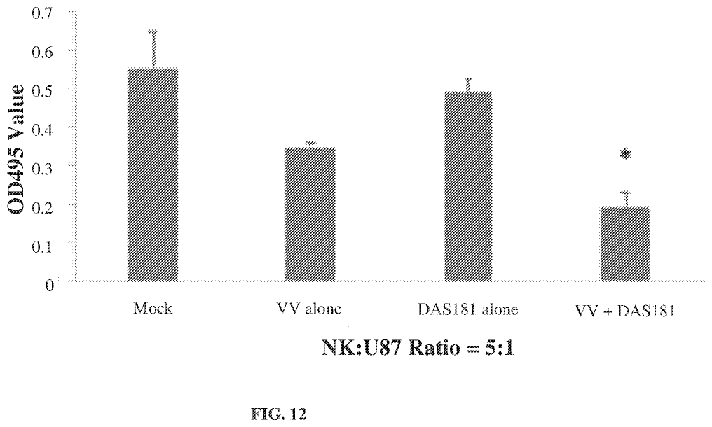

Briefly, tumor cells (U87-GFP) were plated in a 96-well tissue culture plate at 5×10⁴ cells per well (100 ul) in DMEM and incubated overnight at 37° C. On Day 2 the cells were infected with VV at MOI 0.5, 1, or 2 in fetal bovine serum-free medium for 2 hours and then exposed to 1 nM DAS181 or 1 mM DAS185. Tumor cells were then mixed with purified NK cells at Effector:Tumor (E:T)=1:1, 5:1, 10:1. The cells were cultured in medium supplemented with 2% FBS in order to decrease neuraminidase/sialidase background. After 24 hrs, tumor killing were measured by MTS assay (96 well plate), and cell culture medium was collected. Expression of IFN gamma were measured by ELISA. The results of this study are shown in FIG. 11 and FIG. 12 where it can be seen the DAS181, but not inactive DAS185, increased tumor cell killing by oncolytic vaccinia virus.

### Example 4: Impact of DAS181 on DC Maturation and Macrophage Activity in the Presence of Tumor Cells

In this study, the impact of DAS181 on monocyte-derived dendritic cells or macrophages was examined. DAS185, a variant protein lacking sialidase activity was used as a control.

Briefly, monocyte-derived dendritic cells (DC) were prepared by resuspending 5×10⁶ adherent PBMC in 3 ml of medium supplemented with 100 ng/ml of GM-CSF and 50 ng/ml of IL-4. After 48 hrs, 2 ml of fresh medium supplemented with 100 ng/ml of GM-CSF and 50 ng/ml of IL-4 was added to each well. After another 72 hrs, tumor cells (U87-GFP) were plated in 24-well plates in DMEM. The tumor cells were infected with VV at various MOI in FBS free medium for 2 hours. DC cultured in the presence of 1 nM DAS181 or DAS185 were mixed with tumor cells at 1:1 tumor cell:DC ratio. Dendritic cell maturation (expression of CD86, CD80, MHC-II, MHC-I).

In addition, THP-1 cells were cultured in RPMI 1640 medium (Invitrogen) containing 10% heat-inactivated FBS. THP-1 cells in a 6-well plate (3×10e6 cells/well) were stimulated with PMA (20 ng/ml) in the absence and in the presence of 1 nM of Sialidase DAS181 or DAS185. Cell culture medium volume was 2 ml. On day 5, tumor cells (U87-GFP, DMEM cell culture medium) were plated in a 24-well tissue culture plate. Tumor were infected with VV at various MOI (i.e. 0.5, 1, 2) in FBS free medium for 2 hours. For THP-1 cell culture, 1.5 ml cell culture medium was removed by pipette. The differentiated THP-1 cells were further stimulated for 12 h by ionomycin (1 ug/ml) and PMA (20 ng/ml) also in the absence and in the presence of 1 nM of Sialidase DAS181 or DAS185 and tumor cells-VV at tumor:macrophage ratio of 1:1. The THP-1 cells were cultured in medium supplemented with 2% FBS in order to decrease neuraminidase background. On day 6, the concentration of cytokine in the culture medium was measured by ELISA array.

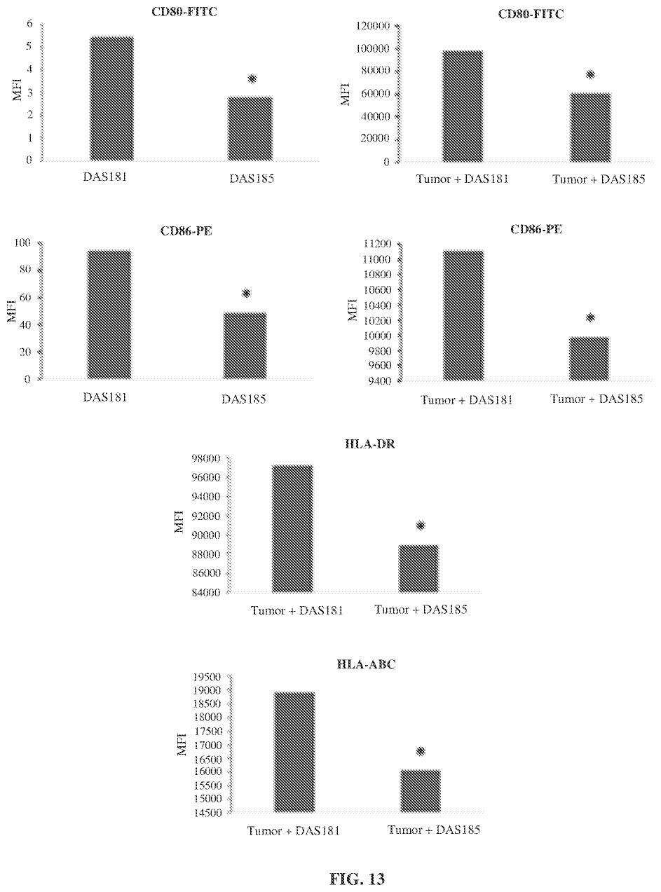

As can be seen in FIG. 13, DAS181 significant enhanced expression of dendritic cell maturation markers whether the cells were cultured alone or with vaccinia virus infected tumor cells.

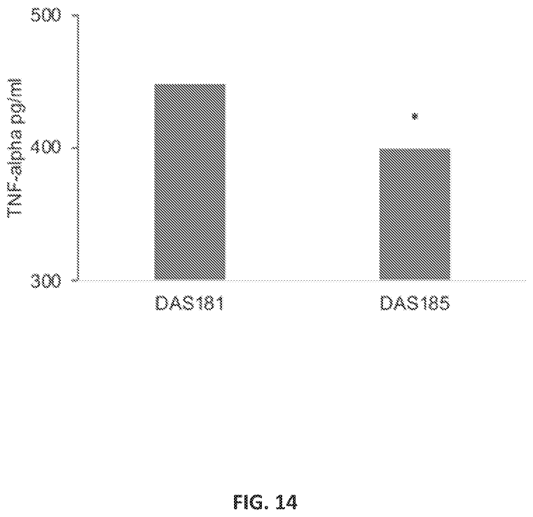

Additionally, the results of this study demonstrate that exposure to DAS181 increased and increased TNF-alpha secretion by THP-1 derived macrophage (FIG. 14).

### Example 5: DAS181 Increases Oncolytic Adenovirus Tumor Cell Killing in the Absence of Immune Cells

This Example provides unexpected results demonstrating that treatment with DAS181 increases oncolytic virus tumor cell killing, even in the absence of immune cells.

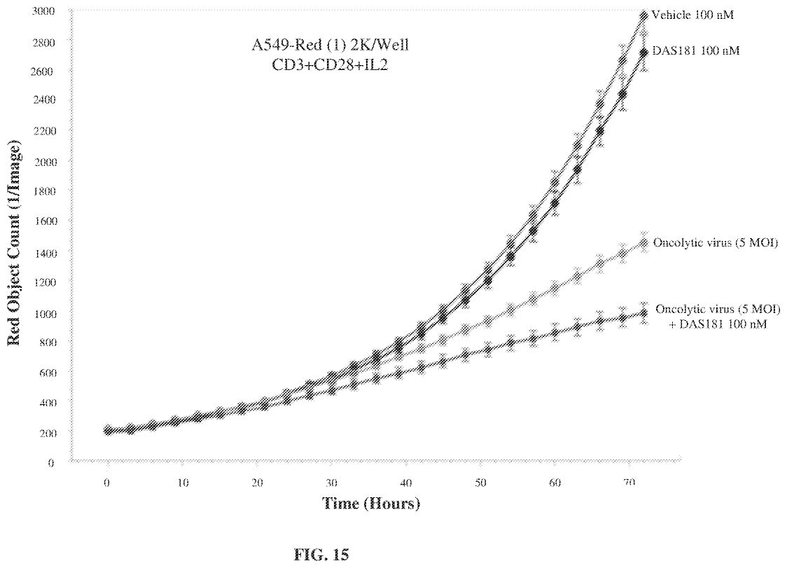

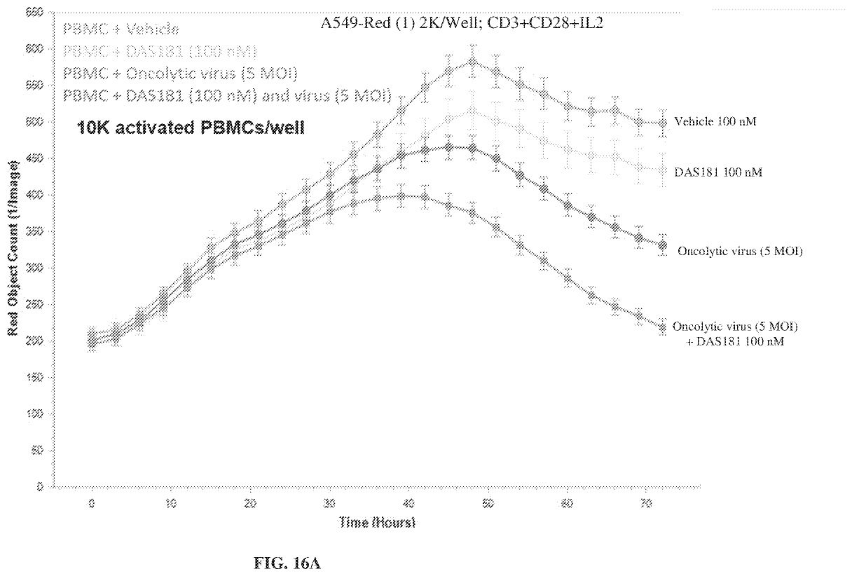

A549 cells were genetically labelled with red fluorescent protein (A549-red). Tumor cell proliferation and killing by oncolytic adenovirus (Ad5) in the presence or absence of DAS181 was monitored by live cell imaging and quantification with IncuCyte. The cell culture medium was collected for ELISA measurement of cytokine production by PBMCs. As shown in FIG. 15, DAS181 increased oncolytic adenovirus-mediated tumor cell killing and growth inhibition.

### Example 6: DAS181 Increases Oncolytic Adenovirus Tumor Cell Killing in the Presence of PBMC

As shown in Example 5, treatment with DAS181 increases killing of tumor cells by an oncolytic virus in the absence of immune cells. Example 6 provides results demonstrating that treatment with DAS181 also increases tumor cell killing when present together with oncolytic virus in the presence of PBMC

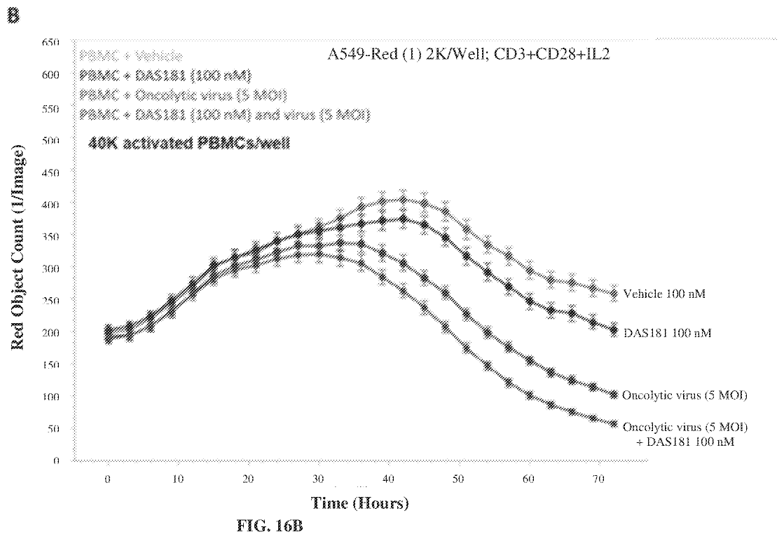

A549 cells were genetically labelled by a red fluorescent protein (A549-red). Fresh human PBMCs were harvested and stimulated with proper cytokine and antibody combinations to activate effector T cells. Activated PBMCs were then co-cultured with A549-red cells that have been treated with DAS181 with or without the oncolytic adenovirus (Ad5). Tumor cell killing by PBMCs was monitored by live cell imaging and quantification with IncuCyte. The cell culture medium was collected for ELISA measurement of cytokine production by PBMCs. As shown in FIG. 16, DAS181 significantly increased tumor cell killing when present together with oncolytic adenovirus in the presence of PBMC.

### Example 7: Construction and Characterization of an Oncolytic Virus Expressing DAS181

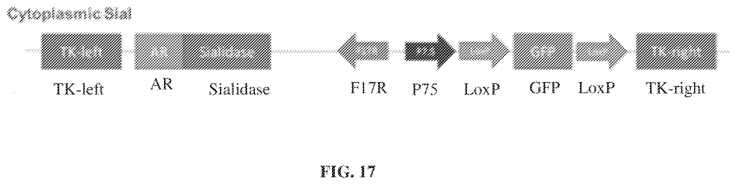

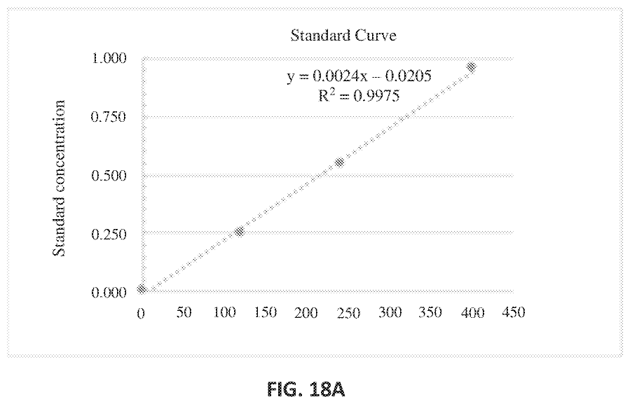

A construct designed for expression of DAS181 is depicted schematically in FIG. 17.

To generate a recombinant VV expressing DAS181, a pSEM-1 vector was modified to include a sequence encoding DAS181 as well as two loxP sites (loxP site sequences are shown in SEQ ID NO: 62) with the same orientation flanking the sequence encoding the GFP protein (the GFP coding sequence is shown in SEQ ID NO: 63). (pSEM-1-TK-DAS181-GFP). DAS181 expression is under the transcriptional control of the F17R late promoter in order to limit the expression within tumor tissue. The sequence of a portion of an exemplary construct is shown in SEQ ID NO: 65.

Western Reserve VV was used as the parental virus. VV expressing DAS181 was generated by recombination with pSEM-1-TK-DAS181-GFP into the TK gene of Western Reserve VV to generated VV-DAS181.

Recombinant virus can be generated as follows.

### Transfection:

Seed CV-1 cells in 6-well plate at 5×10⁵ cells/2 ml DMEM-10% FBS/well and grow overnight. Prepare parent VV virus (1 ml/well) by diluting a virus stock in DMEM/2% FBS at MOI 0.05. Remove medium from CV-1 wells and immediately add VV, and culture for 1-2 hours. CV-1 cells should be 60-80% confluent at this point. Transfection mix in 1.5 ml tubes. For each Transfection, dilute 9 μl Genejuice in 91 ul serum-free DMEM and incubate at room temperature for 5 min. Add 3 ug pSEM-1-TK-DAS181-GFP DNA gently by pipetting up and down two or three times. Leave at room temperature for 15 min. Aspirate VV virus from the CV-1 well and wash the cells once with 2 ml serum-free DMEM. Add 2 ml DMEM-2% FBS and add the DNA-Genejuice solution drop-by-drop. Incubate at 37° C. for 48-72 hr or until all the cells round up. Harvest the cells by pipetting repeatedly. Release the virus from cells by repeated freeze-thawing of the harvested cells by first placing them in dry-ice/ethanol bath and then thawing them in a 37° C. water bath and vortexing. Repeat the freeze-thaw cycling three times. The cell lysate can be stored at −80° C.

### Plaque Isolation:

Seed CV-1 cells in 6-well plates at 5×10⁵ cells/2 ml DMEM-10% FBS/well and grow overnight. CV-1 cells should be 60-80% confluent when receiving cell lysate. Sonicate the cell lysate on ice using sonic dismembrator with an ultrasonic convertor probe for 4 cycles of 30 s until the material in the suspension is dispersed. Make 10-fold serial dilutions of the cell lysate in DMEM-2% FBS. Add 1 ml of the cell lysate-medium per well at dilutions 10⁻², 10⁻³, 10⁻⁴, incubate at 37° C. Pick well-separated GFP+ plaques using pipet tip. Rock the pipet tip slightly to scrape and detach cells in the plaque. Gently transfer to a microcentrifuge tube containing 0.5 ml DMEM medium. Freeze-thaw three times and sonicate. Repeat the same process of plaque isolation 3-5 times.

### Virus Amplification:

Seed CV-1 cells 5×10⁵ cells/2 ml DMEM-10% FBS/well and grow overnight in 6-well plate. CV-1 should be confluent when starting the experiment. Infect I well with 250 ul of plaque lysate/1 ml DMEM-2% FBS, and incubate at 37° C. for 2 h. Remove the plaque lysate and add 2 ml fresh DMEM-2% FBS, and incubate for 48-72 hr until cells round up. Collect the cells by repeatedly pipetting, freeze-thaw 3 times and sonicate. Add half of the cell lysate in 4 ml DMEM-2% FBS and infect CV-1 cells in 75-CM2 flask, after 2 h, remove virus and add 12 ml DMEM-2% FBS and culture 48-72 h (until cell round up). Harvest the cells, spin down 5 min at 1800 G, and discard supernatant and resuspend in 1 ml DMEM-2.5% FBS.

### Virus Titration:

Seed CV-1 cells 5×10⁵ cells/2 ml DMEM-10% FBS/well and grow overnight in 6-well plate. Dilute virus in DMEM-2% FBS, 50 ul virus/4950 ul DMEM-2% FBS (A, 10⁻²), 500 ul A/4500 ul medium (B, 10⁻³), and 500 ul B/4500 ul medium (C, 10⁻⁴), 10⁻⁷ to 10⁻¹⁰ for virus stock. Remove medium and wash 1× with PBS, and cells were infected with 1 ml virus dilution in duplicate. Incubate the cells for 1 h, rock the plate every 10 min. 1 h later, remove the virus and add 2 ml DMEM-10% FBS and incubate 48 h. Remove the medium, add 1 ml of 0.1% crystal violet in 20% ethanol for 15 min at room temperature. Remove the medium and allow to dry at room temperature for 24 hr. Count the plaque and express as plaque forming units (pfu) per ml.

### Detection of DAS181 Expression by VV-DAS181:

CV-1 cells were infected with VV-DAS181 at MOI 0.2. 48 hours later, CV-1 cells were collected. DNA was extracted using Wizard SV Genomic DAN Purification System and used as template for DAS181 PCR amplification. PCR was conducted using standard PCR protocol and primer sequences (SialF: GGCGACCACCCACAGGCAACACCAGCACCTGCCCCA (SEQ ID NO: 56) and SialR: CCGGTTGCGCCTATTCTTGCCGTTCTTGCCGCC (SEQ ID NO: 57)). The expected PCR product (1251 bp) was found.

### Example 8: DAS181 Expressed by Vaccinia Virus is Active In Vitro

Example 8 provides results demonstrating that delivery of DAS181 to cells using an oncolytic virus results in sialidase activity equivalent to treatment with approximately 0.78 nM-1.21 nM of purified DAS181 in 1 ml medium.

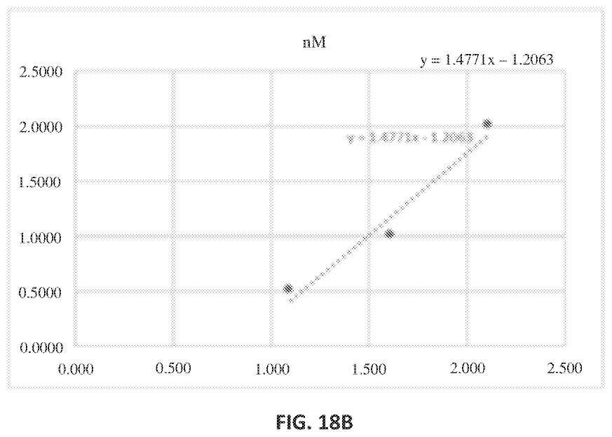

CV-1 cells were plated in six well plate. The cells were transduced with Sialidase-VV or control VV at MOI 0.1 or MOI 1. After 24 hrs, transfected cells were collected, and single cell suspension were made in PBS at 3×10⁶/500 μl. Cell lysate was prepared using Sigma's Mammalian cell lysis kit for protein extraction (Sigma, MCL 1-1KT), and supernatant was collected. The sialidase (DAS181) activity was measured using Neuraminidase Assay Kit (Abcam, ab138888) according to manufacturer's instruction. 1 nM, 2 nM, and 10 nM DAS181 was added to the VV-cell lysate as control and generated the standard curve. 1×10⁶ cells infected with Sialidase-VV express DAS181 equivalent to 0.78 nM-1.21 nM of DAS181 in 1 ml medium. As shown in FIG. 18, the DAS181 has sialidase activity in vitro.

### Example 9: Vaccinia Virus-Sialidase Promotes Dendritic Cell Maturation

Example 9 provides results demonstrating that an oncolytic virus encoding a sialidase promotes dendritic cell maturation compared to an oncolytic virus without a sialidase.

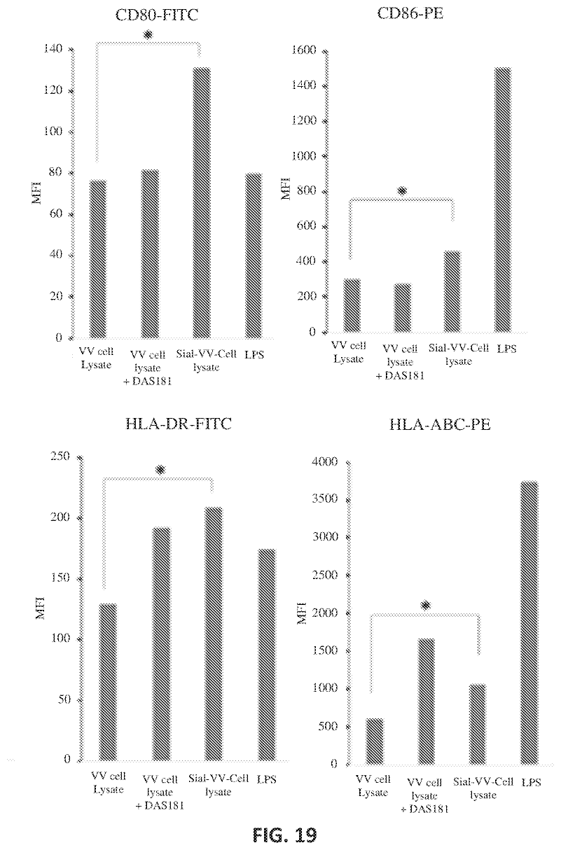

To determine if Sialidase-VV can promote DC activation and maturation, adherent human PBMC were re-suspend at 5×10⁶ cells in 3 ml medium supplemented with 100 ng/ml of GM-CSF and 50 ng/ml of IL-4 then cultured in 6-well plates with 2 ml per well of fresh medium supplemented with same concentrations of GM-CSF and IL-4. 6 days post cell culture, the cells were cultured in the presence of Sialidase-VV infected tumor cell lysate. VV-infected tumor cell lysate, VV-infected tumor cell lysate plus synthetic DAS181 protein, or LPS (positive control). After another 24 hrs, expression of CD86, CD80, MHC-II, MHC-I were determined by flow cytometry. As shown in FIG. 19, Sialidase-VV promotes the expression of markers indicative of dendritic cell activation and maturation compared to treatment with VV alone.

### Example 10: Sialidase-VV Enhances T Lymphocyte-Mediated Cytokine Production and Oncolytic Activity

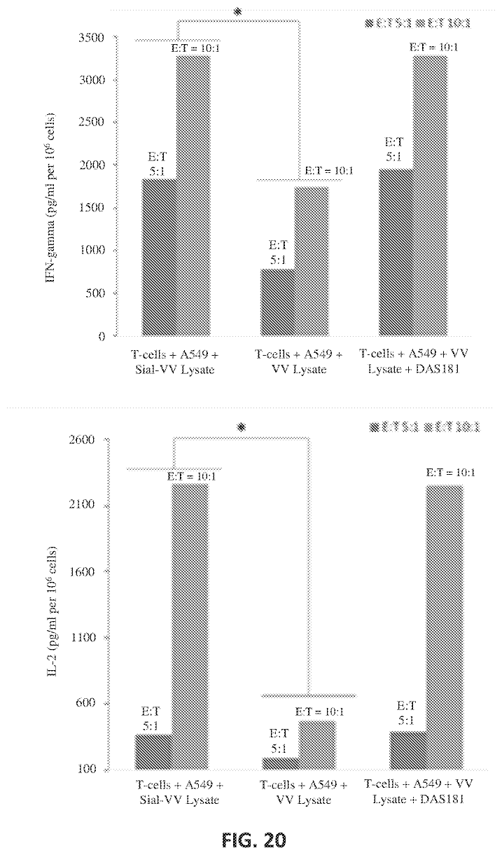

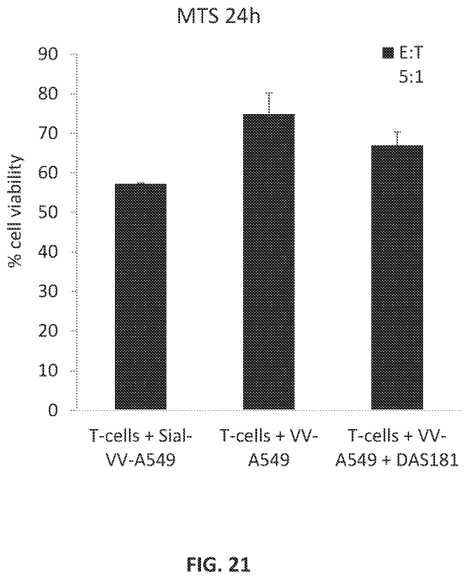

To assess whether DAS181 can activate human T cells by inducing IFN-gamma (IFNr) and IL-2 expressing, human PBMCs were activated by adding CD3 antibody at 10 μg/ml, proliferation was further stimulated by adding IL-2 by every 48 hrs. On day 15, tumor cells (A549) were infected with VVs at MOI 0.5, 1, or 2 in 2.5% FBS medium for 2 hours. Activated T cells were added to the culture at effector:target ratio of 5:1 or 10:1 in the presence of CD3 antibody at 1 ug/ml. After another 24 hrs, tumor cytotoxicity was measured, and cell culture medium was collected for cytokine array. As can be seen in FIG. 20, Sialidase-VV induces a significantly greater IL-2 and IFN-gamma expression by CD3 activated T cells than does VV. In addition, as can be seen in FIG. 21, Sialidase-VV elicits stronger anti-tumor response than VV at an E:T of 5:1.

### Example 11: Generation of Expression Constructs for Secreted and Transmembrane DAS181

Secreted and transmembrane forms of DAS181 were created to examine impact on sialidase activity. As a negative control, secreted and transmembrane forms of a point mutant that very substantially reduces sialidase activity were also created. Finally, secreted and transmembrane forms of Neu2, an alternative sialidase, were also constructed.

To facilitate the secretion of DAS181 from cells, a DNA sequence encoding the signal peptide of the mouse Immunoglobulin kappa chain was added to the N-terminus of DAS181 sequence by gene synthesis and then together cloned into a mammalian expression vector pcDNA3.4. To restrict the DAS181 sialidase activity on the cell surface, a DNA sequence encoding the DAS181 catalytic domain was synthesized and cloned in-frame with the human PDGFR beta transmembrane domain in a mammalian expression vector pDisplay. For controls, DNA sequences encoding secreted and transmembrane versions of DAS185, a mutant protein lacking sialidase activity, were similarly synthesized and cloned into pcDNA3.4 and pDisplay vectors, respectively. In addition, constructs expressing secreted and transmembrane versions of human Neu2 sialidases were generated in the same manner. The sequences for the following constructs were shown: construct 1 (secreted DAS181; SEQ ID NO: 34), construct 4 (transmembrane DAS181; SEQ ID NO: 37), construct 2 (secreted DAS185: SEQ ID NO: 35), construct 5 (transmembrane DAS185; SEQ ID NO: 38), construct 3 (secreted human Neu2; SEQ ID NO: 36) and construct 6 (transmembrane human Neu2: SEQ ID NO: 39).

### Example 12: Enzymatic Activity of Secreted and Transmembrane Sialidases

For ectopic expression, mammalian expression vectors (detailed in Example 11) were transfected into HEK293 cells using jetPRIME transfection reagent (Polyplus Transfection #114-15) following the manufacturer's protocol. Briefly, Human embryonic kidney cells (HEK293) were plated at ˜2×10⁵ live cells per well in 6-well tissue culture plates and grown to confluency by incubation at 37° C., 5% CO2, and 95% relative humidity (typically overnight). Two microliters equivalent to 2 micrograms of DNA was diluted into 200 microliters jetPRIME Buffer followed by 4 microliters of jetPRIME reagent. Tubes were vortexed, briefly centrifuged at 1,000×g (˜10 seconds) and incubated for 10 minutes at room temperature. During the incubation, the media on all wells was replenished with fresh culture media (MEM+10% FBS). Transfections were added to individual wells and the plate returned to the incubator for 24 hours. Following incubation, supernatants were reserved. Single cell suspensions were created using non-enzymatic cell dissociation reagent Versene (Gibco #15040-066). Monolayers were washed 1 time with DPBS and 500 microliters Versene was added the plate incubated until cells dissociated from vessel surface: 500 microliters complete media was added and the cells were centrifuged for 5 minutes at 300×g. The supernatant was aspirated and cells were suspended in 300 microliters compete media for enzymatic assay.

For each of the resulting transfection cultures, supernatant and resuspended cells were evaluated for activity utilizing the ability of the sialidase to enzymatically cleave the fluorogenic substrate, 2′-(4-Methylumbelliferyl)-α-D-N-acetylneuraminic acid sodium salt hydrate (MuNaNa) to release the fluorescent molecule 4-methylumbelliferone (4-Mu). The resulting free 4-Mu is excited at 365 nM and the emission is read at 445 nm using a fluorescent plate reader. Briefly, 100 μl of each sample was plated into a black, non-treated 96-well plate. The plate was incubated in a water bath at 37° C. for approximately 30 minutes and subsequently mixed with pre-incubated (37° C., 30 minutes) 100 μM MuNaNa. The fluorescence was kinetically measured at 30 second intervals for 60 minutes using a Molecular Devices SpectraMax M5e multi-mode plate reader. The amount of 4-Mu generated by cleavage was quantified by comparison to a standard curve of pure 4-Mu, ranging from 100-5 μM. Reaction rates were determined for each sample by dividing the amount of 4-Mu produced (≤20 μM) by the time (seconds) required to do so. The observed reaction rates were compared to determine the approximate relative activity of each sample solution (Table 6). It was shown that the supernatant from the secreted DAS181 transfection and the resuspended cells from the transmembrane DAS181 transfection were the most active and approximately equal. All DAS185 and Neu2 sample solutions showed negligible activity compared to the DAS181 sample solutions. The Neu2 sample solutions were equivalent to the background. Furthermore, the observed reaction rates were compared to a standard curve of known concentrations of DAS181, ranging from 1000-60 pM. The supernatant from the secreted DAS181 transfection and the resuspended cells from the transmembrane DAS181 transfection were extrapolated to be approximately equivalent to 4000 pM DAS181. All other samples were observed to be approximately equivalent to or less than 90 pM DAS181.

TABLE 6

| | | | Conditioned | | Concentrated | |
|---|---|---|---|---|---|---|
| | | | Media* | | Cells** | |
| | | | | DAS181 | | DAS181 |
| | | | | Eq. | | Eq. |
| | | | Activity | Concen- | Activity | Concen- |
| | | | (pM 4- | tration | (pM 4- | tration |
| | | | Mu/sec) | (pM) | Mu/sec) | (pM) |
| DAS181 | Secreted | Construct 1 | 60797 | 4370 | 25326 | 1857 |
| | TM | Construct 4 | 1451 | 166 | 55261 | 3978 |
| DAS185 | Secreted | Construct 2 | 89 | N/A | 47 | N/A |
| | TM | Construct 5 | 1.9 | N/A | 48 | N/A |
| Neuz | Secreted | Construct 3 | 2.2 | N/A | 0.3 | N/A |
| | TM | Construct 6 | 0.6 | N/A | 0.0 | N/A |

*Conditioned Media samples were spun down to remove any debris and tested neat.
**Cells were harvested, spun down and resuspended in 300 μL media.
All values are adjusted to remove background activity from the media.
All values determined describe the specific sample tested. Values between samples cannot be directly compared because the enzyme concentrations will vary.

### Example 13: Secreted DAS181 and Transmembrane DAS181 Reduce Surface Sialic Acids on Tumor Cells

The effect of secreted and transmembrane sialidases on cell surface sialic acid removal and galactose exposure were examined by imaging and flow cytometry after transient transfection of various expression constructs into A549-red cells using Fugene HD (Promega) following the instructions provided by the manufactures. Briefly, A549-Red cell were plated at 2×10⁵ cells per well in 2 ml of A549-Red complete growth medium in 6-well plates. For each well of cells to be transfected, 3 μg of plasmid DNA and 9 μl of Fugene HD were diluted into 150 μl of Opti-MEMA, I Reduced Serum Medium, mixed gently and incubated for 5 minutes at room temperature to form DNA-Fugene HD complexes. The above DNA-Fugene HD complexes directly to each well containing cells and the cells were incubated at 37° C. in a CO₂ incubator overnight before further experiments.

For imaging experiments, transfected cells were re-seeded as 8,000 cells per well in 96-well plates. Then cells were fixed and stained for α2,3-sialic acid; α2,6-sialic acid: and galactose following cell culture for 24 hr, 48 hr or 72 hr. Cells were incubated with SNA-FITC at 40 μg/ml, PNA-FITC at 20 μg/ml for 1 h at room temperature to stain α2,6-sialic acid, and galactose, separately. For α2,3-sialic acid, cells were incubated with Biotinylated MA II at 40 μg/ml for 1 hr, followed by FITC-Streptavidin for an additional 1 hr. To detect HA-tag expression, cells are incubated with HA-Tag rabbit mAb (1:200) for 1 hr at room temperature, followed by Donkey anti-rabbit-Alexa Fluor647 for an additional 1 hr. The images are taken by Keyence Fluorescent Microscopy.

Images taken 24 hr post transfection showed that, similar to recombinant DAS181 treatment, secreted DAS181 (Construct 1) and transmembrane DAS181 (Construct 4) transfection removed both α2,3 and α2,6 sialic acids from cell surface with a concomitant increased galactose staining. Cell transfected with enzyme-inactive DAS185 (Constructs 2, 5) or human Neu2 (Constructs 3, 6) showed similar staining pattern as vehicle control cells, consistent with the enzyme activity results.

Images taken 72 hr post transfection more evidently demonstrated that only secreted and transmembrane DAS181 transfections were capable of efficiently removing tumor cell surface sialic acids. However, it is possible that human Neu2 was not expressed well by the cells as staining of HA tag present in the transmembrane constructs was only positive in the cells transfected with the DAS181 and DAS185 constructs.

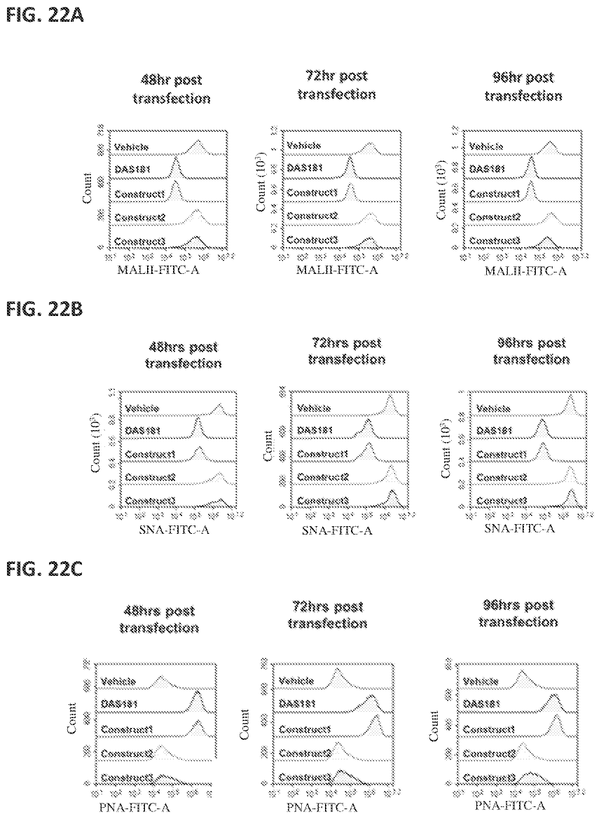

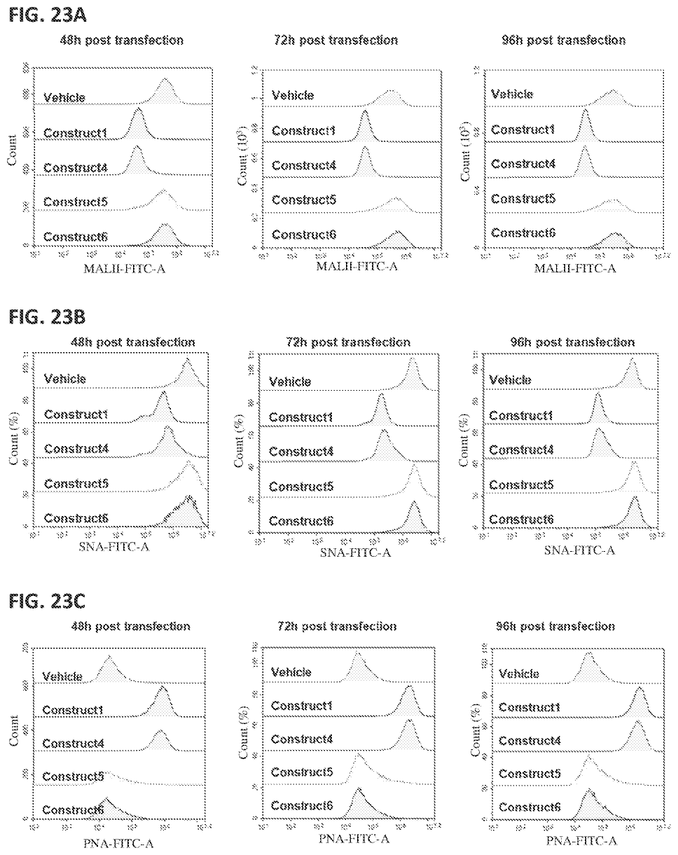

For flow cytometry analysis, transfected cells were re-seeded at 1×10⁵ cells per well in 24-well plates. Then cells are fixed and stained for α2,3-sialic acid, α2,6-sialic acid, and galactose following cell culture for 24 hr, 48 hr or 72 hr. Results were analyzed using Acea Flow cytometer system. The results of secreted construct transfections, with recombinant DAS181 treatment as control, are shown in FIGS. 22A-22C for α2,3 (FIG. 22A) and α2,6 (FIG. 22B) sialic acids, and galactose (FIG. 22C). The results of transmembrane construct transfections, with secreted DAS181 transfection as control, are shown in FIGS. 23A-23C for α2,3 (FIG. 23A) and α2,6 (FIG. 23B) sialic acids, and galactose (FIG. 23C). Consistent with the imaging study results, secreted DAS181 and transmembrane DAS181 transfections led to removal of cell surface α2,3 and α2,6 sialic acids, and exposure of galactose, whereas transfections with secreted and transmembrane DAS185 or human Neu2 had little effect.

### Example 14: Secreted DAS181 and Transmembrane DAS181 Increase Tumor Cell Killing Mediated by PBMC and Oncolytic Virus

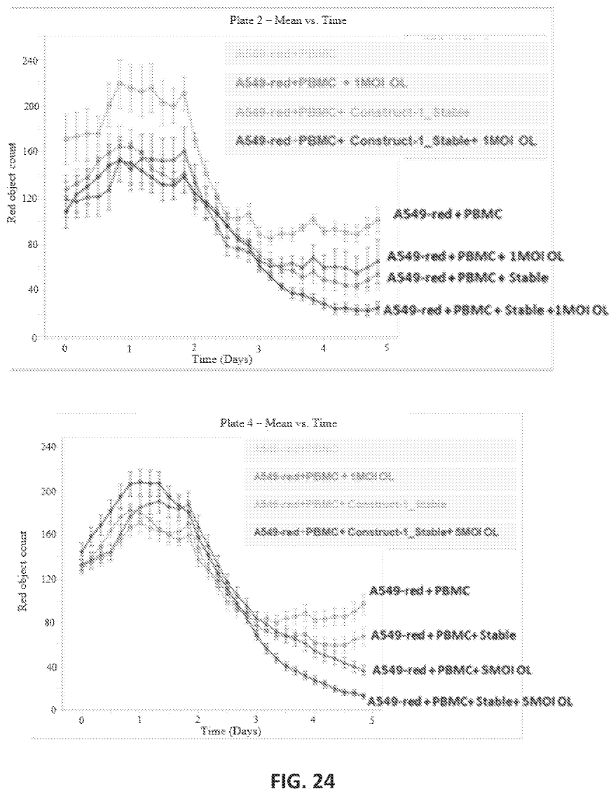

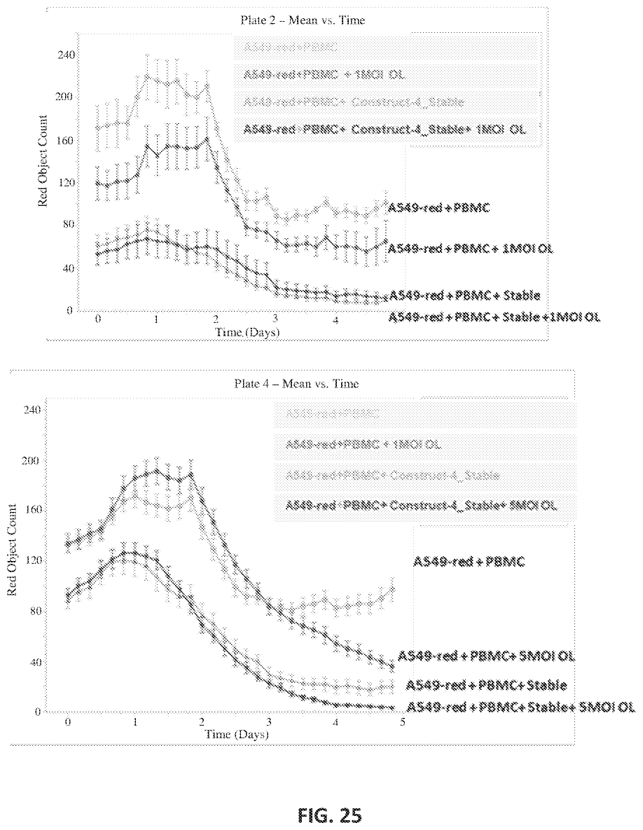

Because secreted DAS181 and transmembrane DAS181 were shown to remove cell surface sialic acid efficiently, their effect on PBMC and oncolytic virus-mediated tumor cell killing were evaluated with cells transfected with secreted and transmembrane DAS181. Because transient transfection can have deleterious effect on cell growth, stable pool cells for secreted and transmembrane DAS181 were generated by culturing the transfected A549-red cells in the presence of 1 mg/ml G418 for 3 weeks until the control non-transfected cells were completely killed off. Stable pool transfected A549-red cells with DAS181 were seeded into 96-well plate at density of 2000 cells per well. A549-red parental cells were seeded as controls. The next day, the complete growth medium was removed and replaced with 50 ul of medium with or without oncolytic virus. Freshly isolated PBMC were counted and resuspended at 200,000/ml in A549 complete medium with anti-CD3/anti-CD28/1L2, then 50 μl freshly PBMC were added to the cells. The cell growth was monitored by Essen Incucyte up to 5 days based on the counted red objects. As shown in FIG. 24, secreted DAS181 expression sensitized activated PBMC-mediated tumor cells killing and increased oncolytic virus associated PBMC-mediated cell killing at both MOI of 1 and 5. As shown in FIG. 25, transmembrane DAS181 expression significantly sensitized A549-red cells to activated PBMC killing. A far greater effect was virus was observed at MOI of 5, than at MOI of 1. It is possible that the potency of sialidase activity and oncolytic virus as single agent could be masking the additive effect when they were combined together under certain experimental conditions.

### Example 15: Generation of Sialidase-Armed Oncolytic Vaccinia Virus

This Example demonstrates generation of exemplary oncolytic virus constructs encoding sialidase. Constructs were successfully generated for Endo-Sial-VV, SP-Sial-VV, and TM-Sial-VV.

1.1. Design of pSEM-1-Sialidase-GFP/RFP.

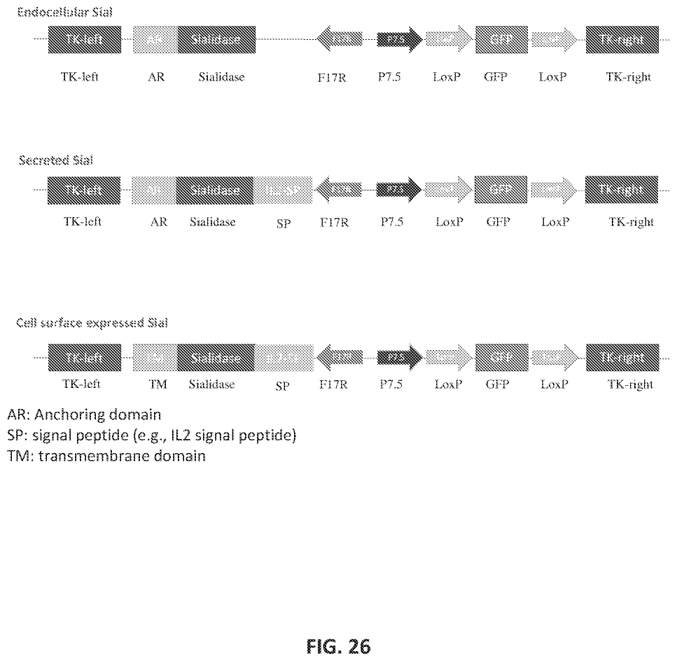

To generate the recombinant VV expressing Sialidase, pSEM-1 vectors were created using gene synthesis. The construct comprises of the gene encoding Sialidase, the gene encoding GFP or RFP, and two loxP sites with the same orientation flanking GFP/RFP (pSEM-1-Sialidase-GFP/RFP). The inserted Sialidase is under the transcriptional control of the F17R late promoter in order to limit the Sialidase expression within tumor tissue. The simplified design of the plasmids is as show in FIG. 26.

### 1.2. Generation of SP-Sial-VV and TM-Sial-VV.

Vaccinia virus (VV) strain WR was used as the parental virus for recombination with Sialidase to create VVs that expresses Sialidase in three different isoforms: i) constrained to the intracellular compartment (Endo-Sial-VV); ii) secreted to the extracellular environment (SP-Sial-VV); or iii) localized at the cell surface (TM-Sial-VV).

Sialidase-VVs were generated by insertion of pSEM-1-TK-Sialidase-GFP, pSEM-1-TK-SP-Sialidase-RFP or pSEM-1-TK-TM-Sialidase-GFP into the TK gene of VV through homologous recombination. All the viruses were produced and quantified by titration on CV-1 cells.

1.2.1. VV, endo-Sial-VV, SP-Sial-VV and TM-Sial-VV Quantification by Titration

After obtaining the recombinant viruses and having their stocks amplified, infectious particles were titrated by plaque assay. Briefly. CV-1 cells seeded in a 12-well plate were infected with serial dilutions of VV, endo-Sial-VV. SP-Sial-VV or TM-Sial-VV. After 48 h of infection, cells were fixed and stained with 20% Ethanol/0.1% Crystal Violet and virus plaques were counted. We prepared aliquots of 10⁶ of each virus stock in 100 μl of 10 mM Tris-HCl pH 9.0 for shipping. Therefore, all viruses are at 10⁷ pfu/ml.

### 1.2.2 Detection of Virus Recombination by PCR

In order to confirm that Sialidase isoforms were successfully inserted into VV genome, PCR was performed according to standard protocols to amplify the constructs using each virus stock as the template DNA. To do so, PCR primers were designed to specifically bind to the regions shown in FIG. 2. These primers will be able to confirm that: i) the constructs were successfully inserted into VV genome; ii) the constructs maintained their respective modifications during recombination (i.e. secretion and transmembrane domains). The primer sequences used were the following.

Sia1-fwd:
(SEQ ID NO: 56)
5′ - GGCCACACTGCTCGCCCAGCCAGTTCATG
Sia1-rev:
(SEQ ID NO: 57)
5′ - ATGCCTCCACCGAGCTGCCAGCAAGCATG
SP-Sia1-rev:
(SEQ ID NO: 83)
5′ - TCCTGTCTTGCATTGCACTAAGTCTTG
TM-Sia1-fwd:
(SEQ ID NO: 84)
5′ - TCATCACTAACGTGGCTTCTTCTGCCAAAGCATG

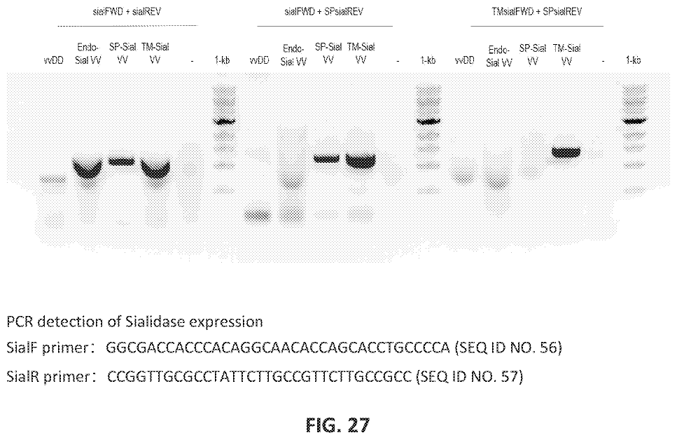

A band of the predicted size of Sialidase was detected in all three isoforms, demonstrating successful generation of the sialidase VV constructs (FIG. 27). When sialFWD+SPsialREV primer pair was used, only SP-Sial-VV and TM-Sial-VV showed bands of the expected size for SP-Sial, which confirms that these viruses have the secretion signal. Finally, when TM-sial-fwd+SP-Sial-rev primer was used, only TM-sial-VV showed a strong band of the predicted size of TM-Sialidase. This data confirms that VV recombinants were successfully generated and that the constructs for the three isoforms are intact within the virus genome.

### Example 16: Sialidase-VVs' are Able to Infect, Replicate in, and Lyse Tumor Cells In Vitro

This Example provides results demonstrating that Endo-Sial-VV, SP-Sial-VV, and TM-Sial-VV have comparable infectivity and replication activity in CV-1 and U87 cells, and comparable lytic activity in U87 and A549 cells to parental vaccinia virus, indicating the transgene didn't impair the VV's infectivity, replication, and lytic ability. Tumor cells were infected with Sialidase-VV, or parental VV at increasing MOIs. At various time points (24, 48, 72 or 96 hours) post infection, the cells were harvested and subjected to plaque assay and MTS assays to determine virus replication.

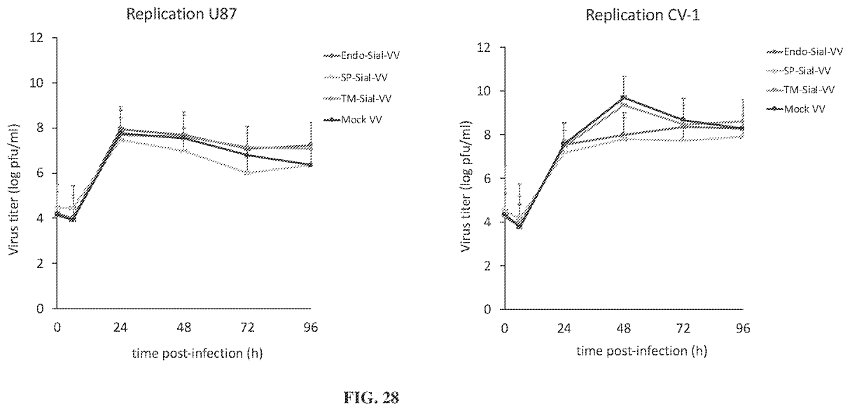

As shown in FIG. 28, the replication ability of the virus was not affected by modification with sialidase. CV-1 or U87 cells were plated in 12-well tissue culture plate and infected with Sialidase-VVs or VV at MOIs 0.1 in 2.5% FBS medium for 2 hours followed by culturing in complete medium. At various time points post infection (24, 48, 72, or 96 hours), the cells were harvested and virus replication was determined by plaque assay using CV-1 cells.

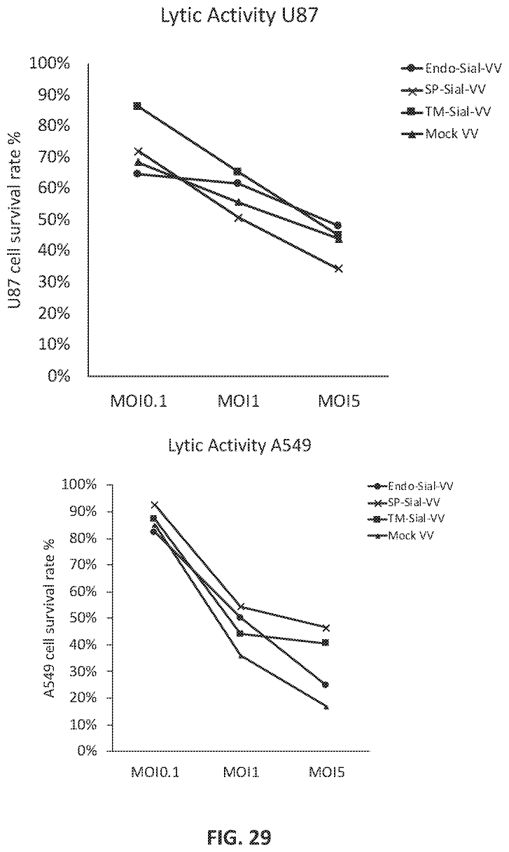

Furthermore, as shown in FIG. 29, the lytic activity of the modified vaccinia viruses was comparable to that of parental vaccinia virus in U87 and A549 cells, as shown in FIG. 29 and Tables 7-9 below.

TABLE 7
Percent (%) U87 cell survival

| | MOI0.1 | MOI1 | MOI5 |
|---|---|---|---|
| Endo-Sial-VV | 64.5% | 61.5% | 48.0% |
| SP-Sial-VV | 72.0% | 50.7% | 34.2% |
| TM-Sial-VV | 86.2% | 65.3% | 45.0% |
| Mock VV | 68.4% | 55.7% | 43.9% |

TABLE 8
Percent (%) A549 cell survival

| | MOI0.1 | MOI1 | MOI5 |
|---|---|---|---|
| Endo-Sial-VV | 82.4% | 50.2% | 24.7% |
| SP-Sial-VV | 92.5% | 54.5% | 46.4% |
| TM-Sial-VV | 87.2% | 44.0% | 40.6% |
| Mock VV | 85.0% | 36.0% | 16.7% |

### Example 17: Sialidase-VVs Enhance Dendritic Cell Maturation In Vitro

This Example provides results demonstrating: SP-, & TM-Sial-VV activated human DC by enhancing the its expression of maturation markers. Both SP-Sial-VV and TM-Sial-VV induced activation of DC effectively in vitro.

The effect of oncolytic viruses encoding a sialidase on maturation of DCs was evaluated. GM-CSF/IL4 derived human DC (Astarte, WA) were cultured with VV-U87 tumor cells (ATCC, VA) for 24 hours. DC were collected and stained with antibodies against DC maturation markers CD86, CD80, HLA-ABC, HLA-Dr on DCs were determined by flow cytometry. Cell were collected and stained with HLA-Dr-FITC (ab193620, Abcam, MA) and HLA-ABC-PE (ab155381, Abcam, MA), or CD80-FITC (ab18279, Abcam, MA) and CD86-PE (ab234226, Abcam, MA) antibodies and subjected to flow analysis (Sony SA3800).

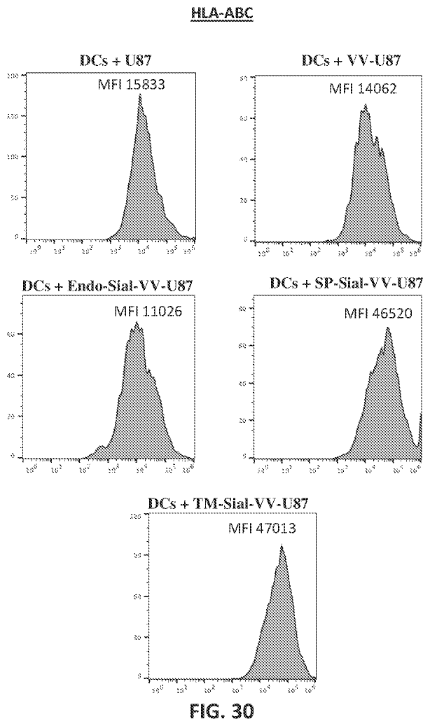

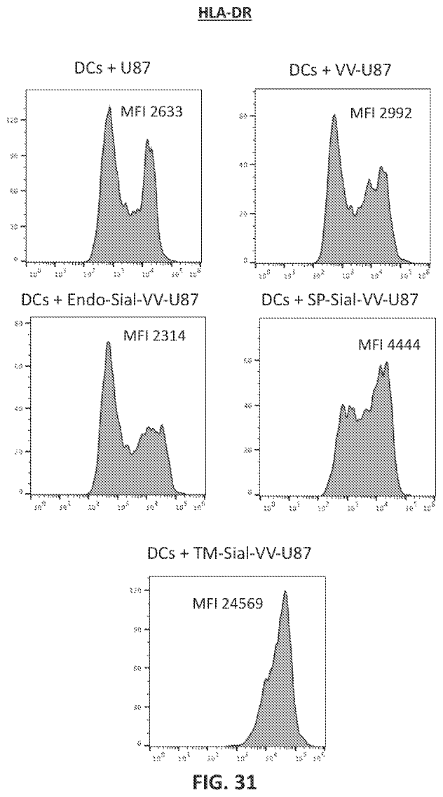

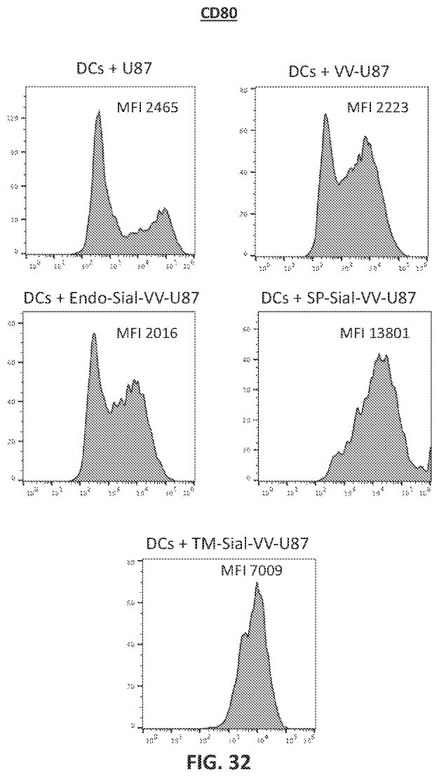

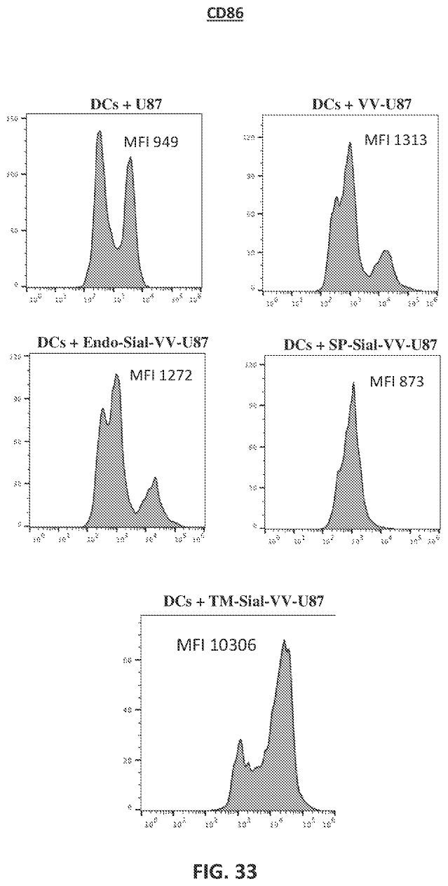

FIGS. 30-33 show expression of DC maturation markers HLA-ABC, HLA-DR, CD80, and CD86, respectively. Culturing DCs together with U87 tumor cells infected with SP-Sial-VV or TM-Sial-VV enhanced expression of DC maturation markers compared to that of DC cells cultures with U87 infected with VV or U87 alone.

### Example 18: Sialidase-VVs Enhance NK-Mediated Tumor Cell Killing In Vitro

This Example provides results demonstrating that Sial-VVs enhance NK-mediated cytotoxicity. VV-infected tumor cells were co-cultured with NK, and specific lysis of the tumor cells was determined.

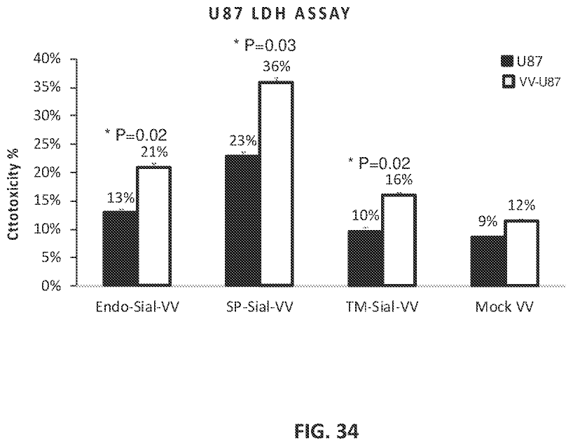

Protocol: Negative selected human NK cells (Astarte, WA) and VV-U87 cells (ATCC, VA) were co-cultured, and tumor killing efficacy was measured by LDH assay (Abcam, MA). As shown in FIG. 34, the results suggested that Sial-VVs enhanced NK cell-mediated U87 tumor killing in vitro. (* P value, the Sial-VV vs Mock VV in U87 and NK culture)

Specific lysis was calculated as:

$$\% = 100 \% \times \frac{\begin{matrix}experimental target cell release - \\ target cells spontaneous release\end{matrix}}{\begin{matrix}target cells maximum release - \\ target cells spontaneous release\end{matrix}}$$

Example 19
Sialidase-VVs inhibit tumor growth in vivo

| | Endo- | SP- | TM- | Mock |
|---|---|---|---|---|
| | Sial-VV | Sial-VV | Sial-VV | VV |
| U87 | 13% | 23% | 10% | 9% |
| U87 + NK | 21% | 36% | 16% | 12% |
| P value | 0.02 | 0.03 | 0.02 | |
| (Sial-VV vs VV) | | | | |
| P value | 0.04 | 0.03 | 0.01 | |
| (NK vs no NK) | | | | |

*P-value: T. Test were used with 1 tail and type 1 analysis.

### Example 19: Sialidase-VVs Inhibit Tumor Growth In Vivo

The Examples above demonstrate the surprising beneficial effects of Sialidase-VVs in vitro in promoting immune cell activation and cytotoxicity. Example 19 provides results demonstrating that Sialidase-VVs significantly inhibit tumor growth in vitro compared to control VV.

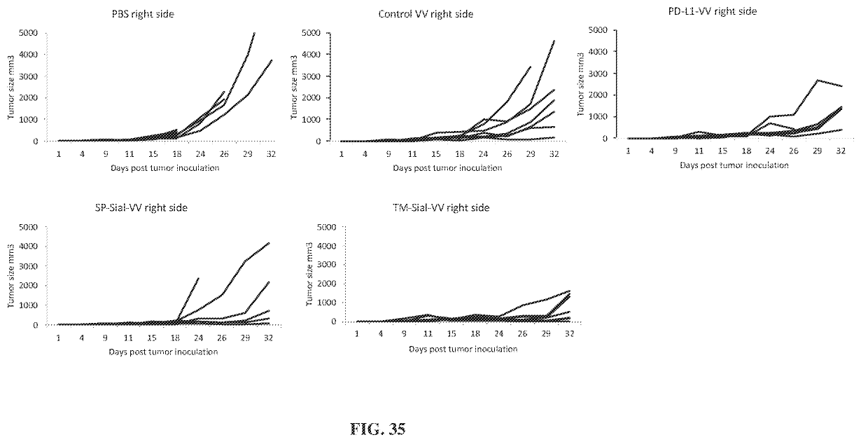

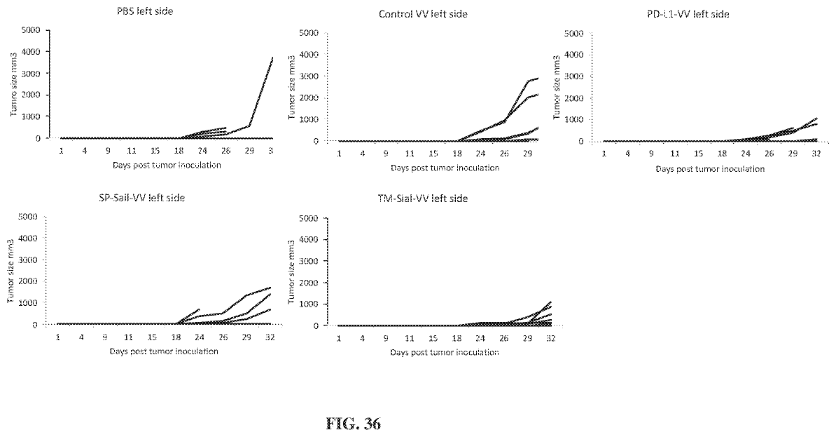

To test the effect of Sialidase-VVs on tumor growth in vivo, 2×10⁵ and 2×10⁴ B16-F10 tumor cells were inoculated on the right or left flank of C57 mice. When the tumor size on the right or left flank reached 100 mm (14 days), 4×10⁷ pfu VVs were injected intratumorally into the tumor on the right or left site every other day for 3 doses. Tumor size was measured. FIG. 35 shows the tumor size on the right flank. The results indicated that TM-sial-VV significantly inhibited tumor growth compared to control VV. SP-sial VV inhibited tumor growth, albeit to a lesser extent. FIG. 36 shows the tumor size on the left flank. The results indicated that TM-sial-VV significantly inhibited tumor growth compared to control VV.

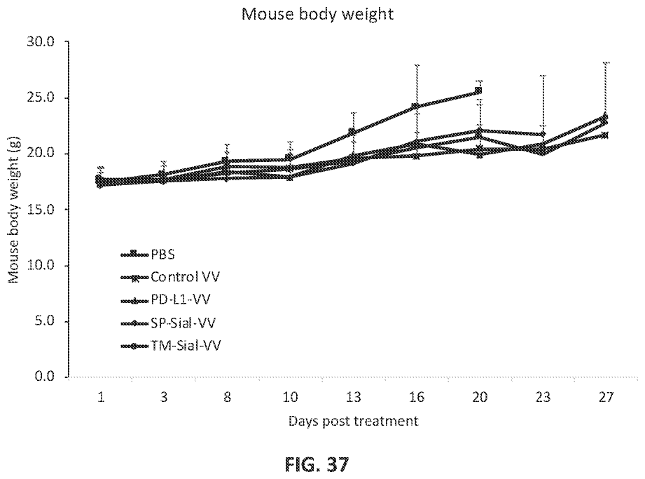

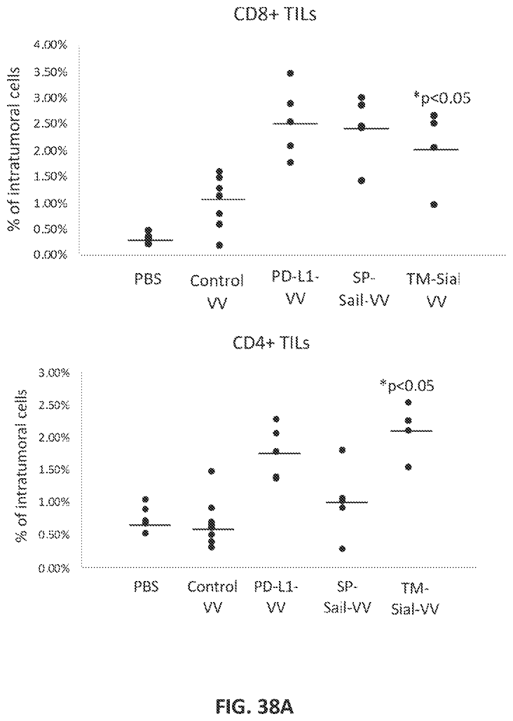

FIG. 37 shows that there was no significant difference in mouse body weight for mice treated with the various VVs or PBS control. 2×10⁵ and 2×10⁴ B16-F10 tumor cells were inoculated on the right or left flank of C57 mice. When the right tumor size reached 100 mm (14 days), 4×10⁷ pfu VVs were injected intratumorally every other day for 3 doses.

Sialidase Armed Oncolytic Vaccinia Virus Significantly Enhances CD8+ and CD4+ T Cell Infiltration within Tumor

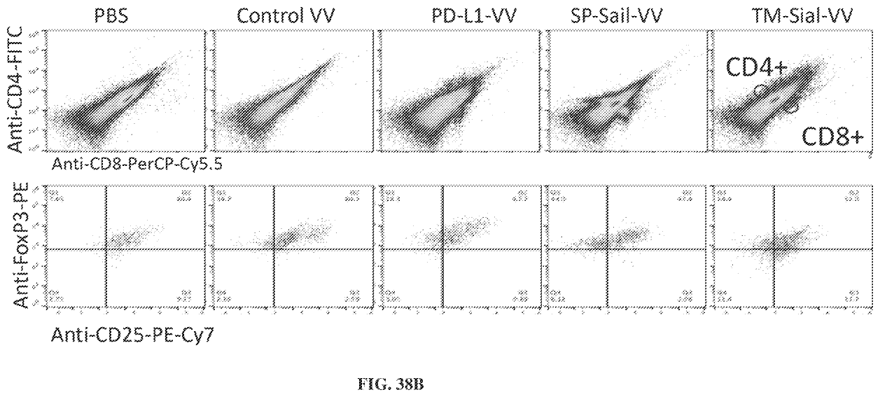

Tumor cells were inoculated on the right flank of C57 mice, and the resulting tumors were intratumorally injected with VVs as described above (every other day for 3 doses). 7 days after the first VV treatment, tumor tissues (n=6) were collected and subjected to flow analysis to analyze CD8+ and CD4+ T cell infiltration within the tumor. * p value: treatment group vs control VV group. FIG. 38A shows quantification of the results and p values demonstrating significant enhancement of CD8+ and CD4+ T cell infiltration by sialidase armed oncolytic vaccinia virus. FIG. 38B shows the FACS plots. The results demonstrated that sialidase armed oncolytic vaccinia virus significantly enhanced CD8+ and CD4+ T cell infiltration within tumor compared to control vaccinia virus.

Sialidase Armed Oncolytic Vaccinia Virus Significantly Decreased the Ratio of Treg/CD4+ T Cells within the Tumor

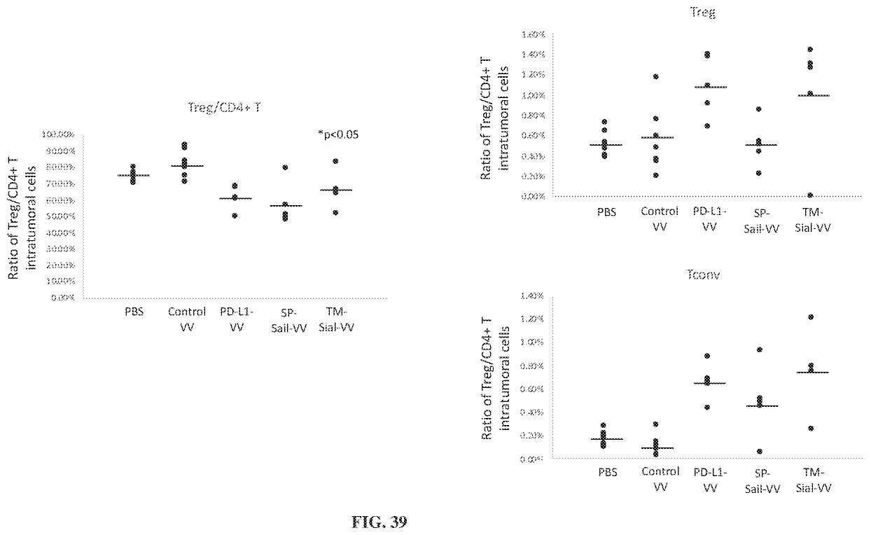

Tumor cells were inoculated on the right flank of C57 mice, and the resulting tumors were intratumorally injected with VVs as described above (every other day for 3 doses). 7 days after the first VV treatment, tumor tissues (n=6) were collected and subjected to flow analysis to determine the ratio of Treg/CD4+ T cells within the tumor. As shown in FIG. 39, TM-Sial-VV decreased the ratio of Treg/CD4+ T cells within the tumor, compared to control VV. * p value: treatment group vs control VV group.

Sialidase Armed Oncolytic Vaccinia Virus Significantly Enhances NK and NKT Cell Infiltration within Tumor

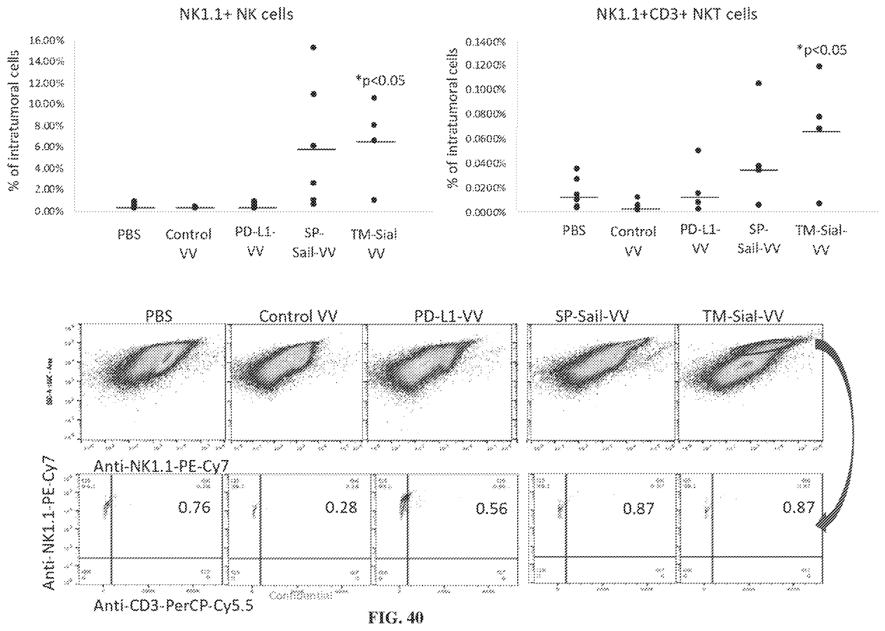

Tumor cells were inoculated on the right flank of C57 mice, and the resulting tumors were intratumorally injected with VVs as described above (every other day for 3 doses). 7 days after the first VV treatment, tumor tissues (n=6) were collected and subjected to flow analysis to determine the number of NK1.1+NK cells. As shown in FIG. 40, sialidase armed oncolytic vaccinia virus significantly enhanced NK and NKT cell infiltration within tumor. * p value, treatment group vs control VV group.

Sialidase Armed Oncolytic Vaccinia Virus Significantly Enhances NK and NKT Cell Infiltration within Tumor

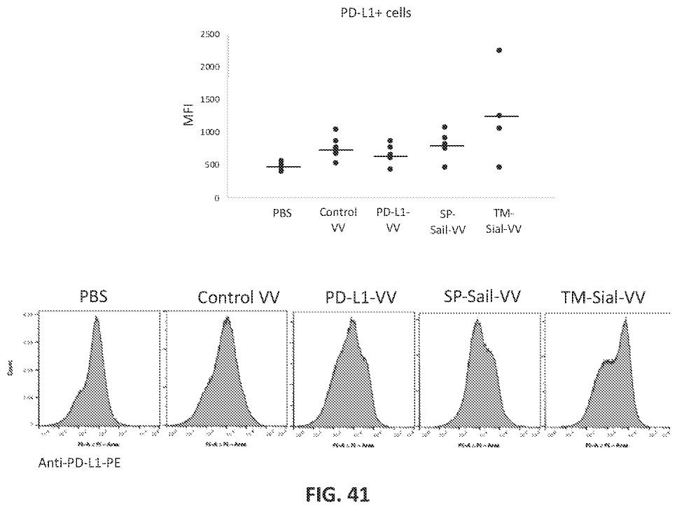

Tumor cells were inoculated on the right flank of C57 mice, and the resulting tumors were intratumorally injected with VVs as described above (every other day for 3 doses). 7 days after the first VV treatment, tumor tissues (n=6) were collected and subjected to flow analysis to determine expression of PD-L1. As shown in FIG. 41, transmembrane bound sialidase armed oncolytic virus significantly increased PD-L1 expression within tumor cells (p<0.05, TM-Sial-VV vs Control VV).

SEQUENCE LISTING: EXEMPLARY SEQUENCES

SEQ ID NO: 3 Human Neu1 sialidase
MTGERPSTALPDRRWGPRILGFWGGCRVWVFAAIFLLLSLAASWSKAENDFGLVQP
LVTMEQLLWVSGRQIGSVDTFRIPLITATPRGTLLAFAEARKMSSSDEGAKFIALRRS
MDQGSTWSPTAFIVNDGDVPDGLNLGAVVSDVETGVVFLFYSLCAHKAGCQVAST
MLVWSKDDGVSWSTPRNLSLDIGTEVFAPGPGSGIQKQREPRKGRLIVCGHGTLERD
GVFCLLSDDHGASWRYGSGVSGIPYGQPKQENDFNPDECQPYELPDGSVVINARNQ
NNYHCHCRIVLRSYDACDTLRPRDVTFDPELVDPVVAAGAVVTSSGIVFFSNPAHPE
FRVNLTLRWSFSNGTSWRKETVQLWPGPSGYSSLATLEGSMDGEEQAPQLYVLYEK
GRNHYTESISVAKISVYGTL
SEQ ID NO: 4 Human Neu2 sialidase
MASLPVLQKESVFQSGAHAYRIPALLYLPGQQSLLAFAEQRASKKDEHAELIVLRRG
DYDAPTHQVQWQAQEVVAQARLDGHRSMNPCPLYDAQTGTLFLFFIAIPGQVTEQQ
QLQTRANVTRLCQVTSTDHGRTWSSPRDLTDAAIGPAYREWSTFAVGPGHCLQLHD
RARSLVVPAYAYRKIHPIQRPIPSAFCFLSHDHGRTWARGHFVAQDTLECQVAEVET
GEQRVVTLNARSHLRARVQAQSTNDGLDFQESQLVKKLVEPPPQGCQGSVISFPSPR
SGPGSPAQWLLYTHPTHSWQRADLGAYLNPRPPAPEAWSEPVLLAKGSCAYSDLQS
MGTGPDGSPLFGCLYEANDYEEIVFLMFTLKQAFPAEYLPQ
SEQ ID NO: 5 Human Neu3 sialidase
MEEVTTCSFNSPLFRQEDDRGITYRIPALLYIPPTHTFLAFAEKRSTRRDEDALHLVLR
RGLRIGQLVQWGPLKPLMEATLPGHRTMNPCPVWEQKSGCVFLFFICVRGHVTERQ
QIVSGRNAARLCFIYSQDAGCSWSEVRDLTEEVIGSELKHWATFAVGPGHGIQLQSG
RLVIPAYTYYIPSWFFCFQLPCKTRPHSLMIYSDDLGVTWHHGRLIRPMVTVECEVAE
VTGRAGHPVLYCSARTPNRCRAEALSTDHGEGFQRLALSRQLCEPPHGCQGSVVSFR
PLEIPHRCQDSSSKDAPTIQQSSPGSSLRLEEEAGTPSESWLLYSHPTSRKQRVDLGIY
LNQTPLEAACWSRPWILHCGPCGYSDLAALEEEGLFGCLFECGTKQECEQIAFRLFT
HREILSHLQGDCTSPGRNPSQFKSN
SEQ ID NO: 6 Human Neu4 sialidase
MGVPRTPSRTVLFERERTGLTYRVPSLLPVPPGPTLLAFVEQRLSPDDSHAHRLVLRR
GTLAGGSVRWGALHVLGTAALAEHRSMNPCPVHDAGTGTVFLFFIAVLGHTPEAVQ
IATGRNAARLCCVASRDAGLSWGSARDLTEEAIGGAVQDWATFAVGPGHGVQLPS
GRLLVPAYTYRVDRRECFGKICRTSPHSFAFYSDDHGRTWRCGGLVPNLRSGECQLA
AVDGGQAGSFLYCNARSPLGSRVQALSTDEGTSFLPAERVASLPETAWGCQGSIVGF
PAPAPNRPRDDSWSVGPGSPLQPPLLGPGVHEPPEEAAVDPRGGQVPGGPFSRLQPR
GDGPRQPGPRPGVSGDVGSWTLALPMPFAAPPQSPTWLLYSHPVGRRARLHMGIRL
SQSPLDPRSWTEPWVIYEGPSGYSDLASIGPAPEGGLVFACLYESGARTSYDEISFCTF
SLREVLENVPASPKPPNLGDKPRGCCWPS
SEQ ID NO: 7 Human Neu4 isoform 2 sialidase
MMSSAAFPRWLSMGVPRTPSRTVLFERERTGLTYRVPSLLPVPPGPTLLAFVEQRLSP
DDSHAHRLVLRRGTLAGGSVRWGALHVLGTAALAEHRSMNPCPVHDAGTGTVFLF
FIAVLGHTPEAVQIATGRNAARLCCVASRDAGLNWGSARDLTEEAIGGAVQDWATF
AVGPGHGVQLPSGRLLVPAYTYRVDRRECFGKICRTSPHSFAFYSDDHGRTWRCGG
LVPNLRSGECQLAAVDGGQAGSFLYCNARSPLGSRVQALSTDEGTSFLPAERVASLP
ETAWGCQGSIVGFPAPAPNRPRDDSWSVGPGSPLQPPLLGPGVHEPPEEAAVDPRGG
QVPGGPFSRLQPRGDGPRQPGPRPGVSGDVGSWTLALPMPFAAPPQSPTWLLYSHPV
GRRARLHMGIRLSQSPLDPRSWTEPWVIYEGPSGYSDLASIGPAPEGGLVFACLYESG
ARTSYDEISFCTFSLREVLENVPASPKPPNLGDKPRGCCWPS
SEQ ID NO: 8 Human Neu4 isoform 3 sialidase
MMSSAAFPRWLQSMGVPRTPSRTVLFERERTGLTYRVPSLLPVPPGPTLLAFVEQRL
SPDDSHAHRLVLRRGTLAGGSVRWGALHVLGTAALAEHRSMNPCPVHDAGTGTVF
LFFIAVLGHTPEAVQIATGRNAARLCCVASRDAGLSWGSARDLTEEAIGGAVQDWA
TFAVGPGHGVQLPSGRLLVPAYTYRVDRRECFGKICRTSPHSFAFYSDDHGRTWRCG
GLVPNLRSGECQLAAVDGGQAGSFLYCNARSPLGSRVQALSTDEGTSFLPAERVASL
PETAWGCQGSIVGFPAPAPNRPRDDSWSVGPGSPLQPPLLGPGVHEPPEEAAVDPRG
GQVPGGPFSRLQPRGDGPRQPGPRPGVSGDVGSWTLALPMPFAAPPQSPTWLLYSHP
VGRRARLHMGIRLSQSPLDPRSWTEPWVIYEGPSGYSDLASIGPAPEGGLVFACLYES
GARTSYDEISFCTFSLREVLENVPASPKPPNLGDKPRGCCWPS
SEQ ID NO: 9 A. viscosus nanH sialidase
MTSHSPFSRRRLPALLGSLPLAATGLIAAAPPAHAVPTSDGLADVTITQVNAPADGLY
SVGDVMTFNITLTNTSGEAHSYAPASTNLSGNVSKCRWRNVPAGTTKTDCTGLATH
TVTAEDLKAGGFTPQIAYEVKAVEYAGKALSTPETIKGATSPVKANSLRVESITPSSS
QENYKLGDTVSYTVRVRSVSDKTINVAATESSFDDLGRQCHWGGLKPGKGAVYNC
KPLTHTITQADVDAGRWTPSITLTATGTDGATLQTLTATGNPINVVGDHPQATPAPA
PDASTELPASMSQAQHLAANTATDNYRIPAIPPPPMGTCSSPTTSARRTTATAAATTP
NPNHIVQRRSTDGGKTWSAPTYIHQGTETGKKVGYSDPSYVVDHQTGTIFNFHVKSY
DQGWGGSRGGTDPENRGIIQAEVSTSTDNGWTWTHRTITADITKDKPWTARFAASG
QGIQIQHGPHAGRLVQQYTIRTAGGPVQAVSVYSDDHGKTWQAGTPIGTGMDENKV
VELSDGSLMLNSRASDGSGFRKVAHSTDGGQTWSEPVSDKNLPDSVDNAQIIRAFPN
AAPDDPRAKVLLLSHSPNPRPWCRDRGTISMSCDDGASWTTSKVFHEPFVGYTTIAV
QSDGSIGLLSEDAHNGADYGGIWYRNFTMNWLGEQCGQKPAEPSPGRRRRRHPQRH
RRRSRPRRPRRALSPRRHRHHPPRPSRALRPSRAGPGAGAHDRSEHGAHTGSCAQSA
PEQTDGPTAAPAPETSSAPAAEPTQAPTVAPSVEPTQAPGAQPSSAPKPGATGRAPSV
VNPKATGAATEPGTPSSSASPAPSRNAAPTPKPGMEPDEIDRPSDGTMAQPTGAPAR
RVPRRRRRRRPAAGCLARDQRAADPGPCGCRGCRRVPAAAGSPFEELNTRRAGHPA
LSTD
SEQ ID NO: 10 A. viscosus nanA sialidase
MTTTKSSALRRLSALAGSLALAVTGIIAAAPPAHATPTSDGLADVTITQTHAPADGIY
AVGDVMTFDITLTNTSGQARSFAPASTNLSGNVLKCRWSNVAAGATKTDCTGLATH
TVTAEDLKAGGFTPQIAYEVKAVGYKGEALNKPEPVTGPTSQIKPASLKVESFTLASP
KETYTVGDVVSYTVRIRSLSDQTINVAATDSSFDDLARQCHWGNLKPGQGAVYNCK
PLTHTITQADADHGTWTPSITLAATGTDGAALQTLAATGEPLSVVVERPKADPAPAP
DASTELPASMSDAQHLAENTATDNYRIPAITTAPNGDLLVSYDERPRDNGNNGGDSP
NPNHIVQRRSTDGGKTWSAPSYIHQGVETGRKVGYSDPSYVVDNQTGTIFNFHVKSF
DQGWGHSQAGTDPEDRSVIQAEVSTSTDNGWSWTHRTITADITRDNPWTARFAASG
QGIQIHQGPHAGRLVQQYTIRTADGVVQAVSVYSDDHGQTWQAGTPTGTGMDENK
VVELSDGSLMLNSRASDGTGFRKVATSTDGGQTWSEPVPDKNLPDSVDNAQIIRPFP
NAAPSDPRAKVLLLSHSPNPRPWSRDRGTISMSCDNGASWVTGRVFNEKFVGYTTIA
VQSDGSIGLLSEDGNYGGIWYRNFTMGWVGDQCSQPRPEPSPSPTPSAAPSAEPTSEP
TTAPAPEPTTAPSSEPSVSPEPSSSAIPAPSQSSSATSGPSTEPDEIDRPSDGAMAQPTGG
AGRPSTSVTGATSRNGLSRTGTNALLVLGVAAAAAAGGYLVLRIRRARTE
SEQ ID NO: 11 S. oralis nanA sialidase
MNYKSLDRKQRYGIRKFAVGAASVVIGTVVFGANPVLAQEQANAAGANTETVEPG
QGLSELPKEASSGDLAHLDKDLAGKLAAAQDNGVEVDQDHLKKNESAESETPSSTE
TPAEEANKEEESEDQGAIPRDYYSRDLKNANPVLEKEDVETNAANGQRVDLSNELD
KLKQLKNATVHMEFKPDASAPRFYNLFSVSSDTKENEYFTMSVLDNTALIEGRGAN
GEQFYDKYTDAPLKVRPGQWNSVTFTVEQPTTELPHGRVRLYVNGVLSRTSLKSGN
FIKDMPDVNQAQLGATKRGNKTVWASNLQVRNLTVYDRALSPDEVQTRSQLFERG
ELEQKLPEGAKVTEKEDVFEGGRNNQPNKDGIKSYRIPALLKTDKGTLIAGTDERRL
HHSDWGDIGMVVRRSSDNGKTWGDRIVISNPRDNEHAKHADWPSPVNIDMALVQD
PETKRIFAIYDMFLESKAVFSLPGQAPKAYEQVGDKVYQVLYKQGESGRYTIRENGE
VFDPQNRKTDYRVVVDPKKPAYSDKGDLYKGNELIGNIYFEYSEKNIFRVSNTNYL
WMSYSDDDGKTWSAPKDITHGIRKDWMHFLGTGPGTGIALRTGPHKGRLVIPVYTT
NNVSYLSGSQSSRVIYSDDHGETWQAGEAVNDNRPVGNQTIHSSTMNNPGAQNTES
TVVQLNNGDLKLFMRGLTGDLQVATSHDGGATWDKEIKRYPQVKDVYVQMSAIHT
MHEGKEYILLSNAGGPGRNNGLVHLARVEENGELTWLKHNPIQSGKFAYNSLQELG
NGEYGLLYEHADGNQNDYTLSYKKFNWDFLSRDRISPKEAKVKYAIQKWPGIIAME
FDSEVLVNKAPTLQLANGKTATFMTQYDTKTLLFTIDPEDMGQRITGLAEGAIESMH
NLPVSLAGSKLSDGINGSEAAIHEVPEFTGGVNAEEAAVAEIPEYTGPLATVGEEVAP
TVEKPEFTGGVNAEEAPVAEMPEYTGPLSTVGEEVAPTVEKPEFTGGVNAVEAAVH
ELPEFKGGVNAVLAASNELPEYRGGANFVLAASNDLPEYIGGVNGAEAAVHELPEY
KGDTNLVLAAADNKLSLGQDVTYQAPAAKQAGLPNTGSKETHSLISLGLAGVLLSL
FAFGKKRKE
SEQ ID NO: 12 S. oralis nanH sialidase
MSDLKKYEGVIPAFYACYDDQGEVSPERTRALVQYFIDKGVQGLYVNGSSGECIYQS
VEDRKLILEEVMAVAKGKLTIIAHVACNNTKDSMELARHAESLGVDAIATIPPIYFRL
PEYSVAKYWNDISAAAPNTDYVIYNIPQLAGVALTPSLYTEMLKNPRVIGVKNSSMP
VQDIQTFVSLGGEDHIVFNGPDEQFLGGRLMGAKAGIGGTYGAMPELFLKLNQLIAE
KDLETARELQYAINAIIGKLTSAHGNMYGVIKEVLKINEGLNIGSVRSPLTPVTEEDRP
VVEAAAQLIRETKERFL
SEQ ID NO: 13 S. mitis nanA sialidase
MNQRHFDRKQRYGIRKFTVGAASVVIGAVVFGVAPALAQEAPSTNGETAGQSLPEL
PKEVETGNLTNLDKELADKLSTATDKGTEVNREELQANPGSEKAAETEASNETPATE
SEDEKEDGNIPRDFYARELENVNTVVEKEDVETNPSNGQRVDMKEELDKLKKLQNA
TIHMEFKPDASAPRFYNLFSVSSDTKVNEYFTMAILDNTAIVEGRDANGNQFYGDYK
TAPLKIKPGEWNSVTFTVERPNADQPKGQVRVYVNGVLSRTSPQSGRFIKDMPDVN
QVQIGTTKRTGKNFWGSNLKVRNLTVYDRALSPEEVKKRSQLFERGELEKKLPEGA
KVTDKLDVFQGGENRKPNKDGIASYRIPALLKTDKGTLIAGADERRLHHSDWGDIG
MVVRRSDDKGKTWGDRIVISNPRDNENARRAHAGSPVNIDMALVQDPKTKRIFSIFD
MFVEGEAVRDLPGKAPQAYEQIGNKVYQVLYKKGEAGHYTIRENGEVFDPENRKTE
YRVVVDPKKPAYSDKGDLYKGEELIGNVYFDYSDKNIFRVSNTNYLWMSYSDDDG
KTWSAPKDITYGIRKDWMHFLGTGPGTGIALHSGPHKGRLVIPAYTTNNVSYLGGSQ
SSRVIYSDDHGETWHAGEAVNDNRPIGNQTIHSSTMNNPGAQNTESTVVQLNNGDL
KLFMRGLTGDLQVATSKDGGATWEKDVKRYADVKDVYVQMSAIHTVQEGKEYIIL
SNAGGPGRYNGLVHVARVEANGDLTWIKHNPIQSGKFAYNSLQDLGNGEFGLLYEH
ATATQNEYTLSYKKFNWDFLSKDGVAPTKATVKNAVEMSKNVIALEFDSEVLVNQP
PVLKLANGNFATFLTQYDSKTLLFAASKEDIGQEITEIIDGAIESMHNLPVSLEGAGVP
GGKNGAKAAIHEVPEFTGAVNGEGTVHEDPAFEGGINGEEAAVHDVPDFSGGVNGE
VAAIHEVPEFTGGINGEEAAKLELPSYEGGANAVEAAKSELPSYEGGANAVEAAKLE
LPSYESGAHEVQPASSNLPTLADSVNKAEAAVHKGKEYKANQSTAVQAMAQEHTY
QAPAAQQHLLPKTGSEDKSSLAIVGFVGMFLGLLMIGKKRE
SEQ ID NO: 14 S mitis nanA_1 sialidase
MNQSSLNRKNRYGIRKFTIGVASVAIGSVLFGITPALAQETTTNIDVSKVETSLESGAP
VSEPVTEVVSGDLNHLDKDLADKLALATNQGVDVNKHNLKEETSKPEGNSEHLPVE
SNTGSEESIEHHPAKIEGADDAVVPPRDFFARELTNVKTVFEREDLATNTGNGQRVD
LAEELDQLKQLQNATIHMEFKPDANAPQFYNLFSVSSDKKKDEYFSMSVNKGTAMV
EARGADGSHFYGSYSDAPLKIKPGQWNSVTFTVERPKADQPNGQVRLYVNGVLSRT
NTKSGRFIKDMPDVNKVQIGATRRANQTMWGSNLQIRNLTVYNRALTIEEVKKRSH
LFERNDLEKKLPEGAEVTEKKDIFESGRNNQPNGEGINSYRIPALLKTDKGTLIAGGD
ERRLHHFDYGDIGMVIRRSQDNGKTWGDKLTISNLRDNPEATDKTATSPLNIDMVLV
QDPTTKRIFSIYDMFPEGRAVFGMPNQPEKAYEEIGDKTYQVLYKQGETERYTLRDN
GEIFNSQNKKTEYRVVVNPTEAGFRDKGDLYKNQELIGNIYFKQSDKNPFRVANTSY
LWMSYSDDDGKTWSAPKDITPGIRQDWMKFLGTGPGTGIVLRTGAHKGRILVPAYT
TNNISHLGGSQSSRLIYSDDHGQTWHAGESPNDNRPVGNSVIHSSNMNKSSAQNTES
TVLQLNNGDVKLFMRGLTGDLQVATSKDGGVTWEKTIKRYPEVKDAYVQMSAIHT
MHDGKEYILLSNAAGPGRERKNGLVHLARVEENGELTWLKHNPIQNGEFAYNSLQE
LGGGEYGLLYEHRENGQNYYTLSYKKFNWDFVSKDLISPTEAKVSQAYEMGKGVF
GLEFDSEVLVNRAPILRLANGRTAVFMTQYDSKTLLFAVDKKDIGQEITGIVDGSIES
MHNLTVNLAGAGIPGGMNAAESVEHYTEEYTGVLGTSGVEGVPTISVPEYEGGVNS
ELALVSEKEDYRGGVNSASSVVTEVLEYTGPLSTVGSEDAPTVSVLEYEGGVNIDSP
EVTEAPEYKEPIGTSGYELAPTVDKPAYTGTIEPLEKEENSGAIIEEGNVSYITENNNK
PLENNNVTTSSIISESSKLKHTLKNATGSVQIHASEEVLKNVKDVKIQEVKVSSLSSLN
YKAYDIQLNDASGKAVQPKGTVIVTFAAEQSVENVYYVDSKGNLHTLEFLQKDGEV
TFETNHFSIYAMTFQLSLDNVVLDNHREDKNGEVNSASPKLLSINGHSQSSQLENKV
SNNEQSKLPNTGEDKSISTVLLGFVGVILGAMIFYRRKDSEG
SEQ ID NO: 15 S. mitis nanA_2 sialidase
MDKKKIILTSLASVAVLGAALAASQPSLVKAEEQPTASQPAGETGTKSEVTSPEIKQA
EADAKAAEAKVTEAQAKVDTTTPVADEAAKKLETEKKEADEADAAKTKAEEAKKT
ADDELAAAKEKAAEADAKAKEEAKKEEDAKKEEADSKEALTEALKQLPDNELLDK
KAKEDLLKAVEAGDLKASDILAELADDDKKAEANKETEKKLRNKDQANEANVATT
PAEEAKSKDQLPADIKAGIDKAEKADAARPASEKLQDKADDLGENVDELKKEADAL
KAEEDKKAETLKKQEDTLXEAKEALKSAKDNGFGEDITAPLEKAVTATEKERDAAQ
NAFDQAASDTKAVADELNKLTDEYNKTLEEVKAAKEKEANEPAKPVEEEPAKPAEK
TEAEKAAEAKTEADAKVAELQKKADEAKTKADEATAKATKEAEDVKAAEKAKEE
ADKAKTDAEAELAKAKEEAEKAKAKVEELKKEEKDNLEALKAALDQLEKDIDADA
TITNKEEAKKALGKEDILAAVEKGDLTAGDVLKELENQNATAEATKDQDPQADEIG
ATKQEGKPLSELPAADKEKLDAAYNKEASKPIVKKLQDIADDLVEKIEKLTKVADKD
KADATEKAKAVEEKNAALDKQKETLDKAKAALETAKKNQADQAIQDGLQDAVTK
LEASFASAKTAADEAQAKFDEVNEVVKAYKAAIDELTDDYNATLGHIENLKEVPKG
EEPKDFSGGVNDDEAPSSTPNTNEFTGGANDADAPTAPNANEFAGGVNDEEAPTTE
NKPEFNGGVNDEEAPTVPNKPEGEAPKPTGENAKDAPVVKLPEFGANNPEIKKILDEI
AKVKEQIKDGEENGSEDYYVEGLKERLADLEEAFDTLSKNLPAVNKVPEYTGPVTPE
NGQTQPAVNTPGGQQGGSSQQTPAVQQGGSGQQAPAVQQGGSNQQVPAVQQTNTP
AVAGTSQDNTYQAPAAKEEDKKELPNTGGQESAALASVGFLGLLLGALPFVKRKN
SEQ ID NO: 16 S. mitis nanA_3 sialidase
MKYRDFDRKRRYGIRKFAVGAASVVIGTVVFGANPVLAQEQANAAGANTETVEPG
QGLSELPKEASSGDLAHLDKDLAGKLAAAQDNGVEVDQDHLKKNESAESETPSSTE
TPAEGTNKEEESEDQGAIPRDYYSRDLKNANPVLEKEDVETNAANGQRVDLSNELD
KLKQLKNATVHMEFKPDASAPRFYNLFSVSSDTKENEYFTISVLDNTALIEGRGANG
EQFYDKYTDAPLKVRPGQWNSVTFTVEQPTTELPHGRVRLYVNGVLSRTSLKSGNFI
KDMPDVNQAQLGATKRGNKTVWASNLQVRNLTVYDRALSPDEVQTRSQLFERGEL
EQKLPEGAKVTEKEDVFEGGRNNQPNKDGIKSYRIPALLKTDKGTLIAGTDERRLHH
SDWGDIGMVVRRSSDNGKTWGDRIVISNPRDNEHAKHADWPSPVNIDMALVQDPE
TKRIFAIYDMFLESKAVFSLPGQAPKAYEQVGDKVYQVLYKQGESGRYTIRENGEVF
DPQNRKTDYRVVVDPKKPAYSDKGDLYKGNELIGNIYFEYSEKNIFRVSNTNYLWM
SYSDDDGKTWSAPKDITHGIRKDWMHFLGTGPGTGIALRTGPHKGRLVIPVYTTNN
VSYLSGSQSSRVIYSDDHGETWQAGEAVNDNRPVGNQTIHSSTMNNPGAQNTESTV
VQLNNGDLKLFMRGLTGDLQVATSHDGGATWDKEIKRYPQVKDVYVQMSAIHTM
HEGKEYILLSNAGGPGRNNGLVHLARVEENGELTWLKHNPIQSGKFAYNSLQDLGN
GEYGLLYEHADGNQNDYTLSYKKFNWDFLTKDWISPKEAKVKYAIEKWPGILAMEF
DSEVLVNKAPTLQLANGKTARFMTQYDTKTLLFTVDSEDMGQKVTGLAEGAIESM
HNLPVSVAGTKLSNGMNGSEAAVHEVPEYTGPLGTAGEEPAPTVEKPEFTGGVNGE
EAAVHEVPEYTGPLGTSGEEPAPTVEKPEFTGGVNAVEAAAHEVPEYTGPLGTSGKE
PAPTVEKPEYTGGVNAVEAAVHEVPEYTGPLATVGEEAAPKVDKPEFTGGVNAVEA
AVHELPEYTGGVNAADAAVHEIAEYKGADSLVTLAAEDYTYKAPLAQQTLPDTGN
KESSLLASLGLTAFFLGLFAMGKKREK
SEQ ID NO: 17 S. mitis nanA_4 sialidase
MEKIWREKSCRYSIRKLTVGTASVLLGAVFLASHTVSADTIKVKQNESTLEKTTAKT
DTVTKTTESTEHTQPSEAIDHSKQVLANNSSSESKPTEAKVASATTNQASTEAIVKPN
ENKETEKQELPVTEQSNYQLNYDRPTAPSYDGWEKQALPVGNGEMGAKVFGLIGEE
RIQYNEKTLWSGGPRPDSTDYNGGNYRERYKILAEIRKALEDGDRQKAKRLAEQNL
VGPNNAQYGRYLAFGDIFMVFNNQKKGLDTVTDYHRGLDITEATTTTSYTQDGTTF
KRETFSSYPDDVTVTHLTQKGDKKLDFTVWNSLTEDLLANGDYSAEYSNYKSGHVT
TDPNGILLKGTVKDNGLQFASYLGIKTDGKVTVHEDSLTITGASYATLLLSAKTNFA
QNPKTNYRKDIDLEKTVKGIVEAAQGKYYETLKRNHIKDYQSLFNRVKLNLGGSNIA
QTTKEALQTYNPTKGQKLEELFFQYGRYLLISSSRDRTDALPANLQGVWNAVDNPP
WNADYHLNVNLQMNYWPAYMSNLAETAKPMINYIDDMRYYGRIAAKEYAGIESK
DGQENGWLVHTQATPFGWTTPGWNYYWGWSPAANAWMMQNVYDYYKFTKDET
YLKEKIYPMLKETAKFWNSFLHYDQASDRWVSSPSYSPEHGTITIGNTFDQSLVWQL
FHDYMEVANHLNVDKDLVTEVKAKFDKLKPLHINKEGRIKEWYEEDSPQFTNEGIE
NNHRHVSHLVGLFPGTLFSKDQAEYLEAARATLNHRGDGGTGWSKANKINLWARL
LDGNRAHRLLAEQLKYSTLENLWDTHAPFQIDGNFGATSGIAEMLLQSHTGYIAPLP
ALPDAWKDGQVSGLVARGNFEVSMQWKDKNLQSLSFLSNVGGDLVVDYPNIEASQ
VKVNGKPVKATVLKDGRIQLATQKGDVITFEHFSGRVTSLTAVRQNGVTAELTFNQ
VEGATHYVIQRQVKDESGQTSATREFVTNQTHFIDRSLDPQLAYTYTVKAMLGNVS
TQVSEKANVETYNQLMDDRDSRIQYGSAFGNWADSELFGGTEKFADLSLGNYTDK
DATATIPFNGVGIEIYGLKSSQLGIAEVKIDGKSVGELDFYTAGATEKGSLIGRFTGLS
DGAHVMTITVKQEHKHRGSERSKISLDYFKVLPGQGTTIEKMDDRDSRIQYGSQFKD
WSDTELYKSTEKYADINNSDPSTASEAQATIPFTGTGIRIYGLKTSALGKALVTLDGK
EMPSLDFYTAGATQKATLIGEFTNLTDGNHILTLKVDPNSPAGRKKISLDSFDVIKSP
AVSLDSPSIAPLKKGDKNISLTLPAGDWEAIAVTFPGIKDPLVLRRIDDNHLVTTGDQ
TVLSIQDNQVQIPIPDETNRKIGNAIEAYSIQGNTTSSPVVAVFTKKDEKKVENQQPTT
SKGDDPAPIVEIPEYTKPIGTAGLEQPPTVSIPEYTQPIGTAGLEQAPTVSIPEYTKPVG
TAGIEQAPTVSIPEYTKPIGTAGLEQAPTVSIPEYTQPIGTAGLEQPPTVSIPEYTKSIGT
AGLEQPPVVNVPEYTQPIGTAGIEQPPTVSIPEYTKPIGTAGQEQALTVSIPEYTKPIGT
AGQEQAPTVSVPEYKLRVLKDERTGVEIIGGATDLEGISHISSRRVLAQELFGKTYDA
YDLHLKNSTDQSLQPKGSVLVRLPISSAVENVYYLTPSKELQALDFTIREGMAEFTTS
HFSTYAVVYQANGASTTAEQKPSETDIKPLANSSEQVSSSPDLVQSTNDSPKEQLPAT
GETSNPLLFLSGLSLVLTATFLLKSKKDESN
SEQ ID NO: 18 S. mitis nanA_5 sialidase
MKQYFLEKGRIFSIRKLTVGVASVAVGLTFFASGNVAASELVTEPKLEVDGQSKEVA
DVKHEKEEAVKEEAVKEEVTEKTELTAEKATEEAKTAEVAGDVLPEEIPDRAYPDTP
VKKVDTAAIVSEQESPQVETKSILKPTEVAPTEGEKENRAVINGGQDLKRINYEGQPA
TSAAMVYTIFSSPLAGGGSQRYLNSGSGIFVAPNIMLTVAHNFLVKDADTNAGSIRG
GDTTKFYYNVGSNTAKNNSLPTSGNTVLFKEKDIHFWNKEKFGEGIKNDLALVVAP
VPLSIASPNKAATFTPLAEHREYKAGEPVSTIGYPTDSTSPELKEPIVPGQLYKADGVV
KGTEKLDDKGAVGITYRLTSVSGLSGGGIINGDGKVIGIHQHGTVDNMNIAEKDRFG
GGLVLSPEQLAWVKEIIDKYGVKGWYQGDNGNRYYFTPEGEMIRNKTAVIGKNKYS
FDQNGIATLLEGVDYGRVVVEHLDQKDNPVKENDTFVEKTEVGTQFDYNYKTEIEK
TDFYKKNKEKYEIVSIDGKAVNKQLKDTWGEDYSVVSKAPAGTRVIKVVYKVNKG
SFDLRYRLKGTDQELAPATVDNNDGKEYEVSFVHRFQAKEITGYRAVNASQEATIQ
HKGVNQVIFEYEKIEDPKPATPATPVVDPKDEETEIGNYGPLPSKAQLDYHKEELAAF
IHYGMNTYTNSEWGNGRENPQNFNPTNLDTDQWIKTLKDAGFKRTIMVVKHHDGF
VIYPSQYTKHTVAASPWKDGKGDLLEEISKSATKYDMNMGVYLSPWDANNPKYHV
STEKEYNEYYLNQLKEILGNPKYGNKGKFIEVWMDGARGSGAQKVTYTFDEWFKYI
KKAEGDIAIFSAQPTSVRWIGNERGIAGDPVWHKVKKAKITDDVKNEYLNHGDPEG
DMYSVGEADVSIRSGWFYHDNQQPKSIKDLMDIYFKSVGRGTPLLLNIPPNKEGKFA
DADVARLKEFRATLDQMYATDFAKGATVTASSTRKNHLYQASNLTDGKDDTSWAL
SNDAKTGEFTVDLGQKRRFDVVELKEDIAKGQRISGFKVEVELNGRWVPYGEGSTV
GYRRIVQGQPVEAQKIRVTITNSQATPITNESVYKTPSSIEKTDGYPLGLDYHSNTT
ADKANTTWYDESEGIRGTSMWTNKKDASVTYRFNGTKAYVVSTVDPNHGEMSVY
VDGQKVADVQTNNAARKRSQMVYETDDLAPGEHTIKLVNKTGKAIATEGIYTLNN
AGKGMFELKETTYEVQKGQPVTVTIKRVGGSKGAATVHVVTEPGTGVHGKVYKDT
TADLTFQDGETEKTLTIPTIDFTEQADSIFDFKVKMTSASDNALLGFASEATVRVMKA
DLLQKDQVSHDDQASQLDYSPGWHHETNSAGKYQNTESWASFGRLNEEQKKNASV
TAYFYGTGLEIKGFVDPGHGIYKVTLDGKELEYQDGQGNATDVNGKKYFSGTATTR
QGDQTLVRLTGLEEGWHAVTLQLDPKRNDTSRNIGIQVDKFITRGEDSALYTKEELV
QAMKNWKDELAKFDQTSLKNTPEARQAFKSNLDKLSEQLSASPANAQEILKIATAL
QAILDKEENYGKEDTPTSEQPEEPNYDKAMASLSEAIQNKSKELSSDKEAKKKLVEL
SEQALTAIQEAKTQDAVDKALQAALTSINQLQATPKEEVKPSQPEEPNYDKAMASLA
EAIQNKSKELGSDKESKKKLVELSEQALTAIQEAKTQDAVDKALQAALTSINQLQAT
PKEEAKPSQPEEPNYDKAMASLAEAIQNKSKELGSDKEAKKKLVELSEQALTAIQEA
KTQDAVDKALQAALTSINQLQATPKEEVKHSIVPTDGDKELVQPQPSLEVVEKVINF
KKVKQEDSSLPKGETRVTQVGRAGKERILTEVAPDGSRTIKLREVVEVAQDEIVLVG
TKKEESGKIASSVHEVPEFTGGVIDSEATIHNLPEFTGGVTDSEAAIHNLPEFTGGVTD
SEAAIHNLPEFTGGMTDSEAAIHNLPEFTGGMTDSEGVAHGVSNVEEGVPSGEATSH
QESGFTSDVTDSETTMNEIVYKNDEKSYVVPPMLEDKTYQAPANRQEVLPKTGSED
GSAFASVGIIGMFLGMIGIVKRKKD
SEQ ID NO: 19 S. mitis nanH sialidase
MSGLKKYEGVIPAFYACYDDAGEVSPERTRALVQYFIDKGVQGLYVNGSSGECIYQS
VEDRKLILEEVMAVAKGKLTIIAHVACNNTKDSIELARHAESLGVDAIATIPPIYFRLP
EYSVAKYWNDISAAAPNTDYVIYNIPQLAGVALTPSLYTEMLKNPRVIGVKNSSMPV
QDIQTFVSLGGDDHIVFNGPDEQFLGGRLMGAKAGIGGTYGAMPELFLKLNQLIADK
DLETARELQYAINAIIGKLTAAHGNMYCVIKEVLKINEGLNIGSVRSPLTPVTEEDRPV
VEAAAQLIRESKERFL
SEQ ID NO: 20 P. gingivalis sialidase
MANNTLLAKTRRYVCLVVFCCLMAMMHLSGQEVTMWGDSHGVAPNQVRRTLVK
VALSESLPPGAKQIRIGFSLPKETEEKVTALYLLVSDSLAVRDLPDYKGRVSYDSFPIS
KEDRTTALSADSVAGRCFFYLAADIGPVASFSRSDTLTARVEELAVDGRPLPLKELSP
ASRRLYREYEALFVPGDGGSRNYRIPSILKTANGTLIAMADRRKYNQTDLPEDIDIVM
RRSTDGGKSWSDPRIIVQGEGRNHGFGDVALVQTQAGKLLMIFVGGVGLWQSTPDR
PQRTYISESRDEGLTWSPPRDITHFIFGKDCADPGRSRWLASFCASGQGLVLPSGRVM
FVAAIRESGQEYVLNNYVLYSDDEGGTWQLSDCAYHRGDEAKLSLMPDGRVLMSV
RNQGRQESRQRFFALSSDDGLTWERAKQFEGIHDPGCNGAMLQVKRNGRNQMLHS
LPLGPDGRRDGAVYLFDHVSGRWSAPVVVNSGSSAYSDMTLLADGTIGYFVEEDDE
ISLVFIRFVLDDLFDARQ
SEQ ID NO: 21 T. forsythia siaHI sialidase
MTKKSSISRRSFLKSTALAGAAGMVGTGGAATLLTSCGGGASSNENANAANKPLKE
PGTYYVPELPDMAADGKELKAGIIGCGGRGSGAAMNFLAAANGVSIVALGDTFQDR
VDSLAQKLKDEKNIDIPADKRFVGLDAYKQVIDSDVDVVIVATPPNFRPIHFQYAVE
KSKHCFLEKPICVDAVGYRTIMATAKQAQAKNLCVITGTQRHHQRSYIASYQQIMN
GAIGEITGGTVYWNQSMLWYRERQAGWSDCEWMIRDWVNWKWLSGDHIVEQHV
HNIDVFTWFSGLKPVKAVGFGSRQRRITGDQYDNFSIDFTMENGIHLHSMCRQIDGC
ANNVSEFIQGTKGSWNSTDMGIKDLAGNVIWKYDVEAEKASFKQNDPYTLEHVNWI
NTIRAGKSIDQASETAVSNMAAIMGRESAYTGEETTWEAMTAAALDYTPADLNLGK
MDMKPFVVPVPGKPLEKK
SEQ ID NO: 22 T. forsythia nanH sialidase
MKKFFWIIGLFISMLTTRAADSVYVQNPQIPILIDRTDNVLFRIRIPDATKGDVLNRLTI
RFGNEDKLSEVKAVRLFYAGTEAGTKGRSRFAPVTYVSSHNIRNTRSANPSYSVRQD
EVTTVANTLTLKTRQPMVKGINYFWVSVEMDRNTSLLSKLTPTVTEAVINDKPAVIA
GEQAAVRRMGIGVRHAGDDGSASFRIPGLVTTNEGTLLGVYDVRYNNSVDLQEHID
VGLSRSTDKGQTWEPMRIAMSFGETDGLPSGQNGVGDPSILVDERTNTVWVVAAW
THGMGNARAWTNSMPGMTPDETAQLMMVKSTDDGRTWSEPINITSQVKDPSWCFL
LQGPGRGITMRDGTLVFPIQFIDSLRVPHAGIMYSKDRGETWHIHQPARTNTTEAQV
AEVEPGVLMLNMRDNRGGSRAVSITRDLGKSWTEHSSNRSALPESICMASLISVKAK
DNIIGKDLLFFSNPNTTEGRHHITIKASLDGGVTWLPAHQVLLDEEDGWGYSCLSMID
RETVGIFYESSVAHMTFQAVKIKDLIR
SEQ ID NO: 23 A. muciniphila sialidase
MTWLLCGRGKWNKVKRMMNSVFKCLMSAVCAVALPAFGQEEKTGFPTDRAVTVF
SAGEGNPYASIRIPALLSIGKGQLLAFAEGRYKNTDQGENDIIMSVSKNGGKTWSRPR
AIAKAHGATFNNPCPVYDAKTRTVTVVFQRYPAGVKERQPNIPDGWDDEKCIRNFM
IQSRNGGSSWTKPQEITKTTKRPSGVDIMASGPNAGTQLKSGAHKGRLVIPMNEGPF
GKWVISCIYSDDGGKSWKLGQPTANMKGMVNETSIAETDNGGVVMVARHWGAGN
CRRIAWSQDGGETWGQVEDAPELFCDSTQNSLMTYSLSDQPAYGGKSRILFSGPSAG
RRIKGQVAMSYDNGKTWPVKKLLGEGGFAYSSLAMVEPGIVGVLYEENQEHIKKLK
FVPITMEWLTDGEDTGLAPGKKAPVLK
SEQ ID NO: 24 A. muciniphila sialidase
MGLGLLCALGLSIPSVLGKESFEQARRGKFTTLSTKYGLMSCRNGVAEIGGGGKSGE
ASLRMFGGQDAELKLDLKDTPSREVRLSAWAERWTGQAPFEFSIVAIGPNGEKKIYD
GKDIRTGGFHTRIEASVPAGTRSLVFRLTSPENKGMKLDDLFLVPCIPMKVNPQVEM
ASSAYPVMVRIPCSPVLSLNVRTDGCLNPQFLTAVNLDFTGTTKLSDIESVAVIRGEE
APIIHHGEEPFPKDSSQVLGTVKLAGSARPQISVKGKMELEPGDNYLWACVTMKEGA
SLDGRVVVRPASVVAGNKPVRVANAAPVAQRIGVAVVRHGDFKSKFYRIPGLARSR
KGTLLAVYDIRYNHSGDLPANIDVGVSRSTDGGRTWSDVKIAIDDSKIDPSLGATRG
VGDPAILVDEKTGRIWVAATWSHRHSIWGSKSGDNSPEACGQLVLAYSDDDGLTWS
SPINITEQTKNKDWRILFNGPGNGICMKDGTLVFAAQYWDGKGVPWSTIVYSKDRG
KTWHCGTGVNQQTTEAQVIELEDGSVMINARCNWGGSRIVGVTKDLGQTWEKHPT
NRTAQLKEPVCQGSLLAVDGVPGAGRVVLFSNPNTTSGRSHMTLKASTNDAGSWPE
DKWLLYDARKGWGYSCLAPVDKNHVGVLYESQGALNFLKIPYKDVLNAKNAR
SEQ ID NO: 25 B. thetaiotaomicron sialidase
MKRNHYLFTLILLLGCSIFVKASDTVFVHQTQIPILIERQDNVLFYFRLDAKESRMMD
EIVLDFGKSVNLSDVQAVKLYYGGTEALQDKGKKRFAPVDYISSHRPGNTLAAIPSY
SIKCAEALQPSAKVVLKSHYKLFPGINFFWISLQMKPETSLFTKISSELQSVKIDGKEAI
CEERSPKDIIHRMAVGVRHAGDDGSASFRIPGLVTSNKGTLLGVYDVRYNSSVDLQE
YVDVGLSRSTDGGKTWEKMRLPLSFGEYDGLPAAQNGVGDPSILVDTQTNTIWVVA
AWTHGMGNQRAWWSSHPGMDLYQTAQLVMAKSTDDGKTWSKPINITEQVKDPSW
YFLLQGPGRGITMSDGTLVFPTQFIDSTRVPNAGIMYSKDRGKTWKMHNMARTNTT
EAQVVETEPGVLMLNMRDNRGGSRAVAITKDLGKTWTEHPSSRKALQEPVCMASLI
HVEAEDNVLDKDILLFSNPNTTRGRNHITIKASLDDGLTWLPEHQLMLDEGEGWGYS
CLTMIDRETIGILYESSAAHMTFQAVKLKDLIR
SEQ ID NO: 26 A. viscosus sialidase
MTSHSPFSRRHLPALLGSLPLAATGLIAAAPPAHAVPTSDGLADVTITQVNAPADGLY
SVGDVMTFNITLTNTSGEAHSYAPASTNLSGNVSKCRWRNVPAGTTKTDCTGLATH
TVTAEDLKAGGFTPQIAYEVKAVEYAGKALSTPETIKGATSPVKANSLRVESITPSSS
KEYYKLGDTVTYTVRVRSVSDKTINVAATESSFDDLGRQCHWGGLKPGKGAVYNC
KPLTHTITQADVDAGRWTPSITLTATGTDGTALQTLTATGNPINVVGDHPQATPAPA
PDASTELPASMSQAQHVAPNTATDNYRIPAITTAPNGDLLISYDERPKDNGNGGSDA
PNPNHIVQRRSTDGGKTWSAPTYIHQGTETGKKVGYSDPSYVVDHQTGTIFNFHVKS
YDHGWGNSQAGTDPENRGIIQAEVSTSTDNGWTWTHRTITADITKDNPWTARFAAS
GQGIQIQHGPHAGRLVQQYTIRTAGGAVQAVSVYSDDHGKTWQAGTPVGTGMDEN
KVVELSDGSLMLNSRASDSSGFRKVAHSTDGGQTWSEPVSDKNLPDSVDNAQIIRAF
PNAAPDDPRAKVLLLSHSPNPKPWSRDRGTISMSCDDGASWTTSKVFHEPFVGYTTI
AVQSDGSIGLLSEDAHDGANYGGIWYRNFTMNWLGEQCGQKPAEPSPAPSPTAAPS
AAPSEQPAPSAAPSTEPTQAPAPSSAPEPSAVPEPSSAPAPEPTTAPSTEPTPTPAPSSAP
EPSAGPTAAPAPETSSAPAAEPTQAPTVAPSAEPTQVPGAQPSAAPSEKPGAQPSSAP
KPDATGRAPSVVNPKATAAPSGKASSSASPAPSRSATATSKPGMEPDEIDRPSDGAM
AQPTGGASAPSAAPTQAAKAGSRLSRTGTNALLVLGLAGVAVVGGYLLLRARRSKN
SEQ ID NO: 27 DAS181 without initial Met and without anchoring domain
GDHPQATPAPAPDASTELPASMSQAQHLAANTATDNYRIPAITTAPNGDLLISYDERP
KDNGNGGSDAPNPNHIVQRRSTDGGKTWSAPTYIHQGTETGKKVGYSDPSYVVDH
QTGTIFNFHVKSYDQGWGGSRGGTDPENRGIIQAEVSTSTDNGWTWTHRTITADITK
DKPWTARFAASGQGIQIQHGPHAGRLVQQYTIRTAGGAVQAVSVYSDDHGKTWQA
GTPIGTGMDENKVVELSDGSLMLNSRASDGSGFRKVAHSTDGGQTWSEPVSDKNLP
DSVDNAQIIRAFPNAAPDDPRAKVLLLSHSPNPRPWSRDRGTISMSCDDGASWTTSK
VFHEPFVGYTTIAVQSDGSIGLLSEDAHNGADYGGIWYRNFTMNWLGEQCGQKPA
SEQ ID NO: 28 Construct 1: mIg-K_DAS181 Protein sequence
METDTLLLWVLLLWVPGSTGDGDHPQATPAPAPDASTELPASMSQAQHLAANTAT
DNYRIPAITTAPNGDLLISYDERPKDNGNGGSDAPNPNHIVQRRSTDGGKTWSAPTYI
HQGTETGKKVGYSDPSYVVDHQTGTIFNFHVKSYDQGWGGSRGGTDPENRGIIQAE
VSTSTDNGWTWTHRTITADITKDKPWTARFAASGQGIQIQHGPHAGRLVQQYTIRTA
GGAVQAVSVYSDDHGKTWQAGTPIGTGMDENKVVELSDGSLMLNSRASDGSGFRK
VAHSTDGGQTWSEPVSDKNLPDSVDNAQIIRAFPNAAPDDPRAKVLLLSIISPNPRPW
SRDRGTISMSCDDGASWTTSKVFHEPFVGYTTIAVQSDGSIGLLSEDAHNGADYGGI
WYRNFTMNWLGEQCGQKPAKRKKKGGKNGKNRRNRKKKNP
SEQ ID NO: 29 Construct 2: mIg-K_DAS185 Protein sequence
METDTLLLWVLLLWVPGSTGDGDHPQATPAPAPDASTELPASMSOAOHLAANTAT
DNYRIPAITTAPNGDLLISYDERPKDNGNGGSDAPNPNHIVQRRSTDGGKTWASAPTYI
HQGTETGKKVGYSDPSYVVDHQTGTIFNFHVKSYDQGWGGSRGGTDPENRGIIQAE
VSTSTDNGWTWTHRTITADITKDKPWTARFAASGQGIQIQHGPHAGRLVQQYTIRTA
GGAVQAVSVYSDDHGKTWQAGTPIGTGMDENKVVELSDGSLMLNSRASDGSGFRK
VAHSTDGGQTWSEPVSDKNLPDSVDNAQIIRAFPNAAPDDPRAKVLLLSHSPNPRPW
SRDRGTISMSCDDGASWTTSKVFHEPFVGFTTIAVQSDGSIGLLSEDAHNGADYGGI
WYRNFTMNWLGEQCGQKPAKRKKKGGKNGKNRRNRKKKNP
SEQ ID NO: 30 Construct 3: mIg-K_Neu2-AR Protein sequence
METDTLLLWVLLLWVPGSTGDMASLPVLQKESVFQSGAHAYRIPALLYLPGQQSLL
AFAEQRASKKDEHAELIVLRRGDYDAPTHQVQWQAQEVVAQARLDGHRSMNPCPL
YDAQTGTLFLFFIAIPGQVTEQQQLQTRANVTRLCQVTSTDHGRTWSSPRDLTDAAI
GPAYREWSTFAVGPGHCLQLHDRARSLVVPAYAYRKLHPIQRPIPSAFCFLSHDHGR
TWARGHFVAQDTLECQVAEVETGEQRVVTLNARSHLRARVQAQSTNDGLDFQESQ
LVKKLVEPPPQGCQGSVISFPSPRSGPGSPAQWLLYTHPTHSWQRADLGAYLNPRPP
APEAWSEPVLLAKGSCAYSDLQSMGTGPDGSPLFGCLYEANDYEEIVFLMFTLKQAF
PAEYLPQKRKKKGGKNGKNRRNRKKKNP
SEQ ID NO: 31 Construct 4: DAS181(-AR)_TM Protein Sequence
METDTLLLWVLLLWVPGSTGDYPYDVPDYAGATPARSPGMGDHPQATPAPAPDAS
TELPASMSQAQHLAANTATDNYRIPAITTAPNGDLLISYDERPKDNGNGGSDAPNPN
HIVQRRSTDGGKTWSAPTYIHQGTETGKKVGYSDPSYVVDHQTGTIFNFHVKSYDQ
GWGGSRGGTDPENRGIIQAEVSTSTDNGWTWTHRTITADITKDKPWTARFAASGQGI
QIQHGPHAGRLVQQYTIRTAGGAVQAVSVYSDDHGKTWQAGTPIGTGMDENKVVE
LSDGSLMLNSRASDGSGFRKVAHSTDGGQTWSEPVSDKNLPDSVDNAQIIRAFPNAA
PDDPRAKVLLLSHSPNPRPWSRDRGTISMSCDDGASWTTSKVFHEPFVGFTTIAVQS
DGSIGLLSEDAHNGADYGGIWYRNFTMNWLGEQCGQKPAVDEQKLISEEDLNAVG
QDTQEVIVVPHSLPFKVVVISAILALVVITIISIIILIMLWQKKPR
SEQ ID NO: 32 Construct 5: DAS185(-AR)_TM Protein Sequence
METDTLLLWVLLLWVPGSTGDYPYDVPDYAGATPARSPGMGDHPQATPAPAPDAS
TELPASMSQAQHLAANTATDNYRIPAITTAPNGDLLISYDERPKDNGNGGSDAPNPN
HIVQRRSTDGGKTWSAPTYIHQGTETGKKVGYSDPSYVVDHQTGTIFNFHVKSYDQ
GWGGSRGGTDPENRGIIQAEVSTSTDNGWTWTHRTITADITKDKPWTARFAASGQGI
QIQHGPHAGRLVQQYTIRTAGGAVQAVSVYSDDHGKTWQAGTPIGTGMDENKVVE
LSDGSLMLNSRASDGSGFRKVAHSTDGGQTWSEPVSDKNLPDSVDNAQIIRAFPNAA
PDDPRAKVLLLSHSPNPRPWSRDRGTISMSCDDGASWTTSKVFHEPFVGFTTIAVQS
DGSIGLLSEDAHNGADYGGIWYRNFTMNWLGEQCGQKPAVDEQKLISEEDLNAVG
QDTQEVIVVPHSLPFKVVVISAILALVVLTIISLIILIMLWQKKPR
SEQ ID NO: 33 Construct 6: Neu2_TM Protein Sequence
METDTLLLWVLLLWVPGSTGDYPYDVPDYAGATPARSPGMASLPVLQKESVFQSG
AHAYRIPALLYLPGQQSLLAFAEQRASKKDEHAELIVLRRGDYDAPTHQVQWQAQE
VVAQARLDGHRSMNPCPLYDAQTGTLFLFFIAIPGQVTEQQQLQTRANVTRLCQVTS
TDHGRTWSSPRDLTDAAIGPAYREWSTFAVGPGHCLQLHDRARSLVVPAYAYRKLH
PIQRPIPSAFCFLSHDHGRTWARGHFVAQDTLECQVAEVETGEQRVVTLNARSHLRA
RVQAQSTNDGLDFQESQLVKKLVEPPPQGCQGSVISFPSPRSGPGSPAQWLLYTHPT
HSWQRADLGAYLNPRPPAPEAWSEPVLLAKGSCAYSDLQSMGTGPDGSPLFGCLYE
ANDYEEIVFLMFTLKQAFPAEYLPQVDEQKLISEEDLNAVGQDTQEVIVVPHSLPEKV
VVISAILALVVLTIISLIILIMLWQKKPR
Not underlined = Sialidase Domain
Key to Underlined Sequences:
N-Terminal Portion
METDTLLLWVLLLWVPGSTGD = Signal
YPYDVPDYA= HA Tag
GATPARSPG= Cloning Site
C-Terminal Portion
VD = Cloning Site
EQKLISEEDL = Myc Tag
NAVGQDTQEVIVVPHSLPFKVVVISAILALVVLTIISLIILIMLWQKKPR = TM Domain
SEQ ID NO: 34 Construct 1: mig-K_DAS181 Nucleotide sequence
ATGgagacagacacactcctgctatgggtactgctgctctgggttccaggttccactggtgacGGCGACCACCCACAG
GCAACACCAGCACCTGCCCCAGATGCCTCCACCGAGCTGCCAGCAAGCATGTCC
CAGGCACAGCACCTGGCAGCAAATACCGCAACAGACAACTACAGAATCCCCGCC
ATCACCACAGCCCCAAATGGCGATCTGCTGATCAGCTATGACGAGCGCCCCAAG
GATAACGGAAATGGAGGCTCCGACGCACCAAACCCTAATCACATCGTGCAGCGG
AGATCTACCGATGGCGGCAAGACATGGAGCGCCCCTACCTACATCCACCAGGGC
ACCGAGACAGGCAAGAAGGTCGGCTACTCTGACCCAAGCTATGTGGTGGATCAC
CAGACCGGCACAATCTTCAACTTTCACGTGAAGTCCTATGACCAGGGATGGGGA
GGCTCTAGGGGCGGCACCGATCCTGAGAATCGCGGCATCATCCAGGCCGAGGTG
TCTACCAGCACAGACAACGGCTGGACCTGGACACACCGGACCATCACAGCCGAC
ATCACAAAGGATAAGCCCTGGACCGCAAGATTCGCAGCAAGCGGACAGGGCATC
CAGATCCAGCACGGACCTCACGCAGGCCGGCTGGTGCAGCAGTACACCATCAGA
ACAGCAGGAGGAGCAGTGCAGGCCGTGTCCGTGTATTCTGACGATCACGGCAAG
ACCTGGCAGGCAGGCACCCCAATCGGCACAGGCATGGACGAGAATAAGGTGGTG
GAGCTGAGCGATGGCTCCCTGATGCTGAACTCTAGGGCCAGCGACGGCTCCGGC
TTCCGCAAGGTGGCACACTCTACAGACGGAGGACAGACCTGGTCCGAGCCCGTG
TCTGATAAGAATCTGCCTGACAGCGTGGATAACGCCCAGATCATCCGGGCCTTTC
CTAATGCCGCCCCAGACGATCCCAGAGCCAAGGTGCTGCTGCTGTCCCACTCTCC
AAACCCAAGGCCTTGGAGCCGGGACAGAGGCACAATCAGCATGTCCTGCGACGA
TGGCGCCAGCTGGACCACATCCAAGGTGTTCCACGAGCCATTTGTGGGCTACACC
ACAATCGCCGTGCAGTCTGATGGCAGCATCGGACTGCTGAGCGAGGACGCACAC
AATGGCGCCGATTACGGCGGCATCTGGTATCGGAACTTCACCATGAACTGGCTG
GGCGAGCAGTGTGGCCAGAAGCCAGCCAAGCGGAAGAAGAAGGGCGGCAAGAA
CGGCAAGAATAGGCGCAACCGGAAGAAGAAGAACCCCTGATGA
SEQ ID NO: 35 Construct 2: mIg-K_DASI85 Nucleotide sequence
ATGgagacagacacactectgctatgggtactgctgctctgggttccaggttccactggtgacGGCGACCACCCACAG
GCAACACCAGCACCTGCCCCAGATGCCTCCACCGAGCTGCCAGCAAGCATGTCC
CAGGCACAGCACCTGGCAGCAAATACCGCAACAGACAACTACAGAATCCCCGCC
ATCACCACAGCCCCAAATGGCGATCTGCTGATCAGCTATGACGAGCGCCCCAAG
GATAACGGAAATGGAGGCTCCGACGCACCAAACCCTAATCACATCGTGCAGCGG
AGATCTACCGATGGCGGCAAGACATGGAGCGCCCCTACCTACATCCACCAGGGC
ACCGAGACAGGCAAGAAGGTCGGCTACTCTGACCCAAGCTATGTGGTGGATCAC
CAGACCGGCACAATCTTCAACTTTCACGTGAAGTCCTATGACCAGGGATGGGGA
GGCTCTAGGGGCGGCACCGATCCTGAGAATCGCGGCATCATCCAGGCCGAGGTG
TCTACCAGCACAGACAACGGCTGGACCTGGACACACCGGACCATCACAGCCGAC
ATCACAAAGGATAAGCCCTGGACCGCAAGATTCGCAGCAAGCGGACAGGGCATC
CAGATCCAGCACGGACCTCACGCAGGCCGGCTGGTGCAGCAGTACACCATCAGA
ACAGCAGGAGGAGCAGTGCAGGCCGTGTCCGTGTATTCTGACGATCACGGCAAG
ACCTGGCAGGCAGGCACCCCAATCGGCACAGGCATGGACGAGAATAAGGTGGTG
GAGCTGAGCGATGGCTCCCTGATGCTGAACTCTAGGGCCAGCGACGGCTCCGGC
TTCCGCAAGGTGGCACACTCTACAGACGGAGGACAGACCTGGTCCGAGCCCGTG
TCTGATAAGAATCTGCCTGACAGCGTGGATAACGCCCAGATCATCCGGGCCTTTC
CTAATGCCGCCCCAGACGATCCCAGAGCCAAGGTGCTGCTGCTGTCCCACTCTCC
AAACCCAAGGCCTTGGAGCCGGGACAGAGGCACAATCAGCATGTCCTGCGACGA
TGGCGCCAGCTGGACCACATCCAAGGTGTTCCACGAGCCATTTGTGGGCTTCACC
ACAATCGCCGTGCAGTCTGATGGCAGCATCGGACTGCTGAGCGAGGACGCACAC
AATGGCGCCGATTACGGCGGCATCTGGTATCGGAACTTCACCATGAACTGGCTG
GGCGAGCAGTGFGGCCAGAAGCCAGCCAAGCGGAAGAAGAAGGGCGGCAAGAA
CGGCAAGAATAGGCGCAACCGGAAGAAGAAGAACCCCTGATGA
SEQ ID NO: 36 Construct 3: mIg-K_Neu2-AR Nucleotide Sequence
ATGgagacagacacactcctgctatgggtactgctgctctgggttccaggttccactggtgacATGGCCAGCCTGCCT
GTGCTGCAGAAGGAGAGCGTGTTCCAGTCCGGCGCCCACGCATACAGAATCCCC
GCCCTGCTGTATCTGCCTGGCCAGCAGTCCCTGCTGGCCTTTGCCGAGCAGAGAG
CCTCTAAGAAGGACGAGCACGCAGAGCTGATCGTGCTGAGGAGGGGCGACTACG
ATGCACCAACCCACCAGGTGCAGTGGCAGGCACAGGAGGTGGTGGCACAGGCA
AGGCTGGACGGACACCGCAGCATGAATCCATGCCCCCTGTATGATGCCCAGACC
GGCACACTGTTCCTGTTCTTTATCGCAATCCCCGGCCAGGTGACCGAGCAGCAGC
AGCTGCAGACCAGAGCCAACGTGACAAGACTGTGCCAGGTGACCTCCACAGACC
ACGGCAGGACCTGGAGCAGCCCTCGCGACCTGACAGATGCAGCAATCGGACCAG
CATACAGGGAGTGGTCTACATTCGCCGTGGGCCCTGGCCACTGCCTGCAGCTGCA
CGATCGGGCCAGAAGCCTGGTGGTGCCAGCCTACGCCTATCGGAAGCTGCACCC
CATCCAGAGACCTATCCCATCTGCCTTCTGCTTTCTGAGCCACGACCACGGCAGA
ACTTGGGCCAGAGGCCACTTTGTGGCCCAGGATACACTGGAGTGTCAGGTGGCA
GAGGTGGAGACCGGAGAGCAGAGGGTGGTGACACTGAATGCACGCAGCCACCT
GAGGGCCCGCGTGCAGGCCCAGTCCACCAACGACGGCCTGGATTTCCAGGAGTC
TCAGCTGGTGAAGAAGCTGGTGGAGCCACCTCCACAGGGATGTCAGGGCTCTGT
GATCAGCTTTCCCTCCCCTCGGTCTGGCCCAGGCAGCCCAGCACAGTGGCTGCTG
TACACCCACCCCACACACTCCTGGCAGAGGGCAGACCTGGGAGCATATCTGAAT
CCAAGACCCCCTGCACCAGAGGCCTGGTCCGAGCCTGTGCTGCTGGCCAAGGGC
TCTTGCGCCTACAGCGACCTGCAGAGCATGGGCACCGGACCTGATGGCTCTCCAC
TGTTCGGCTGTCTGTACGAGGCCAACGATTATGAGGAGATCGTGTTCCTGATGTT
TACACTGAAGCAGGCCTTTCCTGCCGAGTATCTGCCACAGAAGCGGAAGAAGAA
GGGCGGCAAGAACGGCAAGAATCGGAGAAACCGGAAGAAGAAGAACCCTTGAT
GA
SEQ ID NO: 37 Construct 4: DAS181(-AR)_TM Nucleotide sequence
atggagacagacacactcctgctatgggtactgctgctctgggttccaggttccactggtgacTATCCA
TATGATGTTCCAGATTATGCTGGGGCCACGCCGGCCAGATCTCCCGGGATGGGCG
ACCACCCACAGGCAACACCAGCACCTGCCCCAGATGCCTCCACCGAGCTGCCAG
CAAGCATGTCCCAGGCACAGCACCTGGCAGCAAATACCGCAACAGACAACTACA
GAATCCCCGCCATCACCACAGCCCCAAATGGCGATCTGCTGATCAGCTATGACG
AGCGCCCCAAGGATAACGGAAATGGAGGCTCCGACGCACCAAACCCTAATCACA
TCGTGCAGCGGAGATCTACCGATGGCGGCAAGACATGGAGCGCCCCTACCTACA
TCCACCAGGGCACCGAGACAGGCAAGAAGGTCGGCTACTCTGACCCAAGCTATG
TGGTGGATCACCAGACCGGCACAATCTTCAACTTTCACGTGAAGTCCTATGACCA
GGGATGGGGAGGCTCTAGGGGCGGCACCGATCCTGAGAATCGCGGCATCATCCA
GGCCGAGGTGTCTACCAGCACAGACAACGGCTGGACCTGGACACACCGGACCAT
CACAGCCGACATCACAAAGGATAAGCCCTGGACCGCAAGATTCGCAGCAAGCGG
ACAGGGCATCCAGATCCAGCACGGACCTCACGCAGGCCGGCTGGTGCAGCAGTA
CACCATCAGAACAGCAGGAGGAGCAGTGCAGGCCGTGTCCGTGTATTCTGACGA
TCACGGCAAGACCTGGCAGGCAGGCACCCCAATCGGCACAGGCATGGACGAGA
ATAAGGTGGTGGAGCTGAGCGATGGCTCCCTGATGCTGAACTCTAGGGCCAGCG
ACGGCTCCGGCTTCCGCAAGGTGGCACACTCFACAGACGGAGGACAGACCTGGT
CCGAGCCCGTGTCTGATAAGAATCTGCCTGACAGCGTGGATAACGCCCAGATCA
TCCGGGCCTTTCCTAATGCCGCCCCAGACGATCCCAGAGCCAAGGTGCTGCTGCT
GTCCCACTCTCCAAACCCAAGGCCTTGGAGCCGGGACAGAGGCACAATCAGCAT
GTCCTGCGACGATGGCGCCAGCTGGACCACATCCAAGGTGTTCCACGAGCCATTT
GTGGGCTACACCACAATCGCCGTGCAGTCTGATGGCAGCATCGGACTGCTGAGC
GAGGACGCACACAATGGCGCCGATTACGGCGGCATCTGGTATCGGAACTTCACC
ATGAACTGGCTGGGCGAGCAGTGTGGCCAGAAGCCAGCCGTCGACGAACAAAA
ACTCATCTCAGAAGAG
GATCTGaatgctgtgggccaggacacgcaggaggtcatcgtggtgccacactccttgccctttaaggtggtggtgatctcagcca
tcctggccctggtggtgctcaccatcatctcccttatcatcctcatcatgctttggcagaagaagccacgt
SEQ ID NO: 38 Construct 5: DAS185(-AR)_TM Nucleotide sequence
atggagacagacacactcctgctatgggtactgctgctctgggttccaggttccactggtgacTATCCATATGATGTTCC
AGATTATGCTGGGGCCACGCCGGCCAGATCTCCCGGGATGGGCGACCACCCACA
GGCAACACCAGCACCTGCCCCAGATGCCTCCACCGAGCTGCCAGCAAGCATGTC
CCAGGCACAGCACCTGGCAGCAAATACCGCAACAGACAACTACAGAATCCCCGC
CATCACCACAGCCCCAAATGGCGATCTGCTGATCAGCTATGACGAGCGCCCCAA
GGATAACGGAAATGGAGGCTCCGACGCACCAAACCCTAATCACATCGTGCAGCG
GAGATCTACCGATGGCGGCAAGACATGGAGCGCCCCTACCTACATCCACCAGGG
CACCGAGACAGGCAAGAAGGTCGGCTACTCTGACCCAAGCTATGTGGTGGATCA
CCAGACCGGCACAATCTTCAACTTTCACGTGAAGTCCTATGACCAGGGATGGGG
AGGCTCTAGGGGCGGCACCGATCCTGAGAATCGCGGCATCATCCAGGCCGAGGT
GTCTACCAGCACAGACAACGGCTGGACCTGGACACACCGGACCATCACAGCCGA
CATCACAAAGGATAAGCCCTGGACCGCAAGATTCGCAGCAAGCGGACAGGGCAT
CCAGATCCAGCACGGACCTCACGCAGGCCGGCTGGTGCAGCAGTACACCATCAG
AACAGCAGGAGGAGCAGTGCAGGCCGTGTCCGTGTATTCTGACGATCACGGCAA
GACCTGGCAGGCAGGCACCCCAATCGGCACAGGCATGGACGAGAATAAGGTGGT
GGAGCTGAGCGATGGCTCCCTGATGCTGAACTCTAGGGCCAGCGACGGCTCCGG
CTTCCGCAAGGTGGCACACTCTACAGACGGAGGACAGACCTGGTCCGAGCCCGT
GTCTGATAAGAATCTGCCTGACAGCGTGGATAACGCCCAGATCATCCGGGCCTTT
CCTAATGCCGCCCCAGACGATCCCAGAGCCAAGGTGCTGCTGCTGTCCCACTCTC
CAAACCCAAGGCCTTGGAGCCGGGACAGAGGCACAATCAGCATGTCCTGCGACG
ATGGCGCCAGCTGGACCACATCCAAGGTGTTCCACGAGCCATTTGTGGGCTTCAC
CACAATCGCCGTGCAGTCTGATGGCAGCATCGGACTGCTGAGCGAGGACGCACA
CAATGGCGCCGATTACGGCGGCATCTGGTATCGGAACTTCACCATGAACTGGCTG
GGCGAGCAGTGTGGCCAGAAGCCAGCCGTCGACGAACAAAAACTCATCTCAGAA
GAGGATCTGaatgctgtgggccaggacacgcaggaggtcatcgtggtgccacactccttgccctttaaggtggtggtgatctc
agccatcctggccctggtggtgctcaccatcatctcccttatcatcctcatcatgctttggcagaagaagccacgt
SEQ ID NO: 39 Construct 6: Neu2_TM Nucleotide sequence
atggagacagacacactcctgctatgggtactgctgctctgggttccaggttccactggtgacTATCCATATGATGTTCC
AGATTATGCTGGGGCCACGCCGGCCAGATCTCCCGGGATGGCCAGCCTGCCTGT
GCTGCAGAAGGAGAGCGTGTTCCAGTCCGGCGCCCACGCATACAGAATCCCCGC
CCTGCTGTATCTGCCTGGCCAGCAGTCCCTGCTGGCCTTTGCCGAGCAGAGAGCC
TCTAAGAAGGACGAGCACGCAGAGCTGATCGTGCTGAGGAGGGGCGACTACGAT
GCACCAACCCACCAGGTGCAGTGGCAGGCACAGGAGGTGGTGGCACAGGCAAG
GCTGGACGGACACCGCAGCATGAATCCATGCCCCCTGTATGATGCCCAGACCGG
CACACTGTTCCTGTTCTTTATCGCAATCCCCGGCCAGGTGACCGAGCAGCAGCAG
CTGCAGACCAGAGCCAACGTGACAAGACTGTGCCAGGTGACCTCCACAGACCAC
GGCAGGACCTGGAGCAGCCCTCGCGACCTGACAGATGCAGCAATCGGACCAGCA
TACAGGGAGTGGTCTACATTCGCCGTGGGCCCTGGCCACTGCCTGCAGCTGCACG
ATCGGGCCAGAAGCCTGGTGGTGCCAGCCTACGCCTATCGGAAGCTGCACCCCA
TCCAGAGACCTATCCCATCTGCCTTCTGCTTTCTGAGCCACGACCACGGCAGAAC
TTGGGCCAGAGGCCACTTTGTGGCCCAGGATACACTGGAGFGTCAGGTGGCAGA
GGTGGAGACCGGAGAGCAGAGGGTGGTGACACTGAATGCACGCAGCCACCTGA
GGGCCCGCGTGCAGGCCCAGrCCACCAACGACGGCCTGGATTTCCAGGAGTCTC
AGCTGGTGAAGAAGCTGGTGGAGCCACCTCCACAGGGATGTCAGGGCTCTGTGA
TCAGCTTTCCCTCCCCTCGGTCTGGCCCAGGCAGCCCAGCACAGTGGCTGCTGTA
CACCCACCCCACACACTCCTGGCAGAGGGCAGACCTGGGAGCATATCTGAATCC
AAGACCCCCTGCACCAGAGGCCTGGTCCGAGCCTGTGCTGCTGGCCAAGGGCTC
TTGCGCCTACAGCGACCTGCAGAGCATGGGCACCGGACCTGATGGCTCTCCACTG
TTCGGCTGTCTGTACGAGGCCAACGATTATGAGGAGATCGTGTTCCTGATGTTTA
CACTGAAGCAGGCCTTTCCTGCCGAGTATCTGCCACAGGTC
GACGAACAAAAACTCATCTCAGAAGAGGATCTGaatgctgtgggccaggacacgcaggaggtcatc
gtggtgccacactccttgccctttaaggtggtggtgatctcagccatcctggccctggtggtgctcaccatcatctcccttatcatcctcat
catgctttggcagaagaagccacgt
SEQ ID NO : 40 Exemplary amino acid secretion sequence
METDTLILWVLLLWVPGSTGD
SEQ ID NO: 41 HAtag amino acid sequence
YPYDVPDYA
SEQ ID NO: 42 N-terminal cloning site amino acid sequence
GATPARSPG
SEQ ID NO: 43 C-terminal cloning site amino acid sequence
VD
SEQ ID NO: 44 Myc Tag amino acid sequence
EQKLISEEDL
SEQ ID NO: 53 Salmonella typhimurium sialidase
TVEKSVVFKAEGEHFTDQKGNTIVGSGSGGTTKYFRIPAMCTTSKGTIVVFADARHN
TASDQSFIDTAAARSTDGGKTWNKKIAIYNDRVNSKLSRVMDPTCIVANIQGRETILV
MVGKWNNNDKTWGAYRDKAPDTDWDLVLYKSTDDGVTFSKVETNIHDIVTKNGTI
SAMLGGVGSGLQLNDGKLVFPVQMVRTKNITTVLNTSFIYSTDGITWSLPSGYCEGF
GSENNIIEFNASLVNNIRNSGLRRSFETKDFGKTWTEFPPMDKKVDNRNHGVQGSTIT
IPSGNKLVAAHSSAQNKNNDYTRSDISLYAHNLYSGEVKLIDDFYPKVGNASGAGYS
CLSYRKNVDKETLYVVYEANGSIEFQDLSRHLPVIKSYN
SEQ ID NO: 54 Vibrio cholera sialidase
MRFKNVKKTALMLAMFGMATSSNAALFDYNATGDTEFDSPAKQGWMQDNTNNGS
GVLTNADGMPAWLVQGIGGRAQWTYSLSTNQHAQASSFGWRMTTEMKVLSGGMI
TNYYANGTQRVLPIISLDSSGNLVVEFEGQTGRTVLATGTAATEYHKFELVFLPGSNP
SASFYFDGKLIRDNIQPTASKQNMIVWGNGSSNTDGVAAYRDIKFEIQGDVIFRGPDR
IPSIVASSVTPGVVTAFAEKRVGGGDPGALSNTNDIITRTSRDGGITWDTELNLTEQIN
VSDEFDFSDPRPIYDPSSNTVLVSYARWPTDAAQNGDRIKPWMPNGIFYSVYDVASG
NWQAPIDVTDQVKERSFQIAGWGGSELYRRNTSLNSQQDWQSNAKIRIVDGAANQI
QVADGSRKYVVTLSIDESGGLVANLNGVSAPIILQSEHAKVHSFHDYELQYSALNHT
TTLFVDGQQITTWAGEVSQENNIQFGNADAQIDGRLHVQKIVLTQQGHNLVEFDAFY
LAQQTPEVEKDLEKLGWTKIKTGNTMSLYGNASVNPGPGHGITLTRQQNISGSQNGR
LIYPAIVLDRFFLNVMSIYSDDGGSNWQTGSTLPIPFRWKSSSILETLEPSEADMVELQ
NGDLLLTARLDFNQIVNGVNYSPRQQFLSKDGGITWSLLEANNANVFSNISTGTVDA
SITRFEQSDGSHFLLFTNPQGNPAGTNGRQNLGLWFSFDEGVTWKGPIQLVNGASAY
SDIYQLDSENAIVIVETDNSNMRILRMPITLLKQKLTLSQN
SEQ ID NO: 55 Lv-CD19-CAR Plasmid DNA sequence
ATGGAGTTTGGACTGAGCTGGCTGTTTCTCGTGGCCATTCTGAAGGGCGTCCAGT
GCAGCAGAGACATCCAGATGACCCAGACAACCAGCTCTCTGAGCGCTAGCCTCG
GAGATAGAGTGACCATTAGCTGTAGAGCCTCCCAAGACATTTCCAAGTACCTCA
ACTGGTACCAGCAGAAGCCCGACGGCACCGTGAAGCTGCTGATCTACCACACCA
GCAGACTGCACTCCGGAGTGCCCTCTAGGTTTTCCGGATCCGGCAGCGGCACAG
ACTACTCTCTGACCATCTCCAATCTGGAGCAAGAGGACATCGCCACCTACTTCTG
CCAGCAAGGCAACACACTGCCTTACACATTCGGCGGCGGAACAAAGCTCGAACT
GAAAAGAGGCGGCGGCGGAAGCGGAGGAGGAGGATCCGGAGGCGGAGGATCCG
GCGGAGGAGGCTCCGAAGTCCAGCTGCAACAAAGCGGACCCGGACTGGTGGCTC
CCAGCCAATCTCTGAGCGTGACATGCACAGTGTCCGGCGTCTCTCTGCCCGACTA
CGGAGTCAGCTGGATTAGACAGCCTCCTAGAAAGGGACTGGAGTGGCTGGGAGT
CATCTGGGGCAGCGAGACCACCTACTATAACTCCGCCCTCAAGTCTAGGCTCACC
ATCATCAAAGACAACAGCAAGAGCCAAGTGTTCCTCAAGATGAACAGCCTCCAG
ACCGACGACACCGCCATCTACTACTGCGCCAAACACTACTACTACGGAGGCAGC
TACGCTATGGATTACTGGGGCCAAGGCACCACAGTCACAGTGAGCAGCTATGTG
ACCGTGAGCAGCCAAGACCCCGCCAAAGATCCCAAGTTCTGGGTGCTGGTCGTG
GTGGGAGGCGTGCTGGCTTGTTATTCTCTGCTGGTGACCGTGGCCTTCATCATCTT
CTGGGTGAGGAGCAAGAGATCCAGACTGCTGCACAGCGACTACATGAACATGAC
ACCTAGAAGGCCCGGCCCCACAAGGAAACATTACCAGCCCTACGCCCCCCCTAG
AGACTTCGCTGCCTATAGATCCAAGAGAGGAAGAAAAAAGCTGCTCTACATCTT
CAAGCAGCCCTTCATGAGGCCCGTGCAAACAACACAAGAGGAGGACGGATGTAG
CTGTAGATTCCCCGAGGAGGAAGAGGGAGGATGCGAGCTGAGAGTGAAGTTCTC
TAGGAGCGCCGATGCTCCCGCTTATCAGCAAGGCCAGAACCAGCTGTACAATGA
GCTGAATCTGGGAAGAAGGGAAGAATACGACGTGCTGGATAAGAGGAGGGGAA
GAGACCCCGAGATGGGAGGCAAGCCTAGAAGGAAGAACCCCCAAGAGGGACTG
TACAACGAGCTCCAAAAGGACAAGATGGCTGAAGCCTACAGCGAGATCGGAATG
AAGGGAGAGAGAAGGAGGGGCAAGGGCCACGATGGACTCTACCAAGGCCTCAG
CACAGCCACCAAGGACACCTACGACGCTCTGCACATGCAAGCTCTGCCCCCAGA
TGATGA
SEQ ID NO: 56 Lv-CD19-CAR Translated amino acid sequence
MEFGLSWLFLVAILKGVQCSRDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLELKRGGGGSGGGGSGGGGSGGGGSEVQLQQSGPGLVAPSQSLSVTCT
VSGVSLPDYGVSWIRQPPRKGLEWLGVIWGSETTYYNSALKSRLTIIKDNSKSQVFL
KMNSLQTDDTAIYYCAKHYYYGGSYAMDYWGQGTTVTVSSYVTVSSQDPAKDPKF
WVLVVVGGVLACYSLLVTVAFIIFWVRSKRSRLLHSDYMNMTPRRPGPTRKHYQPY
APPRDFAAYRSKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNPQEGLY
NELQKDKMAEAYSEIGMKGERRRGKGHDGLYQGLSTATKDTYDALHMQALPPDD
SEQ ID NO: 57 CD19-scFv amino acid sequence
MEFGLSWLFLVAILKGVQCSRDIQMTQTTSSLSASLGDRVTISCRASQDISKYLNWY
QQKPDGTVKLLIYHTSRLHSGVPSRFSGSGSGTDYSLTISNLEQEDIATYFCQQGNTLP
YTFGGGTKLELKRGGGGSGGGGSGGGGSGGGGSEVQLQQSGPGLVAPSQSLSVTCT
VSGVSLPDYGVSWIRQPPRKGLEWLGVIWGSETTYYNSALKSRLTIIKDNSKSQVFL
KMNSLQTDDTAIYYCAKHYYYGGSYAMDYWGQGTTVTVS
SEQ ID NO: 58 CD55-A27 amino acid sequence
MDCGLPPDVPNAQPALEGRTSFPEDTVITYKCEESFVKIPGEKDSVICLKGSQWSDIE
EFCNRSCEVPTRLNSASLKQPYITQNYFPVGTVVEYECRPGYRREPSLSPKLTCLQNL
KWSTAVEFCKKKSCPNPGEIRNGQIDVPGGILFGATISFSCNTGYKLFGSTSSFCLISGS
SVQWSDPLPECREIYCPAPPQIDNGIIQGERDHYGYRQSVTYACNKGFTMIGEHSIYC
TVNNDEGEWSGPPPECRGGGGSGGGGSGGGGSDGTLFPGDDDLAIPATEFFSTKAA
KAPEDKAADAAAAAADDNEETLKQRLTNLEKKITNVTTKFEQIEKCCKRNDEVLFR
LENHAETLRAAMISLAKKIDVQTGRAAAE
TK-left (SEQ ID NO: 59)
agttgataatcggccccatgttttcaggtaaaagtacagaattaattagacgagttagacgttatcaaatagctcaatataaatgcgtgact
ataaaatattctaacgataatagatacggaacgggactatggacgcatgataagaataattttgaagcattggaagcaactaaactatgt
gatgtcttggaatcaattacagatttctccgtgataggtatcgatgaaggacagttctttccagacattgttgaatt
Sialidase (reverse complement): (SEQ ID NO: 60)
tcatcaggggttcttcttcttccggttgcgcctattcttgccgttcttgccgcccttcttcttccgcttggctggcttctggccacactgctcgc
ccagccagttcatggtgaagttccgataccagatgccgccgtaatcggcgccattgtgtgcgtcctcgctcagcagtccgatgctgcca
tcagactgcacggcgattgtggtgtagcccacaaatggctcgtggaacaccttggatgtggtccagctggcgccatcgtcgcaggac
atgctgattgtgcctctgtcccggctccaaggccttgggtttggagagtgggacagcagcagcaccttggctctgggatcgtctgggg
cggcattaggaaaggcccggatgatctgggcgttatccacgctgtcaggcagattcttatcagacacgggctcggaccaggtctgtcc
tccgtctgtagagtgtgccaccttgcggaagccggagccgtcgctggccctagagttcagcatcagggagccatcgctcagctccac
caccttattctcgtccatgcctgtgccgattggggtgcctgcctgccaggtcttgccgtgatcgtcagaatacacggacacggcctgca
ctgctcctcctgctgttctgatggtgtactgctgcaccagccggcctgegtgaggtccgtgctggatctggatgccctgtccgcttgctg
cgaatcttgcggtccagggcttatcctttgtgatgtcggctgtgatggtccggtgtgtccaggtccagccgttgtctgtgctggtagacac
ctcggcctggatgatgccgcgattctcaggatcggtgccgcccctagagcctccccatccctggtcataggacttcacgtgaaagttga
agattgtgccggtctggtgatccaccacatagcttgggtcagagtagccgaccttcttgcctgtctcggtgccctggtggatgtaggtag
gggcgctccatgtcttgccgccatcggtagatctccgctgcacgatgtgattagggtttggtgcgtcggagcctccatttccgttatcctt
ggggcgctcgtcatagctgatcagcagatcgccatttggggctgtggtgatggcggggattctgtagttgtctgttgcggtatttgctgc
caggtgctgtgcctgggacatgcttgctggcagctcggtggaggcatctggggcaggtgctggtgttgcctgtgggtggtcgcccat
F17R: (SEQ ID NO: 61)
gaatttcattttgtttttttctatgctataa
LoxP: (SEQ ID NO: 62)
ataacttcgtataatgtatgctatacgaagttat
GFP: (SEQ ID NO: 63)
Atggtgagcaagggcgaggagctgttcaccggggtggtgcccatcctggtcgagctggacggcgacgtaaacggccacaagttca
gcgtgtccggcgagggcgagggcgatgccacctacggcaagctgaccctgaagttcatctgcaccaccggcaagctgcccgtgcc
ctggcccaccctcgtgaccaccctgacctacggcgtgcagtgcttcagccgctaccccgaccacatgaagcagcacgacttcttcaa
gtccgccatgcccgaaggctacgtccaggagcgcaccatcttcttcaaggacgacggcaactacaagacccgcgccgaggtgaagt
tcgagggcgacaccctggtgaaccgcatcgagctgaagggcatcgacttcaaggaggacggcaacatcctggggcacaagctgga
gtacaactacaacagccacaacgtctatatcatggccgacaagcagaagaacggcatcaaggtgaacttcaagatccgccacaacat
cgaggacggcagcgtgcagctcgccgaccactaccagcagaacacccccatcggcgacggccccgtgctgctgcccgacaacca
ctacctgagcacccagtccgccctgagcaaagaccccaacgagaagcgcgatcacatggtcctgctggagttcgtgaccgccgccg
ggatcactctcggcatggacgagctgtacaag
TK-right: (SEQ ID NO: 64)
aattctgtgagcgtatggcaaacgaaggaaaaatagttatagtagccgcactcgatgggacatttcaacgtaaaccgtttaataatatttt
gaatcttattccattatctgaaatggtggtaaaactaactgctgtgtgtatgaaatgctttaaggaggcttccttttctaaacgattgggtgag
gaaaccgagatagaaataa
SEQ ID NO: 65 Sequence of a portion of a vaccinia virus construct for expressing a sialidase
(DAS181).
atgaacggcggacatattcagttgataatcggccccatgttttcaggtaaaagtacagaattaattagacgagttagacgttatcaaatag
ctcaatataaatgcgtgactataaaatattctaacgataatagatacggaacgggactatggacgcatgataagaataattttgaagcatt
ggaagcaactaaactatgtgatgtcttggaatcaattacagatttctccgtgataggtatcgatgaaggacagttctttccagacattgttg
aattagatcgataaaaattaattaattacccgggtaccacatttgtagaggttttacttgctttaaaaaacctcccacacctccccctgaacc
tgaaacataaaatgaatgcaattgttgttgttaacttgtttattgcagcttataatggttacaaataaagcaatagcatcacaaatttacaaat
aaagcatttttttcactgcattctagttgtggtttgtccaaactcatcaatgtatcttatcatgtctgctcgaagcggccggcctcatcagggg
ttcttcttcttccggttgcgcctattcttgccgttcttgccgcccttcttcttccgcttggctggcttctggccacactgctcgcccagccagtt
catggtgaagttccgataccagatgccgccgtaatcggcgccattgtgtgcgtcctcgctcagcagtccgatgctgccatcagactgca
cggcgattgtggtgtagcccacaaatggctcgtggaacaccttggatgtggtccagctggcgccatcgtcgcaggacatgctgattgt
gcctctgtcccggctccaaggccttgggtttggagagtgggacagcagcagcaccttggctctgggatcgtctggggcggcattagg
aaaggcccggatgatctgggcgttatccacgctgtcaggcagattcttatcagacacgggctcggaccaggtctgtcctccgtctgtag
agtgtgccaccttgcggaagccggagccgtcgctggccctagagttcagcatcagggagccatcgctcagctccaccaccttattctc
gtccatgcctgtgccgattggggtgcctgcctgccaggtcttgccgtgatcgtcagaatacacggacacggcctgcactgctcctcctg
ctgttctgatggtgtactgctgcaccagccggcctgcgtgaggtccgtgctggatctggatgccctgtccgcttgctgcgaatcttgcgg
tccagggcttatcctttgtgatgtcggctgtgatggtccggtgtgtccaggtccagccgttgtctgtgctggtagacacctcggcctggat
gatgccgcgattctcaggatcggtgccgcccctagagcctccccatccctggtcataggacttcacgtgaaagttgaagattgtgccgg
tctggtgatccaccacatagcttgggtcagagtagccgaccttcttgcctgtctcggtgccctggtggatgtaggtaggggcgctccatg
tcttgccgccatcggtagatctccgctgcacgatgtgattagggtttggtgcgtcggagcctccatttccgttatccttggggcgctcgtc
atagctgatcagcagatcgccatttggggctgtggtgatggcggggattctgtagttgtctgttgcggtatttgctgccaggtgctgtgcc
tgggacatgcttgctggcagctcggtggaggcatctggggcaggtgctggtgttgcctgtgggtggtcgcccatttatagcatagaaaa
aaacaaaatgaaattcaagctttcactaattccaaacccacccgctttttatagtaagtttttcacccataaataataaatacaataattaattt
ctcgtaaaagtagaaaatatattctaatttattgcacggtaaggaagtagatcataactcgagataacttcgtataatgtatgctatacgaag
ttatctagcgctaccggtcgccaccatggtgagcaagggcgaggagctgttcaccggggtggtgcccatcctggtcgagctggacgg
cgacgtaaacggccacaagttcagcgtgtccggcgagggcgagggcgatgccacctacggcaagctgaccctgaagttcatctgca
ccaccggcaagctgcccgtgccctggcccaccctcgtgaccaccctgacctacggcgtgcagtgcttcagccgctaccccgaccac
atgaagcagcacgacttcttcaagtccgccatgcccgaaggctacgtccaggagcgcaccatcttcttcaaggacgacggcaactac
aagacccgcgccgaggtgaagttcgagggcgacaccctggtgaaccgcatcgagctgaagggcatcgacttcaaggaggacggc
aacatcctggggcacaagctggagtacaactacaacagccacaacgtctatatcatggccgacaagcagaagaacggcatcaaggt
gaacttcaagatccgccacaacatcgaggacggcagcgtgcagctcgccgaccactaccagcagaacacccccatcggcgacggc
cccgtgctgctgcccgacaaccactacctgagcacccagtccgccctgagcaaagaccccaacgagaagcgcgatcacatggtcct
gctggagttcgtgaccgccgccgggatcactctcggcatggacgagctgtacaagtaatagactagcgctcaataacttcgtataatgt
atgctatacgaagttatgcggccgcttcctcgctcactgacgctagcgccctatagtgagtcgtattacagatccaattctgtgagcgtat
ggcaaacgaaggaaaaatagttatagtagccgcactcgatgggacatttcaacgtaaaccgtttaataatattttgaatcttattccattatc
tgaaatggtggtaaaactaactgctgtgtgtatgaaatgctttaaggaggcttccttttctaaacgattgggtgaggaaaccgagatagaa
ataataggaggtaatgatatgtatcaatcggtgtgtagaaagtgttacatcgactcata
SEQ ID NO: 66 mutant vaccinia virus (VV) H3L protein
MAAAKTPVIVVPVAAALPSETFPNVHEHINDQAAADVADAEVMAAKRNVVVAKDD
PDHYKDYAFIQWTGGNIRNDDKYTHFFSGFCNTMCTEETKRNIARHLALWDSNFFT
ELENKKVEYVVIVENDNVIAAIAFLAPVLKAMHDKKIDILQMAEAITGNAVKTEAAA
DKNHAIFTYTGGYDVSLSAYIIRVTTALNIADEIIKSGGLSSGFYFEIARIENEMKINAQ
ILDNAAKYVEHDPRLVAEHRFANMAAAAWSRIGTAATKRYPGVMYAFTTPLISFFG
LFDINVIGLIVILFIMFMLIFNVKSKLLWFITGTFVTAFI
SEQ ID NO: 67 mutant vaccinia virus (VV) H3L protein
MAAAKTPVIVVPVIDRLPSETFPNVHEHINDQKFDDVKDNEVMAEKRNVVVVKDDP
DHYKDYAFIQWTGGNIRNDDKYTHFFSGFCNTMCTEETKRNIARHLALWDSNFFTE
LENKKVEYVVIVENDNVIEDITFLRPVLKAMHDKKIDILQMREIITGNKVKTELVMD
KNHAIFTYTGGYDVSLSAYIIRVTTALNIVDEIIKSGGLSSGFYFEIARIENEMKINRQIL
DNAAKYVEHDPRLVAEIIRFGWMKPNFWFRTGPATVIRCPGVKNANTAPLISFFGLFD
INVIGLIVILFIMFMLIFNVKSKLLWFLTGTFVTAFI
SEQ ID NO: 68 mutant vaccinia virus (VV) H3L protein
MAAAKTPVIVVPVAAALPSETFPNVHEHINDQAAADVADAEVMAAKRNVVVAKDD
PDHYKDYAFIQWTGGNIRNDDKYTHFFSGFCNTMCTEETKRNIARHLALWDSNFFT
ELENKKVEYVVIVENDNVIAAIAAAAPVLKAMHDKKIDILQMAAAITGNAVKTEAA
ADKNHAIFTYTGGYDVSLSAYIIRVTTALNAADEIIKSGGLSSGFYFEIARIENEMKIN
AQILDNAAKYVEHDPRLVAEHRFAAAAAAAWARIGPATTIRCPGVKNANTAPLISFF
GLFDINVIGLIVILFIMFMLIFNVKSKLLWFLTGTFVTAFI
SEQ ID NO: 69 mutant vaccinia virus (VV) H3L protein
MAAAKTPVIVVPVIDRLPSETFPNVHEHINDQKFDDVKDNEVMAEKRNVVVVKDDP
DHYKDYAFIQWTGGNIRNDDKYTHFFSGFCNTMCTEETKRNIARHLALWDSNFFTE
LENKKVEYVVIVENDNVIEDITFLRPVLKAMHDKKIDILQMREIITGNKVKTELVMD
KNHAIFTYTGGYDVSLSAYIIRVTTALNIVDEIIKSGGLSSGFYFEIARIENEMKINRQIL
DNAAKYVEHDPRLVAEHRFGWMKPNFWFRIGPATVIRCPGVKNANTAPLISFFGLFD
INVTGLIVILFIMFMLIFNVKSKLLWFLTGTFVTAFI
SEQ ID NO: 70 mutant vaccinia virus (VV) DSL protein
MPQQLSPINIETKKAISNARLKPLDIHYNESKPTTIQNTGALVAINFAGGYISGGFLPNE
YVLSSLHIYWGKEDDYGSNHLIDVYKYSGEINLVHWNAKKYSSYEEAAKHDDGLIII
SIFLQVLDHKNVYFQKIVNQLDSIRSANTSAPFDSVFYLDNLLPSKLDYFTYLGTTINH
SADAVWIIFPTPINIHSDQLSKFRTLLSSSNHDGKPHYITENYANPYKLNDDTQVYYS
GEIIRAATTSPARENYFMRWLSDLRETCFSYYQKYIEENKTFAIIAIVFVFILTAILFFM
SRRYSREKQN
SEQ ID NO: 71 mutant vaccinia virus (VV) D8L protein
MPQQLSPINIETKKAISNARLKPLDIHYNESKPTTIQNTGKLFWINFKGGYISGWFLPN
EYVLSSLHIYWGKEDDYGSNHLIDVYKYSGEINLVHWNKKKYSSYEEAKKHDDGLII
ISIFLQVLDHKNVYFQKTVNQLDSIRSTNTSAPFDSVFYLDNLLPSKLDYFSYLGTTIN
HYADAVWIIFPTPINIHSDQLSKYRTLSSSSNHDGKTHYITECYRNLYKLNGDTQVYY
SGEIIRAATTSPARENYFMRWLSDLRETCFSYYQKYIEENKTFAIIAIVFVFILTAILFF
MSRRYSREKQN
SEQ ID NO: 72 mutant vaccinia virus (VV) DSL protein
MPQQLSPINIETKKAISNARLKPLDIHYNESKPTTIQNTGKLAAINFAGGYIAAAFLPN
EYVLSSLHIYWGKEDDYGSNHLIDVYKYSGEINLVHWNAKKYSSYEEAAAHDDGLII
ISIFLQVLDHKNVYFQKIVNQLDSIRSGNTSAPFDSVFYLDNLLPSKLDYFAYLGTTIN
HAADAVWIIFPTPINIHSDQASKARTLASSSAHDGKAHYITEAYANAYKLNADTQVY
YSGEIIRAATTSPARENYFMRWLSDLRETCFSYYQKYIEENKTFAIIAIVFVFILTAILFF
MSRRYSREKQN
SEQ ID NO: 73 mutant vaccinia virus (VV) A27L protein
MDGTLFPGDDDLAIPATEFFSTKAAKAPEDKAADAAAAAADDNEETLKQRLTNLEK
KITNVTTKFEQIEKCCKRNDEVLFRLENHAETLRAAMISLAKKIDVQTGRAAAE
SEQ ID NO: 74 mutant vaccinia virus (VV) L1R protein
MGAAASIQTTVNTLSERISSKLEQAAAASAAAACAIEIGNFYIRQNHGCNLTVKNMC
AAAAAAQLDAVLSAATETYSGLTPEQKAYVPAMFTAALNIQTSVNTVVRDFENYVK
QTCNSSAVVDNALAIQNVIIDECYGAPGSPTNLEFINTGSSKGNCAIKALMQLTTKAT
TQIAPKQVAGTGVQFYMIVIGVIILAALFMYYAKRMLFTSTNDKIKLILANKENVHW
TTYMDTFFRTSPMVIATTDMQN
SEQ ID NO: 75 Sia1F primer
GGCGACCACCCACAGGCAACACCAGCACCTGCCCCA
SEQ ID NO: 76 Sia1R primer
CCGGTTGCGCCTATTCTTGCCGTTCTTGCCGCC
SEQ ID NO: 77 Human Platelet Factor 4 (PF4)
NGRRICLDLQAPTYKKIIKKLLES
SEQ ID NO: 78 Human Interleukin 8 (IL8)
GRELCLDPKENWVQRVVEKFLKRAENS
SEQ ID NO: 79 Human Antithrombin III (AT-III)
QIHFFFAKLNCRLYRKANKSSKLVSANRLFGDKS
SEQ ID NO: 80 Human Apoprotein E (ApoE)
ELRVRLASHLRKLRKRLLRDADDLQKRLAVYQAG
SEQ ID NO: 81 Human Angio-Associated Migratory Cell Protein (AAMP)
RRLRRMESESES
SEQ ID NO: 82 Human Amphiregulin (AR)
KRKKKGGKNGKNTTNTKKKNP
SEQ ID NO: 83 SP-Sia1-rev
TCCTGTCTTGCATTGCACTAAGTCTTG
SEQ ID NO: 84 TM-Sia1-fwd
TCATCACTAACGTGGCTTCTTCTGCCAAAGCATG
SEQ ID NO: 85 mutant vaccinia virus (VV) D8L protein
MPQQLSPINTETKKAISNARLKPLDIHYNESKPTTIQNTGKLLWINFKGGYISGWFLPN
EYVLSSLHIYWGKEDDYGSNHLIDVYKYSGEINLVHWNKKKYSSYEEAKKHDDGLII
ISIFLQVLDHKNVYFQKIVNQLDSIRSTNTSAPFDSVFYLDNLLPSKLDYFSYLGTTIN
HYADAVWIIFPTPINIHSDQLSKYRTLSSSSNHDGKTHYITECYRNLYKLNGDTQVYY
SGEIIRAATTSPARENYFMRWLSDLRETCFSYYQKYIEENKTFAIIAIVFVFILTAILFF
MSRRYSREKQN

While certain embodiments have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1: A recombinant oncolytic virus comprising a nucleotide sequence encoding a sialidase, wherein the nucleotide sequence encoding the sialidase is operably linked to a promoter.

2: The recombinant oncolytic virus of claim 1, wherein said oncolytic virus is a virus selected from the group consisting of: vaccinia virus, reovirus, Seneca Valley virus (SVV), vesicular stomatitis virus (VSV), Newcastle disease virus (NDV), herpes simplex virus (HSV), morbillivirus virus, retrovirus, influenza virus, Sinbis virus, poxvirus, measles virus, cytomegalovirus (CMV), lentivirus, adenovirus, coxsackievirus, and derivatives thereof.

3: The recombinant oncolytic virus of claim 2, wherein said oncolytic virus is a poxvirus, a reovirus, or an adenovirus.

4: The recombinant oncolytic virus of claim 3, wherein said poxvirus is a vaccinia virus of a strain selected from the group consisting of Dryvax, Lister, M63, LIVP, Tian Tan, Modified Vaccinia Ankara, New York City Board of Health (NYCBOH), Dairen, Ikeda, LC16M8, Tashkent, IHD-J, Brighton, Dairen I, Connaught, Wyeth, Copenhagen, Western Reserve, Elstree, CL, Lederle-Chorioallantoic, AS, and derivatives thereof.

5-6. (canceled)

7: The recombinant oncolytic virus of claim 1, wherein the recombinant oncolytic virus comprises one or more mutations that reduce immunogenicity of the virus compared to a corresponding wild-type strain.

8-14. (canceled)

15: The recombinant oncolytic virus of claim 1, wherein the sialidase is a bacterial sialidase or a derivative thereof.

16: The recombinant oncolytic virus of claim 15, wherein the bacterial sialidase is selected from the group consisting of: Clostridium perfringens sialidase, Actinomyces viscosus sialidase, and Arthrobacter ureafaciens sialidase, Salmonella typhimurium sialidase and Vibrio cholera sialidase.

17-19. (canceled)

20: The recombinant oncolytic virus of claim 1, wherein the sialidase comprises an anchoring domain.

21. (canceled)

22: The recombinant oncolytic virus of claim 20, wherein the anchoring domain is a glycosaminoglycan (GAG)-binding domain.

23: The recombinant oncolytic virus of claim 1, wherein the sialidase comprises an amino acid sequence selected from the group consisting of SEQ ID NOs:1-28, 31, or 53-54.

24. (canceled)

25: The recombinant oncolytic virus of claim_, wherein the sialidase is DAS181.

26-27. (canceled)

28: The recombinant oncolytic virus of claim 26, wherein the sialidase comprises a transmembrane domain.

29: The recombinant oncolytic virus of claim 28, wherein the anchoring domain or the transmembrane domain is located at the carboxy terminus of the sialidase.

30: The recombinant oncolytic virus of claim 1, wherein the promotor is a viral early promoter, a viral late promoter, or a hybrid thereof.

31-35. (canceled)

36: The recombinant oncolytic virus of claim 1, further comprising a second nucleotide sequence encoding a heterologous protein.

37. (canceled)

38: The recombinant oncolytic virus of claim 36, wherein the second heterologous protein comprises one or more of: an immune checkpoint inhibitor an inhibitor of an immune suppressive receptor, a multi-specific immune cell engager, a bispecific molecule, a cytokine, a costimulatory molecule, a tumor antigen presenting protein, an anti-angiogenic factor, a tumor-associated antigen, a foreign antigen, a matrix metalloprotease (MMP), or a bacterial polypeptide.

39-42. (canceled)

43: The recombinant oncolytic virus of claim 38, wherein second heterologous protein is the inhibitor of the immune suppressive receptor, and wherein the inhibitor of the immune suppressive receptor is an anti-LILRB antibody.

44-50. (canceled)

51: A pharmaceutical composition comprising a recombinant oncolytic virus of claim 1 and a pharmaceutically acceptable carrier.

52: A carrier cell composition comprising a carrier cell and a recombinant oncolytic virus of claim 1.

53-54. (canceled)

55: A method of treating a cancer in an individual in need thereof, the method comprising administering to the individual an effective amount of a pharmaceutical composition comprising a recombinant oncolytic virus comprising a nucleotide sequence encoding a sialidase, wherein the nucleotide sequence encoding the sialidase is operably linked to a promoter, or a carrier cell, composition comprising a carrier cell and a recombinant oncolytic virus comprising a nucleotide sequence encoding a sialidase, wherein the nucleotide sequence encoding the sialidase is operably linked to a promoter.

56-73. (canceled)


SEQUENCE LISTING (85 sequences)

SEQ ID NO: 1 - protein, 395 aa, Artificial Sequence
  Synthetic Construct
MGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAE

SEQ ID NO: 2 - protein, 415 aa, Artificial Sequence
  Synthetic Construct
MGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAKRKKKGGKNGKNRRNRKKKNP

SEQ ID NO: 3 - protein, 415 aa, Homo sapiens
MTGERPSTALPDRRWGPRILGFWGGCRVWVFAAIFLLLSLAASWSKAENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSGIPYGQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISVYGTL

SEQ ID NO: 4 - protein, 380 aa, Homo sapiens
MASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQ

SEQ ID NO: 5 - protein, 428 aa, Homo sapiens
MEEVTTCSFNSPLFRQEDDRGITYRIPALLYIPPTHTFLAFAEKRSTRRDEDALHLVLRRGLRIGQLVQWGPLKPLMEATLPGHRTMNPCPVWEQKSGCVFLFFICVRGHVTERQQIVSGRNAARLCFIYSQDAGCSWSEVRDLTEEVIGSELKHWATFAVGPGHGIQLQSGRLVIPAYTYYIPSWFFCFQLPCKTRPHSLMIYSDDLGVTWHHGRLIRPMVTVECEVAEVTGRAGHPVLYCSARTPNRCRAEALSTDHGEGFQRLALSRQLCEPPHGCQGSVVSFRPLEIPHRCQDSSSKDAPTIQQSSPGSSLRLEEEAGTPSESWLLYSHPTSRKQRVDLGIYLNQTPLEAACWSRPWILHCGPCGYSDLAALEEEGLFGCLFECGTKQECEQIAFRLFTHREILSHLQGDCTSPGRNPSQFKSN

SEQ ID NO: 6 - protein, 484 aa, Homo sapiens
MGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 7 - protein, 496 aa, Homo sapiens
MMSSAAFPRWLSMGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 8 - protein, 497 aa, Homo sapiens
MMSSAAFPRWLQSMGVPRTPSRTVLFERERTGLTYRVPSLLPVPPGPTLLAFVEQRLSPDDSHAHRLVLRRGTLAGGSVRWGALHVLGTAALAEHRSMNPCPVHDAGTGTVFLFFIAVLGHTPEAVQIATGRNAARLCCVASRDAGLSWGSARDLTEEAIGGAVQDWATFAVGPGHGVQLPSGRLLVPAYTYRVDRRECFGKICRTSPHSFAFYSDDHGRTWRCGGLVPNLRSGECQLAAVDGGQAGSFLYCNARSPLGSRVQALSTDEGTSFLPAERVASLPETAWGCQGSIVGFPAPAPNRPRDDSWSVGPGSPLQPPLLGPGVHEPPEEAAVDPRGGQVPGGPFSRLQPRGDGPRQPGPRPGVSGDVGSWTLALPMPFAAPPQSPTWLLYSHPVGRRARLHMGIRLSQSPLDPRSWTEPWVIYEGPSGYSDLASIGPAPEGGLVFACLYESGARTSYDEISFCTFSLREVLENVPASPKPPNLGDKPRGCCWPS

SEQ ID NO: 9 - protein, 913 aa, Actinomyces viscosus
MTSHSPFSRRRLPALLGSLPLAATGLIAAAPPAHAVPTSDGLADVTITQVNAPADGLYSVGDVMTFNITLTNTSGEAHSYAPASTNLSGNVSKCRWRNVPAGTTKTDCTGLATHTVTAEDLKAGGFTPQIAYEVKAVEYAGKALSTPETIKGATSPVKANSLRVESITPSSSQENYKLGDTVSYTVRVRSVSDKTINVAATESSFDDLGRQCHWGGLKPGKGAVYNCKPLTHTITQADVDAGRWTPSITLTATGTDGATLQTLTATGNPINVVGDHPQATPAPAPDASTELPASMSQAQHLAANTATDNYRIPAIPPPPMGTCSSPTTSARRTTATAAATTPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGPVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWCRDRGTISMSCDDGASWTTSKVFHEPFVGYTTIAVQSDGSIGLLSEDAHNGADYGGIWYRNFTMNWLGEQCGQKPAEPSPGRRRRRHPQRHRRRSRPRRPRRALSPRRHRHHPPRPSRALRPSRAGPGAGAHDRSEHGAHTGSCAQSAPEQTDGPTAAPAPETSSAPAAEPTQAPTVAPSVEPTQAPGAQPSSAPKPGATGRAPSVVNPKATGAATEPGTPSSSASPAPSRNAAPTPKPGMEPDEIDRPSDGTMAQPTGAPARRVPRRRRRRRPAAGCLARDQRAADPGPCGCRGCRRVPAAAGSPFEELNTRRAGHPALSTD

SEQ ID NO: 10 - protein, 793 aa, Actinomyces viscosus
MTTTKSSALRRLSALAGSLALAVTGIIAAAPPAHATPTSDGLADVTITQTHAPADGIYAVGDVMTFDITLTNTSGQARSFAPASTNLSGNVLKCRWSNVAAGATKTDCTGLATHTVTAEDLKAGGFTPQIAYEVKAVGYKGEALNKPEPVTGPTSQIKPASLKVESFTLASPKETYTVGDVVSYTVRIRSLSDQTINVAATDSSFDDLARQCHWGNLKPGQGAVYNCKPLTHTITQADADHGTWTPSITLAATGTDGAALQTLAATGEPLSVVVERPKADPAPAPDASTELPASMSDAQHLAENTATDNYRIPAITTAPNGDLLVSYDERPRDNGNNGGDSPNPNHIVQRRSTDGGKTWSAPSYIHQGVETGRKVGYSDPSYVVDNQTGTIFNFHVKSFDQGWGHSQAGTDPEDRSVIQAEVSTSTDNGWSWTHRTITADITRDNPWTARFAASGQGIQIHQGPHAGRLVQQYTIRTADGVVQAVSVYSDDHGQTWQAGTPTGTGMDENKVVELSDGSLMLNSRASDGTGFRKVATSTDGGQTWSEPVPDKNLPDSVDNAQIIRPFPNAAPSDPRAKVLLLSHSPNPRPWSRDRGTISMSCDNGASWVTGRVFNEKFVGYTTIAVQSDGSIGLLSEDGNYGGIWYRNFTMGWVGDQCSQPRPEPSPSPTPSAAPSAEPTSEPTTAPAPEPTTAPSSEPSVSPEPSSSAIPAPSQSSSATSGPSTEPDEIDRPSDGAMAQPTGGAGRPSTSVTGATSRNGLSRTGTNALLVLGVAAAAAAGGYLVLRIRRARTE

SEQ ID NO: 11 - protein, 1130 aa, Streptococcus oralis
MNYKSLDRKQRYGIRKFAVGAASVVIGTVVFGANPVLAQEQANAAGANTETVEPGQGLSELPKEASSGDLAHLDKDLAGKLAAAQDNGVEVDQDHLKKNESAESETPSSTETPAEEANKEEESEDQGAIPRDYYSRDLKNANPVLEKEDVETNAANGQRVDLSNELDKLKQLKNATVHMEFKPDASAPRFYNLFSVSSDTKENEYFTMSVLDNTALIEGRGANGEQFYDKYTDAPLKVRPGQWNSVTFTVEQPTTELPHGRVRLYVNGVLSRTSLKSGNFIKDMPDVNQAQLGATKRGNKTVWASNLQVRNLTVYDRALSPDEVQTRSQLFERGELEQKLPEGAKVTEKEDVFEGGRNNQPNKDGIKSYRIPALLKTDKGTLIAGTDERRLHHSDWGDIGMVVRRSSDNGKTWGDRIVISNPRDNEHAKHADWPSPVNIDMALVQDPETKRIFAIYDMFLESKAVFSLPGQAPKAYEQVGDKVYQVLYKQGESGRYTIRENGEVFDPQNRKTDYRVVVDPKKPAYSDKGDLYKGNELIGNIYFEYSEKNIFRVSNTNYLWMSYSDDDGKTWSAPKDITHGIRKDWMHFLGTGPGTGIALRTGPHKGRLVIPVYTTNNVSYLSGSQSSRVIYSDDHGETWQAGEAVNDNRPVGNQTIHSSTMNNPGAQNTESTVVQLNNGDLKLFMRGLTGDLQVATSHDGGATWDKEIKRYPQVKDVYVQMSAIHTMHEGKEYILLSNAGGPGRNNGLVHLARVEENGELTWLKHNPIQSGKFAYNSLQELGNGEYGLLYEHADGNQNDYTLSYKKFNWDFLSRDRISPKEAKVKYAIQKWPGIIAMEFDSEVLVNKAPTLQLANGKTATFMTQYDTKTLLFTIDPEDMGQRITGLAEGAIESMHNLPVSLAGSKLSDGINGSEAAIHEVPEFTGGVNAEEAAVAEIPEYTGPLATVGEEVAPTVEKPEFTGGVNAEEAPVAEMPEYTGPLSTVGEEVAPTVEKPEFTGGVNAVEAAVHELPEFKGGVNAVLAASNELPEYRGGANFVLAASNDLPEYIGGVNGAEAAVHELPEYKGDTNLVLAAADNKLSLGQDVTYQAPAAKQAGLPNTGSKETHSLISLGLAGVLLSLFAFGKKRKE

SEQ ID NO: 12 - protein, 305 aa, Streptococcus oralis
MSDLKKYEGVIPAFYACYDDQGEVSPERTRALVQYFIDKGVQGLYVNGSSGECIYQSVEDRKLILEEVMAVAKGKLTIIAHVACNNTKDSMELARHAESLGVDAIATIPPIYFRLPEYSVAKYWNDISAAAPNTDYVIYNIPQLAGVALTPSLYTEMLKNPRVIGVKNSSMPVQDIQTFVSLGGEDHIVFNGPDEQFLGGRLMGAKAGIGGTYGAMPELFLKLNQLIAEKDLETARELQYAINAIIGKLTSAHGNMYGVIKEVLKINEGLNIGSVRSPLTPVTEEDRPVVEAAAQLIRETKERFL

SEQ ID NO: 13 - protein, 1110 aa, Streptococcus mitis
MNQRHFDRKQRYGIRKFTVGAASVVIGAVVFGVAPALAQEAPSTNGETAGQSLPELPKEVETGNLTNLDKELADKLSTATDKGTEVNREELQANPGSEKAAETEASNETPATESEDEKEDGNIPRDFYARELENVNTVVEKEDVETNPSNGQRVDMKEELDKLKKLQNATIHMEFKPDASAPRFYNLFSVSSDTKVNEYFTMAILDNTAIVEGRDANGNQFYGDYKTAPLKIKPGEWNSVTFTVERPNADQPKGQVRVYVNGVLSRTSPQSGRFIKDMPDVNQVQIGTTKRTGKNFWGSNLKVRNLTVYDRALSPEEVKKRSQLFERGELEKKLPEGAKVTDKLDVFQGGENRKPNKDGIASYRIPALLKTDKGTLIAGADERRLHHSDWGDIGMVVRRSDDKGKTWGDRIVISNPRDNENARRAHAGSPVNIDMALVQDPKTKRIFSIFDMFVEGEAVRDLPGKAPQAYEQIGNKVYQVLYKKGEAGHYTIRENGEVFDPENRKTEYRVVVDPKKPAYSDKGDLYKGEELIGNVYFDYSDKNIFRVSNTNYLWMSYSDDDGKTWSAPKDITYGIRKDWMHFLGTGPGTGIALHSGPHKGRLVIPAYTTNNVSYLGGSQSSRVIYSDDHGETWHAGEAVNDNRPIGNQTIHSSTMNNPGAQNTESTVVQLNNGDLKLFMRGLTGDLQVATSKDGGATWEKDVKRYADVKDVYVQMSAIHTVQEGKEYIILSNAGGPGRYNGLVHVARVEANGDLTWIKHNPIQSGKFAYNSLQDLGNGEFGLLYEHATATQNEYTLSYKKFNWDFLSKDGVAPTKATVKNAVEMSKNVIALEFDSEVLVNQPPVLKLANGNFATFLTQYDSKTLLFAASKEDIGQEITEIIDGAIESMHNLPVSLEGAGVPGGKNGAKAAIHEVPEFTGAVNGEGTVHEDPAFEGGINGEEAAVHDVPDFSGGVNGEVAAIHEVPEFTGGINGEEAAKLELPSYEGGANAVEAAKSELPSYEGGANAVEAAKLELPSYESGAHEVQPASSNLPTLADSVNKAEAAVHKGKEYKANQSTAVQAMAQEHTYQAPAAQQHLLPKTGSEDKSSLAIVGFVGMFLGLLMIGKKRE

SEQ ID NO: 14 - protein, 1292 aa, Streptococcus mitis
MNQSSLNRKNRYGIRKFTIGVASVAIGSVLFGITPALAQETTTNIDVSKVETSLESGAPVSEPVTEVVSGDLNHLDKDLADKLALATNQGVDVNKHNLKEETSKPEGNSEHLPVESNTGSEESIEHHPAKIEGADDAVVPPRDFFARELTNVKTVFEREDLATNTGNGQRVDLAEELDQLKQLQNATIHMEFKPDANAPQFYNLFSVSSDKKKDEYFSMSVNKGTAMVEARGADGSHFYGSYSDAPLKIKPGQWNSVTFTVERPKADQPNGQVRLYVNGVLSRTNTKSGRFIKDMPDVNKVQIGATRRANQTMWGSNLQIRNLTVYNRALTIEEVKKRSHLFERNDLEKKLPEGAEVTEKKDIFESGRNNQPNGEGINSYRIPALLKTDKGTLIAGGDERRLHHFDYGDIGMVIRRSQDNGKTWGDKLTISNLRDNPEATDKTATSPLNIDMVLVQDPTTKRIFSIYDMFPEGRAVFGMPNQPEKAYEEIGDKTYQVLYKQGETERYTLRDNGEIFNSQNKKTEYRVVVNPTEAGFRDKGDLYKNQELIGNIYFKQSDKNPFRVANTSYLWMSYSDDDGKTWSAPKDITPGIRQDWMKFLGTGPGTGIVLRTGAHKGRILVPAYTTNNISHLGGSQSSRLIYSDDHGQTWHAGESPNDNRPVGNSVIHSSNMNKSSAQNTESTVLQLNNGDVKLFMRGLTGDLQVATSKDGGVTWEKTIKRYPEVKDAYVQMSAIHTMHDGKEYILLSNAAGPGRERKNGLVHLARVEENGELTWLKHNPIQNGEFAYNSLQELGGGEYGLLYEHRENGQNYYTLSYKKFNWDFVSKDLISPTEAKVSQAYEMGKGVFGLEFDSEVLVNRAPILRLANGRTAVFMTQYDSKTLLFAVDKKDIGQEITGIVDGSIESMHNLTVNLAGAGIPGGMNAAESVEHYTEEYTGVLGTSGVEGVPTISVPEYEGGVNSELALVSEKEDYRGGVNSASSVVTEVLEYTGPLSTVGSEDAPTVSVLEYEGGVNIDSPEVTEAPEYKEPIGTSGYELAPTVDKPAYTGTIEPLEKEENSGAIIEEGNVSYITENNNKPLENNNVTTSSIISESSKLKHTLKNATGSVQIHASEEVLKNVKDVKIQEVKVSSLSSLNYKAYDIQLNDASGKAVQPKGTVIVTFAAEQSVENVYYVDSKGNLHTLEFLQKDGEVTFETNHFSIYAMTFQLSLDNVVLDNHREDKNGEVNSASPKLLSINGHSQSSQLENKVSNNEQSKLPNTGEDKSISTVLLGFVGVILGAMIFYRRKDSEG

SEQ ID NO: 15 - protein, 1004 aa, Streptococcus mitis
  VARIANT 298: Xaa = Any Amino Acid
MDKKKIILTSLASVAVLGAALAASQPSLVKAEEQPTASQPAGETGTKSEVTSPEIKQAEADAKAAEAKVTEAQAKVDTTTPVADEAAKKLETEKKEADEADAAKTKAEEAKKTADDELAAAKEKAAEADAKAKEEAKKEEDAKKEEADSKEALTEALKQLPDNELLDKKAKEDLLKAVEAGDLKASDILAELADDDKKAEANKETEKKLRNKDQANEANVATTPAEEAKSKDQLPADIKAGIDKAEKADAARPASEKLQDKADDLGENVDELKKEADALKAEEDKKAETLKKQEDTLXEAKEALKSAKDNGFGEDITAPLEKAVTAIEKERDAAQNAFDQAASDTKAVADELNKLTDEYNKTLEEVKAAKEKEANEPAKPVEEEPAKPAEKTEAEKAAEAKTEADAKVAELQKKADEAKTKADEATAKATKEAEDVKAAEKAKEEADKAKTDAEAELAKAKEEAEKAKAKVEELKKEEKDNLEALKAALDQLEKDIDADATITNKEEAKKALGKEDILAAVEKGDLTAGDVLKELENQNATAEATKDQDPQADEIGATKQEGKPLSELPAADKEKLDAAYNKEASKPIVKKLQDIADDLVEKIEKLTKVADKDKADATEKAKAVEEKNAALDKQKETLDKAKAALETAKKNQADQAIQDGLQDAVTKLEASFASAKTAADEAQAKFDEVNEVVKAYKAAIDELTDDYNATLGHIENLKEVPKGEEPKDFSGGVNDDEAPSSTPNTNEFTGGANDADAPTAPNANEFAGGVNDEEAPTTENKPEFNGGVNDEEAPTVPNKPEGEAPKPTGENAKDAPVVKLPEFGANNPEIKKILDEIAKVKEQIKDGEENGSEDYYVEGLKERLADLEEAFDTLSKNLPAVNKVPEYTGPVTPENGQTQPAVNTPGGQQGGSSQQTPAVQQGGSGQQAPAVQQGGSNQQVPAVQQTNTPAVAGTSQDNTYQAPAAKEEDKKELPNTGGQESAALASVGFLGLLLGALPFVKRKN

SEQ ID NO: 16 - protein, 1145 aa, Streptococcus mitis
MKYRDFDRKRRYGIRKFAVGAASVVIGTVVFGANPVLAQEQANAAGANTETVEPGQGLSELPKEASSGDLAHLDKDLAGKLAAAQDNGVEVDQDHLKKNESAESETPSSTETPAEGTNKEEESEDQGAIPRDYYSRDLKNANPVLEKEDVETNAANGQRVDLSNELDKLKQLKNATVHMEFKPDASAPRFYNLFSVSSDTKENEYFTISVLDNTALIEGRGANGEQFYDKYTDAPLKVRPGQWNSVTFTVEQPTTELPHGRVRLYVNGVLSRTSLKSGNFIKDMPDVNQAQLGATKRGNKTVWASNLQVRNLTVYDRALSPDEVQTRSQLFERGELEQKLPEGAKVTEKEDVFEGGRNNQPNKDGIKSYRIPALLKTDKGTLIAGTDERRLHHSDWGDIGMVVRRSSDNGKTWGDRIVISNPRDNEHAKHADWPSPVNIDMALVQDPETKRIFAIYDMFLESKAVFSLPGQAPKAYEQVGDKVYQVLYKQGESGRYTIRENGEVFDPQNRKTDYRVVVDPKKPAYSDKGDLYKGNELIGNIYFEYSEKNIFRVSNTNYLWMSYSDDDGKTWSAPKDITHGIRKDWMHFLGTGPGTGIALRTGPHKGRLVIPVYTTNNVSYLSGSQSSRVIYSDDHGETWQAGEAVNDNRPVGNQTIHSSTMNNPGAQNTESTVVQLNNGDLKLFMRGLTGDLQVATSHDGGATWDKEIKRYPQVKDVYVQMSAIHTMHEGKEYILLSNAGGPGRNNGLVHLARVEENGELTWLKHNPIQSGKFAYNSLQDLGNGEYGLLYEHADGNQNDYTLSYKKFNWDFLTKDWISPKEAKVKYAIEKWPGILAMEFDSEVLVNKAPTLQLANGKTARFMTQYDTKTLLFTVDSEDMGQKVTGLAEGAIESMHNLPVSVAGTKLSNGMNGSEAAVHEVPEYTGPLGTAGEEPAPTVEKPEFTGGVNGEEAAVHEVPEYTGPLGTSGEEPAPTVEKPEFTGGVNAVEAAAHEVPEYTGPLGTSGKEPAPTVEKPEYTGGVNAVEAAVHEVPEYTGPLATVGEEAAPKVDKPEFTGGVNAVEAAVHELPEYTGGVNAADAAVHEIAEYKGADSLVTLAAEDYTYKAPLAQQTLPDTGNKESSLLASLGLTAFFLGLFAMGKKREK

SEQ ID NO: 17 - protein, 1797 aa, Streptococcus mitis
MEKIWREKSCRYSIRKLTVGTASVLLGAVFLASHTVSADTIKVKQNESTLEKTTAKTDTVTKTTESTEHTQPSEAIDHSKQVLANNSSSESKPTEAKVASATTNQASTEAIVKPNENKETEKQELPVTEQSNYQLNYDRPTAPSYDGWEKQALPVGNGEMGAKVFGLIGEERIQYNEKTLWSGGPRPDSTDYNGGNYRERYKILAEIRKALEDGDRQKAKRLAEQNLVGPNNAQYGRYLAFGDIFMVFNNQKKGLDTVTDYHRGLDITEATTTTSYTQDGTTFKRETFSSYPDDVTVTHLTQKGDKKLDFTVWNSLTEDLLANGDYSAEYSNYKSGHVTTDPNGILLKGTVKDNGLQFASYLGIKTDGKVTVHEDSLTITGASYATLLLSAKTNFAQNPKTNYRKDIDLEKTVKGIVEAAQGKYYETLKRNHIKDYQSLFNRVKLNLGGSNIAQTTKEALQTYNPTKGQKLEELFFQYGRYLLISSSRDRTDALPANLQGVWNAVDNPPWNADYHLNVNLQMNYWPAYMSNLAETAKPMINYIDDMRYYGRIAAKEYAGIESKDGQENGWLVHTQATPFGWTTPGWNYYWGWSPAANAWMMQNVYDYYKFTKDETYLKEKIYPMLKETAKFWNSFLHYDQASDRWVSSPSYSPEHGTITIGNTFDQSLVWQLFHDYMEVANHLNVDKDLVTEVKAKFDKLKPLHINKEGRIKEWYEEDSPQFTNEGIENNHRHVSHLVGLFPGTLFSKDQAEYLEAARATLNHRGDGGTGWSKANKINLWARLLDGNRAHRLLAEQLKYSTLENLWDTHAPFQIDGNFGATSGIAEMLLQSHTGYIAPLPALPDAWKDGQVSGLVARGNFEVSMQWKDKNLQSLSFLSNVGGDLVVDYPNIEASQVKVNGKPVKATVLKDGRIQLATQKGDVITFEHFSGRVTSLTAVRQNGVTAELTFNQVEGATHYVIQRQVKDESGQTSATREFVTNQTHFIDRSLDPQLAYTYTVKAMLGNVSTQVSEKANVETYNQLMDDRDSRIQYGSAFGNWADSELFGGTEKFADLSLGNYTDKDATATIPFNGVGIEIYGLKSSQLGIAEVKIDGKSVGELDFYTAGATEKGSLIGRFTGLSDGAHVMTITVKQEHKHRGSERSKISLDYFKVLPGQGTTIEKMDDRDSRIQYGSQFKDWSDTELYKSTEKYADINNSDPSTASEAQATIPFTGTGIRIYGLKTSALGKALVTLDGKEMPSLDFYTAGATQKATLIGEFTNLTDGNHILTLKVDPNSPAGRKKISLDSFDVIKSPAVSLDSPSIAPLKKGDKNISLTLPAGDWEAIAVTFPGIKDPLVLRRIDDNHLVTTGDQTVLSIQDNQVQIPIPDETNRKIGNAIEAYSIQGNTTSSPVVAVFTKKDEKKVENQQPTTSKGDDPAPIVEIPEYTKPIGTAGLEQPPTVSIPEYTQPIGTAGLEQAPTVSIPEYTKPVGTAGIEQAPTVSIPEYTKPIGTAGLEQAPTVSIPEYTQPIGTAGLEQPPTVSIPEYTKSIGTAGLEQPPVVNVPEYTQPIGTAGIEQPPTVSIPEYTKPIGTAGQEQALTVSIPEYTKPIGTAGQEQAPTVSVPEYKLRVLKDERTGVEIIGGATDLEGISHISSRRVLAQELFGKTYDAYDLHLKNSTDQSLQPKGSVLVRLPISSAVENVYYLTPSKELQALDFTIREGMAEFTTSHFSTYAVVYQANGASTTAEQKPSETDIKPLANSSEQVSSSPDLVQSTNDSPKEQLPATGETSNPLLFLSGLSLVLTATFLLKSKKDESN

SEQ ID NO: 18 - protein, 2115 aa, Streptococcus mitis
MKQYFLEKGRIFSIRKLTVGVASVAVGLTFFASGNVAASELVTEPKLEVDGQSKEVADVKHEKEEAVKEEAVKEEVTEKTELTAEKATEEAKTAEVAGDVLPEEIPDRAYPDTPVKKVDTAAIVSEQESPQVETKSILKPTEVAPTEGEKENRAVINGGQDLKRINYEGQPATSAAMVYTIFSSPLAGGGSQRYLNSGSGIFVAPNIMLTVAHNFLVKDADTNAGSIRGGDTTKFYYNVGSNTAKNNSLPTSGNTVLFKEKDIHFWNKEKFGEGIKNDLALVVAPVPLSIASPNKAATFTPLAEHREYKAGEPVSTIGYPTDSTSPELKEPIVPGQLYKADGVVKGTEKLDDKGAVGITYRLTSVSGLSGGGIINGDGKVIGIHQHGTVDNMNIAEKDRFGGGLVLSPEQLAWVKEIIDKYGVKGWYQGDNGNRYYFTPEGEMIRNKTAVIGKNKYSFDQNGIATLLEGVDYGRVVVEHLDQKDNPVKENDTFVEKTEVGTQFDYNYKTEIEKTDFYKKNKEKYEIVSIDGKAVNKQLKDTWGEDYSVVSKAPAGTRVIKVVYKVNKGSFDLRYRLKGTDQELAPATVDNNDGKEYEVSFVHRFQAKEITGYRAVNASQEATIQHKGVNQVIFEYEKIEDPKPATPATPVVDPKDEETEIGNYGPLPSKAQLDYHKEELAAFIHYGMNTYTNSEWGNGRENPQNFNPTNLDTDQWIKTLKDAGFKRTIMVVKHHDGFVIYPSQYTKHTVAASPWKDGKGDLLEEISKSATKYDMNMGVYLSPWDANNPKYHVSTEKEYNEYYLNQLKEILGNPKYGNKGKFIEVWMDGARGSGAQKVTYTFDEWFKYIKKAEGDIAIFSAQPTSVRWIGNERGIAGDPVWHKVKKAKITDDVKNEYLNHGDPEGDMYSVGEADVSIRSGWFYHDNQQPKSIKDLMDIYFKSVGRGTPLLLNIPPNKEGKFADADVARLKEFRATLDQMYATDFAKGATVTASSTRKNHLYQASNLTDGKDDTSWALSNDAKTGEFTVDLGQKRRFDVVELKEDIAKGQRISGFKVEVELNGRWVPYGEGSTVGYRRLVQGQPVEAQKIRVTITNSQATPILTNFSVYKTPSSIEKTDGYPLGLDYHSNTTADKANTTWYDESEGIRGTSMWTNKKDASVTYRFNGTKAYVVSTVDPNHGEMSVYVDGQKVADVQTNNAARKRSQMVYETDDLAPGEHTIKLVNKTGKAIATEGIYTLNNAGKGMFELKETTYEVQKGQPVTVTIKRVGGSKGAATVHVVTEPGTGVHGKVYKDTTADLTFQDGETEKTLTIPTIDFTEQADSIFDFKVKMTSASDNALLGFASEATVRVMKADLLQKDQVSHDDQASQLDYSPGWHHETNSAGKYQNTESWASFGRLNEEQKKNASVTAYFYGTGLEIKGFVDPGHGIYKVTLDGKELEYQDGQGNATDVNGKKYFSGTATTRQGDQTLVRLTGLEEGWHAVTLQLDPKRNDTSRNIGIQVDKFITRGEDSALYTKEELVQAMKNWKDELAKFDQTSLKNTPEARQAFKSNLDKLSEQLSASPANAQEILKIATALQAILDKEENYGKEDTPTSEQPEEPNYDKAMASLSEAIQNKSKELSSDKEAKKKLVELSEQALTAIQEAKTQDAVDKALQAALTSINQLQATPKEEVKPSQPEEPNYDKAMASLAEAIQNKSKELGSDKESKKKLVELSEQALTAIQEAKTQDAVDKALQAALTSINQLQATPKEEAKPSQPEEPNYDKAMASLAEAIQNKSKELGSDKEAKKKLVELSEQALTAIQEAKTQDAVDKALQAALTSINQLQATPKEEVKHSIVPTDGDKELVQPQPSLEVVEKVINFKKVKQEDSSLPKGETRVTQVGRAGKERILTEVAPDGSRTIKLREVVEVAQDEIVLVGTKKEESGKIASSVHEVPEFTGGVIDSEATIHNLPEFTGGVTDSEAAIHNLPEFTGGVTDSEAAIHNLPEFTGGMTDSEAAIHNLPEFTGGMTDSEGVAHGVSNVEEGVPSGEATSHQESGFTSDVTDSETTMNEIVYKNDEKSYVVPPMLEDKTYQAPANRQEVLPKTGSEDGSAFASVGIIGMFLGMIGIVKRKKD

SEQ ID NO: 19 - protein, 305 aa, Streptococcus mitis
MSGLKKYEGVIPAFYACYDDAGEVSPERTRALVQYFIDKGVQGLYVNGSSGECIYQSVEDRKLILEEVMAVAKGKLTIIAHVACNNTKDSIELARHAESLGVDAIATIPPIYFRLPEYSVAKYWNDISAAAPNTDYVIYNIPQLAGVALTPSLYTEMLKNPRVIGVKNSSMPVQDIQTFVSLGGDDHIVFNGPDEQFLGGRLMGAKAGIGGTYGAMPELFLKLNQLIADKDLETARELQYAINAIIGKLTAAHGNMYCVIKEVLKINEGLNIGSVRSPLTPVTEEDRPVVEAAAQLIRESKERFL

SEQ ID NO: 20 - protein, 526 aa, Porphyromonas gingivalis
MANNTLLAKTRRYVCLVVFCCLMAMMHLSGQEVTMWGDSHGVAPNQVRRTLVKVALSESLPPGAKQIRIGFSLPKETEEKVTALYLLVSDSLAVRDLPDYKGRVSYDSFPISKEDRTTALSADSVAGRCFFYLAADIGPVASFSRSDTLTARVEELAVDGRPLPLKELSPASRRLYREYEALFVPGDGGSRNYRIPSILKTANGTLIAMADRRKYNQTDLPEDIDIVMRRSTDGGKSWSDPRIIVQGEGRNHGFGDVALVQTQAGKLLMIFVGGVGLWQSTPDRPQRTYISESRDEGLTWSPPRDITHFIFGKDCADPGRSRWLASFCASGQGLVLPSGRVMFVAAIRESGQEYVLNNYVLYSDDEGGTWQLSDCAYHRGDEAKLSLMPDGRVLMSVRNQGRQESRQRFFALSSDDGLTWERAKQFEGIHDPGCNGAMLQVKRNGRNQMLHSLPLGPDGRRDGAVYLFDHVSGRWSAPVVVNSGSSAYSDMTLLADGTIGYFVEEDDEISLVFIRFVLDDLFDARQ

SEQ ID NO: 21 - protein, 465 aa, Tannerella forsythia
MTKKSSISRRSFLKSTALAGAAGMVGTGGAATLLTSCGGGASSNENANAANKPLKEPGTYYVPELPDMAADGKELKAGIIGCGGRGSGAAMNFLAAANGVSIVALGDTFQDRVDSLAQKLKDEKNIDIPADKRFVGLDAYKQVIDSDVDVVIVATPPNFRPIHFQYAVEKSKHCFLEKPICVDAVGYRTIMATAKQAQAKNLCVITGTQRHHQRSYIASYQQIMNGAIGEITGGTVYWNQSMLWYRERQAGWSDCEWMIRDWVNWKWLSGDHIVEQHVHNIDVFTWFSGLKPVKAVGFGSRQRRITGDQYDNFSIDFTMENGIHLHSMCRQIDGCANNVSEFIQGTKGSWNSTDMGIKDLAGNVIWKYDVEAEKASFKQNDPYTLEHVNWINTIRAGKSIDQASETAVSNMAAIMGRESAYTGEETTWEAMTAAALDYTPADLNLGKMDMKPFVVPVPGKPLEKK

SEQ ID NO: 22 - protein, 539 aa, Tannerella forsythia
MKKFFWIIGLFISMLTTRAADSVYVQNPQIPILIDRTDNVLFRIRIPDATKGDVLNRLTIRFGNEDKLSEVKAVRLFYAGTEAGTKGRSRFAPVTYVSSHNIRNTRSANPSYSVRQDEVTTVANTLTLKTRQPMVKGINYFWVSVEMDRNTSLLSKLTPTVTEAVINDKPAVIAGEQAAVRRMGIGVRHAGDDGSASFRIPGLVTTNEGTLLGVYDVRYNNSVDLQEHIDVGLSRSTDKGQTWEPMRIAMSFGETDGLPSGQNGVGDPSILVDERTNTVWVVAAWTHGMGNARAWTNSMPGMTPDETAQLMMVKSTDDGRTWSEPINITSQVKDPSWCFLLQGPGRGITMRDGTLVFPIQFIDSLRVPHAGIMYSKDRGETWHIHQPARTNTTEAQVAEVEPGVLMLNMRDNRGGSRAVSITRDLGKSWTEHSSNRSALPESICMASLISVKAKDNIIGKDLLFFSNPNTTEGRHHITIKASLDGGVTWLPAHQVLLDEEDGWGYSCLSMIDRETVGIFYESSVAHMTFQAVKIKDLIR

SEQ ID NO: 23 - protein, 419 aa, Akkermansia muciniphila
MTWLLCGRGKWNKVKRMMNSVFKCLMSAVCAVALPAFGQEEKTGFPTDRAVTVFSAGEGNPYASIRIPALLSIGKGQLLAFAEGRYKNTDQGENDIIMSVSKNGGKTWSRPRAIAKAHGATFNNPCPVYDAKTRTVTVVFQRYPAGVKERQPNIPDGWDDEKCIRNFMIQSRNGGSSWTKPQEITKTTKRPSGVDIMASGPNAGTQLKSGAHKGRLVIPMNEGPFGKWVISCIYSDDGGKSWKLGQPTANMKGMVNETSIAETDNGGVVMVARHWGAGNCRRIAWSQDGGETWGQVEDAPELFCDSTQNSLMTYSLSDQPAYGGKSRILFSGPSAGRRIKGQVAMSYDNGKTWPVKKLLGEGGFAYSSLAMVEPGIVGVLYEENQEHIKKLKFVPITMEWLTDGEDTGLAPGKKAPVLK

SEQ ID NO: 24 - protein, 674 aa, Akkermansia muciniphila
MGLGLLCALGLSIPSVLGKESFEQARRGKFTTLSTKYGLMSCRNGVAEIGGGGKSGEASLRMFGGQDAELKLDLKDTPSREVRLSAWAERWTGQAPFEFSIVAIGPNGEKKIYDGKDIRTGGFHTRIEASVPAGTRSLVFRLTSPENKGMKLDDLFLVPCIPMKVNPQVEMASSAYPVMVRIPCSPVLSLNVRTDGCLNPQFLTAVNLDFTGTTKLSDIESVAVIRGEEAPIIHHGEEPFPKDSSQVLGTVKLAGSARPQISVKGKMELEPGDNYLWACVTMKEGASLDGRVVVRPASVVAGNKPVRVANAAPVAQRIGVAVVRHGDFKSKFYRIPGLARSRKGTLLAVYDIRYNHSGDLPANIDVGVSRSTDGGRTWSDVKIAIDDSKIDPSLGATRGVGDPAILVDEKTGRIWVAAIWSHRHSIWGSKSGDNSPEACGQLVLAYSDDDGLTWSSPINITEQTKNKDWRILFNGPGNGICMKDGTLVFAAQYWDGKGVPWSTIVYSKDRGKTWHCGTGVNQQTTEAQVIELEDGSVMINARCNWGGSRIVGVTKDLGQTWEKHPTNRTAQLKEPVCQGSLLAVDGVPGAGRVVLFSNPNTTSGRSHMTLKASTNDAGSWPEDKWLLYDARKGWGYSCLAPVDKNHVGVLYESQGALNFLKIPYKDVLNAKNAR

SEQ ID NO: 25 - protein, 544 aa, Bacteroides thetaiotaomicron
MKRNHYLFTLILLLGCSIFVKASDTVFVHQTQIPILIERQDNVLFYFRLDAKESRMMDEIVLDFGKSVNLSDVQAVKLYYGGTEALQDKGKKRFAPVDYISSHRPGNTLAAIPSYSIKCAEALQPSAKVVLKSHYKLFPGINFFWISLQMKPETSLFTKISSELQSVKIDGKEAICEERSPKDIIHRMAVGVRHAGDDGSASFRIPGLVTSNKGTLLGVYDVRYNSSVDLQEYVDVGLSRSTDGGKTWEKMRLPLSFGEYDGLPAAQNGVGDPSILVDTQTNTIWVVAAWTHGMGNQRAWWSSHPGMDLYQTAQLVMAKSTDDGKTWSKPINITEQVKDPSWYFLLQGPGRGITMSDGTLVFPTQFIDSTRVPNAGIMYSKDRGKTWKMHNMARTNTTEAQVVETEPGVLMLNMRDNRGGSRAVAITKDLGKTWTEHPSSRKALQEPVCMASLIHVEAEDNVLDKDILLFSNPNTTRGRNHITIKASLDDGLTWLPEHQLMLDEGEGWGYSCLTMIDRETIGILYESSAAHMTFQAVKLKDLIR

SEQ ID NO: 26 - protein, 911 aa, Actinomyces viscosus
MTSHSPFSRRHLPALLGSLPLAATGLIAAAPPAHAVPTSDGLADVTITQVNAPADGLYSVGDVMTFNITLTNTSGEAHSYAPASTNLSGNVSKCRWRNVPAGTTKTDCTGLATHTVTAEDLKAGGFTPQIAYEVKAVEYAGKALSTPETIKGATSPVKANSLRVESITPSSSKEYYKLGDTVTYTVRVRSVSDKTINVAATESSFDDLGRQCHWGGLKPGKGAVYNCKPLTHTITQADVDAGRWTPSITLTATGTDGTALQTLTATGNPINVVGDHPQATPAPAPDASTELPASMSQAQHVAPNTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDHGWGNSQAGTDPENRGIIQAEVSTSTDNGWTWTHRTITADITKDNPWTARFAASGQGIQIQHGPHAGRLVQQYTIRTAGGAVQAVSVYSDDHGKTWQAGTPVGTGMDENKVVELSDGSLMLNSRASDSSGFRKVAHSTDGGQTWSEPVSDKNLPDSVDNAQIIRAFPNAAPDDPRAKVLLLSHSPNPKPWSRDRGTISMSCDDGASWTTSKVFHEPFVGYTTIAVQSDGSIGLLSEDAHDGANYGGIWYRNFTMNWLGEQCGQKPAEPSPAPSPTAAPSAAPSEQPAPSAAPSTEPTQAPAPSSAPEPSAVPEPSSAPAPEPTTAPSTEPTPTPAPSSAPEPSAGPTAAPAPETSSAPAAEPTQAPTVAPSAEPTQVPGAQPSAAPSEKPGAQPSSAPKPDATGRAPSVVNPKATAAPSGKASSSASPAPSRSATATSKPGMEPDEIDRPSDGAMAQPTGGASAPSAAPTQAAKAGSRLSRTGTNALLVLGLAGVAVVGGYLLLRARRSKN

SEQ ID NO: 27 - protein, 393 aa, Artificial Sequence
  Synthetic Construct
GDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPA

SEQ ID NO: 28 - protein, 435 aa, Artificial Sequence
  Synthetic Construct
METDTLLLWVLLLWVPGSTGDGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGYTTIAVQSDGSIGLLSEDAHNGADYGGIWYRNFTMNWLGEQCGQKPAKRKKKGGKNGKNRRNRKKKNP

SEQ ID NO: 29 - protein, 435 aa, Artificial Sequence
  Synthetic Construct
METDTLLLWVLLLWVPGSTGDGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGFTTIAVQSDGSIGLLSEDAHNGADYGGIWYRNFTMNWLGEQCGQKPAKRKKKGGKNGKNRRNRKKKNP

SEQ ID NO: 30 - protein, 422 aa, Artificial Sequence
  Synthetic Construct
METDTLLLWVLLLWVPGSTGDMASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQKRKKKGGKNGKNRRNRKKKNP

SEQ ID NO: 31 - protein, 495 aa, Artificial Sequence
  Synthetic Construct
METDTLLLWVLLLWVPGSTGDYPYDVPDYAGATPARSPGMGDHPQATPAPAPDASTELPASMSQAQHLAANTATDNYRIPAITTAPNGDLLISYDERPKDNGNGGSDAPNPNHIVQRRSTDGGKTWSAPTYIHQGTETGKKVGYSDPSYVVDHQTGTIFNFHVKSYDQGWGGSRGGTDPENRGIIQAEVSTSTDNGWTWTHRTITADITKDKPWTARFAASGQGIQIQHGPHAGRLVQQYTIRTAGGAVQAVSVYSDDHGKTWQAGTPIGTGMDENKVVELSDGSLMLNSRASDGSGFRKVAHSTDGGQTWSEPVSDKNLPDSVDNAQIIRAFPNAAPDDPRAKVLLLSHSPNPRPWSRDRGTISMSCDDGASWTTSKVFHEPFVGFTTIAVQSDGSIGLLSEDAHNGADYGGIWYRNFTMNWLGEQCGQKPAVDEQKLISEEDLNAVGQDTQEVIVVPHSLPFKVVVISAILALVVLTIISLIILIMLWQKKPR

SEQ ID NO: 32 - protein, 495 aa, Artificial Sequence; identical to SEQ ID NO: 31
  Synthetic Construct

SEQ ID NO: 33 - protein, 481 aa, Artificial Sequence
  Synthetic Construct
METDTLLLWVLLLWVPGSTGDYPYDVPDYAGATPARSPGMASLPVLQKESVFQSGAHAYRIPALLYLPGQQSLLAFAEQRASKKDEHAELIVLRRGDYDAPTHQVQWQAQEVVAQARLDGHRSMNPCPLYDAQTGTLFLFFIAIPGQVTEQQQLQTRANVTRLCQVTSTDHGRTWSSPRDLTDAAIGPAYREWSTFAVGPGHCLQLHDRARSLVVPAYAYRKLHPIQRPIPSAFCFLSHDHGRTWARGHFVAQDTLECQVAEVETGEQRVVTLNARSHLRARVQAQSTNDGLDFQESQLVKKLVEPPPQGCQGSVISFPSPRSGPGSPAQWLLYTHPTHSWQRADLGAYLNPRPPAPEAWSEPVLLAKGSCAYSDLQSMGTGPDGSPLFGCLYEANDYEEIVFLMFTLKQAFPAEYLPQVDEQKLISEEDLNAVGQDTQEVIVVPHSLPFKVVVISAILALVVLTIISLIILIMLWQKKPR

SEQ ID NO: 34 - DNA, 1311 bp, Artificial Sequence
  Synthetic Construct
atggagacagacacactcctgctatgggtactgctgctctgggttccaggttccactggtgacggcgaccacccacaggcaacaccagcacctgccccagatgcctccaccgagctgccagcaagcatgtcccaggcacagcacctggcagcaaataccgcaacagacaactacagaatccccgccatcaccacagccccaaatggcgatctgctgatcagctatgacgagcgccccaaggataacggaaatggaggctccgacgcaccaaaccctaatcacatcgtgcagcggagatctaccgatggcggcaagacatggagcgcccctacctacatccaccagggcaccgagacaggcaagaaggtcggctactctgacccaagctatgtggtggatcaccagaccggcacaatcttcaactttcacgtgaagtcctatgaccagggatggggaggctctaggggcggcaccgatcctgagaatcgcggcatcatccaggccgaggtgtctaccagcacagacaacggctggacctggacacaccggaccatcacagccgacatcacaaaggataagccctggaccgcaagattcgcagcaagcggacagggcatccagatccagcacggacctcacgcaggccggctggtgcagcagtacaccatcagaacagcaggaggagcagtgcaggccgtgtccgtgtattctgacgatcacggcaagacctggcaggcaggcaccccaatcggcacaggcatggacgagaataaggtggtggagctgagcgatggctccctgatgctgaactctagggccagcgacggctccggcttccgcaaggtggcacactctacagacggaggacagacctggtccgagcccgtgtctgataagaatctgcctgacagcgtggataacgcccagatcatccgggcctttcctaatgccgccccagacgatcccagagccaaggtgctgctgctgtcccactctccaaacccaaggccttggagccgggacagaggcacaatcagcatgtcctgcgacgatggcgccagctggaccacatccaaggtgttccacgagccatttgtgggctacaccacaatcgccgtgcagtctgatggcagcatcggactgctgagcgaggacgcacacaatggcgccgattacggcggcatctggtatcggaacttcaccatgaactggctgggcgagcagtgtggccagaagccagccaagcggaagaagaagggcggcaagaacggcaagaataggcgcaaccggaagaagaagaacccctgatga

SEQ ID NO: 35 - DNA, 1311 bp, Artificial Sequence
  Synthetic Construct
atggagacagacacactcctgctatgggtactgctgctctgggttccaggttccactggtgacggcgaccacccacaggcaacaccagcacctgccccagatgcctccaccgagctgccagcaagcatgtcccaggcacagcacctggcagcaaataccgcaacagacaactacagaatccccgccatcaccacagccccaaatggcgatctgctgatcagctatgacgagcgccccaaggataacggaaatggaggctccgacgcaccaaaccctaatcacatcgtgcagcggagatctaccgatggcggcaagacatggagcgcccctacctacatccaccagggcaccgagacaggcaagaaggtcggctactctgacccaagctatgtggtggatcaccagaccggcacaatcttcaactttcacgtgaagtcctatgaccagggatggggaggctctaggggcggcaccgatcctgagaatcgcggcatcatccaggccgaggtgtctaccagcacagacaacggctggacctggacacaccggaccatcacagccgacatcacaaaggataagccctggaccgcaagattcgcagcaagcggacagggcatccagatccagcacggacctcacgcaggccggctggtgcagcagtacaccatcagaacagcaggaggagcagtgcaggccgtgtccgtgtattctgacgatcacggcaagacctggcaggcaggcaccccaatcggcacaggcatggacgagaataaggtggtggagctgagcgatggctccctgatgctgaactctagggccagcgacggctccggcttccgcaaggtggcacactctacagacggaggacagacctggtccgagcccgtgtctgataagaatctgcctgacagcgtggataacgcccagatcatccgggcctttcctaatgccgccccagacgatcccagagccaaggtgctgctgctgtcccactctccaaacccaaggccttggagccgggacagaggcacaatcagcatgtcctgcgacgatggcgccagctggaccacatccaaggtgttccacgagccatttgtgggcttcaccacaatcgccgtgcagtctgatggcagcatcggactgctgagcgaggacgcacacaatggcgccgattacggcggcatctggtatcggaacttcaccatgaactggctgggcgagcagtgtggccagaagccagccaagcggaagaagaagggcggcaagaacggcaagaataggcgcaaccggaagaagaagaacccctgatga

SEQ ID NO: 36 - DNA, 1272 bp, Artificial Sequence
  Synthetic Construct
atggagacagacacactcctgctatgggtactgctgctctgggttccaggttccactggtgacatggccagcctgcctgtgctgcagaaggagagcgtgttccagtccggcgcccacgcatacagaatccccgccctgctgtatctgcctggccagcagtccctgctggcctttgccgagcagagagcctctaagaaggacgagcacgcagagctgatcgtgctgaggaggggcgactacgatgcaccaacccaccaggtgcagtggcaggcacaggaggtggtggcacaggcaaggctggacggacaccgcagcatgaatccatgccccctgtatgatgcccagaccggcacactgttcctgttctttatcgcaatccccggccaggtgaccgagcagcagcagctgcagaccagagccaacgtgacaagactgtgccaggtgacctccacagaccacggcaggacctggagcagccctcgcgacctgacagatgcagcaatcggaccagcatacagggagtggtctacattcgccgtgggccctggccactgcctgcagctgcacgatcgggccagaagcctggtggtgccagcctacgcctatcggaagctgcaccccatccagagacctatcccatctgccttctgctttctgagccacgaccacggcagaacttgggccagaggccactttgtggcccaggatacactggagtgtcaggtggcagaggtggagaccggagagcagagggtggtgacactgaatgcacgcagccacctgagggcccgcgtgcaggcccagtccaccaacgacggcctggatttccaggagtctcagctggtgaagaagctggtggagccacctccacagggatgtcagggctctgtgatcagctttccctcccctcggtctggcccaggcagcccagcacagtggctgctgtacacccaccccacacactcctggcagagggcagacctgggagcatatctgaatccaagaccccctgcaccagaggcctggtccgagcctgtgctgctggccaagggctcttgcgcctacagcgacctgcagagcatgggcaccggacctgatggctctccactgttcggctgtctgtacgaggccaacgattatgaggagatcgtgttcctgatgtttacactgaagcaggcctttcctgccgagtatctgccacagaagcggaagaagaagggcggcaagaacggcaagaatcggagaaaccggaagaagaagaacccttgatga

SEQ ID NO: 37 - DNA, 1485 bp, Artificial Sequence
  Synthetic Construct
atggagacagacacactcctgctatgggtactgctgctctgggttccaggttccactggtgactatccatatgatgttccagattatgctggggccacgccggccagatctcccgggatgggcgaccacccacaggcaacaccagcacctgccccagatgcctccaccgagctgccagcaagcatgtcccaggcacagcacctggcagcaaataccgcaacagacaactacagaatccccgccatcaccacagccccaaatggcgatctgctgatcagctatgacgagcgccccaaggataacggaaatggaggctccgacgcaccaaaccctaatcacatcgtgcagcggagatctaccgatggcggcaagacatggagcgcccctacctacatccaccagggcaccgagacaggcaagaaggtcggctactctgacccaagctatgtggtggatcaccagaccggcacaatcttcaactttcacgtgaagtcctatgaccagggatggggaggctctaggggcggcaccgatcctgagaatcgcggcatcatccaggccgaggtgtctaccagcacagacaacggctggacctggacacaccggaccatcacagccgacatcacaaaggataagccctggaccgcaagattcgcagcaagcggacagggcatccagatccagcacggacctcacgcaggccggctggtgcagcagtacaccatcagaacagcaggaggagcagtgcaggccgtgtccgtgtattctgacgatcacggcaagacctggcaggcaggcaccccaatcggcacaggcatggacgagaataaggtggtggagctgagcgatggctccctgatgctgaactctagggccagcgacggctccggcttccgcaaggtggcacactctacagacggaggacagacctggtccgagcccgtgtctgataagaatctgcctgacagcgtggataacgcccagatcatccgggcctttcctaatgccgccccagacgatcccagagccaaggtgctgctgctgtcccactctccaaacccaaggccttggagccgggacagaggcacaatcagcatgtcctgcgacgatggcgccagctggaccacatccaaggtgttccacgagccatttgtgggctacaccacaatcgccgtgcagtctgatggcagcatcggactgctgagcgaggacgcacacaatggcgccgattacggcggcatctggtatcggaacttcaccatgaactggctgggcgagcagtgtggccagaagccagccgtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 38 - DNA, 1485 bp, Artificial Sequence
  Synthetic Construct
atggagacagacacactcctgctatgggtactgctgctctgggttccaggttccactggtgactatccatatgatgttccagattatgctggggccacgccggccagatctcccgggatgggcgaccacccacaggcaacaccagcacctgccccagatgcctccaccgagctgccagcaagcatgtcccaggcacagcacctggcagcaaataccgcaacagacaactacagaatccccgccatcaccacagccccaaatggcgatctgctgatcagctatgacgagcgccccaaggataacggaaatggaggctccgacgcaccaaaccctaatcacatcgtgcagcggagatctaccgatggcggcaagacatggagcgcccctacctacatccaccagggcaccgagacaggcaagaaggtcggctactctgacccaagctatgtggtggatcaccagaccggcacaatcttcaactttcacgtgaagtcctatgaccagggatggggaggctctaggggcggcaccgatcctgagaatcgcggcatcatccaggccgaggtgtctaccagcacagacaacggctggacctggacacaccggaccatcacagccgacatcacaaaggataagccctggaccgcaagattcgcagcaagcggacagggcatccagatccagcacggacctcacgcaggccggctggtgcagcagtacaccatcagaacagcaggaggagcagtgcaggccgtgtccgtgtattctgacgatcacggcaagacctggcaggcaggcaccccaatcggcacaggcatggacgagaataaggtggtggagctgagcgatggctccctgatgctgaactctagggccagcgacggctccggcttccgcaaggtggcacactctacagacggaggacagacctggtccgagcccgtgtctgataagaatctgcctgacagcgtggataacgcccagatcatccgggcctttcctaatgccgccccagacgatcccagagccaaggtgctgctgctgtcccactctccaaacccaaggccttggagccgggacagaggcacaatcagcatgtcctgcgacgatggcgccagctggaccacatccaaggtgttccacgagccatttgtgggcttcaccacaatcgccgtgcagtctgatggcagcatcggactgctgagcgaggacgcacacaatggcgccgattacggcggcatctggtatcggaacttcaccatgaactggctgggcgagcagtgtggccagaagccagccgtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 39 - DNA, 1443 bp, Artificial Sequence
  Synthetic Construct
atggagacagacacactcctgctatgggtactgctgctctgggttccaggttccactggtgactatccatatgatgttccagattatgctggggccacgccggccagatctcccgggatggccagcctgcctgtgctgcagaaggagagcgtgttccagtccggcgcccacgcatacagaatccccgccctgctgtatctgcctggccagcagtccctgctggcctttgccgagcagagagcctctaagaaggacgagcacgcagagctgatcgtgctgaggaggggcgactacgatgcaccaacccaccaggtgcagtggcaggcacaggaggtggtggcacaggcaaggctggacggacaccgcagcatgaatccatgccccctgtatgatgcccagaccggcacactgttcctgttctttatcgcaatccccggccaggtgaccgagcagcagcagctgcagaccagagccaacgtgacaagactgtgccaggtgacctccacagaccacggcaggacctggagcagccctcgcgacctgacagatgcagcaatcggaccagcatacagggagtggtctacattcgccgtgggccctggccactgcctgcagctgcacgatcgggccagaagcctggtggtgccagcctacgcctatcggaagctgcaccccatccagagacctatcccatctgccttctgctttctgagccacgaccacggcagaacttgggccagaggccactttgtggcccaggatacactggagtgtcaggtggcagaggtggagaccggagagcagagggtggtgacactgaatgcacgcagccacctgagggcccgcgtgcaggcccagtccaccaacgacggcctggatttccaggagtctcagctggtgaagaagctggtggagccacctccacagggatgtcagggctctgtgatcagctttccctcccctcggtctggcccaggcagcccagcacagtggctgctgtacacccaccccacacactcctggcagagggcagacctgggagcatatctgaatccaagaccccctgcaccagaggcctggtccgagcctgtgctgctggccaagggctcttgcgcctacagcgacctgcagagcatgggcaccggacctgatggctctccactgttcggctgtctgtacgaggccaacgattatgaggagatcgtgttcctgatgtttacactgaagcaggcctttcctgccgagtatctgccacaggtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 40 - protein, 21 aa, Artificial Sequence
  Synthetic Construct
METDTLLLWVLLLWVPGSTGD

SEQ ID NO: 41 - protein, 9 aa, Artificial Sequence
  Synthetic Construct
YPYDVPDYA

SEQ ID NO: 42 - protein, 9 aa, Artificial Sequence
  Synthetic Construct
GATPARSPG

SEQ ID NO: 43 - protein, 2 aa, Artificial Sequence
  Synthetic Construct
VD

SEQ ID NO: 44 - protein, 10 aa, Artificial Sequence
  Synthetic Construct
EQKLISEEDL

SEQ ID NO: 45 - protein, 50 aa, Homo sapiens
NAVGQDTQEVIVVPHSLPFKVVVISAILALVVLTIISLIILIMLWQKKPR

SEQ ID NO: 46 - protein, 27 aa, Homo sapiens
FWVLVVVGGVLACYSLLVTVAFIIFWV

SEQ ID NO: 47 - protein, 22 aa, Homo sapiens
MALIVLGGVAGLLLFIGLGIFF

SEQ ID NO: 48 - protein, 21 aa, Homo sapiens
IYIWAPLAGTCGVLLLSLVIT

SEQ ID NO: 49 - protein, 23 aa, Homo sapiens
IYIWAPLAGTCGVLLLSLVITLY

SEQ ID NO: 50 - protein, 24 aa, Homo sapiens
IYIWAPLAGTCGVLLLSLVITLYC

SEQ ID NO: 51 - protein, 27 aa, Homo sapiens
IISFFLALTSTALLFLLFFLTLRFSVV

SEQ ID NO: 52 - protein, 21 aa, Homo sapiens
VVISAILALVVLTIISLIILI

SEQ ID NO: 53 - protein, 381 aa, Salmonella typhimurium
TVEKSVVFKAEGEHFTDQKGNTIVGSGSGGTTKYFRIPAMCTTSKGTIVVFADARHNTASDQSFIDTAAARSTDGGKTWNKKIAIYNDRVNSKLSRVMDPTCIVANIQGRETILVMVGKWNNNDKTWGAYRDKAPDTDWDLVLYKSTDDGVTFSKVETNIHDIVTKNGTISAMLGGVGSGLQLNDGKLVFPVQMVRTKNITTVLNTSFIYSTDGITWSLPSGYCEGFGSENNIIEFNASLVNNIRNSGLRRSFETKDFGKTWTEFPPMDKKVDNRNHGVQGSTITIPSGNKLVAAHSSAQNKNNDYTRSDISLYAHNLYSGEVKLIDDFYPKVGNASGAGYSCLSYRKNVDKETLYVVYEANGSIEFQDLSRHLPVIKSYN

SEQ ID NO: 54 - protein, 781 aa, Vibrio cholera
MRFKNVKKTALMLAMFGMATSSNAALFDYNATGDTEFDSPAKQGWMQDNTNNGSGVLTNADGMPAWLVQGIGGRAQWTYSLSTNQHAQASSFGWRMTTEMKVLSGGMITNYYANGTQRVLPIISLDSSGNLVVEFEGQTGRTVLATGTAATEYHKFELVFLPGSNPSASFYFDGKLIRDNIQPTASKQNMIVWGNGSSNTDGVAAYRDIKFEIQGDVIFRGPDRIPSIVASSVTPGVVTAFAEKRVGGGDPGALSNTNDIITRTSRDGGITWDTELNLTEQINVSDEFDFSDPRPIYDPSSNTVLVSYARWPTDAAQNGDRIKPWMPNGIFYSVYDVASGNWQAPIDVTDQVKERSFQIAGWGGSELYRRNTSLNSQQDWQSNAKIRIVDGAANQIQVADGSRKYVVTLSIDESGGLVANLNGVSAPIILQSEHAKVHSFHDYELQYSALNHTTTLFVDGQQITTWAGEVSQENNIQFGNADAQIDGRLHVQKIVLTQQGHNLVEFDAFYLAQQTPEVEKDLEKLGWTKIKTGNTMSLYGNASVNPGPGHGITLTRQQNISGSQNGRLIYPAIVLDRFFLNVMSIYSDDGGSNWQTGSTLPIPFRWKSSSILETLEPSEADMVELQNGDLLLTARLDFNQIVNGVNYSPRQQFLSKDGGITWSLLEANNANVFSNISTGTVDASITRFEQSDGSHFLLFTNPQGNPAGTNGRQNLGLWFSFDEGVTWKGPIQLVNGASAYSDIYQLDSENAIVIVETDNSNMRILRMPITLLKQKLTLSQN

SEQ ID NO: 55 - DNA, 1520 bp, Artificial Sequence
  Synthetic Construct
atggagtttggactgagctggctgtttctcgtggccattctgaagggcgtccagtgcagcagagacatccagatgacccagacaaccagctctctgagcgctagcctcggagatagagtgaccattagctgtagagcctcccaagacatttccaagtacctcaactggtaccagcagaagcccgacggcaccgtgaagctgctgatctaccacaccagcagactgcactccggagtgccctctaggttttccggatccggcagcggcacagactactctctgaccatctccaatctggagcaagaggacatcgccacctacttctgccagcaaggcaacacactgccttacacattcggcggcggaacaaagctcgaactgaaaagaggcggcggcggaagcggaggaggaggatccggaggcggaggatccggcggaggaggctccgaagtccagctgcaacaaagcggacccggactggtggctcccagccaatctctgagcgtgacatgcacagtgtccggcgtctctctgcccgactacggagtcagctggattagacagcctcctagaaagggactggagtggctgggagtcatctggggcagcgagaccacctactataactccgccctcaagtctaggctcaccatcatcaaagacaacagcaagagccaagtgttcctcaagatgaacagcctccagaccgacgacaccgccatctactactgcgccaaacactactactacggaggcagctacgctatggattactggggccaaggcaccacagtcacagtgagcagctatgtgaccgtgagcagccaagaccccgccaaagatcccaagttctgggtgctggtcgtggtgggaggcgtgctggcttgttattctctgctggtgaccgtggccttcatcatcttctgggtgaggagcaagagatccagactgctgcacagcgactacatgaacatgacacctagaaggcccggccccacaaggaaacattaccagccctacgccccccctagagacttcgctgcctatagatccaagagaggaagaaaaaagctgctctacatcttcaagcagcccttcatgaggcccgtgcaaacaacacaagaggaggacggatgtagctgtagattccccgaggaggaagagggaggatgcgagctgagagtgaagttctctaggagcgccgatgctcccgcttatcagcaaggccagaaccagctgtacaatgagctgaatctgggaagaagggaagaatacgacgtgctggataagaggaggggaagagaccccgagatgggaggcaagcctagaaggaagaacccccaagagggactgtacaacgagctccaaaaggacaagatggctgaagcctacagcgagatcggaatgaagggagagagaaggaggggcaagggccacgatggactctaccaaggcctcagcacagccaccaaggacacctacgacgctctgcacatgcaagctctgcccccagatgatga

SEQ ID NO: 56 - protein, 506 aa, Artificial Sequence
  Synthetic Construct
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSSQDPAKDPKFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPDD

SEQ ID NO: 57 - protein, 268 aa, Artificial Sequence
  Synthetic Construct
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVS

SEQ ID NO: 58 - protein, 375 aa, Artificial Sequence
  Synthetic Construct
MDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGGGGSGGGGSGGGGSDGTLFPGDDDLAIPATEFFSTKAAKAPEDKAADAAAAAADDNEETLKQRLTNLEKKITNVTTKFEQIEKCCKRNDEVLFRLENHAETLRAAMISLAKKIDVQTGRAAAE

SEQ ID NO: 59 - DNA, 259 bp, Artificial Sequence
  Synthetic Construct
agttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaatt

SEQ ID NO: 60 - DNA, 1251 bp, Artificial Sequence
  Synthetic Construct
tcatcaggggttcttcttcttccggttgcgcctattcttgccgttcttgccgcccttcttcttccgcttggctggcttctggccacactgctcgcccagccagttcatggtgaagttccgataccagatgccgccgtaatcggcgccattgtgtgcgtcctcgctcagcagtccgatgctgccatcagactgcacggcgattgtggtgtagcccacaaatggctcgtggaacaccttggatgtggtccagctggcgccatcgtcgcaggacatgctgattgtgcctctgtcccggctccaaggccttgggtttggagagtgggacagcagcagcaccttggctctgggatcgtctggggcggcattaggaaaggcccggatgatctgggcgttatccacgctgtcaggcagattcttatcagacacgggctcggaccaggtctgtcctccgtctgtagagtgtgccaccttgcggaagccggagccgtcgctggccctagagttcagcatcagggagccatcgctcagctccaccaccttattctcgtccatgcctgtgccgattggggtgcctgcctgccaggtcttgccgtgatcgtcagaatacacggacacggcctgcactgctcctcctgctgttctgatggtgtactgctgcaccagccggcctgcgtgaggtccgtgctggatctggatgccctgtccgcttgctgcgaatcttgcggtccagggcttatcctttgtgatgtcggctgtgatggtccggtgtgtccaggtccagccgttgtctgtgctggtagacacctcggcctggatgatgccgcgattctcaggatcggtgccgcccctagagcctccccatccctggtcataggacttcacgtgaaagttgaagattgtgccggtctggtgatccaccacatagcttgggtcagagtagccgaccttcttgcctgtctcggtgccctggtggatgtaggtaggggcgctccatgtcttgccgccatcggtagatctccgctgcacgatgtgattagggtttggtgcgtcggagcctccatttccgttatccttggggcgctcgtcatagctgatcagcagatcgccatttggggctgtggtgatggcggggattctgtagttgtctgttgcggtatttgctgccaggtgctgtgcctgggacatgcttgctggcagctcggtggaggcatctggggcaggtgctggtgttgcctgtgggtggtcgcccat

SEQ ID NO: 61 - DNA, 31 bp, Artificial Sequence
  Synthetic Construct
gaatttcattttgtttttttctatgctataa

SEQ ID NO: 62 - DNA, 34 bp, Artificial Sequence
  Synthetic Construct
ataacttcgtataatgtatgctatacgaagttat

SEQ ID NO: 63 - DNA, 717 bp, Artificial Sequence
  Synthetic Construct
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 64 - DNA, 204 bp, Artificial Sequence
  Synthetic Construct
aattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataa

SEQ ID NO: 65 - DNA, 3110 bp, Vaccinia virus
atgaacggcggacatattcagttgataatcggccccatgttttcaggtaaaagtacagaattaattagacgagttagacgttatcaaatagctcaatataaatgcgtgactataaaatattctaacgataatagatacggaacgggactatggacgcatgataagaataattttgaagcattggaagcaactaaactatgtgatgtcttggaatcaattacagatttctccgtgataggtatcgatgaaggacagttctttccagacattgttgaattagatcgataaaaattaattaattacccgggtaccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcggccggcctcatcaggggttcttcttcttccggttgcgcctattcttgccgttcttgccgcccttcttcttccgcttggctggcttctggccacactgctcgcccagccagttcatggtgaagttccgataccagatgccgccgtaatcggcgccattgtgtgcgtcctcgctcagcagtccgatgctgccatcagactgcacggcgattgtggtgtagcccacaaatggctcgtggaacaccttggatgtggtccagctggcgccatcgtcgcaggacatgctgattgtgcctctgtcccggctccaaggccttgggtttggagagtgggacagcagcagcaccttggctctgggatcgtctggggcggcattaggaaaggcccggatgatctgggcgttatccacgctgtcaggcagattcttatcagacacgggctcggaccaggtctgtcctccgtctgtagagtgtgccaccttgcggaagccggagccgtcgctggccctagagttcagcatcagggagccatcgctcagctccaccaccttattctcgtccatgcctgtgccgattggggtgcctgcctgccaggtcttgccgtgatcgtcagaatacacggacacggcctgcactgctcctcctgctgttctgatggtgtactgctgcaccagccggcctgcgtgaggtccgtgctggatctggatgccctgtccgcttgctgcgaatcttgcggtccagggcttatcctttgtgatgtcggctgtgatggtccggtgtgtccaggtccagccgttgtctgtgctggtagacacctcggcctggatgatgccgcgattctcaggatcggtgccgcccctagagcctccccatccctggtcataggacttcacgtgaaagttgaagattgtgccggtctggtgatccaccacatagcttgggtcagagtagccgaccttcttgcctgtctcggtgccctggtggatgtaggtaggggcgctccatgtcttgccgccatcggtagatctccgctgcacgatgtgattagggtttggtgcgtcggagcctccatttccgttatccttggggcgctcgtcatagctgatcagcagatcgccatttggggctgtggtgatggcggggattctgtagttgtctgttgcggtatttgctgccaggtgctgtgcctgggacatgcttgctggcagctcggtggaggcatctggggcaggtgctggtgttgcctgtgggtggtcgcccatttatagcatagaaaaaaacaaaatgaaattcaagctttcactaattccaaacccacccgctttttatagtaagtttttcacccataaataataaatacaataattaatttctcgtaaaagtagaaaatatattctaatttattgcacggtaaggaagtagatcataactcgagataacttcgtataatgtatgctatacgaagttatctagcgctaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagactagcgctcaataacttcgtataatgtatgctatacgaagttatgcggccgcttcctcgctcactgacgctagcgccctatagtgagtcgtattacagatccaattctgtgagcgtatggcaaacgaaggaaaaatagttatagtagccgcactcgatgggacatttcaacgtaaaccgtttaataatattttgaatcttattccattatctgaaatggtggtaaaactaactgctgtgtgtatgaaatgctttaaggaggcttccttttctaaacgattgggtgaggaaaccgagatagaaataataggaggtaatgatatgtatcaatcggtgtgtagaaagtgttacatcgactcata

SEQ ID NO: 66 - protein, 324 aa, Artificial Sequence
  Synthetic Construct
MAAAKTPVIVVPVAAALPSETFPNVHEHINDQAAADVADAEVMAAKRNVVVAKDDPDHYKDYAFIQWTGGNIRNDDKYTHFFSGFCNTMCTEETKRNIARHLALWDSNFFTELENKKVEYVVIVENDNVIAAIAFLAPVLKAMHDKKIDILQMAEAITGNAVKTEAAADKNHAIFTYTGGYDVSLSAYIIRVTTALNIADEIIKSGGLSSGFYFEIARIENEMKINAQILDNAAKYVEHDPRLVAEHRFANMAAAAWSRIGTAATKRYPGVMYAFTTPLISFFGLFDINVIGLIVILFIMFMLIFNVKSKLLWFLTGTFVTAFI

SEQ ID NO: 67 - protein, 324 aa, Artificial Sequence
  Synthetic Construct
MAAAKTPVIVVPVIDRLPSETFPNVHEHINDQKFDDVKDNEVMAEKRNVVVVKDDPDHYKDYAFIQWTGGNIRNDDKYTHFFSGFCNTMCTEETKRNIARHLALWDSNFFTELENKKVEYVVIVENDNVIEDITFLRPVLKAMHDKKIDILQMREIITGNKVKTELVMDKNHAIFTYTGGYDVSLSAYIIRVTTALNIVDEIIKSGGLSSGFYFEIARIENEMKINRQILDNAAKYVEHDPRLVAEHRFGWMKPNFWFRIGPATVIRCPGVKNANTAPLISFFGLFDINVIGLIVILFIMFMLIFNVKSKLLWFLTGTFVTAFI

SEQ ID NO: 68 - protein, 324 aa, Artificial Sequence
  Synthetic Construct
MAAAKTPVIVVPVAAALPSETFPNVHEHINDQAAADVADAEVMAAKRNVVVAKDDPDHYKDYAFIQWTGGNIRNDDKYTHFFSGFCNTMCTEETKRNIARHLALWDSNFFTELENKKVEYVVIVENDNVIAAIAAAAPVLKAMHDKKIDILQMAAAITGNAVKTEAAADKNHAIFTYTGGYDVSLSAYIIRVTTALNAADEIIKSGGLSSGFYFEIARIENEMKINAQILDNAAKYVEHDPRLVAEHRFAAAAAAAWARIGPATTIRCPGVKNANTAPLISFFGLFDINVIGLIVILFIMFMLIFNVKSKLLWFLTGTFVTAFI

SEQ ID NO: 69 - protein, 324 aa, Artificial Sequence; identical to SEQ ID NO: 67
  Synthetic Construct

SEQ ID NO: 70 - protein, 304 aa, Artificial Sequence
  Synthetic Construct
MPQQLSPINIETKKAISNARLKPLDIHYNESKPTTIQNTGALVAINFAGGYISGGFLPNEYVLSSLHIYWGKEDDYGSNHLIDVYKYSGEINLVHWNAKKYSSYEEAAKHDDGLIIISIFLQVLDHKNVYFQKIVNQLDSIRSANTSAPFDSVFYLDNLLPSKLDYFTYLGTTINHSADAVWIIFPTPINIHSDQLSKFRTLLSSSNHDGKPHYITENYANPYKLNDDTQVYYSGEIIRAATTSPARENYFMRWLSDLRETCFSYYQKYIEENKTFAIIAIVFVFILTAILFFMSRRYSREKQN

SEQ ID NO: 71 - protein, 304 aa, Artificial Sequence
  Synthetic Construct
MPQQLSPINIETKKAISNARLKPLDIHYNESKPTTIQNTGKLFWINFKGGYISGWFLPNEYVLSSLHIYWGKEDDYGSNHLIDVYKYSGEINLVHWNKKKYSSYEEAKKHDDGLIIISIFLQVLDHKNVYFQKIVNQLDSIRSTNTSAPFDSVFYLDNLLPSKLDYFSYLGTTINHYADAVWIIFPTPINIHSDQLSKYRTLSSSSNHDGKTHYITECYRNLYKLNGDTQVYYSGEIIRAATTSPARENYFMRWLSDLRETCFSYYQKYIEENKTFAIIAIVFVFILTAILFFMSRRYSREKQN

SEQ ID NO: 72 - protein, 304 aa, Artificial Sequence
  Synthetic Construct
MPQQLSPINIETKKAISNARLKPLDIHYNESKPTTIQNTGKLAAINFAGGYIAAAFLPNEYVLSSLHIYWGKEDDYGSNHLIDVYKYSGEINLVHWNAKKYSSYEEAAAHDDGLIIISIFLQVLDHKNVYFQKIVNQLDSIRSGNTSAPFDSVFYLDNLLPSKLDYFAYLGTTINHAADAVWIIFPTPINIHSDQASKARTLASSSAHDGKAHYITEAYANAYKLNADTQVYYSGEIIRAATTSPARENYFMRWLSDLRETCFSYYQKYIEENKTFAIIAIVFVFILTAILFFMSRRYSREKQN

SEQ ID NO: 73 - protein, 110 aa, Artificial Sequence
  Synthetic Construct
MDGTLFPGDDDLAIPATEFFSTKAAKAPEDKAADAAAAAADDNEETLKQRLTNLEKKITNVTTKFEQIEKCCKRNDEVLFRLENHAETLRAAMISLAKKIDVQTGRAAAE

SEQ ID NO: 74 - protein, 250 aa, Artificial Sequence
  Synthetic Construct
MGAAASIQTTVNTLSERISSKLEQAAAASAAAACAIEIGNFYIRQNHGCNLTVKNMCAAAAAAQLDAVLSAATETYSGLTPEQKAYVPAMFTAALNIQTSVNTVVRDFENYVKQTCNSSAVVDNALAIQNVIIDECYGAPGSPTNLEFINTGSSKGNCAIKALMQLTTKATTQIAPKQVAGTGVQFYMIVIGVIILAALFMYYAKRMLFTSTNDKIKLILANKENVHWTTYMDTFFRTSPMVIATTDMQN

SEQ ID NO: 75 - DNA, 36 bp, Artificial Sequence
  Synthetic Construct
ggcgaccacccacaggcaacaccagcacctgcccca

SEQ ID NO: 76 - DNA, 33 bp, Artificial Sequence
  Synthetic Construct
ccggttgcgcctattcttgccgttcttgccgcc

SEQ ID NO: 77 - protein, 24 aa, Homo sapiens
NGRRICLDLQAPLYKKIIKKLLES

SEQ ID NO: 78 - protein, 27 aa, Homo sapiens
GRELCLDPKENWVQRVVEKFLKRAENS

SEQ ID NO: 79 - protein, 34 aa, Homo sapiens
QIHFFFAKLNCRLYRKANKSSKLVSANRLFGDKS

SEQ ID NO: 80 - protein, 34 aa, Homo sapiens
ELRVRLASHLRKLRKRLLRDADDLQKRLAVYQAG

SEQ ID NO: 81 - protein, 12 aa, Homo sapiens
RRLRRMESESES

SEQ ID NO: 82 - protein, 21 aa, Homo sapiens
KRKKKGGKNGKNTTNTKKKNP

SEQ ID NO: 83 - DNA, 27 bp, Artificial Sequence
  Synthetic Construct
tcctgtcttgcattgcactaagtcttg

SEQ ID NO: 84 - DNA, 34 bp, Artificial Sequence
  Synthetic Construct
tcatcactaacgtggcttcttctgccaaagcatg

SEQ ID NO: 85 - protein, 304 aa, Artificial Sequence
  Synthetic Construct
MPQQLSPINIETKKAISNARLKPLDIHYNESKPTTIQNTGKLLWINFKGGYISGWFLPNEYVLSSLHIYWGKEDDYGSNHLIDVYKYSGEINLVHWNKKKYSSYEEAKKHDDGLIIISIFLQVLDHKNVYFQKIVNQLDSIRSTNTSAPFDSVFYLDNLLPSKLDYFSYLGTTINHYADAVWIIFPTPINIHSDQLSKYRTLSSSSNHDGKTHYITECYRNLYKLNGDTQVYYSGEIIRAATTSPARENYFMRWLSDLRETCFSYYQKYIEENKTFAIIAIVFVFILTAILFFMSRRYSREKQN